(19) United States
(12) Patent Application Publication
(10) Pub. No.: US 2022/0380314 A1
(43) Pub. Date: Dec. 1, 2022

(54) SUBSTITUTED, SATURATED AND UNSATURATED N-HETEROCYCLIC CARBOXAMIDES AND RELATED COMPOUNDS FOR THEIR USE IN THE TREATMENT OF MEDICAL DISORDERS

(71) Applicants: Bial - R&D Investments, S.A., Coronado (S.Romão e S.Mamede) (PT); Fondazione Istituto Italiano di Tecnologia, Genova (IT)

(72) Inventors: Renato T. Skerlj, West Newton, MA (US); Elyse Marie Josee Bourque, L'etang-Du-Nord (CA); Soumya Ray, Quincy, MA (US); Rita Scarpelli, Genova (IT); Vincenzo Cilibrasi, Genova (IT)

(21) Appl. No.: 17/760,878

(22) PCT Filed: Sep. 17, 2020
(86) PCT No.: PCT/US2020/051308

§ 371 Date: Mar. 16, 2022

Related U.S. Application Data
(60) Provisional application No. 62/901,386, filed on Sep. 17, 2019.

(51) Int. Cl. C07D 217/06; C07D 401/10; C07D 215/08; C07D 241/38; C07D 471/04; C07D 265/36; C07D 498/10; C07D 215/233; C07D 211/16; C07D 211/28; C07D 211/48; C07D 401/04; C07D 405/10; C07D 265/30; C07D 207/06; C07D 221/20; C07D 209/54; C07D 265/34; C07D 211/70; C07C 275/26; C07D 471/08; C07D 405/12; C07D 241/20; C07D 265/32; C07C 271/16; C07D 451/06

(52) U.S. Cl. CPC C07D 217/06; C07D 401/10; C07D 215/08; C07D 241/38; C07D 471/04; C07D 265/36; C07D 498/10; C07D 215/233; C07D 211/16; C07D 211/28; C07D 211/48; C07D 401/04; C07D 405/10; C07D 265/30; C07D 207/06; C07D 221/20; C07D 209/54; C07D 265/34; C07D 211/70; C07C 275/26; C07D 471/08; C07D 405/12; C07D 241/20; C07D 265/32; C07C 271/16; C07D 451/06; C07C 2601/16

(57) ABSTRACT

The invention provides substituted, saturated and unsaturated N-heterocyclic carboxamides and related compounds, compositions containing such compounds, medical kits, and methods for using such compounds and compositions to treat medical disorders, e.g., cancer, lysosomal storage disorder, neurodegenerative disorder, inflammatory disorder, in a patient.

### CROSS-REFERENCE TO RELATED APPLICATIONS

This application claims the benefit of U.S. Provisional Patent Application No. 62/901,386 filed on Sep. 17, 2019, the entire contents of which are incorporated by reference herein.

### FIELD OF THE INVENTION

The invention provides substituted, saturated and unsaturated N-heterocyclic carboxamides and related organic compounds, compositions containing such compounds, medical kits, and methods for using such compounds and compositions to treat medical disorders in a patient.

### BACKGROUND

Sphingolipids, in addition to serving roles in cell membrane structure and dynamics, also serve important signaling functions, for example, in the control of cell growth, cell differentiation, and cell death, and so are important for cell homeostasis and development. Zeidan et al. (2010) CURR. MOL. MED. 10, 454, Proksch et al. (2011) J. LIPIDS Article ID 971618. Ceramide, a key member of this lipid class, has attracted attention in view of its impact on the replication and differentiation of neoplastic cells. Furuya et al. (2011) CANCER METASTASIS REV. 30, 567. For example, lower levels of ceramide have been discovered in several types of human tumors relative to normal tissue, where the level of ceramide appears to correlate inversely with the degree of malignant progression. Realini et al (2013) J. MOL. BIOL. 56, 3518.

Acid ceramidase (AC, also known as N-acylsphingosine anidohydrolase-1, ASAH-1) is a cysteine amidase that catalyzes the hydrolysis of ceramide into sphingosine and fatty acid. Acid ceramidase is believed to be involved in the regulation of ceramide levels in cells and modulates the ability of this lipid messenger to influence the survival, growth and death of certain tumor cells. Doan et al. ONCOTARGET 8(68), 112662-74, 2017. Furthermore, acid ceramidase enzymes are abnormally expressed in various types of human cancer (e.g., prostate, head and neck, and colon) and serum AC levels are elevated in patients with melanoma relative to control subjects. Realini et al. (2015) J. BIOL. CHEM. 291 (5), 2422-34.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimer's, Parkinson's, Huntington's, and amyotrophic lateral sclerosis). See, International Application Publication Nos. WO2016/210116 and WO2016/210120.

Despite the efforts to develop acid ceramidase inhibitors for use in the treatment of various disorders there is still a need for new acid ceramidase inhibitors.

### SUMMARY

The invention provides substituted, saturated and unsaturated N-heterocyclic carboxamides and related organic compounds, compositions containing such compounds, medical kits, and methods for using such compounds and compositions to treat medical disorders, for example, cancer (such as glioblastoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, amyotrophic lateral sclerosis, and Lewy body disease), an inflammatory disorder, and pain. Various aspects and embodiments of the invention are described in further detail below.

In one aspect, provided herein is a compound of formula (I):

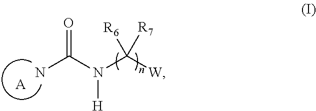

or a pharmaceutically acceptable salt thereof, wherein the variables are as defined herein.

In some embodiments, the compound is a compound of formula (II):

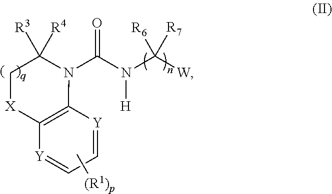

or a pharmaceutically acceptable salt thereof, wherein the variables are as defined herein.

In some embodiments, the compound is a compound of formula (III):

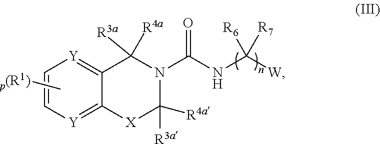

or a pharmaceutically acceptable salt thereof, wherein the variables are as defined herein.

In some embodiments, the compound is a compound of formula (IV):

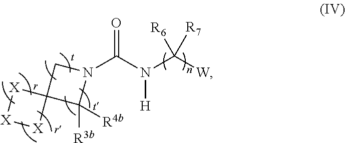

or a pharmaceutically acceptable salt thereof, wherein the variables are as defined herein.

In some embodiments, the compound is a compound of formula (V):

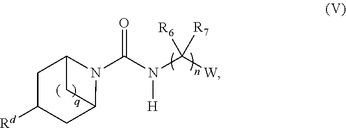

or a pharmaceutically acceptable salt thereof, wherein the variables are as defined herein.

In some embodiments, the compound is a compound of formula (VI):

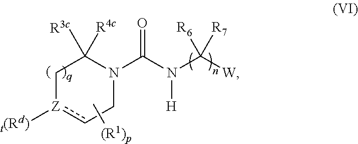

or a pharmaceutically acceptable salt thereof, wherein the variables are as defined herein.

In some embodiments, the compound is a compound of formula (I-A)

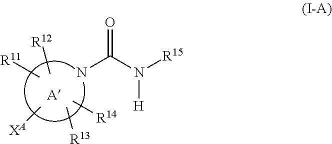

or a pharmaceutically acceptable salt thereof wherein the variables are as defined herein.

In one aspect, provided herein is a compound of formula (I-Aa)

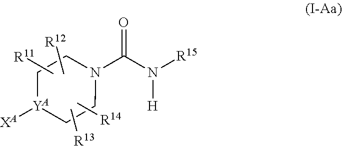

or a pharmaceutically acceptable salt thereof, wherein the variables are as defined herein.

In some embodiments, the compound is a compound of formula (I-Ab)

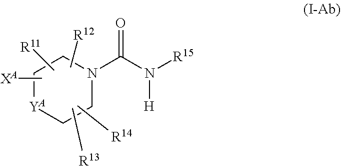

or a pharmaceutically acceptable salt thereof, wherein the variables are as defined herein.

In some embodiments, the compound is a compound of formula (I-B):

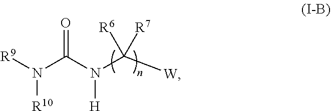

or a pharmaceutically acceptable salt thereof, wherein the variables are as defined herein.

In some embodiments, the compound is a compound of formula (I-C):

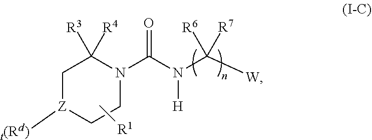

or a pharmaceutically acceptable salt thereof, wherein the variables are as defined herein.

In some embodiments, the compound is a compound of formula (I-D):

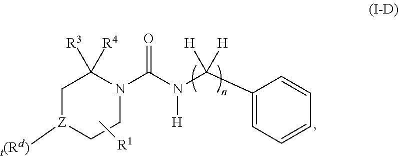

or a pharmaceutically acceptable salt thereof, wherein the variables are as defined herein.

In some embodiments, the compound is a compound of formula (I-E):

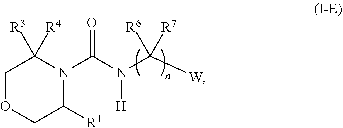

or a pharmaceutically acceptable salt thereof, wherein the variables are as defined herein.

In some embodiments, the compound is a compound of formula (I-F):

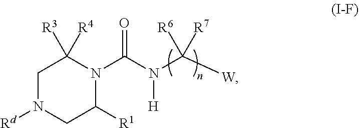

or a pharmaceutically acceptable salt thereof, wherein the variables are as defined herein.

In some embodiments, the compound is a compound of formula (I-G):

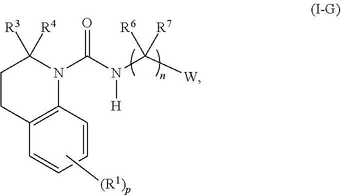

or a pharmaceutically acceptable salt thereof, wherein the variables are as defined herein.

In other embodiments, the compound is a compound of formula (I-H):

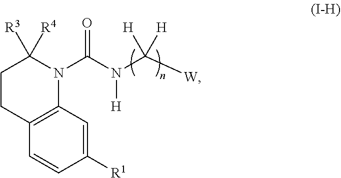

or a pharmaceutically acceptable salt thereof, wherein the variables are as defined herein.

In another aspect, provided herein is a pharmaceutical composition comprising a compound disclosed herein (e.g., a compound of formula (I), (I-A), I-Aa), (I-Ab), (II), (III), (IV), (V), (VI), (I-B), (I-C), (I-D), (I-E), (I-F), (I-G), or (I-H)) and a pharmaceutically acceptable carrier.

In another aspect, the invention provides a method of treating a subject with cancer and in need thereof, the method comprising administering to the subject a therapeutically effective amount of a compound (e.g., a compound of formula (I), (I-A), (I-Aa), (I-Ab), (III), (III), (IV), (V), (VI), (I-B), (I-C), (I-D), (I-E), (I-F), (I-G), or (I-H)) or a pharmaceutical composition disclosed herein.

In another aspect, the invention provides a method of treating a subject with a lysosomal storage disorder and in need thereof, the method comprising administering to the subject a therapeutically effective amount of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition disclosed herein.

In another aspect, the invention provides a method of treating a subject with a neurodegenerative disorder and in need thereof, the method comprising administering to the subject a therapeutically effective amount of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition disclosed herein.

In another aspect, the invention provides a method of treating a subject with an inflammatory disorder and in need thereof, the method comprising administering to the subject a therapeutically effective amount of a compound (e.g., a compound of formula (I), (I-A), (I-Aa), (I-Akb), (II), (III), (IV), (V), (VI), (I-B), (I-C), (I-D), (I-E), (I-F), (I-G), or (I-H)) or a pharmaceutical composition disclosed herein.

In another aspect, the invention provides a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of formula (I), (I-A), (I-Aa), (I-Ab), (II), (III), (IV), (V), (VI), (I-B) (I-C), (I-D), (I-E), (I-F), (I-G), or (I-H)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of formula (I), (I-A), (I-Aa), (I-Ab), (II), (III), (IV), (V), (VI), (I-B), (I-C), (I-D)), (I-E), (I-F), (I-G), or (I-H)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D)), (I-E), (I-F), (I-G), or (I-H)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### DETAILED DESCRIPTION

The invention provides substituted, saturated and unsaturated N-heterocyclic carboxamides and related organic compounds, compositions containing such compounds, medical kits, and methods for using such compounds and compositions to treat medical disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et at, eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group. An exemplary cycloalkylene group is

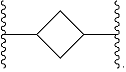

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

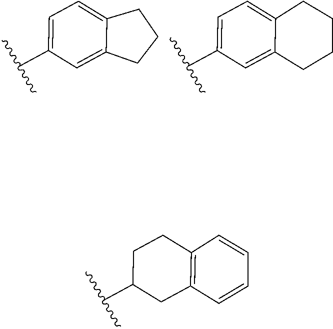

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a fused, spiro, or bridged heterocyclyl group that contains two rings. Representative examples of a bicyclic heterocyclyl include, for example:

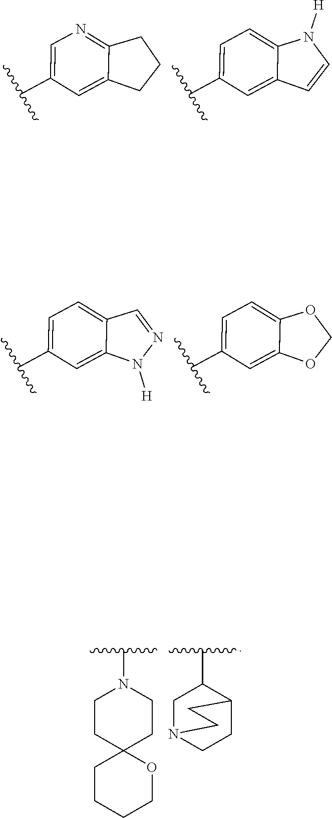

In certain embodiments, the bicyclic heterocyclyl is a carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “heterocyclylene” refers to a diradical of a heterocyclyl group. An exemplary heterocyclylene group is

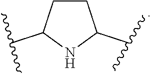

The heterocyclylene may contain, for example, 3-6 ring atom (i.e., a 3-6 membered heterocyclylene). In certain embodiments, the heterocyclylene is a 3-6 membered heterocyclylene containing 1, 2, or 3 three heteroatoms selected from the group consisting of oxygen, nitrogen, and sulfur.

The term “bicyclic heterocyclylene” refers to a diradical of a bicyclic heterocyclyl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and in is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted unless otherwise states. In some embodiments, Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted with 1-4 substituents independently for each occurrence selected from the group consisting of halogen, C₁₋₆alkyl, C₁₋₆haloalkyl, C₁₋₆alkoxy, cyano, N(R^{aa})₂, —CH₂N(R^{aa})₂, and hydroxyl, wherein R^{aa} is independently for each occurrence hydrogen or C₁₋₆alkyl.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include aryl carbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(C))NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclopentane substituted with an oxo group is cyclopentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, an optional substituent may be selected from the group consisting of: C₁₋₆alkyl, cyano, halogen, —O—C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂, wherein R^{a} is selected from the group consisting of hydrogen, C₁₋₆alkyl, phenyl, and 3-7 membered monocyclic heterocyclyl. In some embodiments, an optional substituent may be selected from the group consisting of: C₁₋₆alkyl, cyano, halogen, —O—C₁₋₆alkyl, and —CH₂N(R^{a})₂, wherein R^{a} is selected from the group consisting of hydrogen, C₁₋₆alkyl, phenyl, and 3-7 membered monocyclic heterocyclyl. In some embodiments, an optional substituent may be selected from the group consisting of: C₁₋₆alkyl, cyano, halogen, —O—C₁₋₆alkyl, and —CH₂N(R^{a})₂, wherein R^{a} is hydrogen or C₁₋₆alkyl.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein include diisopropylethylamine (DIPEA); dimethylformamide (DMF); methylene chloride (DCM); tert-butoxycarbonyl (Boc); tetrahydrofuran (THF); trifluoroacetic acid (TFA); triethylamine (TEA); Boc anhydride ((Boc)₂O); dimethylsulfoxide (DMSO); diisopropylethylamine (DIEA); flash column chromatography (FCC); supercritical fluid chromatography (SFC); acetonitrile (ACN); acetic acid (AcOH); ammonium acetate (NH₄OAc); ethylene bridged hybrid (BEH); broadband inverse (BBI); cyclohexane (Cy); dichloroethane (DCE); dimethylamine (NHMe₂); dimethylcyclohexanedicarboxylate (DMCD); ethanol (EtOH); ethylene acetate (EA); in situ chemical oxidation (ISCO); potassium acetate (KOAc); methanol (MeOH); methylmagnesium bromide (MeMgBr); mass spectrometry, electrospray (MS (ES)); methyl tert-butyl ether (MTBE); methyl iodide (MeI); nuclear magnetic resonance spectroscopy (NMR); [1,1′-Bis(diphenylphosphino)ferrocene]dichloropalladium(II), complex with dichloromethane (PdCl₂(dppf)-DCM); photodiode array (PDA); p-toluenesulfonic acid (p-TsOH); room temperature (RT); sodium acetate (NaOAc); sodium triacetoxyborohydride (NaBH(AcO)₃); IST ISOLUTE® SCX packed into SPE cartridges (SCX); solid phase extraction (SPE); thin layer chromatography (TLC); triethylamine (Et₃N); and ultra performance liquid chromatography/mass spectrometry (UPLC/MS).

General purification and analytical methods. Automated column chromatography purifications were done using a Teledyne ISCO apparatus (CombiFlash® Rf) with pre-packed silica gel columns of different sizes (from 4 g until 120 g). Mixtures of increasing polarity of Cy and EA or DCM and MeOH were used as eluents. TLC analyses were performed using Supelco silica gel on TLC Al foils 0.2 mm with fluorescence indicator 254 nm. Purifications of basic compounds were done using IST ISOLUTE® SCX packed into SPE cartridges (SCX). NMR experiments were run on a Bruker Avance III 400 system (400.13 MHz for ¹H), equipped with a BBI probe and Z-gradients. Spectra were acquired at 300 K, using deuterated dimethylsulfoxide (DMSO-d₆) or deuterated chloroform (CDCl₃) as solvents. Chemical shifts for ¹H spectra were recorded in parts per million using the residual non-deuterated solvent as the internal standard (for DMSO-d₆: 2.50 ppm and for CDCl₃: 7.26 ppm, ¹H). Data are reported as follows: chemical shift (ppm), multiplicity (indicated as: bs, broad singlet; s, singlet; d, doublet; t, triplet; q, quartet; p, quintet, sx, sextet; m, multiplet and combinations thereof), coupling constants (J) in Hertz (Hz) and integrated intensity. ¹H-NMR experiments were run on a Bruker Avance III 400 system (400.13 MHz for ¹H), equipped with a BBI probe and Z-gradient coil. Spectra were acquired at 300 K, using deuterated dimethylsulfoxide (DMSO-d₆) or deuterated chloroform (CDCl₃) as solvents. UPLC/MS analyses were run on a Waters ACQUITY UPLC/MS system consisting of a SQD (Single Quadropole Detector) Mass Spectrometer equipped with an Electrospray Ionization interface and a Photodiode Array Detector. PDA range was 210-400 nm. Analyses were performed on an ACQUITY UPLC BEH C18 column (50×2.1 mmID, particle size 1.7 μm) with a VanGuard BEH C18 pre-column (5×2.1 mmID, particle size 1.7 μm). Mobile phase was either 10 mM N₄OAc in 1-120 at pH 5 adjusted with AcOH (A) and 10 mM NH₄OAc in CH₃CN—H₂O (95:5) at pH 5 (B). Electrospray ionization in positive and negative mode was applied. Analyses were performed with method A-C as indicated in each case. Method A: Gradient: 5 to 100% B over 3 min. Flow rate 0.5 mL/min. Temperature 40° C. Method B: Gradient: 50 to 100% B over 3 min. Flow rate 0.5 mL/min. Temperature 40° C. Method C: Gradient: 0 to 100% B over 3 min. Flow rate 0.5 mL/min. Temperature 40° C. Analyses by chiral HPLC were run on a Waters Alliance HPLC instrument consisting of an e2695 Separation Module and a 2998 Photodiode Array Detector. PDA range was 210-400 nm. Analyses were performed isocratic on a Daicel ChiralPak AD column (250×4.6 mmID, particle size 10 m). Mobile phase was Heptane/2-Propanol (98:2). Separations by preparative chiral HPLC were run on a Waters Alliance HPLC instrument consisting of a 1525 Binary HPLC Pump, Waters Fraction Collector III and a 2998 Photodiode Array Detector. UV detection was at 215 nm. Purifications were performed isocratic on a Daicel ChiralPak AD column (250×10 mmID, particle size 10 μm). Mobile phase was Heptane/2-Propanol (98:2). Hydrogenation reactions were also performed using H-Cube® (H-Cube) continuous hydrogenation equipment (SS-reaction line version), employing disposable catalyst cartridges (CatCart@) preloaded with the required heterogeneous catalyst. Microwave heating was performed using Explorer®-48 positions instrument (CEM). Optical rotations were measured on a Rudolf Research Analytical Autopol II Automatic polarimeter using a sodium lamp (589 nm) as the light source; concentrations expressed in g/100 mL using CHCl₃ as a solvent and a 1 dm cell. All final compounds displayed ≥95% purity as determined by NMR and UPLC/MS analysis.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Substituted, Saturated and Unsaturated N-Heterocyclic Carboxamides and Related Compounds

### Compounds

One aspect of the invention provides substituted, saturated and unsaturated N-heterocyclic carboxamides and related organic compounds. The substituted, saturated and unsaturated N-heterocyclic carboxamides and related organic compounds are contemplated to be useful in the methods, compositions, and kits described herein. In certain embodiments, substituted, saturated and unsaturated N-heterocyclic carboxamide and related organic compound is a compound embraced by formula (I):

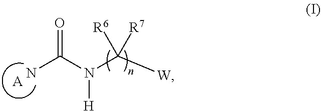

or a pharmaceutically acceptable salt there of, wherein:

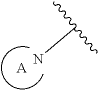

is selected from a monocyclic or bicyclic (e.g., fused, spiro, or bridged bicyclic) heterocyclyl containing at least one N (including the depicted nitrogen), or —NR⁹R¹⁰; wherein the monocyclic or bicyclic heterocyclyl is optionally substituted, for example, with one or more substituents selected from the group consisting of hydrogen, C₁₋₆alkyl, methylene (i.e.,

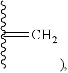

halogen, cyano, oxo, phenyl, 3-7 membered monocyclic heterocyclyl, C₃₋₇cycloalkyl, 5-6 membered heteroaryl, and (3-7 membered monocyclic heterocyclylene)-(3-7 membered monocyclic heterocyclyl), wherein the aforementioned C₁₋₆alkyl, methylene, —O—R^{c}, —C(O)OR^{f}, —N(R^{f})₂, phenyl, 3-7 membered monocyclic heterocyclyl, C₃₋₇cycloalkyl, 5-6 membered heteroaryl, and (3-7 membered monocyclic heterocyclylene)-(3-7 membered monocyclic heterocyclyl) may be optionally substituted with one or more substituents (e.g., with one or more substituents independently selected from the group consisting of halogen, C₁₋₆alkyl, —O—C₁₋₆alkyl, —C(O)C₁₋₆alkyl, and phenyl);

R^{a} is independently, for each occurrence, selected from the group consisting of hydrogen, C₁₋₆alkyl, phenyl, and 3-7 membered monocyclic heterocyclyl;

R^{c} is independently, for each occurrence, selected from the group consisting of C₁₋₆alkyl, C₁₋₆haloalkyl, phenyl, C₃₋₇cycloalkyl, 3-7 membered monocyclic heterocyclyl optionally substituted with C₁₋₆alkyl, 5-6 membered heteroaryl, phenyl, and C₁₋₆alkylene-N(R^{a})₂;

R^{f} is independently, for each occurrence, selected from the group consisting of hydrogen, C₁₋₆alkyl, —(C₁₋₆alkylene)-phenyl, and phenyl;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl;

n is an integer selected from 0 to 6; and

when n is an integer selected from 1 to 6, W is selected from the group consisting of methyl, methylene (i.e.,

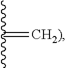

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),

when n is 0, W is selected from the group consisting of methyl, methylene (i.e.,

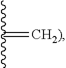

halogen, C₃₋₇cycloalkyl, 3-7 membered saturated monocyclic heterocycyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);

wherein the compound is not a compound selected from the group consisting of:

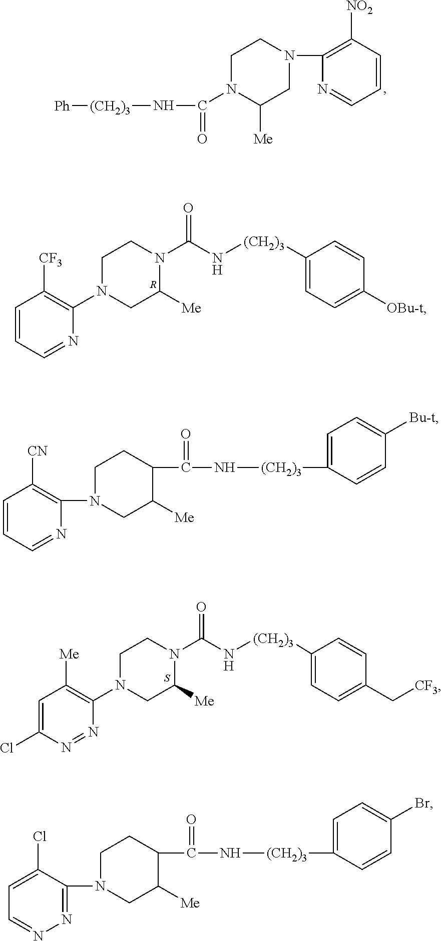

or a pharmaceutically acceptable salt thereof.

In some embodiments,

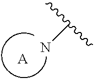

is a bicyclic heterocyclyl containing at least one N.

In some embodiments, the bicyclic heterocyclyl is a fused bicyclic heterocyclyl.

In some embodiments, the bicyclic heterocyclyl is an 8-12 membered heterocyclyl.

In some embodiments, the bicyclic heterocyclyl is a 10 membered heterocyclyl.

In some embodiments, the compound is a compound of formula (II):

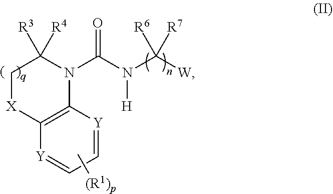

or a pharmaceutically acceptable salt thereof, wherein:

R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl;

p is an integer selected from 0 to 2;

R³ and R⁴ are independently selected from hydrogen or C₁₋₂alkyl, or R³ and R⁴ can be taken together to form C₃₋₄cycloalkyl;

X is selected from the group consisting of CR^{b}₂, NR^{a}, and O;

each Y is independently selected from C(R²)₂ or N;

R² is selected from the group consisting of hydrogen, C₁₋₆alkyl, halogen, cyano, —O—R^{c}, phenyl, 3-7 membered monocyclic heterocyclyl, C₃₋₇cycloalkyl, and 5-6 membered heteroaryl;

R^{b} is independently, for each occurrence, selected from the group consisting of hydrogen, C₁₋₆alkyl, phenyl, —O—C₁₋₆alkyl, —O-phenyl, —O-(3-7 membered heterocyclyl), 3-7 membered monocyclic heterocyclyl, (3-7 membered monocyclic heterocyclylene)-(3-7 membered monocyclic heterocyclyl) or two R^{b} can be taken together to form oxo;

q is an integer selected from 0 or 1; and

R⁶, R⁷, R^{a}, R^{c}, n, and W are as defined in the compound of formula (I);

wherein any aforementioned 3-7 membered monocyclic heterocyclyl and 5-6 membered heteroaryl are optionally substituted.

In some embodiments, R³ and R⁴ are hydrogen or methyl, or R³ and R⁴ are taken together to form cyclopropylene.

In some embodiments, R³ and R⁴ are methyl.

In some embodiments, R³ and R⁴ are hydrogen.

In some embodiments, the compound is a compound of formula (III):

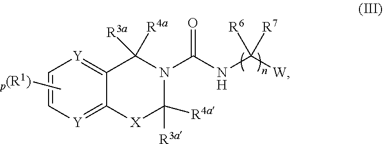

or a pharmaceutically acceptable salt thereof, wherein:

R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl;

p is an integer selected from 0 to 2;

R^{3a} and R^{4a} are independently selected from C₁₋₂alkyl, or R^{3a} and R^{4a} can be taken together to form C₃₋₄cycloalkyl; and R^{3a′} and R^{4a′} are independently selected from hydrogen and C₁₋₂alkyl or R^{3a′} and R^{4a′} can be taken together to form C₃₋₄cycloalkyl; or

R^{3a′} and R^{4′} are independently selected from C₁₋₂alkyl, or R^{3a′} and R^{4a′} can be taken together to form C₃₋₄cycloalkyl; and R^{3a} and R^{4a} are independently selected from hydrogen and C₁₋₂alkyl or R^{3a} and R^{4a} can be taken together to form C₃₋₄cycloalkyl;

X is selected from the group consisting of CR^{b}₂, NR^{a}, and O;

each Y is independently selected from C(R²)₂ and N;

R² is selected from the group consisting of hydrogen, C₁₋₆alkyl, halogen, cyano, —O—R^{c}, phenyl, 3-7 membered monocyclic heterocyclyl, C₃₋₇cycloalkyl, and 5-6 membered heteroaryl;

R^{b} is independently, for each occurrence, selected from the group consisting of hydrogen, C₁₋₆alkyl, phenyl, —O—C₁₋₆alkyl, —O-phenyl, —O-(3-7 membered monocyclic heterocyclyl), 3-7 membered monocyclic heterocyclyl, (3-7 membered monocyclic heterocyclylene)-(3-7 membered monocyclic heterocyclyl) or two R^{b} can be taken together to form oxo; and

R⁶, R⁷, R^{a}, R^{c}, n, and W are as defined in the compound of formula (I);

wherein any aforementioned 3-7 membered monocyclic heterocyclyl and 5-6 membered heteroaryl are optionally substituted.

In some embodiments, R^{3a} and R^{4a} are methyl, and R^{3a′} and R^{4a′} are hydrogen.

In some embodiments, R^{3a′} and R^{4a′} are methyl, and R^{3a} and R^{4b} are hydrogen.

In some embodiments, p is 1.

In some embodiments, R¹ is selected from the group consisting of cyano, halogen, 3-7 membered monocyclic heterocyclyl, and 5-6 membered heteroaryl, wherein the 3-7 membered monocyclic heterocyclyl and 5-6 membered heteroaryl are optionally substituted with one or more substituents selected from the group consisting of methyl, —C(O)CH₃, or 3-7 membered monocyclic heterocyclyl.

In some embodiments, R¹ is selected from the group consisting of cyano, 3-7 membered monocyclic heterocyclyl, and 5-6 membered heteroaryl, wherein the 3-7 membered monocyclic heterocyclyl and 5-6 membered heteroaryl are optionally substituted with methyl.

In some embodiments, R¹ is selected from the group consisting of cyano, halogen,

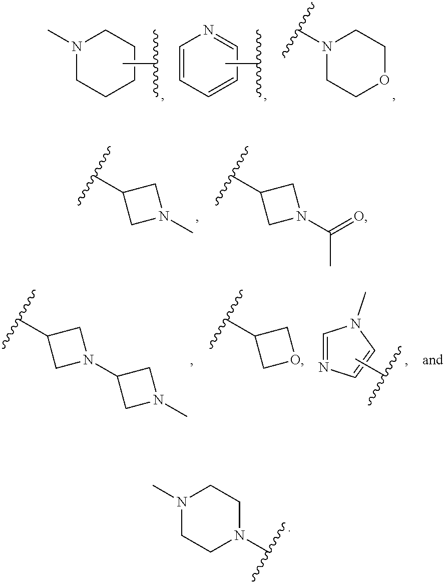

In some embodiments, R¹ is selected from the group consisting of halogen,

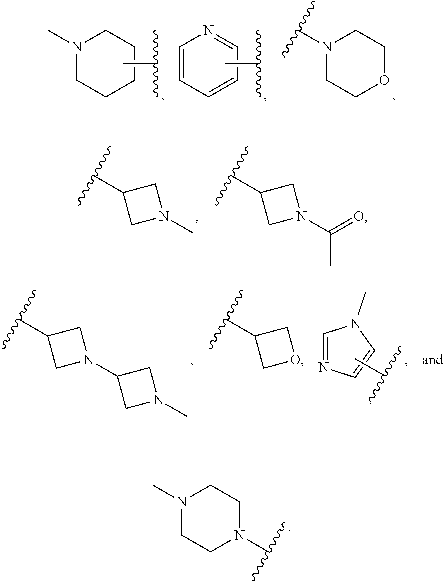

In some embodiments, R¹ is selected from the group consisting of cyano, halogen,

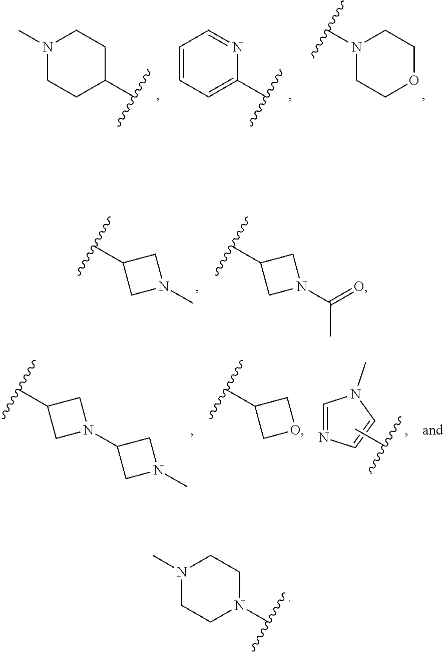

In some embodiments, R¹ is 3-7 membered monocyclic heterocyclyl, wherein the 3-7 membered monocyclic heterocyclyl is optionally substituted with methyl.

In some embodiments, R¹ is

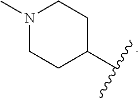 [SMILES: *C1CCN(C)CC1]

In some embodiments, X is selected from the group consisting of CH₂, NCH₃, O, CH—O—C₁₋₆alkyl, C═O, and N-(3-7 membered monocyclic heterocyclyl), CH-(3-7 membered monocyclic heterocyclyl), wherein the 3-7 membered monocyclic heterocyclyl is optionally substituted with methyl.

In some embodiments, X is selected from the group consisting of CH₂, NCH₃, O, CH-OMe, and C═O.

In some embodiments, X is CR^{b}₂.

In some embodiments, X is CH₂.

In some embodiments, each Y is C(R²)₂.

In some embodiments, one Y is C(R²)₂ and the other is N.

In some embodiments, wherein each R² is independently selected from the group consisting of hydrogen, cyano, fluorine, —OCH₃,

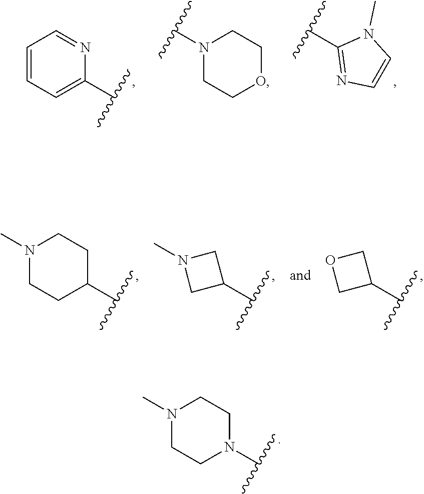

In some embodiments, each Y is N.

In some embodiments, each Y is independently CH or N.

In some embodiments, the bicyclic heterocyclyl is a spiro-bicyclic heterocyclyl.

In some embodiments, the bicyclic heterocyclyl is a 6-12 membered spiro-bicyclic heterocyclyl.

In some embodiments, the compound is a compound of formula (IV):

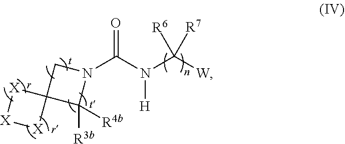

or a pharmaceutically acceptable salt thereof, wherein

R^{3b} and R^{4b} are independently, for each occurrence, selected from hydrogen and C₁₋₂alkyl; wherein at least one of R^{3b} and R^{4b} on the carbon adjacent to the nitrogen is selected from C₁₋₂alkyl;

X is independently, for each occurrence, selected from the group consisting of CR^{b}₂, NR^{a}, and O;

R^{b} is independently, for each occurrence, selected from the group consisting of hydrogen, C₁₋₆alkyl, phenyl, —O—C₁₋₆alkyl, —O-phenyl, —O-(3-7 membered heterocyclyl), 3-7 membered monocyclic heterocyclyl, (3-7 membered monocyclic heterocyclylene)-(3-7 membered monocyclic heterocyclyl) or two R^{b} can be taken together to form oxo;

r, r′, t, and t′ are independently, for each occurrence, 1 or 2; and

R⁶, R⁷, R^{a}, n, and W are as defined in the compound of formula (I);

wherein any aforementioned 3-7 membered monocyclic heterocyclyl and 5-6 membered heteroaryl are optionally substituted.

In some embodiments,

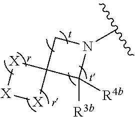

is selected from the group consisting of:

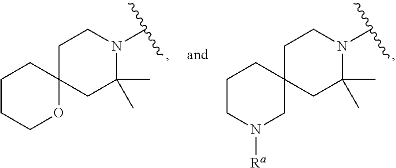

wherein R^{a} is as defined herein.

In some embodiments,

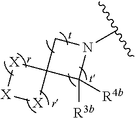

is

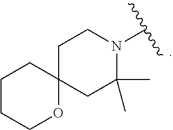 [SMILES: *N1CCC2(CCCCO2)CC1(C)C]

In some embodiments, R^{a} is selected from methyl and

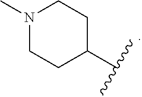 [SMILES: *C1CCN(C)CC1]

In some embodiments, each of R^{3b} and R^{4b} on the carbon adjacent to the nitrogen is methyl.

In some embodiments, X is independently for each occurrence selected from the group consisting of CH₂, O, and NR^{a}.

In some embodiments, the bicyclic heterocyclyl is a bridged bicyclic heterocyclyl.

In some embodiments, the bicyclic heterocyclyl is 8-membered bridged bicyclic heterocyclyl.

In some embodiments, the compound is a compound of formula (V):

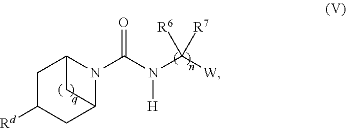

or a pharmaceutically acceptable salt thereof, wherein
q is an integer selected from 1 and 2;
R^{d} is independently, for each occurrence, selected from the group consisting of hydrogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, or (3-7 membered monocyclic heterocyclylene)-(3-7 membered monocyclic heterocyclyl); and

R⁶, R⁷, R^{f}, n, and W are as defined in the compound of formula (I);

wherein any aforementioned C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), and (3-7 membered monocyclic heterocyclylene)-(3-7 membered monocyclic heterocyclyl) are optionally substituted.

In some embodiments, q is 1.

In some embodiments, q is 2.

In some embodiments, R^{d} is selected from phenyl and

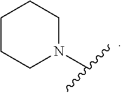 [SMILES: *N1CCCCC1]

In some embodiments,

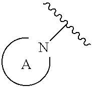

is a monocyclic heterocyclyl containing at least one N (including the depicted nitrogen).

In some embodiments, the monocyclic heterocyclyl is a 4-8 membered heterocyclyl.

In some embodiments, the monocyclic heterocyclyl is a 6-membered heterocyclyl.

In some embodiments, the compound is a compound of formula (VI):

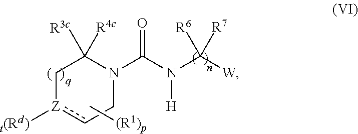

or a pharmaceutically acceptable salt thereof, wherein

[symbol] denotes a single bond or a double bond;

R¹ is selected from the group consisting of C₁₋₆alkyl, halogen, cyano, oxo, —O—R^{c}, phenyl, —(C₁₋₆alkylene)-phenyl, —(C₁₋₆alkenyl)-phenyl, 3-7 membered monocyclic heterocyclyl, C₃₋₇cycloalkyl, and 5-6 membered heteroaryl;

R^{3c} and R^{4c} are independently selected from hydrogen or C₁₋₃alkyl, or R^{3c} and R^{4c} can be taken together to form C₃₋₆cycloalkyl;

Z is selected from the group consisting of C, CH, N, and O, wherein when Z is C, t=1 or 2, when Z is CH, t=1, when Z is N, t=1, and when Z is O, t=0;

R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;

R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered heterocyclyl), —C(O)OR^{f}, —N(R^{f})₂, and (3-7 membered monocyclic heterocyclylene)-(3-7 membered monocyclic heterocyclyl);

p is an integer selected from 0 to 3;

q is an integer selected from 0 or 1;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

R^{f} is independently, for each occurrence, selected from the group consisting of hydrogen, C₁₋₆alkyl, —(C₁₋₆alkylene)-phenyl, and phenyl;

n is an integer selected from 0 to 6; and

when n is an integer selected from 1 to 6, W is selected from the group consisting of methyl, methylene (i.e.,

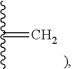

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),

when n is 0, W is selected from the group consisting of methyl, methylene (i.e.,

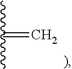

halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);

wherein any aforementioned C₁₋₆alkyl, phenyl, C₃₋₇cycloalkyl, 5-6 membered heteroaryl, 3-7 membered monocyclic heterocyclyl, —(C₁₋₆alkylene)-phenyl, —(C₁₋₆alkenyl)-phenyl, —O—C₁₋₆alkyl, —O-phenyl, —O-(3-7 membered heterocyclyl), and (3-7 membered monocyclic heterocyclylene)-(3-7 membered monocyclic heterocyclyl) are optionally substituted.

In another embodiment, the compound is a compound of formula (VI):

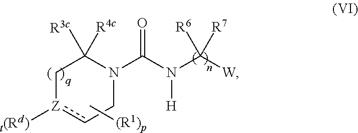

or a pharmaceutically acceptable salt thereof, wherein

[symbol] denotes a single bond or a double bond;

R¹ is selected from the group consisting of C₁₋₆alkyl, halogen, cyano, oxo, —O—R^{c}, phenyl, —(C₁₋₆alkylene)-phenyl, —(C₁₋₆alkenyl)-phenyl, 3-7 membered monocyclic heterocyclyl, C₃₋₇cycloalkyl, and 5-6 membered heteroaryl;

R^{3c} and R^{4c} are independently selected from hydrogen or C₁₋₃alkyl, wherein at least one of R^{3c} or R^{4c} is C₁₋₃alkyl, or R^{3c} and R^{4c} can be taken together to form C₃₋₆cycloalkyl;

Z is selected from the group consisting of CH, N, and O, wherein when Z is C, t=1 or 2, when Z is CH, t=1, when Z is N, t=1, and when Z is O, t=0;

R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;

R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, or (3-7 membered monocyclic heterocyclylene)-(3-7 membered monocyclic heterocyclyl);

p is an integer selected from 0 to 3;

q is an integer selected from 0 or 1;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene:

R^{f} is independently, for each occurrence, selected from the group consisting of hydrogen, C₁₋₆alkyl, —(C₁₋₆alkylene)-phenyl, and phenyl;

n is an integer selected from 0 to 6; and

when n is an integer selected from 1 to 6, W is selected from the group consisting of methyl, methylene (i.e.,

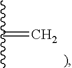

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, or

when n is 0, W is selected from the group consisting of methyl, methylene (i.e.,

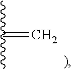

halogen, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃-cycloalkyl, and —O—(C₁₋₆alkylene)-phenyl.

In some embodiments, R¹ is selected from the group consisting of, cyano, halogen, methyl, oxo, phenyl,

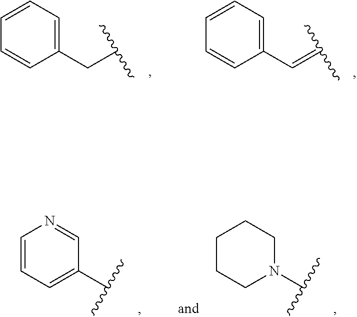 [SMILES: *Cc1ccccc1 ; *=Cc1ccccc1 ; *c1cccnc1 ; *N1CCCCC1]

wherein the aforementioned phenyl,

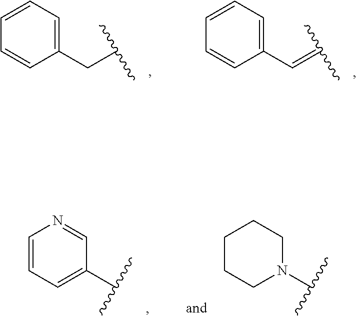 [SMILES: *Cc1ccccc1 ; *=Cc1ccccc1 ; *c1cccnc1 ; *N1CCCCC1]

are optionally substituted with 1-2 substituents independently, for each occurrence, selected from the group consisting of halogen, C₁-C₆alkyl, and —O—C₁-C₆alkyl.

In some embodiments, R¹ is selected from the group consisting of cyano, fluorine, methyl, oxo, phenyl,

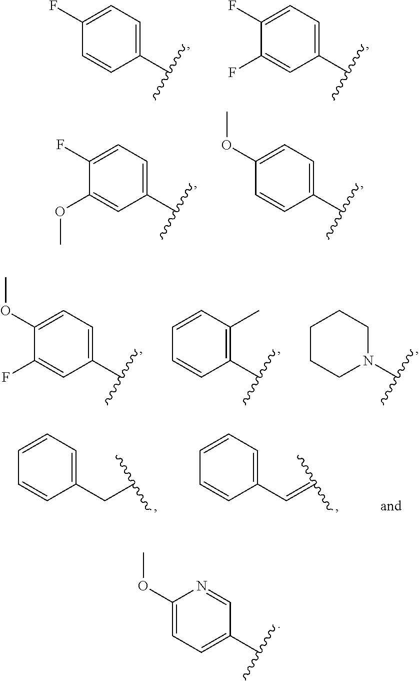

In some embodiments, R^{3c} and R^{4c} are independently selected from hydrogen and C₁₋₃alkyl, or R^{3c} and R^{4c} can be taken together to form C₃₋₅cycloalkyl;

In some embodiments, R^{3c} and R^{4c} are independently selected from hydrogen, methyl and isopropyl.

In some embodiments, each of R^{3c} and R^{4c} is methyl.

In some embodiments, R^{3c} and R^{4c} are taken together to form cyclobutyl or cyclopentyl.

In some embodiments, Z is CH.

In some embodiments, Z is N.

In some embodiments, R^{d} is selected from the group consisting of hydrogen, methyl, —O—(C₁₋₆alkyl),

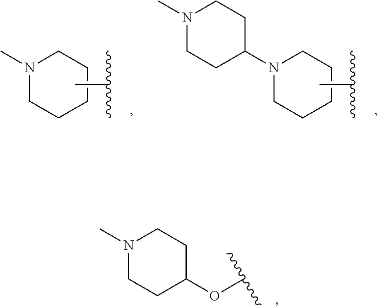

O-phenyl, phenyl,

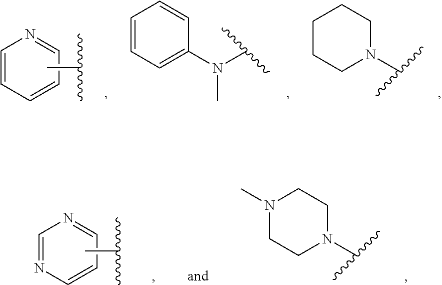

wherein the aforementioned —O—(C₁₋₆alkyl),

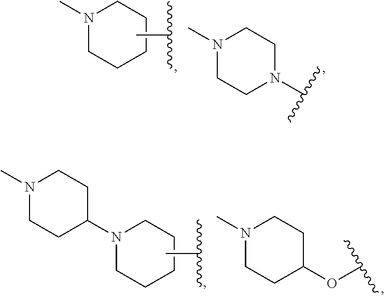

phenyl, O-phenyl,

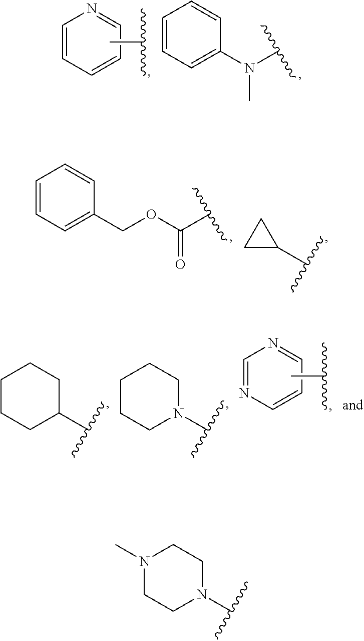

are optionally substituted with 1-2 substituents independently, for each occurrence, selected from the group consisting of halogen, C₁₋₆alkyl, —O—C₁₋₆alkyl, and phenyl.

In some embodiments, R^{d} is selected from the group consisting of hydrogen, methyl, —O—CH₃, phenyl, O-phenyl,

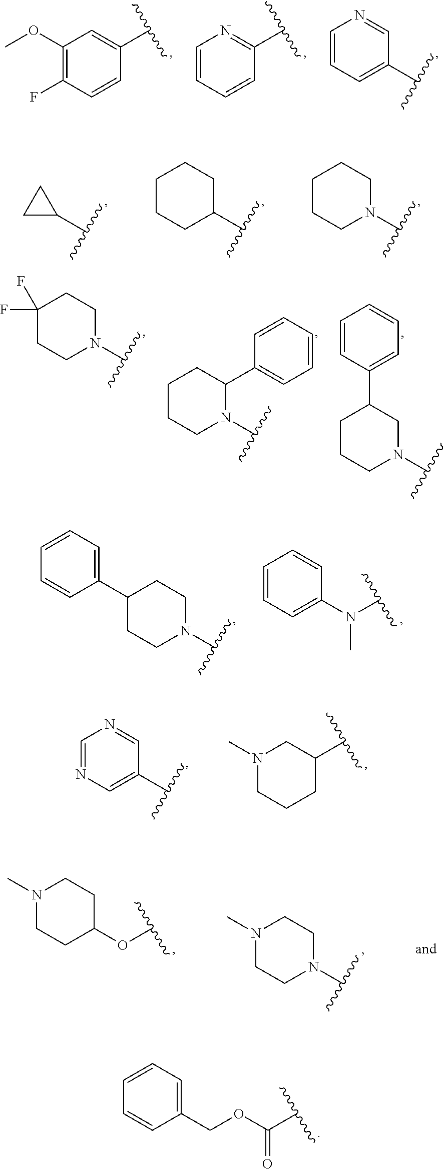

In some embodiments, R^{d} is phenyl.

In some embodiments, Z is C.

In some embodiments, R^{d} is fluorine or oxo.

In some embodiments, Z is O.

In some embodiments,

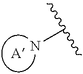 [SMILES: *N]

is NR⁹R¹⁰, wherein R⁹ and R¹⁰ are as defined herein.

In another aspect, the present disclosure provides a compound is a compound of formula (I-A)

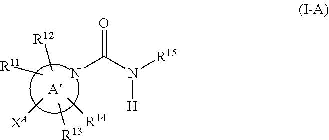

or a pharmaceutically acceptable salt thereof, wherein

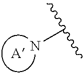 [SMILES: *N]

is selected from a monocyclic or bicyclic (e.g., fused, spiro, or bridged) heterocyclyl containing at least one N (including the depicted nitrogen);

X^{A} is independently selected from hydrogen, optionally substituted C₁-C₆alkyl, optionally substituted C₃-C₇cycloalkyl, optionally substituted 3-7 membered monocyclic heterocyclyl, phenyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), and (3-7 membered monocyclic heterocyclylene)-(3-7 membered monocyclic heterocyclyl), wherein X can be attached to any carbon or nitrogen atom of the ring to which it is connected;

R¹¹, R¹², R¹³ and R¹⁴ are independently selected from hydrogen, cyano, oxo, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, oxo (═O), —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring, wherein R¹¹, R¹², R¹³ and R¹⁴ are not all hydrogen when R¹¹, R¹², R¹³ and R¹⁴ are attached to the carbon atoms linked to the nitrogen of the urea;

or any of R¹¹, R¹², R¹³ and/or R¹⁴ can be taken together with the carbon atoms to which they are attached to form an optionally substituted 3-6 membered spiro carbocyclic or spiro heterocyclic ring,

or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,

or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and

R¹⁵ is independently selected from optionally substituted C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), optionally substituted C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

In some embodiments, the compound is a compound of formula (I-Aa)

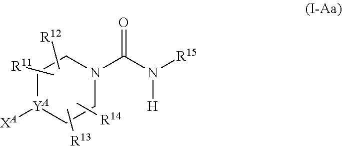

or a pharmaceutically acceptable salt thereof, wherein Y^{A} is independently selected from CH, N and R¹¹, R¹², R¹³ R¹⁴, R¹⁵ and X^{A} are as defined in the compound of formula (I-A).

In another aspect, provided herein is a compound of formula (I-Aa):

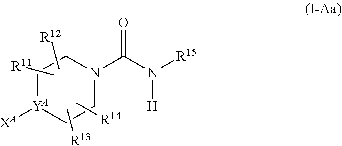

or a pharmaceutically acceptable salt thereof, wherein

Y^{A} is independently selected from CH and N;

X^{A} is independently selected from hydrogen, C₁-C₆alkyl, optionally substituted C₃-C₇cycloalkyl, optionally substituted 3-7 membered monocyclic heterocyclyl, phenyl, C₁-C₆alkyl-(5-6 membered aryl), 5-6 membered heteroaryl, optionally substituted C₁-C₆alkyl-(5-6 membered heteroaryl), and (3-7 membered monocyclic heterocyclylene)-(3-7 membered monocyclic heterocyclyl);

R¹¹, R¹², R¹³ and R¹⁴ are independently selected from hydrogen, cyano, C₁-C₆alkyl, optionally substituted C₃-C₇cycloalkyl, optionally substituted 3-7 membered monocyclic heterocyclyl, optionally substituted 5-6 membered aryl, C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring, wherein R¹¹, R¹², R¹³ and R¹⁴ are not all hydrogen when R¹¹, R¹², R¹³ and R¹⁴ are attached to the carbon atoms linked to the nitrogen of the urea;

or any of R¹¹, R¹², R¹³ and/or R¹⁴ can be taken together with the carbon atoms to which they are attached to form an optionally substituted 3-6 membered spiro carbocyclic or spiro heterocyclic ring,

or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,

or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and

R¹⁵ is independently selected from C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

In some embodiments, R¹⁵ is C₁-C₆alkyl or C₁-C₆alkyl-(5-6 membered aryl).

In some embodiments, R¹¹, R¹², R¹³, and R¹⁴ are independently selected from hydrogen, C₁-C₆alkyl, and optionally substituted phenyl.

In some embodiments, X^{A} is selected from the group consisting of: C₁-C₆alkyl, C₃-C₇cycloalkyl, and phenyl.

In some embodiments, the compound is a compound of formula (I-Ab)

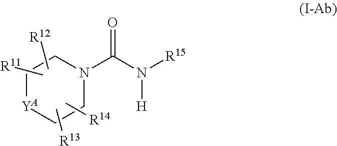

or a pharmaceutically acceptable salt thereof, wherein Y^{A} is independently selected from CH₂, oxo (═O), O, NR^{Ac} wherein R^{Ac} is independently selected from H, optionally substituted (C₁-C₆)alkyls, optionally substituted (C₃-C₆)cycloalkyls, optionally substituted (C₃-C₆)heterocycloalkyls, and R¹¹, R¹², R¹³ R¹⁴, R¹⁵ and X^{A} are as defined as in the compound of formula (I-A).

In another aspect, provided herein is a compound of formula (I-Ab):

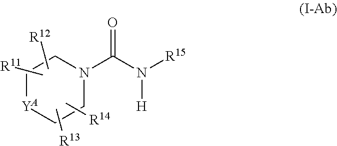

or a pharmaceutically acceptable salt thereof, wherein

Y^{A} is independently selected from CH₂, —C═O, O, and NR^{Ac}, wherein R^{Ac} is independently selected from H, optionally substituted C₁-C₆alkyl, optionally substituted C₃-C₆cycloalkyls, optionally substituted C₃-C₆heterocycloalkyls;

X^{A} is independently selected from hydrogen, C₁-C₆alkyl, optionally substituted C₃-C₆cycloalkyl, optionally substituted 3-7 membered monocyclic heterocyclyl, phenyl, C₁-C₆alkyl-(5-6 membered aryl), optionally substituted 5-6 membered heteroaryl, optionally substituted C₁-C₆alkyl-(5-6 membered heteroaryl), and (3-7 membered monocyclic heterocyclylene)-(3-7 membered monocyclic heterocyclyl);

R¹¹, R¹², R¹³ and R¹⁴ are independently selected from hydrogen, cyano, C₁-C₆alkyl, optionally substituted C₃-C₇cycloalkyl, optionally substituted 3-7 membered monocyclic heterocyclyl, optionally substituted 5-6 membered aryl, C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring, wherein R¹¹, R¹², R¹³ and R¹⁴ are not all hydrogen when R¹¹, R¹², R¹³ and R¹⁴ are attached to the carbon atoms linked to the nitrogen of the urea;

or any of R¹¹, R¹², R¹³ and/or R¹⁴ can be taken together with the carbon atoms to which they are attached to form an optionally substituted 3-6 membered spiro carbocyclic or spiro heterocyclic ring,

or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,

or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and

R¹⁵ is independently selected from C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

In some embodiments, R¹⁵ is C₁-C₆alkyl or C₁-C₆alkyl-(5-6 membered aryl).

In some embodiments, R¹¹, R¹², R¹³, and R¹⁴ are independently selected from hydrogen, C₁-C₆alkyl, and optionally substituted phenyl.

In some embodiments, X^{A} is selected from the group consisting of: C₁-C₆alkyl, C₃-C₇cycloalkyl, and phenyl.

In another aspect, provided herein is a compound selected from the group consisting of a compound of formula (I-C), formula (I-D), formula (I-E), formula (I-F), formula (I-G), formula (I-H), and formula (I-B):

wherein the compound of formula (I-C) is:

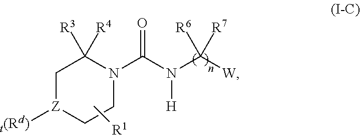

or a pharmaceutically acceptable salt thereof, wherein:

Z is selected from the group consisting of C, CH, N, and O; wherein

(i) when Z is C, t=1 (if R^{d} is oxo) or 2, when Z is CH, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered heterocyclyl), or (3-7 membered monocyclic heterocyclylene)-(3-7 membered monocyclic heterocyclyl) is optionally substituted; and
at least one of R¹ and R⁴ is cyclyl or substituted cyclyl;

(ii) when Z is O, t is 0;
R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;

(iii) when Z is N, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is selected from the group consisting of hydrogen, C₁₋₃alkyl, C₁₋₆alkyl, phenyl, C₃₋₇cycloalkyl, 5-6 membered heteroaryl, 3-7 membered monocyclic saturated heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 3-7 membered monocyclic saturated heterocyclyl, —O—C₁₋₆alkyl, —O-phenyl, or (3-7 membered monocyclic heterocyclylene)-(3-7 membered monocyclic heterocyclyl) is optionally substituted:

wherein at least one of R¹ and R^{d} is cyclyl or substituted cyclyl, wherein R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted, or R^{d} is selected from the group consisting of phenyl, C₃₋₇cycloalkyl, 5-6 membered heteroaryl, 3-7 membered monocyclic heterocyclyl, and (3-7 membered monocyclic heterocyclylene)-(3-7 membered monocyclic heterocyclyl), wherein the phenyl, C₃₋₇cycloalkyl, 5-6 membered heteroaryl, 3-7 membered monocyclic heterocyclyl, or (3-7 membered monocyclic heterocyclylene)-(3-7 membered monocyclic heterocyclyl) is optionally substituted;

R³ is optionally substituted C₁₋₆alkyl;

R⁴ is hydrogen or C₁₋₃alkyl; or

R³ and R⁴ can be taken together to form C₃₋₆cycloalkyl;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 3 to 5; and

W is selected from the group consisting of methyl, an optionally substituted phenyl, and an optionally substituted C₃₋₇cycloalkyl;

wherein the compound of formula (I-D) is:

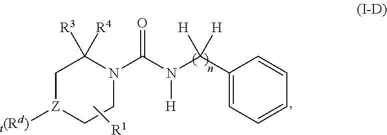

or a pharmaceutically acceptable salt thereof, wherein:

Z is selected from the group consisting of C, CH, N, and O; wherein

when Z is C, t=1 (if R^{d} is oxo), or 2, when Z is CH, t=1, and when Z is O, t=0;

when Z is C, CH, or N, R¹ is hydrogen or optionally substituted phenyl;

when Z is O, R¹ is optionally substituted phenyl;

R³ is C₁₋₃alkyl;

R⁴ is hydrogen or C₁₋₃alkyl;

R^{d} is independently, for each occurrence, selected from the group consisting of hydrogen, C₁₋₆alkyl, phenyl, C₃₋₇cycloalkyl, 5-6 membered heteroaryl, 3-7 membered monocyclic heterocyclyl, wherein the C₁₋₆alkyl, phenyl, C₃₋₇cycloalkyl, 5-6 membered heteroaryl, 3-7 membered monocyclic heterocyclyl is optionally substituted;

n is 4;

wherein at least one of R¹ and R^{d} is cyclyl or substituted cyclyl, wherein R¹ is optionally substituted phenyl, or R^{d} is optionally substituted phenyl or optionally substituted 3-7 membered monocyclic heterocyclyl;

wherein the compound of formula (I-E) is:

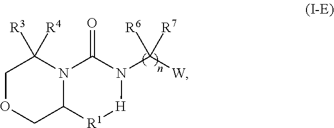

or a pharmaceutically acceptable salt thereof, wherein:

R¹ is C₁₋₆alkyl or optionally substituted phenyl;

R³ is C₁₋₆alkyl;

R⁴ is hydrogen or C₁₋₆alkyl;

R⁶ and R⁷ are independently, for each occurrence, selected from the group consisting of hydrogen, C₁₋₆alkyl, C₁₋₆haloalkyl, and halogen;

n is an integer selected from 1 to 5; and

W is selected from the group consisting of methyl, an optionally substituted phenyl, and an optionally substituted C₃₋₇cycloalkyl;

wherein the compound of formula (I-F) is:

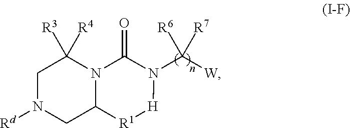

or a pharmaceutically acceptable salt thereof, wherein:

R¹ is C₁₋₆alkyl or optionally substituted phenyl;

R³ is C₁₋₆alkyl;

R⁴ is C₁₋₆alkyl or hydrogen;

R⁶ and R⁷ are independently, for each occurrence, selected from the group consisting of hydrogen, C₁₋₆alkyl, C₁₋₆haloalkyl, and halogen;

n is an integer selected from 1 to 6;

W is selected from the group consisting of methyl, an optionally substituted phenyl, and an optionally substituted C₃₋₇cycloalkyl; and

R^{d} is selected from phenyl, C₃₋₇cycloalkyl, 5-6 membered heteroaryl, wherein the phenyl, C₃₋₇cycloalkyl, 5-6 membered heteroaryl, is optionally substituted;

wherein the compound of formula (I-G) is:

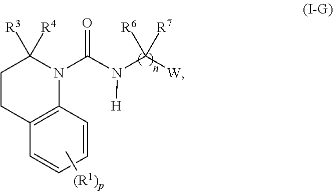

or a pharmaceutically acceptable salt thereof, wherein:

R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl; wherein at least one of R¹ is selected from the group consisting of phenyl, 3-7 membered monocyclic heterocyclyl, and 5-6 membered heteroaryl;

p is an integer selected from 1 to 2; wherein,

R³ is C₁₋₂alkyl;

R⁴ is hydrogen or C₁₋₂alkyl;

wherein R³ and R⁴ can be taken together to form C₃₋₅cycloalkyl;

R^{a} is independently, for each occurrence, selected from the group consisting of hydrogen, C₁₋₆alkyl, phenyl, and 3-7 membered monocyclic heterocyclyl;

R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and
when n is an integer selected from 1 to 6, W is selected from the group consisting of methyl, methylene (i.e.,

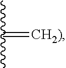

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e.,

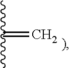

halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered monocyclic heterocyclyl and 5-6 membered heteroaryl are optionally substituted;

wherein the compound of formula (I-H) is:

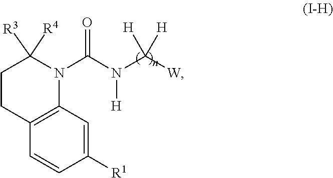

or a pharmaceutically acceptable salt thereof, wherein:

R¹ is an optionally substituted 3-7 membered monocyclic heterocyclyl (e.g., 3-7 membered monocyclic heterocyclyl optionally substituted with C₁₋₆alkyl);

R³ and R⁴ are independently C₁₋₂alkyl; wherein R³ and R⁴ can be taken together to form C₃₋₅cycloalkyl;

n is 1 to 6; and

W is an optionally substituted phenyl;

wherein the compound of formula (I-B) is:

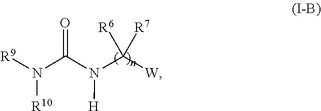

or a pharmaceutically acceptable salt thereof, wherein:

R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl; and

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and
when n is an integer selected from 1 to 6, W is selected from the group consisting of methyl, methylene (i.e.,

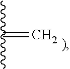

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e.,

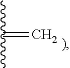

halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and −O—(C₁₋₆alkylene)-phenyl are optionally substituted (e.g., with one or more halogens or CF₃);

wherein any aforementioned 3-7 membered monocyclic heterocyclyl and 5-6 membered heteroaryl are optionally substituted.

In another aspect, provided herein is a compound selected from the group consisting of a compound of formula (I-C), formula (I-D), formula (I-E), formula (I-F), formula (I-G), formula (I-H), and formula (I-B):

wherein the compound of formula (I-C) is:

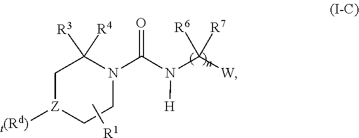

or a pharmaceutically acceptable salt thereof, wherein:

Z is selected from the group consisting of C, CH, N, and O; wherein

(i) when Z is C, t=1 (if R^{d} is oxo) or 2, when Z is CH, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered heterocyclyl), or (3-7 membered monocyclic heterocyclylene)-(3-7 membered monocyclic heterocyclyl) is optionally substituted; and
at least one of R¹ and R^{d} is cyclyl or substituted cyclyl:

(ii) when Z is O, t is 0;
R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;

(iii) when Z is N, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is selected from the group consisting of hydrogen, C₁₋₃alkyl, C₁₋₆alkyl, phenyl, C₃₋₇cycloalkyl, 5-6 membered heteroaryl, 3-7 membered monocyclic saturated heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 3-7 membered monocyclic saturated heterocyclyl, —O—C₁₋₆alkyl, —O-phenyl, or (3-7 membered monocyclic heterocyclylene)-(3-7 membered monocyclic heterocyclyl) is optionally substituted;

wherein at least one of R¹ and R^{d} is cyclyl or substituted cyclyl, wherein R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted, or R^{d} is selected from the group consisting of phenyl, C₃₋₇cycloalkyl, 5-6 membered heteroaryl, 3-7 membered monocyclic heterocyclyl, and (3-7 membered monocyclic heterocyclylene)-(3-7 membered monocyclic heterocyclyl), wherein the phenyl, C₃₋₇cycloalkyl, 5-6 membered heteroaryl, 3-7 membered monocyclic heterocyclyl, or (3-7 membered monocyclic heterocyclylene)-(3-7 membered monocyclic heterocyclyl) is optionally substituted;

R³ is optionally substituted C₁₋₆alkyl;

R⁴ is hydrogen or C₁₋₃alkyl; or

R³ and R⁴ can be taken together to form C₃₋₆cycloalkyl;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 3 to 5; and

W is an optionally substituted phenyl;

wherein the compound of formula (I-D) is:

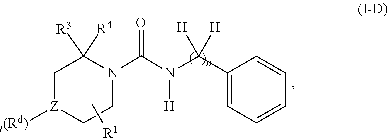

or a pharmaceutically acceptable salt thereof, wherein:

Z is selected from the group consisting of C, CH, N, and O; wherein

when Z is C, t=1 (if R^{d} is oxo), or 2, when Z is CH, t=1, and when Z is O, t=0;

when Z is C, CH, or N, R¹ is hydrogen or optionally substituted phenyl;

when Z is O, R¹ is optionally substituted phenyl;

R³ is C₁₋₃alkyl;

R⁴ is hydrogen or C₁₋₃alkyl;

R^{d} is independently, for each occurrence, selected from the group consisting of hydrogen, C₁₋₆alkyl, phenyl, C₃₋₇cycloalkyl, 5-6 membered heteroaryl, 3-7 membered monocyclic heterocyclyl, wherein the C₁₋₆alkyl, phenyl, C₃₋₇cycloalkyl, 5-6 membered heteroaryl, 3-7 membered monocyclic heterocyclyl is optionally substituted;

n is 4;

wherein at least one of R¹ and R^{d} is cyclyl or substituted cyclyl, wherein R¹ is optionally substituted phenyl, or R^{d} is optionally substituted phenyl or optionally substituted 3-7 membered monocyclic heterocyclyl;

wherein the compound of formula (I-E) is:

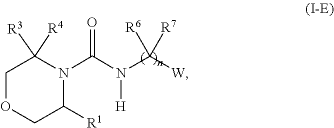

or a pharmaceutically acceptable salt thereof, wherein:

R¹ is C₁₋₆alkyl or optionally substituted phenyl;

R³ and R⁴ are independently C₁₋₆alkyl;

R⁶ and R⁷ are independently, for each occurrence, selected from the group consisting of hydrogen, C₁₋₆alkyl, C₁₋₆haloalkyl, and halogen;

n is an integer selected from 1 to 5; and

W is selected from the group consisting of methyl, an optionally substituted phenyl, and an optionally substituted C₃₋₇cycloalkyl;

wherein the compound of formula (I-F) is:

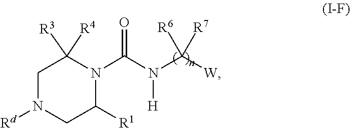

or a pharmaceutically acceptable salt thereof, wherein:

R¹ is C₁₋₆alkyl;

R³ is C₁₋₆alkyl;

R⁴ is C₁₋₆alkyl or hydrogen;

R⁶ and R⁷ are independently, for each occurrence, selected from the group consisting of hydrogen, C₁₋₆alkyl, C₁₋₆haloalkyl, and halogen;

n is an integer selected from 1 to 6;

W is selected from the group consisting of methyl, an optionally substituted phenyl, and an optionally substituted C₃₋₇cycloalkyl; and

R^{d} is C₃₋₇cycloalkyl optionally substituted with C₁₋₆alkyl or halogen;

wherein the compound of formula (I-G) is:

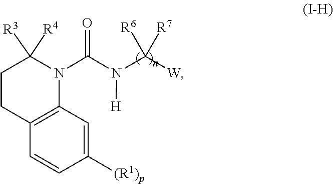

or a pharmaceutically acceptable salt thereof, wherein:

R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl; wherein at least one of R¹ is selected from the group consisting of phenyl, 3-7 membered monocyclic heterocyclyl, and 5-6 membered heteroaryl;

p is an integer selected from 1 to 2; wherein,

R³ is C₁₋₂alkyl;

R⁴ is hydrogen or C₁₋₂alkyl;

wherein R³ and R⁴ can be taken together to form C₃₋₅cycloalkyl;

R^{a} is independently, for each occurrence, selected from the group consisting of hydrogen, C₁₋₆alkyl, phenyl, and 3-7 membered monocyclic heterocyclyl;

R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and
when n is an integer selected from 1 to 6, W is selected from the group consisting of methyl, methylene (i.e.,

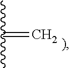

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
when n is 0, W is selected from the group consisting of methyl, methylene (i.e.,

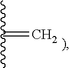

halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered monocyclic heterocyclyl and 5-6 membered heteroaryl are optionally substituted;

wherein the compound of formula (I-H) is:

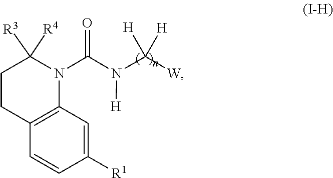

or a pharmaceutically acceptable salt thereof, wherein:

R¹ is an optionally substituted 3-7 membered monocyclic heterocyclyl (e.g., 3-7 membered monocyclic heterocyclyl optionally substituted with C₁₋₆alkyl);

R³ and R⁴ are independently C₁₋₂alkyl; wherein R³ and R⁴ can be taken together to form C₃₋₅cycloalkyl;

n is 1 to 6; and

W is an optionally substituted phenyl;

wherein the compound of formula (I-B) is:

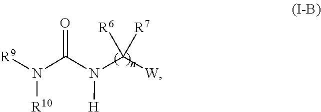

or a pharmaceutically acceptable salt thereof, wherein:

R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl; and

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and
when n is an integer selected from 1 to 6, W is selected from the group consisting of methyl, methylene (i.e.,

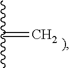

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e.,

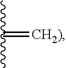

halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇ cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);

wherein any aforementioned 3-7 membered monocyclic heterocyclyl and 5-6 membered heteroaryl are optionally substituted.

In some embodiments, wherein the compound is a compound of formula (I-C) or formula (I-D), R⁴ is hydrogen or methyl.

In some embodiments, wherein the compound is a compound of formula (I-C) or formula (I-D), R^{d} is selected from the group consisting of hydrogen, methyl, phenyl, and

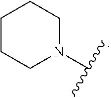 [SMILES: *N1CCCCC1]

In some embodiments, wherein the compound is a compound of formula (I-C) or formula (I-D), Z is CH and R^{d} is selected from the group consisting of hydrogen, phenyl, and

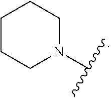 [SMILES: *N1CCCCC1]

In some embodiments, wherein the compound is a compound of formula (I-C) or formula (I-D), Z is N and R^{d} is methyl or phenyl.

In some embodiments, wherein the compound is a compound of formula (I-C) or formula (I-D), R³ is methyl.

In some embodiments, wherein the compound is a compound of formula (I-C) or formula (I-D), R¹ is hydrogen or phenyl.

In some embodiments, wherein the compound is a compound of formula (I-C), n is 4.

In some embodiments, wherein the compound is a compound of formula (I-C), W is phenyl.

In some embodiments, wherein the compound is a compound of formula (I-E), R¹ is methyl or phenyl optionally substituted with halogen or —OCH₃.

In some embodiments, wherein the compound is a compound of formula (I-E), W is methyl or cyclopropyl.

In some embodiments, wherein the compound is a compound of formula (I-E), n is 4.

In some embodiments, wherein the compound is a compound of formula (I-E), n is 1.

In some embodiments, wherein the compound is a compound of formula (I-F), R¹ is methyl.

In some embodiments, wherein the compound is a compound of formula (I-F), R^{d} is cyclopropyl.

In some embodiments, wherein the compound is a compound of formula (I-F), n is 4.

In some embodiments, wherein the compound is a compound of formula (I-F), W is methyl.

In some embodiments, wherein the compound is a compound of formula (I-G) or formula (I-H), n is 2.

In some embodiments, wherein the compound is a compound of formula (I-G) or formula (I-H), W is phenyl.

In some embodiments, wherein the compound is a compound of formula (I-G) or formula (I-H), R¹ is

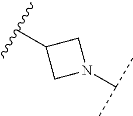

In some embodiments, the compound is a compound of formula (I-B):

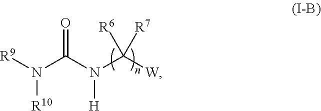

or a pharmaceutically acceptable salt thereof, wherein:

R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl; and

R⁶, R⁷, n, and W are as defined in the compound of formula (I);

wherein any aforementioned 3-7 membered monocyclic heterocyclyl and 5-6 membered heteroaryl are optionally substituted.

In some embodiments, R⁹ is C₁₋₆alkyl.

In some embodiments, R⁹ is methyl.

In some embodiments, R¹⁰ is selected from the group consisting of C₁₋₆alkylene-phenyl, 3-7 membered monocyclic heterocyclyl, 7-8 membered bridged bicyclic cycloalkyl, and 7-8 membered bridged bicyclic heterocyclyl, wherein the 3-7 membered monocyclic heterocyclyl is optionally substituted with methyl.

In some embodiments, R¹⁰ is selected from the group consisting of

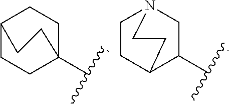

In some embodiments of formula (I), the compound is a compound of formula (I-C):

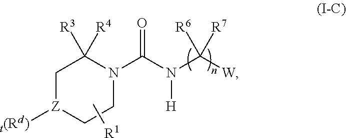

or a pharmaceutically acceptable salt thereof, wherein:

Z is selected from the group consisting of C, CH, N, and O; wherein

(i) when Z is C, t=1 (if R^{d} is oxo) or 2, when Z is CH, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered heterocyclyl), or (3-7 membered monocyclic heterocyclylene)-(3-7 membered monocyclic heterocyclyl) is optionally substituted;

(ii) when Z is O, t is 0;
R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;

(iii) when Z is N, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is selected from the group consisting of hydrogen, C₁₋₃alkyl, C₁₋₆alkyl, phenyl, C₃₋₇cycloalkyl, 5-6 membered heteroaryl, 3-7 membered monocyclic saturated heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 3-7 membered monocyclic saturated heterocyclyl, —O—C₁₋₆alkyl, —O-phenyl, or (3-7 membered monocyclic heterocyclylene)-(3-7 membered monocyclic heterocyclyl) is optionally substituted:

wherein at least one of R¹ and R^{b} is cyclyl or substituted cyclyl, wherein R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted, or R^{d} is selected from the group consisting of phenyl, C₃₋₇cycloalkyl, 5-6 membered heteroaryl, 3-7 membered monocyclic heterocyclyl, and (3-7 membered monocyclic heterocyclylene)-(3-7 membered monocyclic heterocyclyl), wherein the phenyl, C₃₋₇cycloalkyl, 5-6 membered heteroaryl, 3-7 membered monocyclic heterocyclyl, or (3-7 membered monocyclic heterocyclylene)-(3-7 membered monocyclic heterocyclyl) is optionally substituted;

R³ is optionally substituted C₁₋₆alkyl;

R⁴ is hydrogen or C₁₋₃alkyl; or

R³ and R⁴ can be taken together to form C₃₋₆cycloalkyl;

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 3 to 5; and

W is an optionally substituted phenyl.

In some embodiments, R¹ is hydrogen. In some embodiments, R¹ is phenyl.

In some embodiments, Z is CH. In some embodiments, Z is C. In some embodiments, Z is O. In some embodiments, Z is N.

In some embodiments, R^{d} is hydrogen. In some embodiments, R^{d} is phenyl. In some embodiments, R^{d} is 3-7 membered heterocyclyl.

In some embodiments, R³ and R⁴ are methyl. In some embodiments, R³ is hydrogen and R⁴ is methyl.

In some embodiments, n is 3. In some embodiments, n is 4. In some embodiments, n is 5.

In some embodiments, W is phenyl.

In some embodiments, the compound is a compound of formula (I-D):

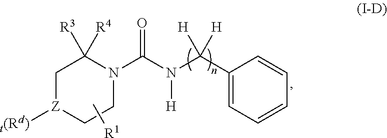

or a pharmaceutically acceptable salt thereof, wherein:

Z is selected from the group consisting of C, CH, N, and O; wherein

when Z is C, t=1 (if R^{d} is oxo), or 2, when Z is CH, t=1, and when Z is O, t=0;

when Z is C, CH, or N, R¹ is hydrogen or optionally substituted phenyl;

when Z is O, R¹ is optionally substituted phenyl;

R³ is C₁₋₃alkyl;

R⁴ is hydrogen or C₁₋₃alkyl;

R^{d} is independently, for each occurrence, selected from the group consisting of hydrogen, C₁₋₆alkyl, phenyl, 3-7 membered monocyclic heterocyclyl, wherein the C₁₋₆alkyl, phenyl, or 3-7 membered monocyclic heterocyclyl is optionally substituted; and

n is 4;

wherein at least one of R¹ and R^{b} is cyclyl or substituted cyclyl, wherein R¹ is optionally substituted phenyl, or R^{d} is optionally substituted phenyl or optionally substituted 3-7 membered monocyclic heterocyclyl.

In some embodiments, Z is CH. In some embodiments, Z is C. In some embodiments, Z is O. In some embodiments, Z is N.

In some embodiments, R^{d} is hydrogen. In some embodiments, R^{d} is phenyl. In some embodiments, R^{d} is 3-7 membered heterocyclyl.

In some embodiments, R³ and R⁴ are methyl. In some embodiments, R³ is hydrogen and R⁴ is methyl. In some embodiments, R⁴ is hydrogen or methyl.

In some embodiments, R^{d} is selected from the group consisting of hydrogen, methyl, phenyl, and

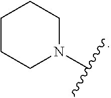 [SMILES: *N1CCCCC1]

In certain embodiments, Z is CH and R^{d} is selected from the group consisting of hydrogen, phenyl, and

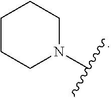 [SMILES: *N1CCCCC1]

In other embodiments, Z is N and R^{d} is methyl or phenyl.

In some embodiments of formula (I), the compound is a compound of formula (I-E):

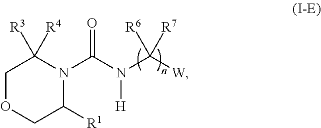

or a pharmaceutically acceptable salt thereof, wherein:

R¹ is C₁₋₆alkyl or optionally substituted phenyl;

R³ and R⁴ are independently C₁₋₆alkyl;

R⁶ and R⁷ are independently, for each occurrence, selected from the group consisting of hydrogen, C₁₋₆alkyl, C₁₋₆haloalkyl, and halogen;

n is an integer selected from 1 to 5; and

W is selected from the group consisting of methyl, an optionally substituted phenyl, and an optionally substituted C₃₋₇cycloalkyl.

In some embodiments, R¹ is methyl. In some embodiments, R¹ is phenyl. In some embodiments, R¹ is fluoride substituted phenyl.

In some embodiments, R³ and R⁴ are methyl.

In some embodiments, n is 4. In some embodiments, n is 1.

In some embodiments, W is phenyl. In some embodiments, W is methyl. In some embodiments, W is cyclopropyl.

In some embodiments of formula (I), the compound is a compound of formula (I-F):

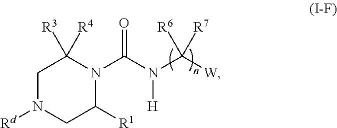

or a pharmaceutically acceptable salt thereof, wherein:

R¹ is C₁₋₆alkyl;

R³ is C₁₋₆alkyl;

R⁴ is C₁₋₆alkyl or hydrogen;

R⁶ and R⁷ are independently, for each occurrence, selected from the group consisting of hydrogen, C₁₋₆alkyl, C₁₋₆haloalkyl, and halogen;

n is an integer selected from 1 to 6;

W is selected from the group consisting of methyl, an optionally substituted phenyl, and an optionally substituted C₃₋₇cycloalkyl; and

R^{d} is selected from phenyl, C₃₋₇cycloalkyl, 5-6 membered heteroaryl, wherein the phenyl, C₃₋₇cycloalkyl, 5-6 membered heteroaryl, is optionally substituted.

In some embodiments, R¹ is methyl. In some embodiments, R³ is methyl. In some embodiments, R⁴ is methyl. In some embodiments, n is 4. In some embodiments, W is methyl. In some embodiments, R^{d} is cyclopropyl.

In some embodiments of formula (I), the compound is a compound of formula (I-G):

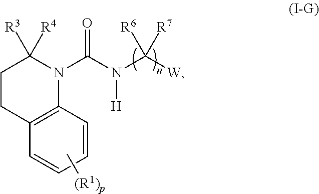

or a pharmaceutically acceptable salt thereof, wherein:

R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl; wherein at least one of R¹ is selected from the group consisting of phenyl, 3-7 membered monocyclic heterocyclyl, and 5-6 membered heteroaryl;

p is an integer selected from 1 to 2; wherein,

R³ is C₁₋₂alkyl;

R⁴ is hydrogen or C₁₋₂alkyl;

wherein R³ and R⁴ can be taken together to form C₃₋₅cycloalkyl;

R^{a} is independently, for each occurrence, selected from the group consisting of hydrogen, C₁₋₆alkyl, phenyl, and 3-7 membered monocyclic heterocyclyl;

R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂,

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and

when n is an integer selected from 1 to 6, W is selected from the group consisting of methyl, methylene (i.e.,

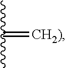

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),

when n is 0, W is selected from the group consisting of methyl, methylene (i.e., halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein

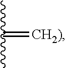

the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);

wherein any aforementioned 3-7 membered monocyclic heterocyclyl and 5-6 membered heteroaryl are optionally substituted.

In other embodiments, the compound is a compound of formula (I-H):

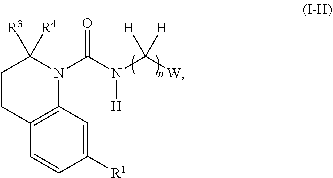

or a pharmaceutically acceptable salt thereof, wherein:

R¹ is an optionally substituted 3-7 membered monocyclic heterocyclyl (e.g., 3-7 membered monocyclic heterocyclyl optionally substituted with C₁₋₆alkyl);

R³ and R⁴ are independently C₁₋₂alkyl; wherein R³ and R⁴ can be taken together to form C₃₋₅cycloalkyl;

n is 1 to 6; and

W is an optionally substituted phenyl.

In some embodiments, R¹ is

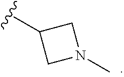

In certain embodiments, R³ and R⁴ are methyl.

In some embodiments, n is 2.

In some embodiments, W is phenyl.

In some embodiments of each of the foregoing compounds of formula (I), (I-A), (I-Aa), (I-Ab), (II), (III), (IV), (V), (VI), (I-B), (I-C), (I-D), (I-E), (I-F), (I-G), or (I-H), R⁶ and R⁷ are selected from the group consisting of hydrogen, methyl, and halogen.

In some embodiments of each of the foregoing compounds of formula (I), (I-A), (I-Aa), (I-Ab), (II), (III), (IV), (V), (VI), (I-B), (I-C), (I-D), (I-E), (I-F), (I-G), or (I-H), R⁶ and R⁷ are selected from the group consisting of hydrogen, and methyl.

In some embodiments of each of the foregoing compounds of formula (I), (I-A), (I-Aa), (I-Ab), (II), (II), (IV), (V), (VI), (I-B), (I-C), (I-D), (I-E), (I-F), (I-G), or (I-H), R⁶ and R⁷ are hydrogen.

In some embodiments of each of the foregoing compounds (I), (I-A), (I-Aa), (I-Ab), (II), (I), (IV), (V), (VI), (I-B), (I-E), (I-F), (I-G), or (I-H), n is 2. In some embodiments of each of the foregoing compounds (I), (I-A), (I-Aa), (I-Ab), (II), (III), (IV), (V), (VI), or (I-B), or (I-G), n is 0. In some embodiments of each of the foregoing compounds of formula (I), (I-A), (I-Aa), (I-Ab), (II), (III), (IV), (V), (VI), (I-B), (I-E), (I-F), (I-G), or (I-H), n is 1. In some embodiments of each of the foregoing compounds of formula (I), (I-A), (I-Aa), (I-Ab), (II), (III), (IV), (V), (VI), (I-B), (I-C), (I-E), (I-F), (I-G), or (I-H), n is 3. In some embodiments of each of the foregoing compounds of formula (I), (I-A), (I-Aa), (I-Ab), (II), (III), (IV), (V), (VI), (I-B), (I-C), (I-E), (I-F), (I-G), or (I-H), n is 4. In some embodiments of each of the foregoing compounds of formula (I), (I-A), (I-Aa), (I-Ab), (II), (III), (IV), (V), (VI), (I-B), (I-C), (I-E), (I-F), (I-G), or (I-H), n is 5. In some embodiments of each of the foregoing compounds of formula (I), (I-A), (I-Aa), (I-Ab), (II), (III), (IV), (V), (VI), (I-B), (I-D), (I-E), (I-F), (I-G), or (I-H), n is 6.

In some embodiments of each of the foregoing compounds of formula (I), (I-A), (II), (III), (IV), (V), (VI), or (I-B), n is selected from 0, 1, 2, 3, 4, and 6.

In some embodiments of each of the foregoing compounds of formula (I), (I-A), (II), (III), (IV), (V), (VI), or (I-B), n is 2 or 4.

In some embodiments of each of the foregoing compounds of formula (I), (I-A), (II), (I), (IV), (V), (VI), or (I-B), W is selected from the group consisting of methyl, ethenyl, halogen, phenyl, C₃₋₇cycloalkyl, 3-7 membered heterocyclyl, 5-6 membered heteroaryl, —O—C₁₋₆alkyl, —O—(C₁₋₆alkylene)-C₃₋₇cycloalkyl, —O-phenyl, and —O—(C₁₋₆alkylene)-phenyl, wherein methyl, ethenyl, phenyl, C₃₋₇cycloalkyl, 3-7 membered heterocyclyl, 5-6 membered heteroaryl, —O—C₁₋₆alkyl, —O—(C₁₋₆alkylene)-C₃₋₇cycloalkyl, —O-phenyl, and —O—(C₁₋₆alkylene)-phenyl are optionally substituted with halogen.

In some embodiments of each of the foregoing compounds of formula (I), (I-A), (II), (III), (IV), (V), (VI), or (I-B), W is selected from the group consisting of methyl, ethenyl, fluorine, —CF₃, cyclopropyl, cyclohexyl, phenyl, —O-phenyl,

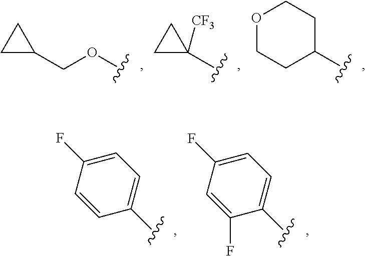

—OCH₂Ph and —OCH₃.

In some embodiments of each of the foregoing compounds of formula (I), (I-A), (II), (III), (IV), (V), (VI), or (I-B), W is selected from the group consisting of methyl, —CH₂F, —CF₃, cyclopropyl, cyclohexyl, phenyl, —O-phenyl,

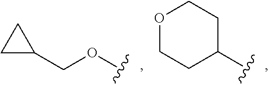

### and —OCH₃.

In some embodiments of each of the foregoing compounds of formula (I), (I-A), (II), (III), (IV), (V), (VI), or (I-B), W is phenyl.

In some embodiments of each of the foregoing compounds of formula (I), (I-A), (I-Aa), (I-Ab), (II), (III), (IV), (V), (VI) (I-B), (I-C), (I-D), (I-E), (I-F), (I-G), and (I-H), unless otherwise specified, any aforementioned 3-7 membered monocyclic heterocyclyl, and 5-6 membered heteroaryl are optionally substituted with 1-4 substituents independently, for each occurrence, selected from the group consisting of —CH₂N(R^{a})₂, cyano, C₁₋₆alkyl, halogen, and —O—C₁₋₆alkyl, wherein R^{a} is selected from the group consisting of hydrogen, C₁₋₆alkyl, phenyl, and 3-7 membered monocyclic heterocyclyl;

In some embodiments of each of the foregoing compounds of formula (I), (I-A), (I-Aa), (I-Ab), (II), (III), (IV), (V), (VI) (I-B), (I-C), (I-D), (I-E), (I-F), (I-G), and (I-H), unless otherwise specified, any aforementioned 3-7 membered monocyclic heterocyclyl, and 5-6 membered heteroaryl at R^{a}, R^{b}, R^{d}, R¹, R², R⁹, or R¹⁰ are optionally substituted with 1-3 substituents independently, for each occurrence, selected from the group consisting of —CH₂N(R^{a})₂, cyano, C₁₋₆alkyl, halogen, and —O—C₁₋₆alkyl, wherein R^{a} is as defined herein.

In some embodiments of each of the foregoing compounds of formula (I), (I-A), (I-Aa), (I-Ab), (II), (III), (IV), (V), (VI), (I-B), (I-C), (I-D), (I-E), (I-F), (I-G), and (I-H), unless otherwise specified, any 3-7 membered monocyclic heterocyclyl and 5-6 membered heteroaryl at R^{a}, R^{b}, R^{d}, R¹, R², R⁹, or R¹⁰ are optionally substituted with methyl.

In certain embodiments, the compound is a compound described in the Examples, or a pharmaceutically acceptable salt thereof. In certain other embodiments, the compound is one of the compounds listed in Table 1 below or a pharmaceutically acceptable salt thereof.

### Methods of Preparing Compounds

Methods for preparing compounds described herein are illustrated in the following synthetic schemes. These schemes are given for the purpose of illustrating the invention and should not be regarded in any manner as limiting the scope or the spirit of the invention. Starting materials shown in the schemes can be obtained from commercial sources or can be prepared based on procedures described in the literature.

### Synthesis of Compounds of Formula I-A.

Described herein are methods of synthesizing the compounds represented by the general Formula I-A

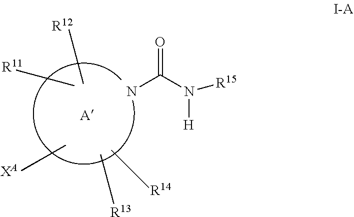

or pharmaceutically acceptable salts or solvates thereof wherein

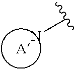

is selected from a monocyclic or bicyclic (e.g., fused, spiro, or bridged) heterocyclyl containing at least one N (including the depicted nitrogen);

X^{A} is independently selected from hydrogen, optionally substituted C₁-C₆alkyl, optionally substituted C₃-C₇cycloalkyl, optionally substituted 3-7 membered monocyclic heterocyclyl, phenyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), and (3-7 membered monocyclic heterocyclylene)-(3-7 membered monocyclic heterocyclyl), wherein X can be attached to any carbon or nitrogen atom of the ring to which it is connected;

R¹¹, R¹², R¹³ and R¹⁴ are independently selected from hydrogen, cyano, oxo, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, oxo (═O), —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring,

or any of R¹¹, R¹², R¹³ and/or R¹⁴ can be taken together with the carbon atoms to which they are attached to form an optionally substituted 3-6 membered spiro carbocyclic or spiro heterocyclic ring,

or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,

or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and

R¹⁵ is independently selected from optionally substituted C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), optionally substituted C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

According to Scheme 1 (Method A), compounds of Formula I-A wherein X^{A}, R¹¹, R¹², R¹³, R¹⁴ and R¹⁵, are defined above can be prepared, by reaction of substituted amines of Formula II-A, wherein

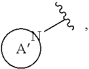

X^{A}, R¹¹, R¹², R¹³, R¹⁴ are as defined in Formula I-A, and isocyanates of Formula III-A, wherein R¹⁵ is as defined in Formula I-A, in the presence of catalytic amount of a base, e.g. 4-dimethylamino pyridine (DMAP), in polar solvent, such as acetonitrile (CH₃CN).

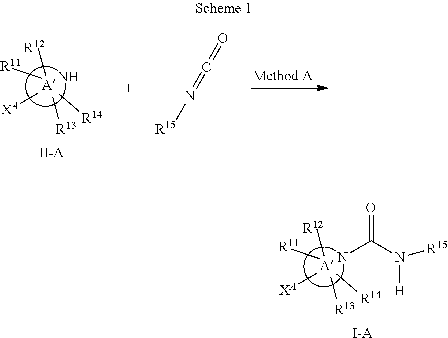

Isocyanates of Formula III-A, wherein R¹⁵ is as defined in Formula I-A, are commercially available or can be prepared from commercially available compounds according to general synthetic procedures described for instance in Michael Smith, Jerry March—March's Advanced Organic Chemistry: reactions mechanism and structure—6th Edition, John Wiley & Sons Inc., 2007, or in Molina P., Tarraga A., Arques A. in Katritzky A. R., Taylor R. J. K., Comprehensive Organic Functional Group Transformations II, Elsevier, 2004, Vol. 5, pag. 949-973, and references cited therein, which are herein incorporated by reference.

Alternatively, according to Scheme 2 (Method B), compounds of Formula I-A,

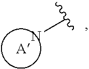

wherein, X^{A}, R¹¹, R¹², R¹³, R¹⁴ and R¹⁵, are as defined above, can be prepared by reaction of compounds of Formula II-A wherein X^{A}, R¹¹, R¹², R¹³ and R¹⁴ are as defined in Formula I-A, and amines of Formula IV-A, wherein R¹⁵ is as defined in Formula I-A.

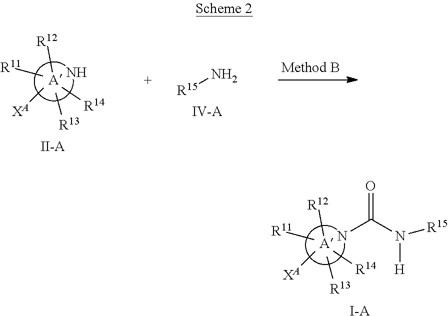

Triphosgene, phenylchloroformate, p-nitrophenylchloroformate, 1,1′-carbonyldiimidazole (CDI), and the like, are here used as activating agents of the amines. Such reactions are carried out in the presence of base such as triethylamine (Et3N), diisopropylethylamine (DIPEA) or pyridine (py) and in organic aprotic solvent, such as dichloromethane (DCM), CH₃CN, tetrahydrofuran (THF) or mixtures thereof.

Amines of Formula IV-A, wherein R¹⁵ is as defined in Formula I-A, are commercially available or can be prepared from commercially available compounds according to general synthetic procedures described for instance in Michael Smith, Jerry March—March's Advanced Organic Chemistry: reactions mechanisms and structure—6th Edition, John Wiley & Sons Inc., 2007, and references cited therein, which is herein incorporated by reference.

### Synthesis of Compounds of Formula I-Aa and Formula I-Ab

According to Scheme 3, compounds of Formula I-Aa, wherein Y^{A} is CH and R¹¹, R¹², R¹³, R¹⁴, R¹⁵ and X^{A} are as defined above, or compounds of Formula I-Ab, wherein Y^{A} is CH₂ and R¹¹, R¹², R¹³, R¹⁴, R¹⁵ and X^{A} are as defined above, can be prepared starting from compounds of Formula V-A.

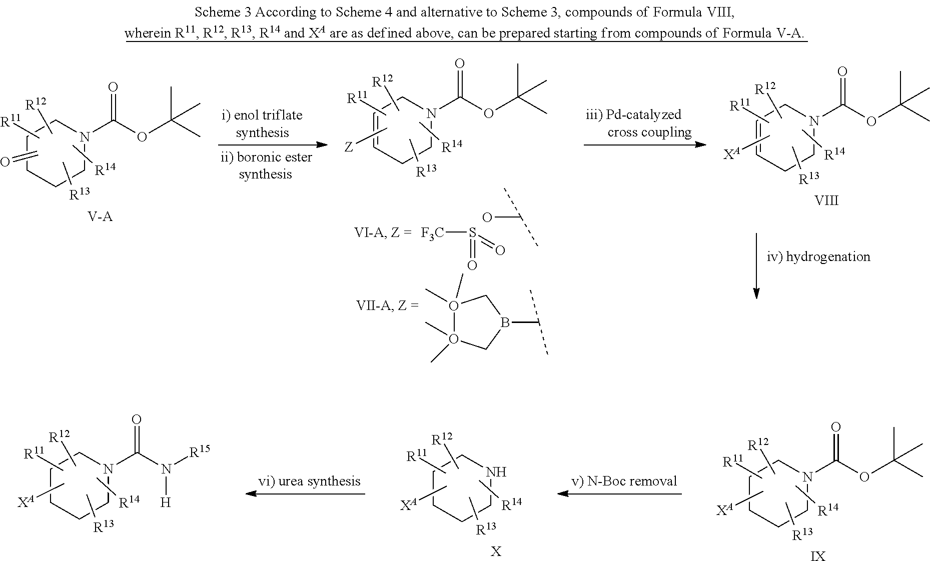

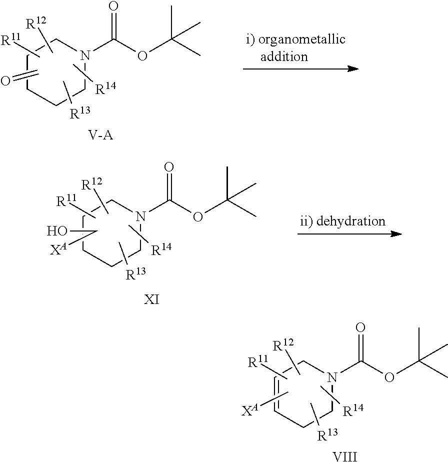

In accordance with certain embodiments, compounds of Formula V-A are selected from compounds of Formula V-Aa-i as those substituted ketones herein represented:

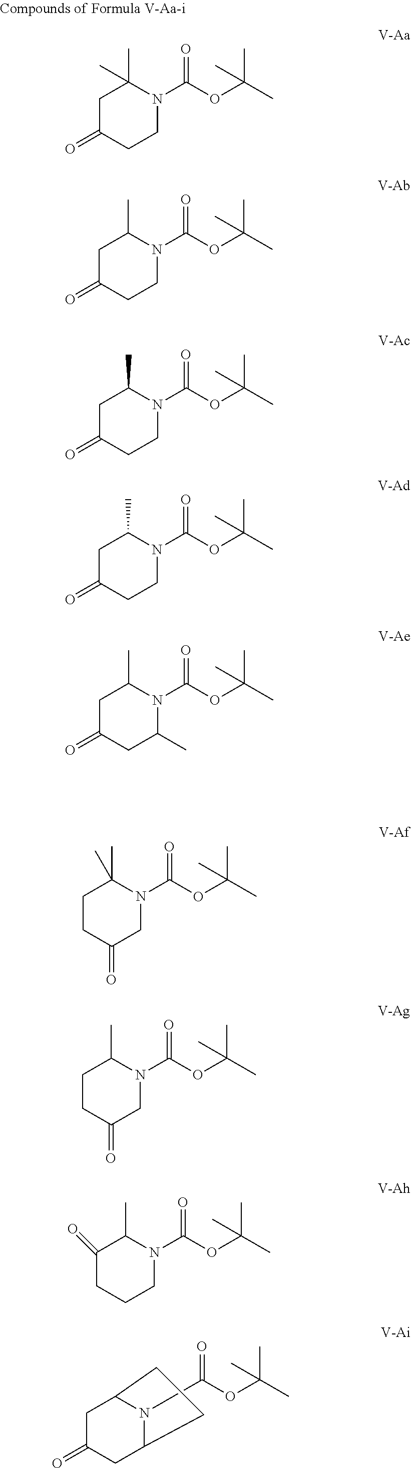

In accordance with certain embodiments, compounds of Formula VI-A are selected from compounds of Formula VI-Aa-i as those substituted enol triflates herein represented:

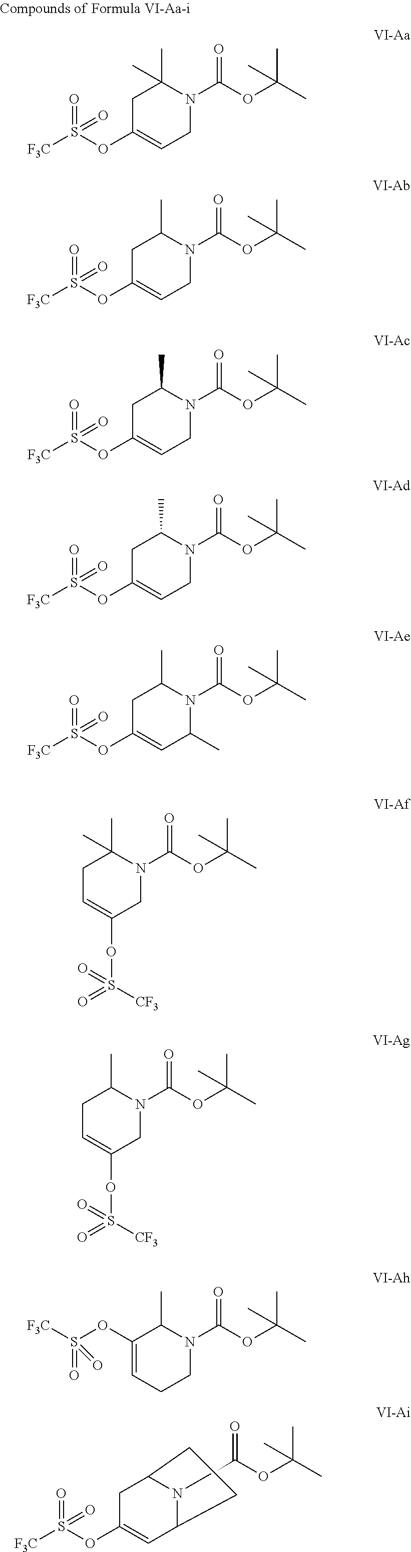

In accordance with certain embodiments, compounds of Formula VII are selected from of compounds of Formula VIIa-i as those substituted boronic esters herein represented:

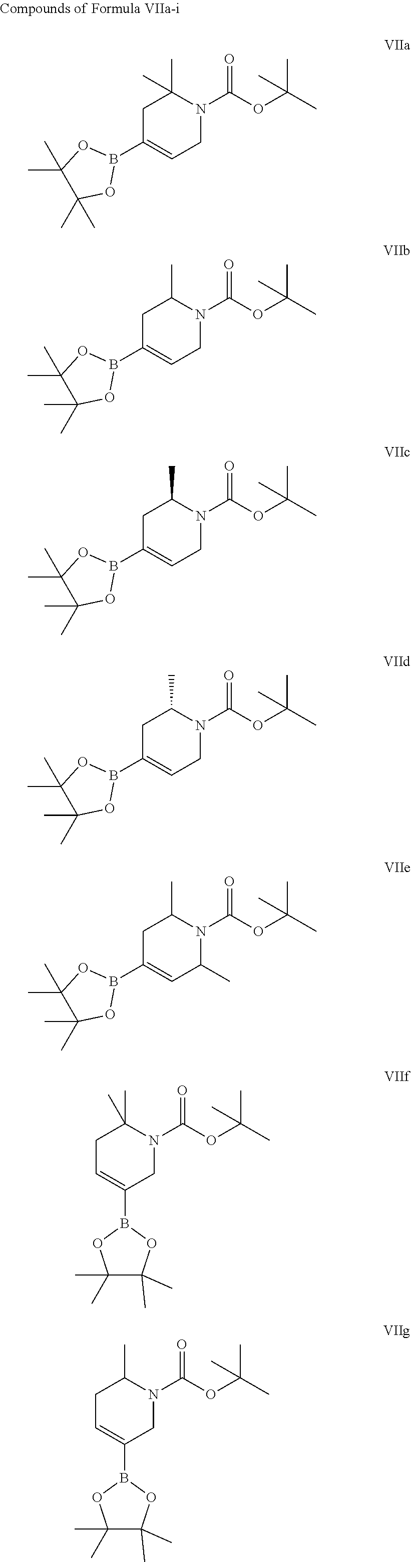

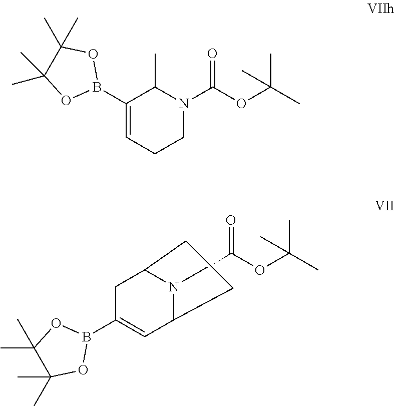

In accordance with certain embodiments, compounds of Formula VIII are selected from compounds of Formula VIIIa-h as those substituted piperidines herein represented:

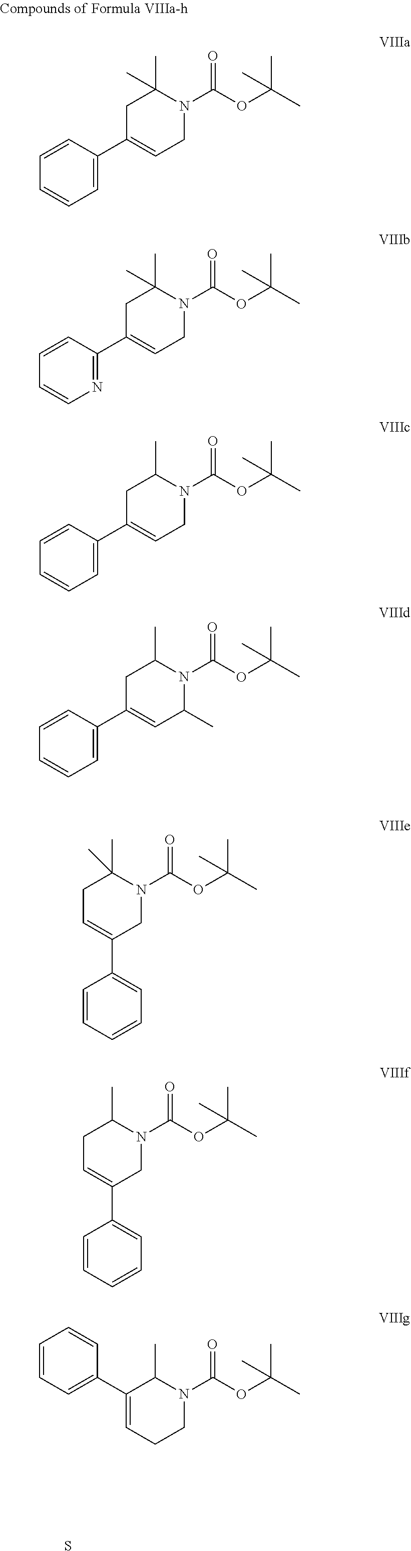

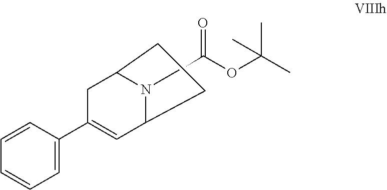 [SMILES: CC(C)(C)OC(=O)CN1C2C=C(c3ccccc3)CC1CCC2]

In accordance with certain embodiments, compounds of Formula IX are selected from compounds of Formula IXa-h as those substituted piperidines herein represented:

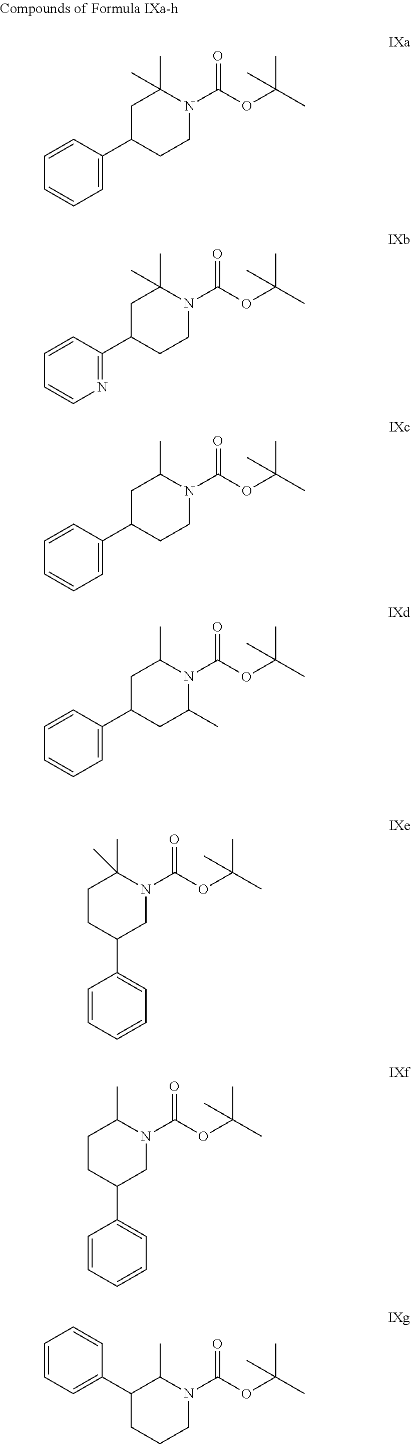

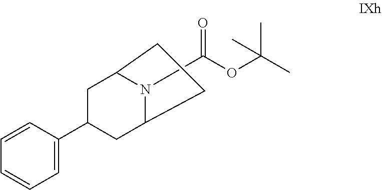 [SMILES: CC(C)(C)OC(=O)CN1C2CCCC1CC(c1ccccc1)C2]

In accordance with certain embodiments, compounds of Formula X are selected from compounds of Formula Xa-h as those substituted piperidines herein represented:

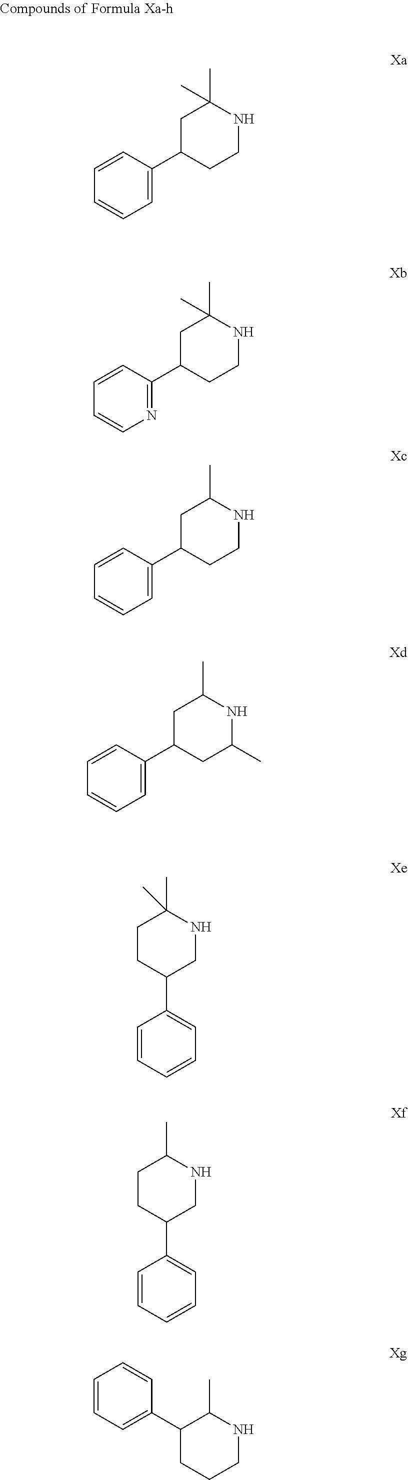

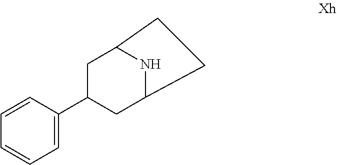 [SMILES: c1ccc(C2CC3CCCC(C2)N3)cc1]

In accordance with certain embodiments, compounds of Formula XI are selected from compounds of Formula XIa-h as those tertiary alcohols herein represented:

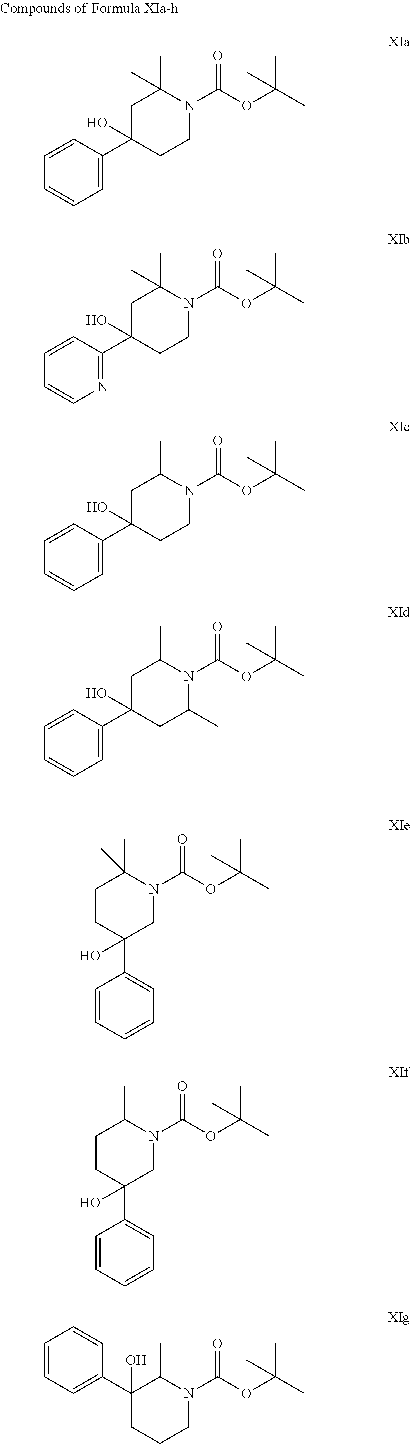

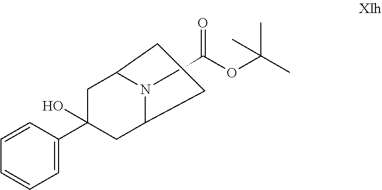 [SMILES: CC(C)(C)OC(=O)CN1C2CCCC1CC(O)(c1ccccc1)C2]

According to Scheme 5, compounds of Formula I-Aa, wherein R¹¹, R¹², R¹³, R¹⁴, R¹⁵ and X^{A} are as defined above, can be prepared starting from compounds of Formula V-A.

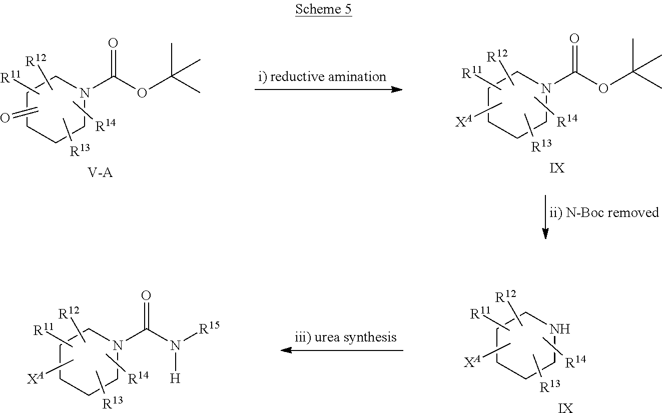

In accordance with certain embodiments, compounds of Formula IX are selected from compounds of Formula IXi-w as those substituted piperidines herein represented:

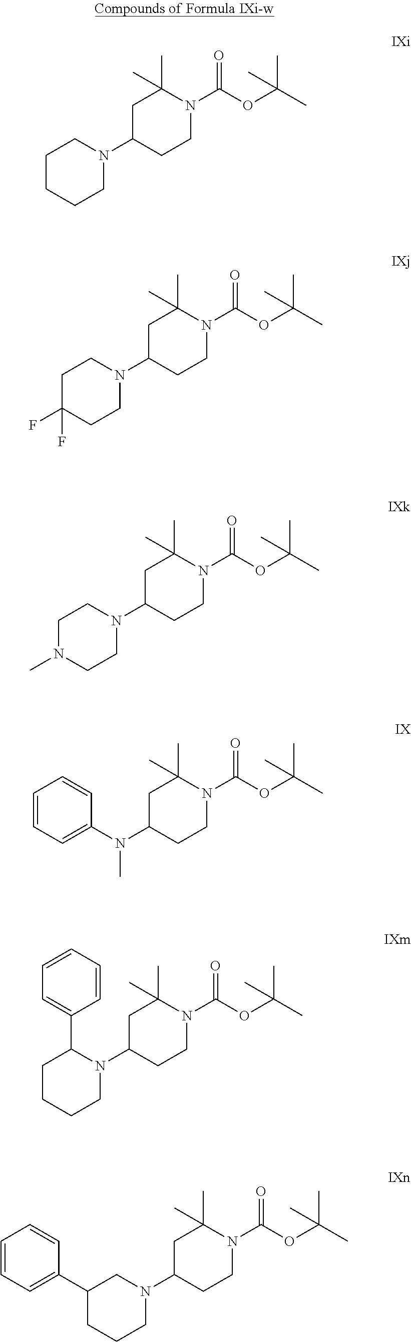

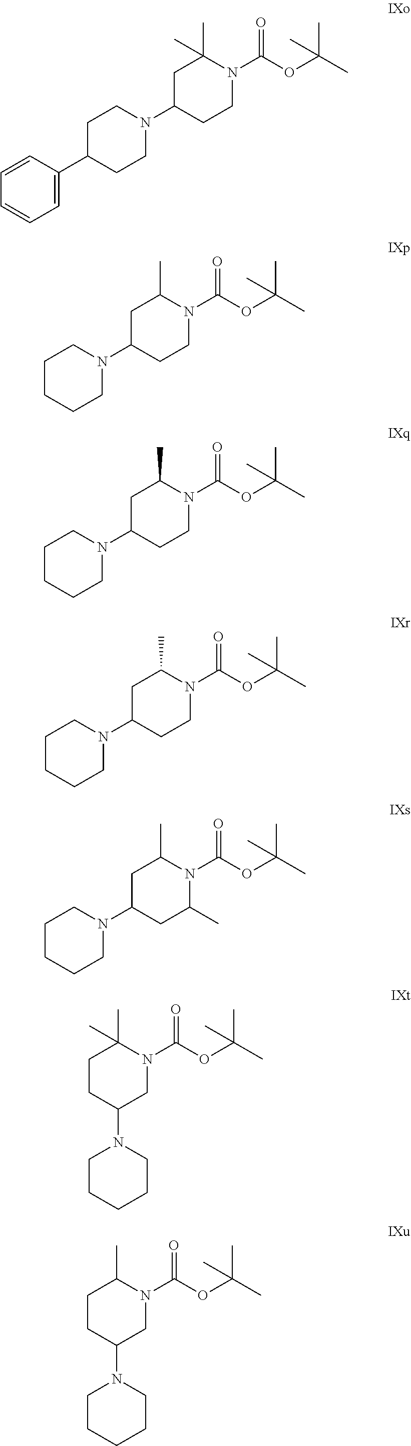

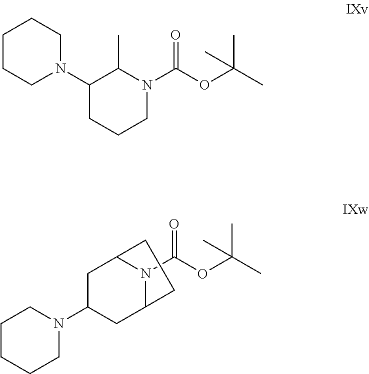 [SMILES: CC1C(N2CCCCC2)CCCN1C(=O)OC(C)(C)C ; CC(C)(C)OC(=O)N1C2CCC1CC(N1CCCCC1)C2]

In accordance with certain embodiments, compounds of Formula X are selected from compounds of Formula Xi-w as those substituted piperidines herein represented:

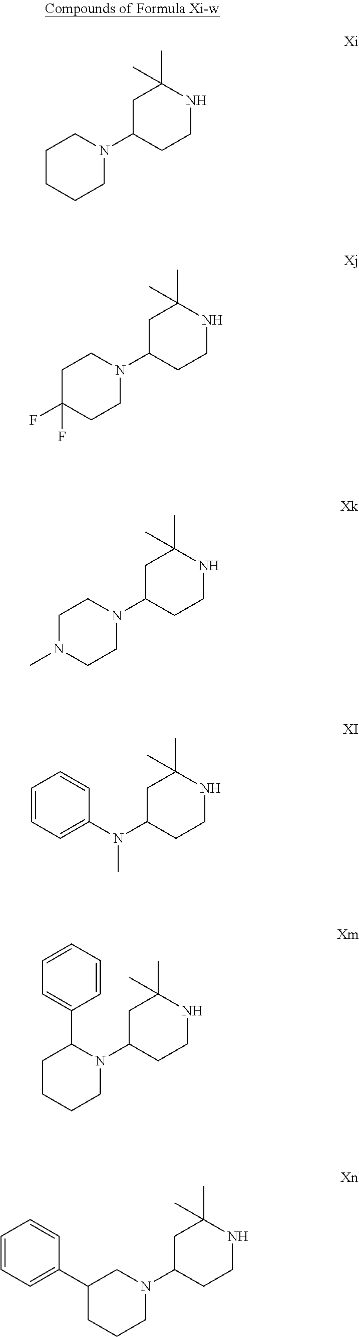 [SMILES: CC1(C)CC(N2CCCCC2)CCN1 ; CC1(C)CC(N2CCC(F)(F)CC2)CCN1 ; CN1CCN(C2CCNC(C)(C)C2)CC1 ; CN(c1ccccc1)C1CCNC(C)(C)C1 ; CC1(C)CC(N2CCCCC2c2ccccc2)CCN1 ; CC1(C)CC(N2CCCC(c3ccccc3)C2)CCN1]

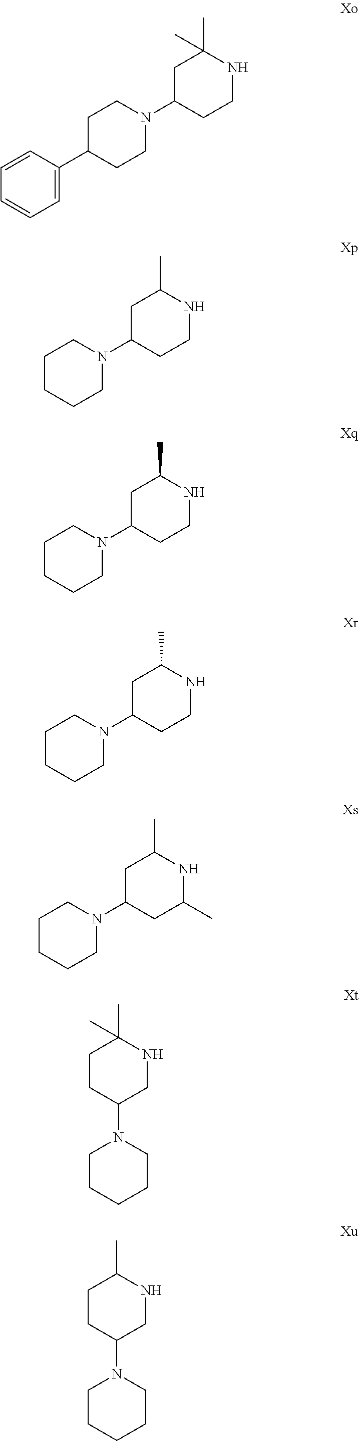

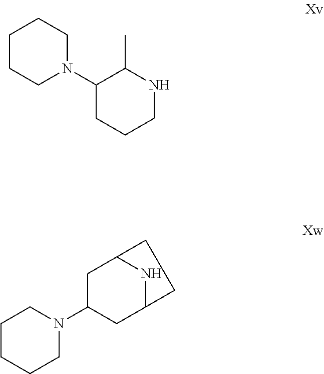

According to Scheme 6, compounds of Formula I-Aa, wherein R¹¹ and R¹², R¹³, R¹⁴, R¹⁵ and X^{A} are as defined above, can be prepared, starting from compounds of Formula XII.

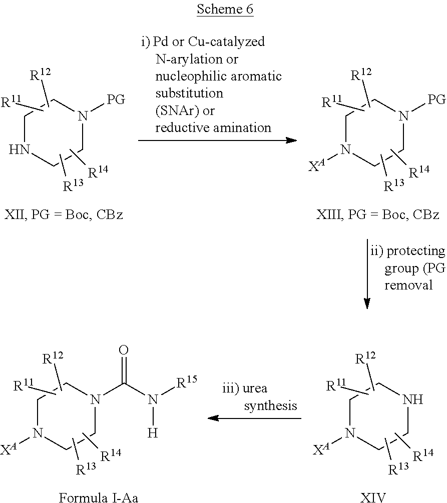

In accordance with certain embodiments, compounds of Formula XII are selected from compounds of Formula XIIa-h as those substituted piperazines herein represented:

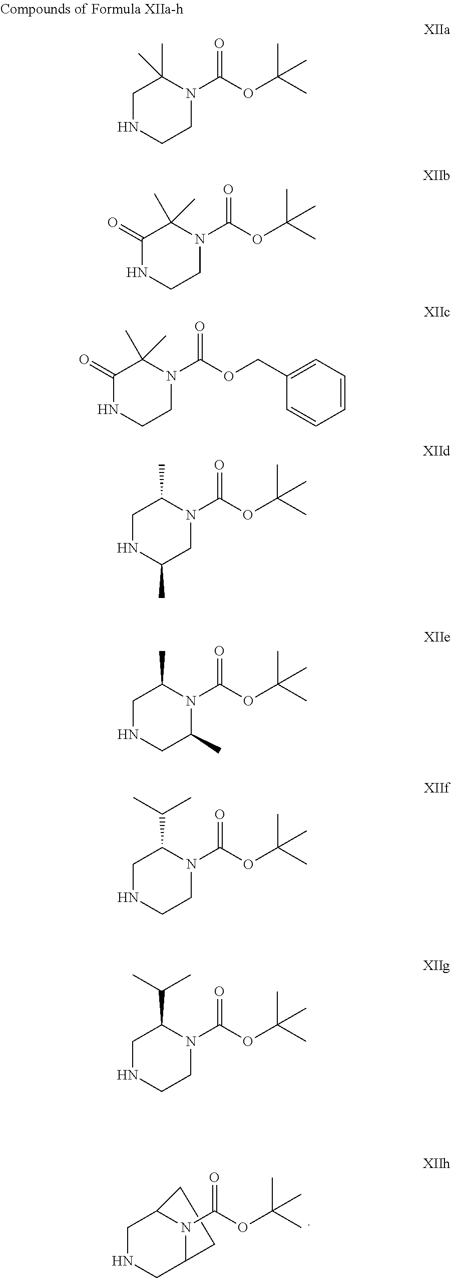

In accordance with certain embodiments, compounds of Formula XIII are selected from compounds of Formula XIIIa-l as those substituted piperazines herein represented:

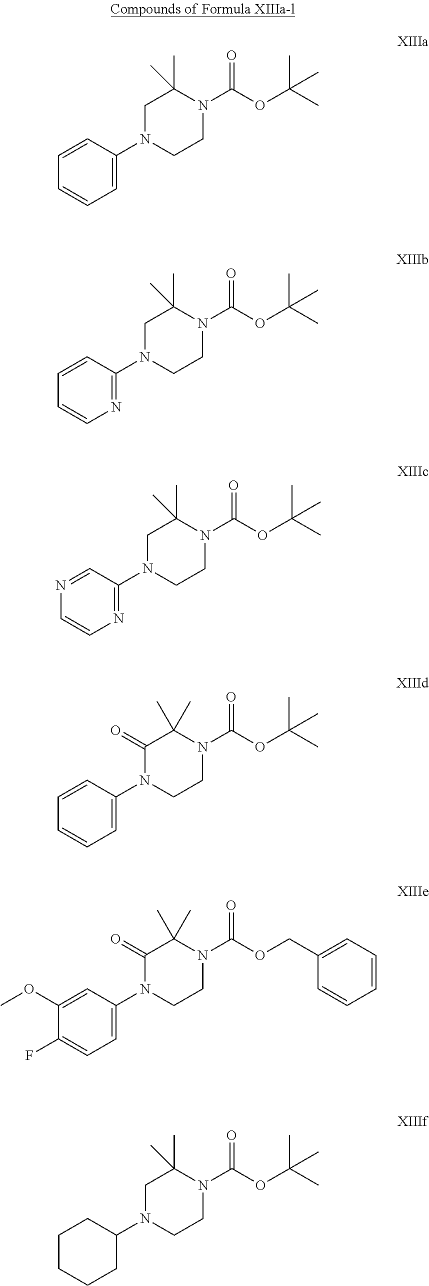

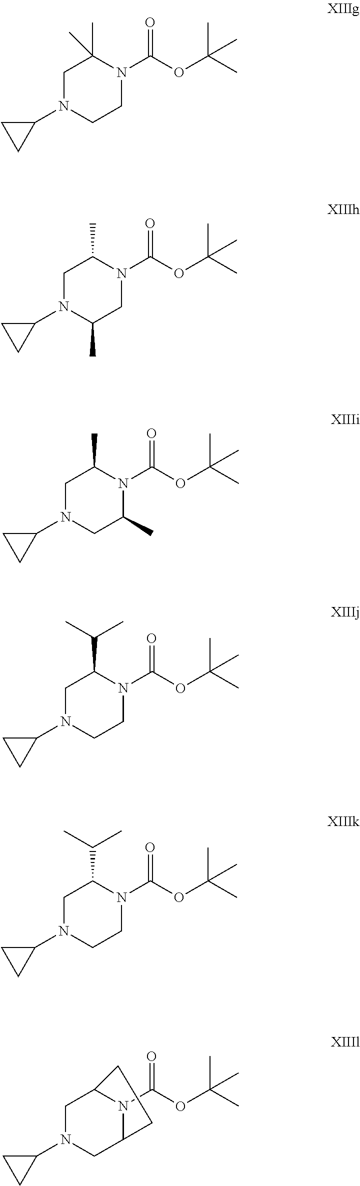 [SMILES: CC(C)(C)OC(=O)N1CCN(C2CC2)CC1(C)C ; C[C@@H]1CN(C(=O)OC(C)(C)C)[C@@H](C)CN1C1CC1 ; C[C@@H]1CN(C2CC2)C[C@H](C)N1C(=O)OC(C)(C)C ; CC(C)[C@@H]1CN(C2CC2)CCN1C(=O)OC(C)(C)C ; CC(C)[C@H]1CN(C2CC2)CCN1C(=O)OC(C)(C)C ; CC(C)(C)OC(=O)N1C2CCC1CN(C1CC1)C2]

In accordance with certain embodiments, compounds of Formula XIV are selected from compounds of Formula XIVa-l as those substituted piperazines herein represented:

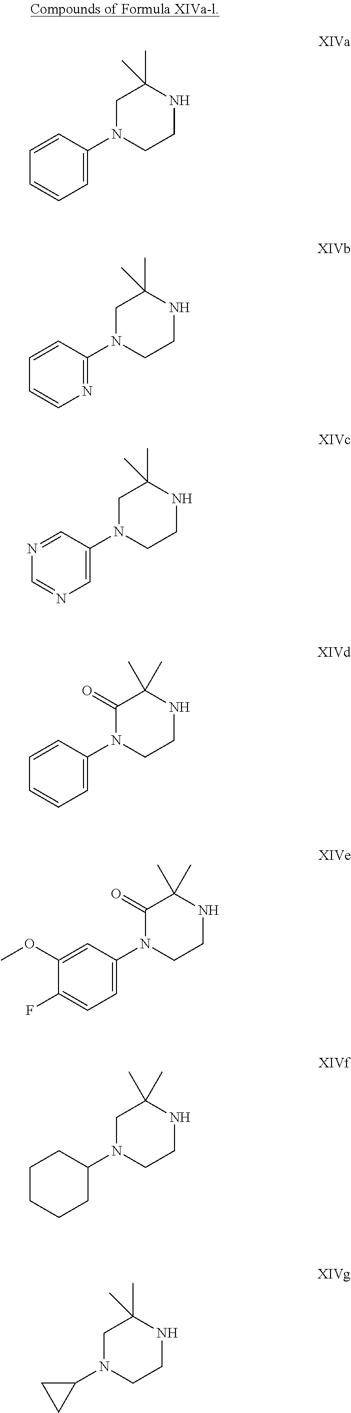

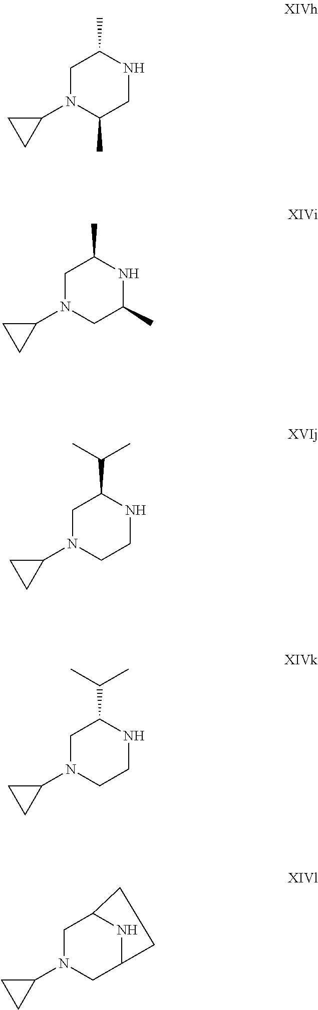

According to Scheme 7, compounds of Formula I-Aa wherein R¹¹ and R¹² are both CH₃, X^{A} is H and R¹³ and R¹⁴ are independently selected from H, optionally substituted (C1-C6)alkyls, optionally substituted aryls, and optionally substituted (C1-C6)alkyl-aryls, and R¹⁵ is as defined above, can be prepared starting from compounds of Formula V-Aa.

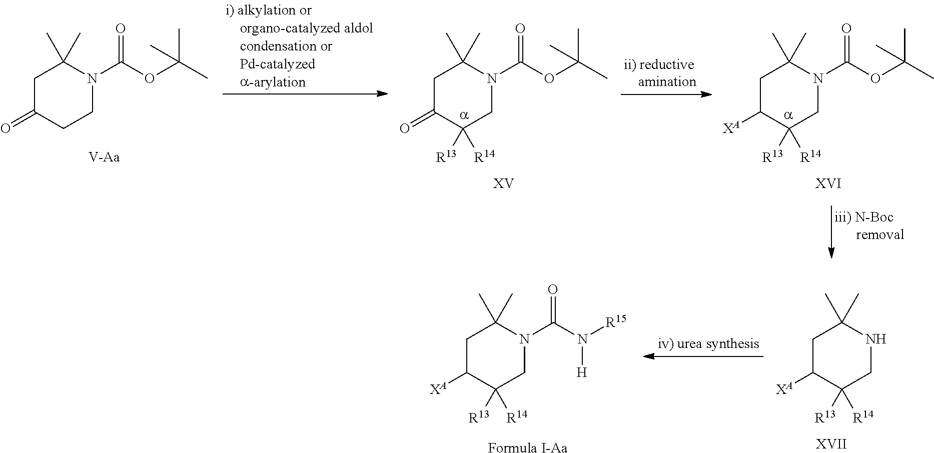

In accordance with certain embodiments, compounds of Formula XV are selected from compounds of Formula XVa-d as those substituted piperidones herein represented:

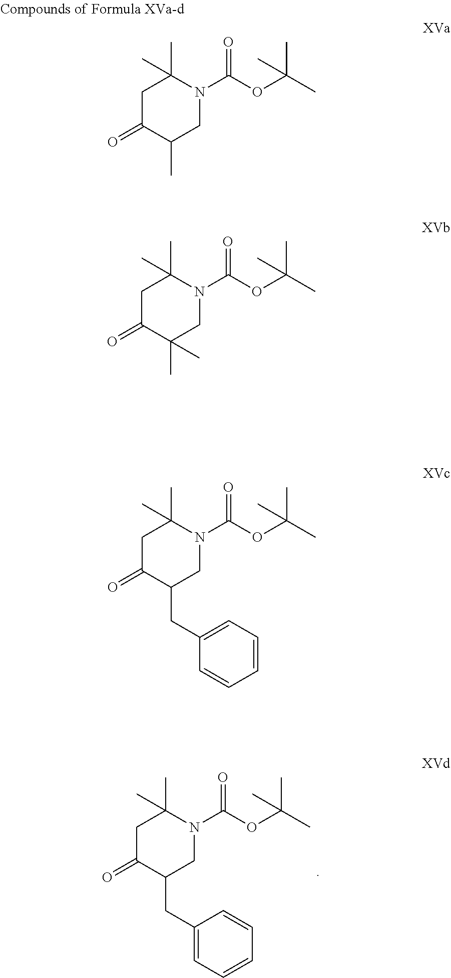 [SMILES: CC1CN(C(=O)OC(C)(C)C)C(C)(C)CC1=O ; CC(C)(C)OC(=O)N1CC(C)(C)C(=O)CC1(C)C ; CC(C)(C)OC(=O)N1CC(Cc2ccccc2)C(=O)CC1(C)C ; CC(C)(C)OC(=O)N1CC(Cc2ccccc2)C(=O)CC1(C)C ; "."]

In accordance with certain embodiments, compounds of Formula XVI are selected from compounds of Formula XVIa-c as those substituted piperidines herein represented:

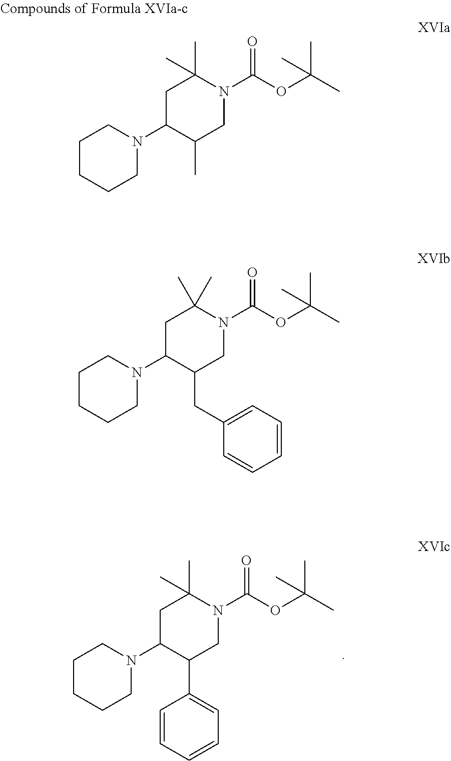 [SMILES: CC1CN(C(=O)OC(C)(C)C)C(C)(C)CC1N1CCCCC1 ; CC(C)(C)OC(=O)N1CC(Cc2ccccc2)C(N2CCCCC2)CC1(C)C ; CC(C)(C)OC(=O)N1CC(c2ccccc2)C(N2CCCCC2)CC1(C)C ; "."]

In accordance with certain embodiments, compounds of Formula XVII are selected from compounds of Formula XVIIa-c as those substituted piperidines herein represented:

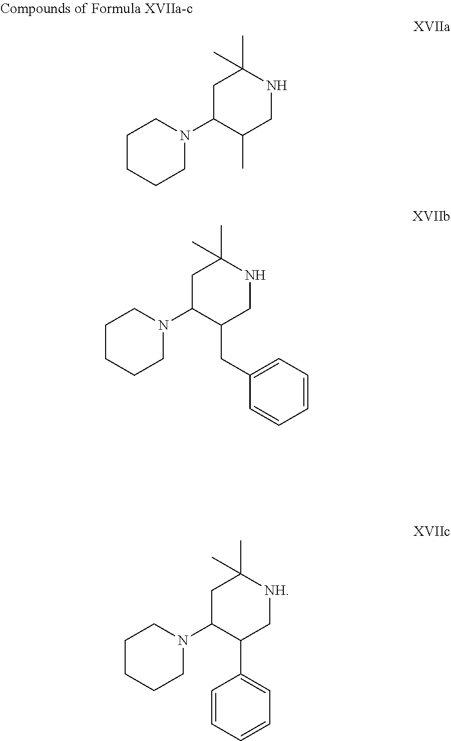 [SMILES: CC1CNC(C)(C)CC1N1CCCCC1 ; CC1(C)CC(N2CCCCC2)C(Cc2ccccc2)CN1 ; CC1(C)CC(N2CCCCC2)C(c2ccccc2)CN1]

According to Scheme 8, compounds of Formula I-Aa wherein R¹¹ and R¹² are both selected from CH₃, R¹³ and R¹⁴ are independently selected from H and ═CR^{Aa}R^{Ab}, and X^{A}, R^{Aa}, R^{Ab} and R¹⁵ are defined above can be prepared, starting from compounds of Formula V-Aa.

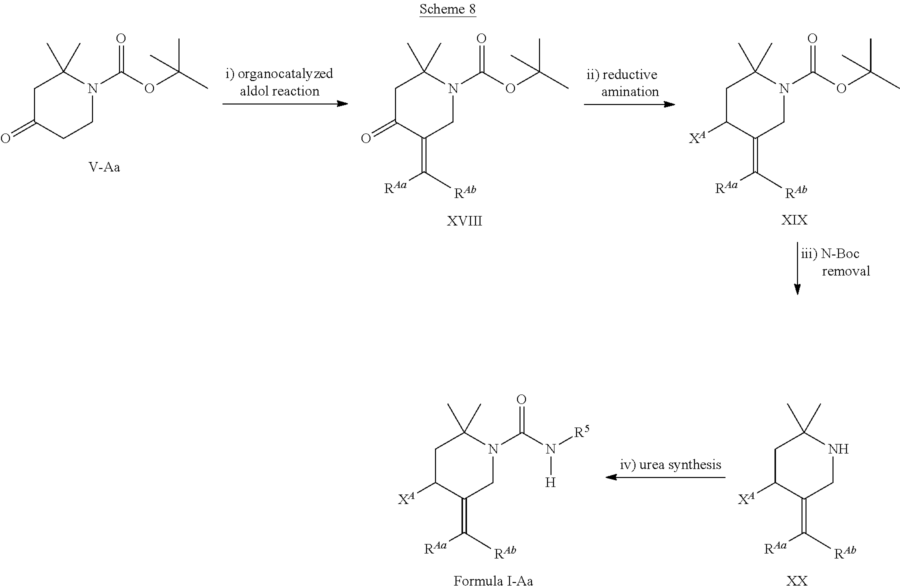

In accordance with certain embodiments, compounds of Formula XVIII, XIX and XX are selected from compounds of Formula XVIIIa, XIXa and XXa as those substituted piperidines herein represented:

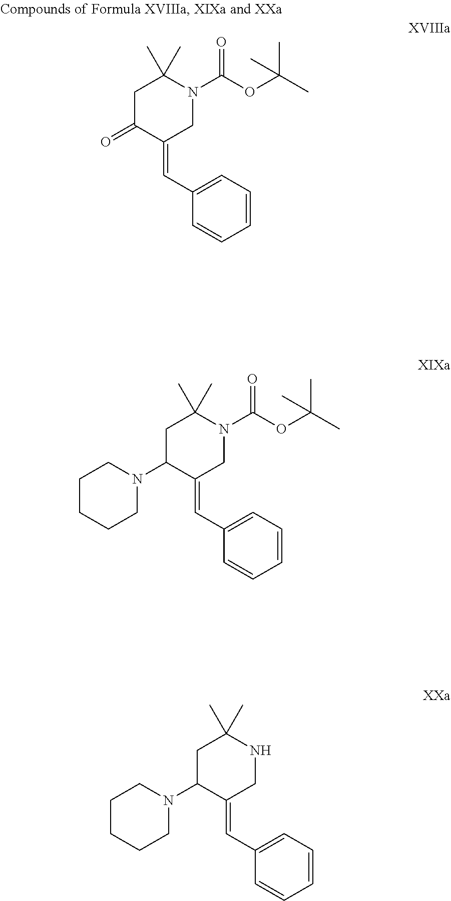 [SMILES: CC(C)(C)OC(=O)N1C/C(=C\c2ccccc2)C(=O)CC1(C)C ; CC(C)(C)OC(=O)N1C/C(=C\c2ccccc2)C(N2CCCCC2)CC1(C)C ; CC1(C)CC(N2CCCCC2)/C(=C/c2ccccc2)CN1]

According to Scheme 9, compounds of Formula I-Ab, wherein R¹¹ and R¹² are both selected from CH₃, and R¹³ and R¹⁴ are independently selected from H, optionally substituted (C1-C6)alkyls, optionally substituted aryls, and optionally substituted (C1-C6)alkyl-aryls, and R¹⁵ is as defined above, can be prepared starting from compounds of Formula V-Aa.

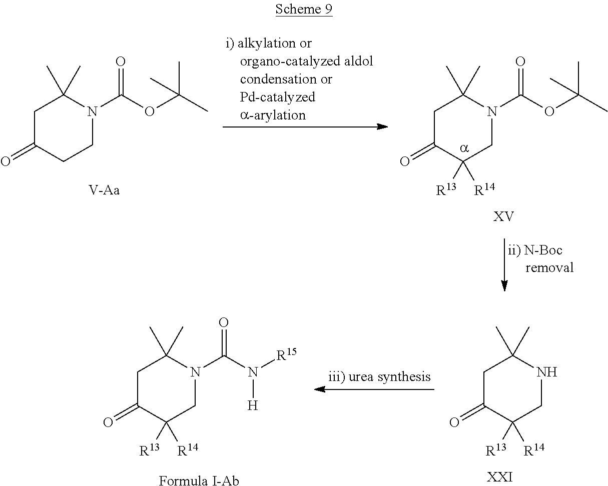

In accordance with certain embodiments, compounds of Formula XXI are selected from compounds of Formula XXIa-d as those substituted ketones herein represented:

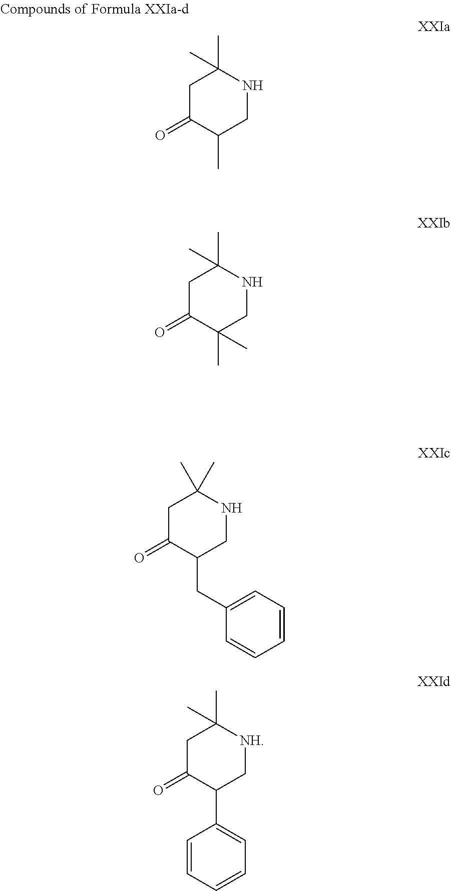

According to Scheme 10, compounds of Formula I-Ab wherein R¹¹ and R¹² are both selected from CH₃, and R¹³ and R¹⁴ are independently selected from H and ═CR^{Aa}R^{Ab}, and R^{Aa}, R^{Ab} and R¹⁵ are as defined above, can be prepared, starting from compounds of Formula V-Aa.

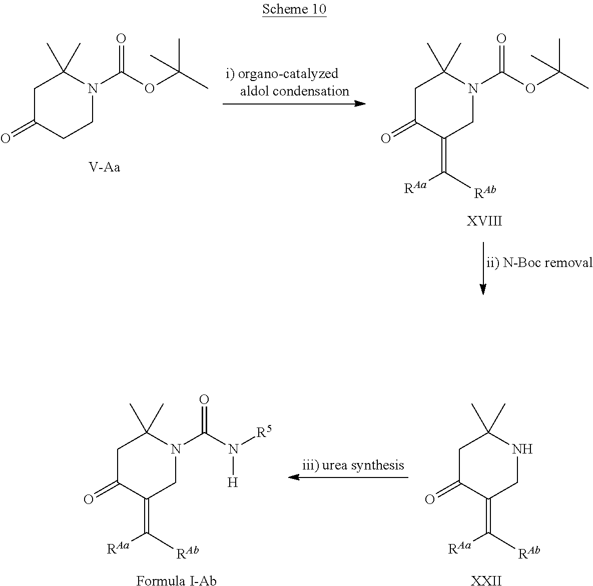

In accordance with certain embodiments, compounds of Formula XVIII and XXII are selected from compounds of Formula XVIIIa and XXIIa as those substituted ketones herein represented:

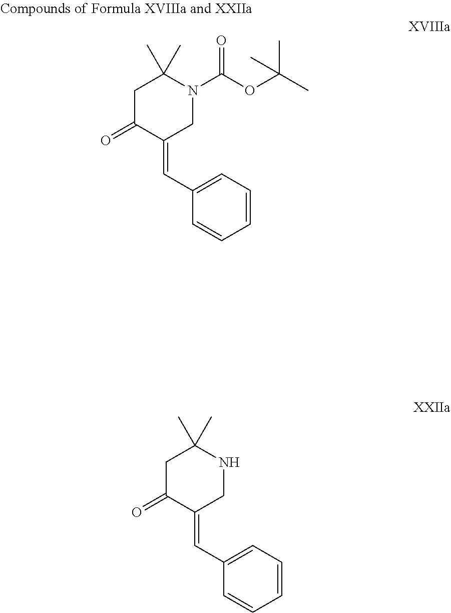 [SMILES: CC(C)(C)OC(=O)N1C/C(=C\c2ccccc2)C(=O)CC1(C)C ; CC1(C)CC(=O)/C(=C/c2ccccc2)CN1]

According to Scheme 11, compounds of Formula I-Ab wherein Y^{A} is selected from CH₂, O, and N-Cbz, and R^{Ac}, R¹¹, R¹², R¹³, R¹⁴, R¹⁵ and X^{A} are as defined above, can be prepared, starting from compounds of Formula XXIII.

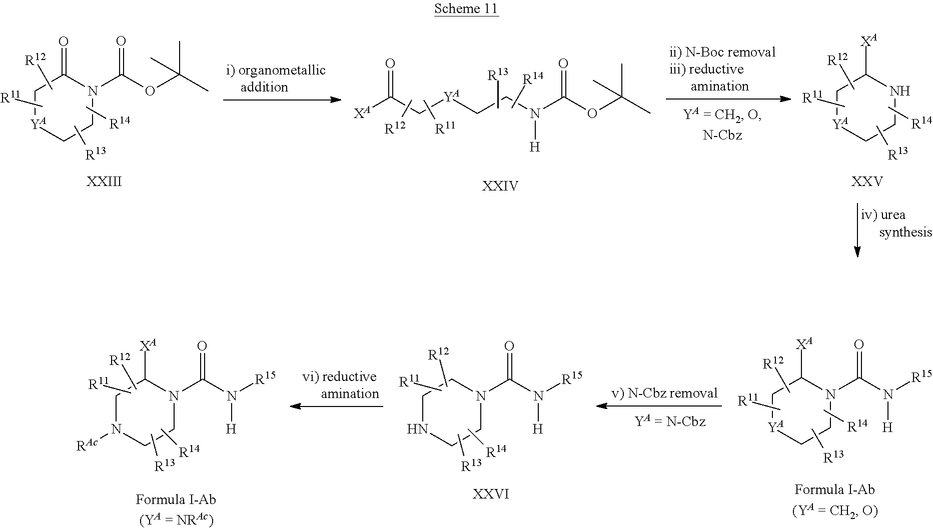

Alternative to Scheme 11 and according to Scheme 12, compounds of Formula I-Ab wherein Y^{A} is N-Cbz, and R^{Ac}, R¹¹, R¹², R¹³, R¹⁴, R¹⁵ and X^{A} are as defined above, can be prepared, starting from compounds of Formula XXIII wherein Y^{A}=CBz.

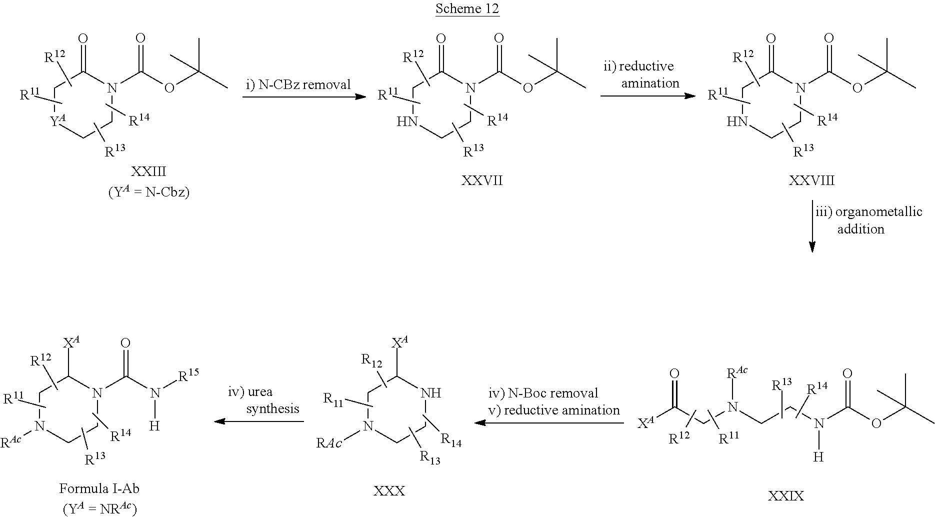

According to Scheme 13, compounds of Formula I-Ab wherein Y^{A} is selected from N-Cbz, and R^{Ac}, R¹¹, R¹², R¹³, R¹⁴, R¹⁵ and X^{A} are as defined above, can be prepared, starting from compounds of Formula XXV.

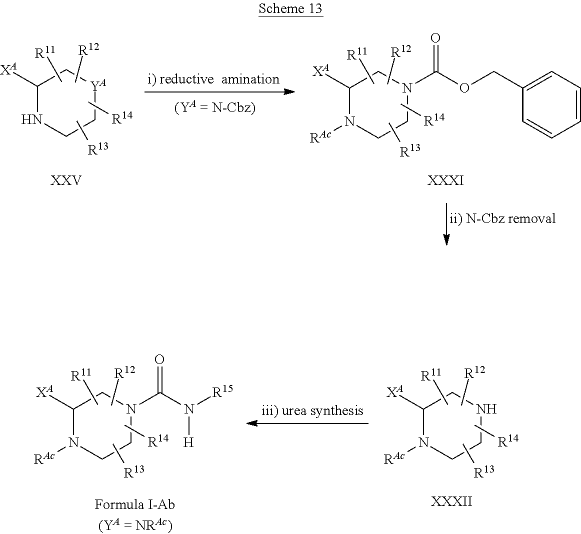

In accordance with certain embodiments, compounds of Formula XXIII are selected from compounds of Formula XXIIIa-e as those substituted N-Boc lactames herein represented:

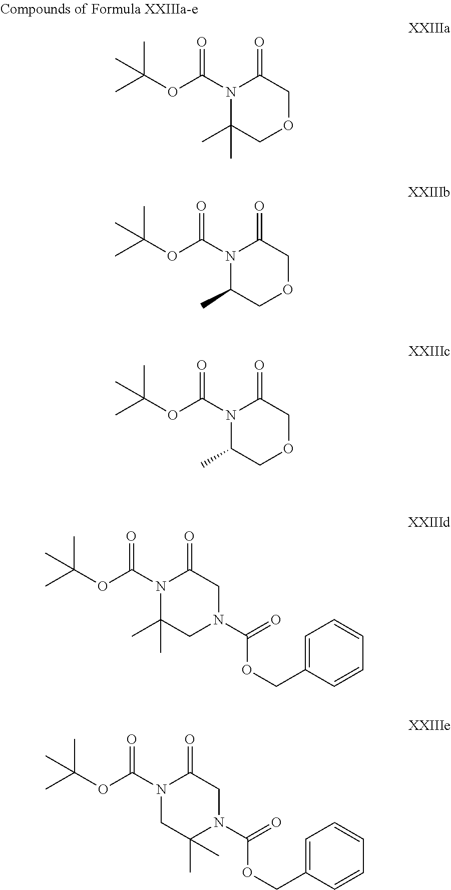 [SMILES: CC(C)(C)OC(=O)N1C(=O)COCC1(C)C ; C[C@@H]1COCC(=O)N1C(=O)OC(C)(C)C ; C[C@H]1COCC(=O)N1C(=O)OC(C)(C)C ; CC(C)(C)OC(=O)N1C(=O)CN(C(=O)OCc2ccccc2)CC1(C)C ; CC(C)(C)OC(=O)N1CC(C)(C)N(C(=O)OCc2ccccc2)CC1=O]

In accordance with certain embodiments, compounds of Formula XXIV and XXIX are selected from compounds of Formula XXIVa-n and XXIXa as those ketones herein represented:

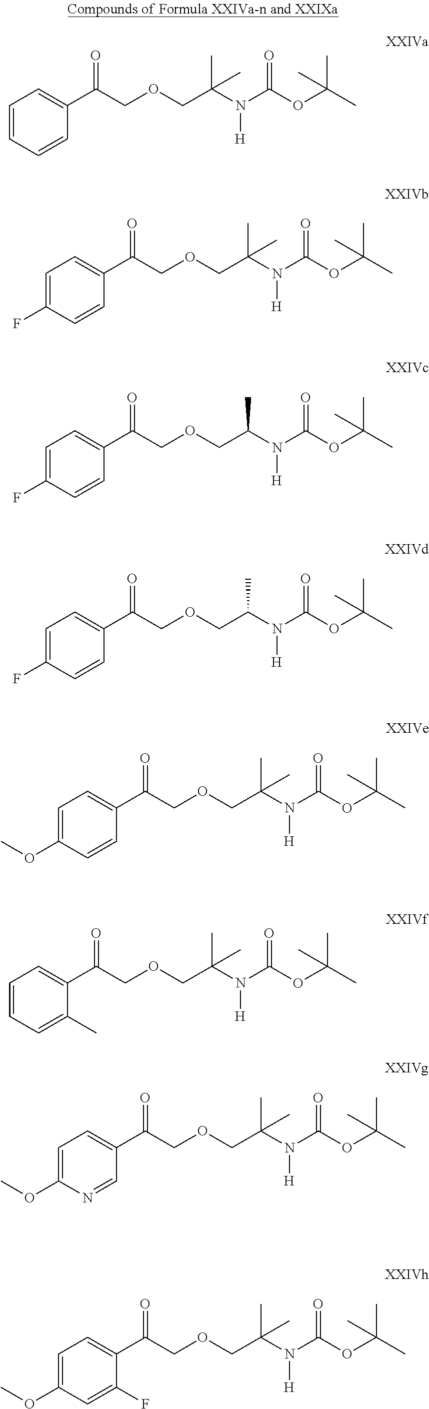

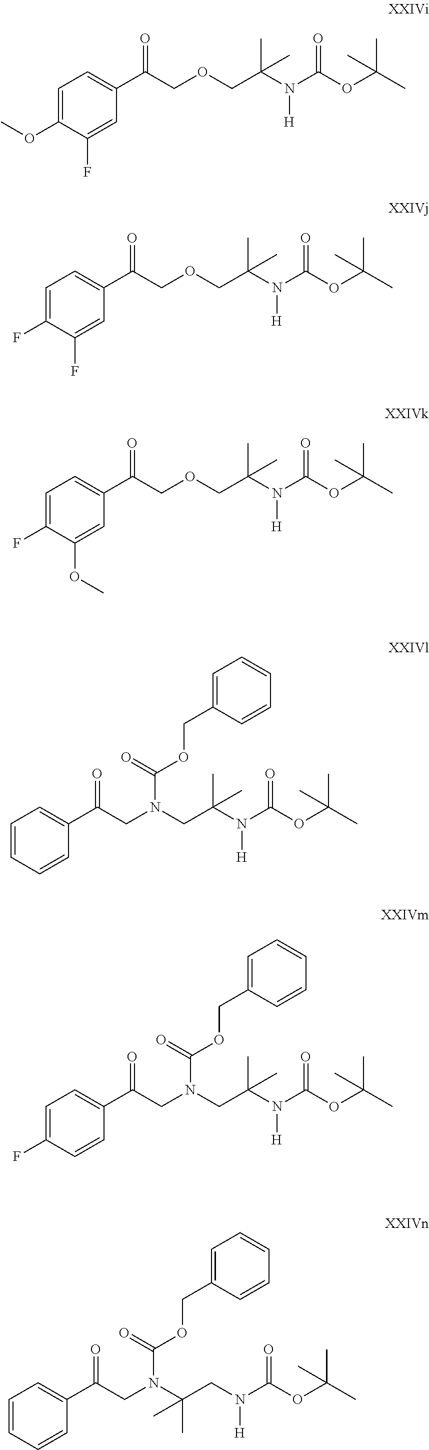 [SMILES: COc1ccc(C(=O)COCC(C)(C)NC(=O)OC(C)(C)C)cc1F ; CC(C)(COCC(=O)c1ccc(F)c(F)c1)NC(=O)OC(C)(C)C ; COc1cc(C(=O)COCC(C)(C)NC(=O)OC(C)(C)C)ccc1F ; CC(C)(CN(CC(=O)c1ccccc1)C(=O)OCc1ccccc1)NC(=O)OC(C)(C)C ; CC(C)(CN(CC(=O)c1ccc(F)cc1)C(=O)OCc1ccccc1)NC(=O)OC(C)(C)C ; CC(C)(C)OC(=O)NCC(C)(C)N(CC(=O)c1ccccc1)C(=O)OCc1ccccc1]

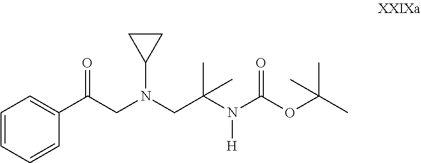 [SMILES: CC(C)(CN(CC(=O)c1ccccc1)C1CC1)NC(=O)OC(C)(C)C]

In accordance with certain embodiments, compounds of Formula XXV are selected from compounds of Formula XXVa-n as those morpholines and piperazines herein represented:

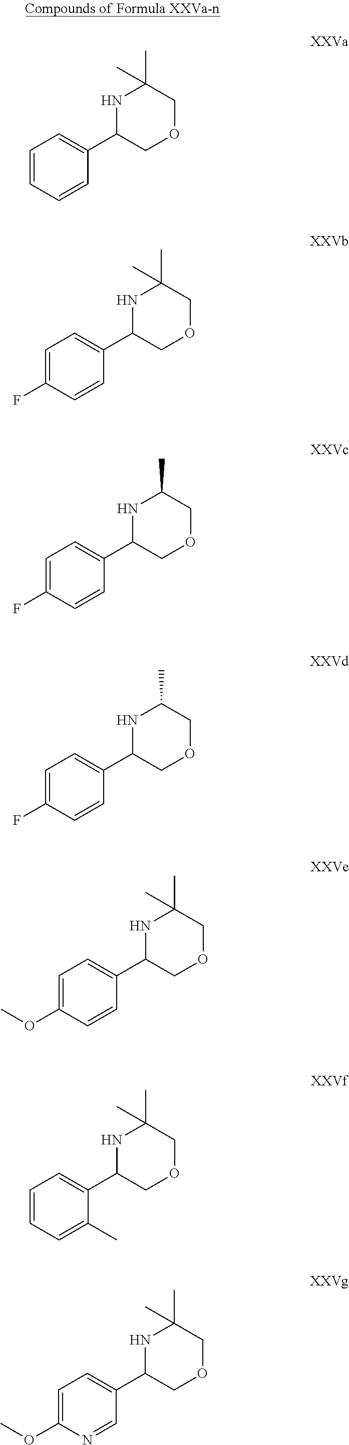

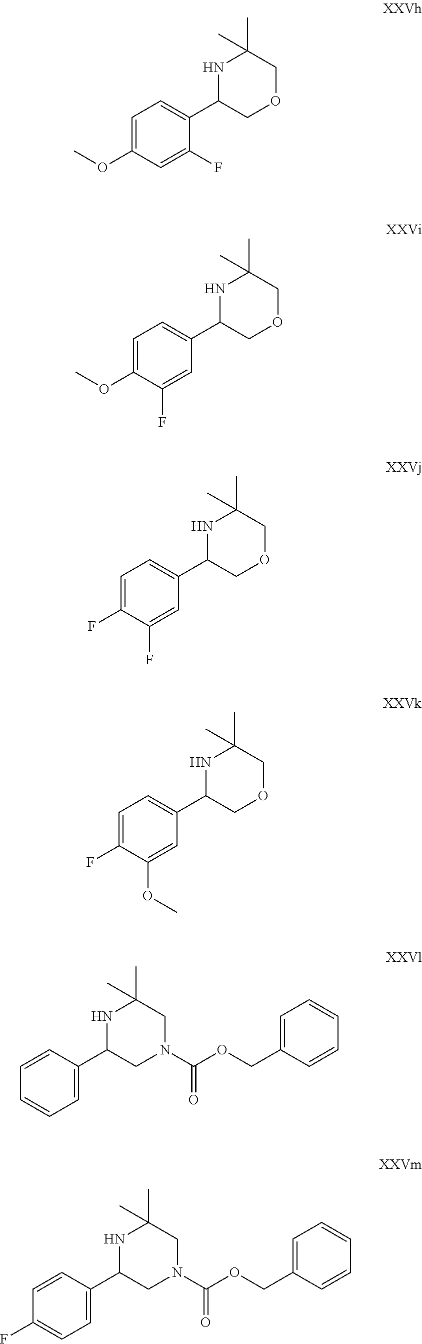 [SMILES: COc1ccc(C2COCC(C)(C)N2)c(F)c1 ; COc1ccc(C2COCC(C)(C)N2)cc1F ; CC1(C)COCC(c2ccc(F)c(F)c2)N1 ; COc1cc(C2COCC(C)(C)N2)ccc1F ; CC1(C)CN(C(=O)OCc2ccccc2)CC(c2ccccc2)N1 ; CC1(C)CN(C(=O)OCc2ccccc2)CC(c2ccc(F)cc2)N1]

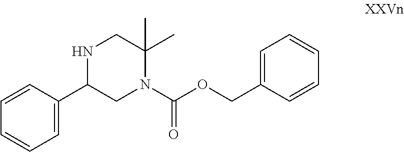 [SMILES: CC1(C)CNC(c2ccccc2)CN1C(=O)OCc1ccccc1]

In accordance with certain embodiments, compounds of Formula XXVI and XXXI are selected from compounds of Formula XXVIa-c, XXXIa and XXXIIa-c as those piperazines herein represented:

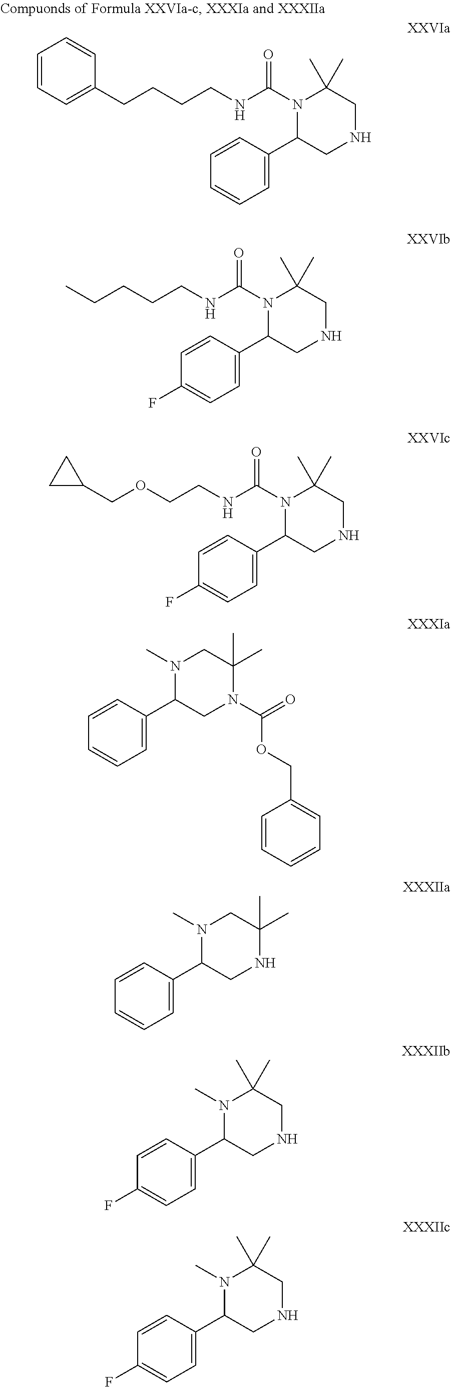

In accordance with certain embodiments, compounds of Formula XXVII and XXVIII are selected from compounds of Formula XXVIIa and XXVIIIa as those substituted N-Boc lactames herein represented:

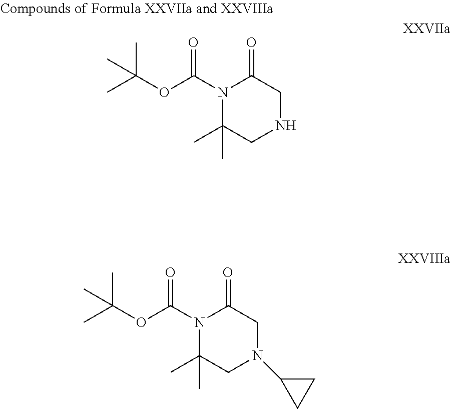 [SMILES: CC(C)(C)OC(=O)N1C(=O)CNCC1(C)C ; CC(C)(C)OC(=O)N1C(=O)CN(C2CC2)CC1(C)C]

### Synthesis of Compounds of Formula (I-B).

Also described herein are methods of synthesizing the compounds represented by formula (I-B):

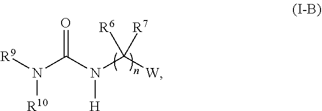

or a pharmaceutically acceptable salt thereof, wherein:

R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;

n is an integer selected from 0 to 6; and

W is selected from the group consisting of methyl, methylene (i.e.,

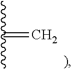

halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃); and

R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl.

According to Scheme 14 (Method A′), compounds of Formula (I-B), wherein R⁶, R⁷, n, W, R⁹, and R¹⁰, are as defined above can be prepared, by reaction of substituted amines A1, wherein R⁹ and R¹⁰ are as defined in Formula (I-B), and isocyanates A2, wherein R⁶, R⁷, n, and W are as defined in Formula (I-B), in the presence of catalytic amount of a base, e.g., 4-dimethylamino pyridine (DMAP), in polar solvent, such as acetonitrile (CH₃CN).

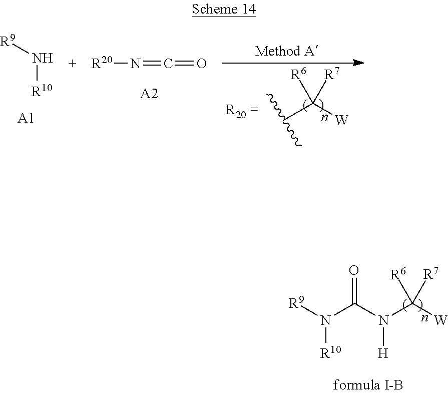

Amines A1, wherein R⁹ and R¹⁰ are as defined in Formula (I-B), and isocyanates A2, wherein R⁶, R⁷, n, and W are as defined in Formula (I-B) are commercially available or can be prepared from commercially available compounds according to general synthetic procedures described for instance in Michael Smith, Jerry March—March's Advanced Organic Chemistry: reactions mechanism and structure—6th Edition, John Wiley & Sons Inc., 2007, or in Molina P., Tarraga A., Arques A. in Katritzky A. R., Taylor R. J. K., Comprehensive Organic Functional Group Transformations II, Elsevier, 2004, Vol. 5, pag. 949-973, and references cited therein, which are herein incorporated by reference.

### III. Pharmaceutical Compositions

The invention provides pharmaceutical compositions comprising a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)) or related compound described herein. In certain embodiments, the pharmaceutical compositions preferably comprise a therapeutically-effective amount of one or more of a compound described herein (e.g., a compound of Formula (I), (I-Aa), (I-Ab), (II), (II), (IV), (V), (VI), (I-B) (I-B), (I-C), (I-D), (I-E), (I-F), (I-G), or (I-H)), formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like, and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention (e.g., a compound of Formula (I), (I-Aa), (I-Ab), (II), (III), (IV), (V), (VI), (I-B) (I-B), (I-C), (I-D), (I-E), (I-F), (I-G), or (I-H)).

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### IV. Methods of Use

Sphingolipids are a family of membrane lipids derived from the aliphatic amino alcohol sphingosine and its related sphingoid bases. They are present in eukaryote membranes, where they exert important structural roles in the regulation of fluidity and subdomain structure of the lipid bilayer. In addition to serving roles in cell membrane structure and dynamics, sphingolipids also serve important signaling functions, for example, in the control of cell growth, cell differentiation, and cell death, and can be important for cell homeostasis and development. Zeidan et al. (2010) supra, Proksch et al. (2011) supra. Ceramide, a key member of this lipid class, has attracted attention in view of its impact on the replication and differentiation of neoplastic cells. Furuya et al. (2011) supra. For example, lower levels of ceramide have been discovered in several types of human tumors relative to normal tissue, where the level of ceramide appears to correlate inversely with the degree of malignant progression. Realini et al. (2013) supra.

Acid ceramidase is a cysteine amidase that catalyzes the hydrolysis of ceramide into sphingosine and fatty acid and is believed to be involved in the regulation of ceramide levels in cells and modulates the ability of this lipid messenger to influence the survival, growth and death of certain tumor cells. Id. Furthermore, acid ceramidase enzymes are abnormally expressed in various types of human cancer (e.g., prostate, head and neck, and colon) and serum AC levels are elevated in patients with melanoma relative to control subjects. Id.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimer's, Parkinson's, Huntington's, and amyotrophic lateral sclerosis). See, International Application Publication Nos. WO2016/210116 and WO2016/210120.

It is contemplated that the compounds, compositions, and methods disclosed herein can be used to treat various disorders associated or correlated with elevated levels of acid ceramidase activity. The invention provides administering to a subject in need thereof an effective amount of a compound or composition disclosed herein, either alone or in a combination with another therapeutic agent to treat the disorder.

In certain embodiments, the compound or composition used in one or more of the methods described herein is one of the generic or specific compounds described in Section II, such as a compound of Formula (I), a compound embraced by one of the further embodiments describing definitions for certain variables of Formula (I), a compound of Formula (I-Aa) or (I-Ab), (II), (III), (IV), (V), (VI), (I-B), (I-C), (I-D), (I-E), (I-F), (I-G), or (I-H), or a compound embraced by one of the further embodiments describing definitions for certain variables of Formula (II), (III), (IV), (V), (VI), (I-B), (I-C), (I-D), (I-E), (I-F), (I-G), or (I-H).

In certain embodiments, a method or composition described herein, is administered in combination with one or more additional therapies, e.g., surgery, radiation therapy, or administration of another therapeutic preparation. In certain embodiments, the additional therapy may include an additional therapeutic agent. The invention embraces combination therapy, which includes the administration of a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or composition described herein and a second treatment and/or agent as part of a specific treatment regimen intended to provide the beneficial effect from the co-action of the foregoing. The beneficial effect of the combination may include pharmacokinetic or pharmacodynamic co-action resulting from the foregoing combination of agents and/or treatments.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

I. Cancer. Inflammation and Other Disorders

The compositions and methods disclosed herein can be used to treat various disorders associated or otherwise correlated with elevated levels of acid ceramidase activity. Exemplary disorders include cancer, inflammation, pain and inflammatory pain, or a pulmonary disease.

In certain embodiments, the compositions and methods disclosed herein can be used to treat cancer or inhibit cancer growth in a subject in need thereof. The invention provides a method of treating a cancer in a subject. The method comprises administering to the subject an effective amount of a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein, either alone or in a combination with another therapeutic agent to treat the cancer in the subject.

Exemplary cancers include, but are not limited to, pre-malignant conditions, for example hyperplasia, metaplasia or dysplasia, cancer metastasis, benign tumors, angiogenesis, hyperproliferative disorders and benign dysproliferative disorders. The treatment may be prophylactic or therapeutic. The subject to be treated may be human or a non-human animal (e.g., a non-human primate or a non-human mammal).

In certain embodiments, a compound disclosed herein (e.g., a compound of Formula (I), (I-Aa), (I-Ab), (II), (III), (IV), (V), (VI), (I-B) (I-B), (I-C), (I-D), (I-E), (I-F), (I-G), or (I-H)) or a pharmaceutical composition containing such a compound, can be used to treat a disorder involving primary and/or metastatic neoplastic disease.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma)

In certain embodiments, the present invention provides a compound disclosed herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein for the use in the treatment and/or prevention of brain cancer, breast cancer, colon cancer, head and neck cancer, liver cancer, lung cancer (e.g., alveolar cancer), pancreatic cancer, prostate cancer, skin cancer (e.g., melanoma).

It is contemplated that the compounds disclosed can be used in combination with other treatments and/or therapeutic agents. The invention embraces combination therapy, which includes the administration of a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or related compound described herein and a second treatment and/or agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

In certain embodiments, a compound or pharmaceutical composition described herein, is administered in combination with one or more additional cancer therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or pharmaceutical composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody-based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MED14736, avelumab, and BMS 936559 (Bristol Myers Squibb Co.).

In certain embodiments, a compound or pharmaceutical composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

Exemplary cytotoxic agents that can be administered in combination with a compound or pharmaceutical composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a compound or pharmaceutical composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In certain embodiments, a compound disclosed herein (e.g., a compound of Formula (I), (I-Aa), (I-A b), (II), (III), (IV), (V), (VI), (I-B) (I-B), (I-C), (I-D), (I-E), (I-F), (I-G), or (I-H)) or a pharmaceutical composition containing such a compound, can be used to treat an inflammatory condition, such as rheumatoid arthritis and ulcerative cholitis. The invention provides a method of treating an inflammatory condition. The method comprises administering to the subject an effective amount of a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein, either alone or in a combination with another therapeutic agent to treat the inflammatory condition in the subject.

As used herein, an inflammatory condition is a disease or condition characterized, in whole or in part, by inflammation or an inflammatory response in the patient. Typically, one or more of the symptoms of the inflammatory disease or condition is caused or exacerbated by an inappropriate, misregulated, or overactive inflammatory response. Inflammatory diseases or conditions may be chronic or acute. In certain embodiments, the inflammatory disease or condition is an autoimmune disorder.

Inflammatory conditions treatable using a compound or pharmaceutical composition disclosed herein may be characterized, for example, based on the primary tissue affected, the mechanism of action underlying the condition, or the portion of the immune system that is misregulated or overactive. Examples of inflammatory conditions, as well categories of diseases and conditions are provided herein. In certain embodiments, examples of inflammatory conditions that may be treated include inflammation of the lungs, joints, connective tissue, eyes, nose, bowel, kidney, liver, skin, central nervous system, vascular system, heart, or adipose tissue. In certain embodiments, inflammatory conditions which may be treated include inflammation due to the infiltration of leukocytes or other immune effector cells into affected tissue. In certain embodiments, inflammatory conditions which may be treated include inflammation mediated by IgE antibodies. Other relevant examples of inflammatory conditions which may be treated by the present disclosure include inflammation caused by infectious agents, including but not limited to viruses, bacteria, fungi, and parasites. In certain embodiments, the inflammatory condition that is treated is an allergic reaction. In certain embodiments, the inflammatory condition is an autoimmune disease.

Inflammatory lung conditions include asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Inflammatory eye conditions include uveitis (including iritis), conjunctivitis, scleritis, and keratoconjunctivitis sicca. Inflammatory bowel conditions include Crohn's disease, ulcerative colitis, inflammatory bowel disease, and distal proctitis. Inflammatory skin conditions include conditions associated with cell proliferation, such as psoriasis, eczema, and dermatitis (e.g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis). Inflammatory conditions of the endocrine system include, but are not limited to, autoimmune thyroiditis (Hashimoto's disease), Type I diabetes, inflammation in liver and adipose tissue associated with Type II diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, atherosclerosis, and vascular disease associated with Type II diabetes. Inflammatory conditions of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia. Inflammatory conditions of the liver include, but are not limited to, hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis. In certain embodiments, the inflammatory condition is an autoimmune disease, for example, rheumatoid arthritis, lupus, alopecia, autoimmune pancreatitis, Celiac disease, Behcet's disease, Cushing syndrome, and Grave's disease. In certain embodiments, the inflammatory condition is a rheumatoid disorder, for example, rheumatoid arthritis, juvenile arthritis, bursitis, spondylitis, gout, scleroderma, Still's disease, and vasculitis.

In certain embodiments, the present invention provides a compound disclosed herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition containing a compound disclosed herein for use in the treatment of a pain syndrome, disorder, disease or condition characterized by nociceptive pain, neuropathic pain, inflammatory pain, non-inflammatory pain, pain associated with acute conditions such as post-operative or post-traumatic stress disorders, pain associated with chronic conditions such as diabetes. The invention provides a method of treating pain. The method comprises administering to the subject an effective amount of a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)) or a pharmaceutical composition disclosed herein, either alone or in a combination with another therapeutic agent to treat the pain in the subject.

A compound or composition described herein can be useful for the treatment (including prevention and/or alleviation) of chronic and/or acute pain, in particular non-inflammatory musculoskeletal pain such as back pain, fibromyalgia and myofascial pain, more particularly for reduction of the associated muscular hyperalgesia or muscular allodynia. Non-limiting examples of types of pain that can be treated by a compound or composition disclosed includes chronic conditions such as musculoskeletal pain, including fibromyalgia, myofascial pain, back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS (chronic pain syndrome), central pain, neuropathic pain such as trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiency or alcoholism; and acute pain such as pain after injuries, postoperative pain, pain during acute gout or pain during operations, such as jaw surgery.

In certain embodiments, the present invention provides a compound disclosed herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein for use in the treatment of a pulmonary disease, such as asthma, chronic obstructive pulmonary disease (COPD), adult respiratory disease, acute respiratory distress syndrome, chronic bronchitis, and emphysema. The invention provides a method of treating a pulmonary disease. The method comprises administering to the subject an effective amount of a compound disclosed herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein, either alone or in a combination with another therapeutic agent to treat the pulmonary disease in the subject.

II. Lysosomal Storage Disorders

Lysosomal storage disorders (LSDs) are a group of more than 50 clinically-recognized, rare inherited metabolic disorders that result from defects in lysosomal function (Walkley, J. (2009) INHERIT. METAB. DIS., 32(2): 181-9). LSDs are caused by dysfunction of the cell's lysosomes, which are heterogeneous subcellular organelles containing specific hydrolases that allow targeted processing or degradation of proteins, nucleic acids, carbohydrates, and lipids (HARRISON'S PRINCIPLES OF INTERNAL MEDICINE, 16^{th} Edition, vol. II, Chapter 20, pp. 2315-2319). The lysosome encloses an acidic environment and contains enzymes that catalyze the hydrolysis of biological macromolecules.

Individually, LSDs occur with incidences of less than 1:100,000, however, as a group the incidence is as high as 1 in 1,500 to 7,000 live births (Staretz-Chacham, et al. (2009) PEDIATRICS, 123(4): 1191-207). LSDs typically are caused by inborn genetic errors. Affected individuals generally appear normal at birth, however the diseases are progressive. The development of clinical disease may not occur until years or decades later but is typically fatal.

It is believed that sphingosine-containing analogs (for example, glucosylsphingosine, galactosphingosine, lactosylsphingosine, GB3-sphingosine, and GM2-sphingosine) may accumulate in cells of subjects with certain lysosomal storage disorders or LSDs (for example, Gaucher's disease, Krabbe disease, multiple sclerosis, Fabry's disease, and Tay Sachs disease, respectively) and that the accumulation of these sphingosine-containing analogs may contribute to the disease phenotype. See, e.g., International Application Publication No. WO2016/210116. Given that such sphingosine-containing analogs are often produced by acid ceramidase enzymes in the lysosomal compartments of cells in subjects with LSDs, the accumulation of the sphingosine-containing analogs to detrimental levels can be prevented or reduced by the use of an effective amount of one or more of the acid ceramidase inhibitors described herein.

In certain embodiments, a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)), or pharmaceutical composition containing a compound disclosed herein can be used to treat a LSD in a subject in need thereof. The invention provides a method of treating a LSD in a subject. The method comprises administering to the subject an effective amount of a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein, either alone or in a combination with another therapeutic agent to treat the LSD in the subject.

Exemplary LSDs include, for example, Krabbe disease, Fabry disease, Tay-Sachs disease, Sandhoff Variant A, or B, Pompe disease, Hunter's syndrome, Niemann Pick disease Types A and B, and Gaucher's disease.

It is contemplated that the compounds disclosed can be used in combination with other treatments and/or therapeutic agents. The invention embraces combination therapy, which includes the administration of a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or related compound described herein and a second treatment and/or agent as part of a specific treatment regimen intended to provide the beneficial effect from the co-action of these therapeutic agents. Exemplary second agents for use in treating Gaucher disease include, for example, imiglucerase (CEREZYME®), taliglucerase alfa (ELELYSO®), velaglucerase alfa (VPRIV®), eliglustat (CERDELGA®), and miglustat (ZAVESCA®) or a glucocerebrosidase activator such as one or more of the compounds described in International Application Publication No. WO2012/078855. Exemplary second agents for use in treating Fabry disease include, for example, alpha-galactosidase A (FABRAZYME®). Additional acid ceramidase inhibitors for use in combination therapies include, for example, those described in International Patent Application Publications WO 2015/173168 and WO 2015/173169, each of which are hereby incorporated by reference.

III. Neurodegenerative Disorders

Neurodegenerative disorders often are associated with reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Examples of neurodegenerative disorders include, for example, Alzheimer's disease, Parkinson's disease, Huntington's disease, amyotrophic lateral sclerosis (ALS; also known as Lou Gehrig's disease or motor neuron disease), multiple sclerosis, and diffuse Lewy body disease. Once clinical expression occurs, the neurodegenerative disorder may be associated with impairment of motor function, for example, as observed in subjects with Parkinson's disease, Huntington's disease multiple sclerosis, or ALS. Alternatively or in addition, neurodegenerative disorders may be associated with cognitive impairment and/or the loss of cognitive function, for example, as observed in subjects with Alzheimer's disease.

Alzheimer's disease is a central nervous system (CNS) disorder that results in memory loss, unusual behavior, personality changes, and a decline in thinking abilities. These losses are related to the death of specific types of brain cells and the breakdown of connections and their supporting network (e.g., glial cells) between them. The earliest symptoms include loss of recent memory, faulty judgment, and changes in personality. Parkinson's disease is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. Huntington's disease is another neurodegenerative disease that causes uncontrolled movements, loss of intellectual faculties, and emotional disturbance.

It has been observed that subjects with certain mutant alleles in genes encoding β-glucocerebrosidase activity (the GBA gene; Aharon-Peretz (2004) NEW. ENG. J. MED. 351: 1972-1977; Gan-Or et al. (2008) NEUROLOGY 70:2277-2283; Gan-Or et al. (2015) NEUROLOGY 3:880-887) and sphinomyelinase activity (the SMPD1 gene, Gan-Or et al. (2013) NEUROLOGY 80:1606-1610) have been associated with, and identified as a risk factor for, Parkinson's Disease. As a result, defects with, or deficiencies in the activities of these enzymes, as in the case of Gaucher's disease and Niemann Pick types A and B, can cause an accumulation of glucosylceramide and sphingomyelin, which can then be converted to glucosylsphingosine or lyso-sphingomyelin, respectively, via acid ceramidase activity. The accumulation of glucosylsphingosine or lyso-sphingomyelin may thus be implicated in the development of Parkinson's disease. It is contemplated that the administration of an acid ceramidase inhibitor, which slows down, stops or reverses the accumulation of glucosylsphingosine and/or lyso-sphingomyelin can be used to treat Parkinson's Disease. For example, an acid ceramidase inhibitor can be used to improve motor and/or memory impairments symptomatic of Parkinson's disease.

Similarly, it has been observed that lactosylceramide (LacCer) is upregulated in the central nervous system of mice during chronic experimental autoimmune encephalomyelitis (EAE), a model of multiple sclerosis (Lior et al. (2014) NATURE MEDICINE 20:1147-1156). It is contemplated that the increase in LacCer may also result in an increase in lactosylsphingosine (LacSph) via conversion by an acid ceramidase (a lactosylceramide to lactosylsphingosine converting enzyme). Given the accumulation of lactosylsphingosine to a toxic or otherwise detrimental level or concentration in the lysosomal compartments of cells in subjects with multiple sclerosis, it is contemplated that the administration of an acid ceramidase inhibitor can reduce the accumulation of lactosylsphingosine thereby treating multiple sclerosis, which includes ameliorating a symptom associated with multiple sclerosis.

It has been observed that the level and activity of acid ceramidase can be elevated in subjects with Alzheimer's disease (Huang et al. (2004) EUROPEAN J. NEUROSCI. 20:3489-3497). Given that the accumulation of sphingosine or sphingosine analogs to a toxic or otherwise detrimental level or concentration in the lysosomal compartments of cells in subjects with Alzheimer's disease, it is contemplated that the administration of an acid ceramidase inhibitor can reduce the accumulation of the sphingosine or sphingosine analogs thereby treating Alzheimer's disease, which includes ameliorating a symptom associated with Alzheimer's disease.

Furthermore, given that a number of the foregoing neurodegenerative disorders, for example, Alzheimer's disease, are associated with a level of cognitive impairment and/or some decrease or loss of cognitive function, it is contemplated that the administration of an effective of an acid ceramidase inhibitor to a subject in need thereof may be reduce, stabilize, or reverse cognitive impairment and/or the loss of cognitive function. Cognitive function generally refers to the mental processes by which one becomes aware of, perceives, or comprehends ideas. Cognitive function involves all aspects of perception, thinking, learning, reasoning, memory, awareness, and capacity for judgment. Cognitive impairment generally refers to conditions or symptoms involving problems with thought processes. This may manifest itself in one or more symptoms indicating a decrease in cognitive function, such as impairment or decrease of higher reasoning skills, forgetfulness, impairments to memory, learning disabilities, concentration difficulties, decreased intelligence, and other reductions in mental functions.

Cognitive function and cognitive impairment may be readily evaluated using tests well known in the art. Performance in these tests can be compared over time to determine whether a treated subject is improving or whether further decline has stopped or slowed, relative to the previous rate of decline of that patient or compared to an average rate of decline. Tests of cognitive function, including memory and learning for evaluating human patients are well known in the art and regularly used to evaluate and monitor subjects having or suspected of having cognitive disorders such as Alzheimer's disease including the clock-drawing test (Agrell & Dehlin (1998) AGE & AGING 27:399-403). Even in healthy individuals, these and other standard tests of cognitive function can be readily used to evaluate beneficial affects over time.

In certain embodiments, a compound (e.g., a compound of Formula (I), (I-Aa), (I-Ab), (II), (III), (IV), (V), (VI), (I-B) (I-B), (I-C), (I-D), (I-E), (I-F), (I-G), or (I-H)) or a pharmaceutical composition containing a compound disclosed herein can be used to treat a neurodegenerative disorder in a subject in need thereof. The invention provides a method of treating a neurodegenerative disorder in a subject. The method comprises administering to the subject an effective amount of a (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a pharmaceutical composition disclosed herein, either alone or in a combination with another therapeutic agent to treat the neurodegenerative disorder in the subject.

Exemplary neurodegenerative disorders include, for example, Alzheimer's disease, Parkinson's disease, Huntington's disease, amyotrophic lateral sclerosis, Lewy body disease, dementia (e.g., frontotemporal dementia), multisystem atrophy, multiple sclerosis, epilepsy, bipolar disorder, schizophrenia, anxiety disorders (e.g., a panic disorder, social anxiety disorder or generalized anxiety disorder) or progressive supranuclear palsy.

It is contemplated that the compounds disclosed can be used in combination with other treatments and/or therapeutic agents. The invention embraces combination therapy, which includes the administration of a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or related compound described herein and a second treatment and/or agent as part of a specific treatment regimen intended to provide the beneficial effect from the co-action of these therapeutic agents.

During the treatment of Parkinson's disease, the acid ceramidase inhibitor can be administered in combination with carbidopa and/or levadopa, a dopamine agonist, a monoamine oxidase B inhibitor, a catchetol O-methyltransferase inhibitor, an anticholingeric, or amantadine. During the treatment of Alzheimer's disease, the acid ceramidase inhibitor can be administered in combination with a cholinesterase inhibitor and/or memantine. During the treatment of Huntington's disease, the acid ceramidase inhibitor can be administered in combination with tetrabenazine; an antipsychotic drug such as haloperidol, chlorpromazine, quetiapine, risperidone, and olanzapine; a chorea-suppressing medication such as amantadine, levetiracetam, and clonazempam; an antidepressant such as citalopram, fluoxetine, and sertraline; and a mood-stabilizing drug such as valproate, carbamazepine, and lamotrigine.

During the treatment of amyotrophic lateral sclerosis, the acid ceramidase inhibitor can be administered in combination with riluzole; an agent for ameliorating muscle cramps and spasms such as cyclobenzaprine HCL, metaxalone, and robaxin; an agent for ameliorating spasticity such as tizanidine HCl, baclofen, and dantrolene; an agent for ameliorating fatigue such as caffeine, caffeine citrate, or caffeine benzoate injection; an agent for ameliorating excessive salivation such as glycopyrrolate, propantheline, amitriptyline, nortriplyline HCL and scopolamine; an agent for ameliorating excessive phlegm such as guaifenesin, albuterol inhalation, and acetylcysteine; an agent for ameliorating pain such as an opioid; an anticonvulsant or antiepileptic; a serotonin reuptake inhibitor; an antidepressant; an agent for ameliorating sleep disorders such as a benzodiazepine, a non-benzodiazepine hypnotic, a melatonin receptor stimulator, an anti-narcoleptic, and an orexin receptor antagonist; and an agent pseudobulbar affect such as dextromethorphan/quinidine.

During the treatment of multiple sclerosis, the acid ceramidase inhibitor can be administered in combination with a corticosteroid, p interferon, glatiramer acetate, dimethyl fumarate, fingolimod, teriflunomide, natalizumab, mitoxantrone, baclofen, and tizanidine. During the treatment of diffuse Lewy body disease, the acid ceramidase inhibitor can be administered in combination with a cholinesterase inhibitor, a Parkinson's disease medication such as carbidopa and/or levodopa, and an anti-psychotic medication such as quetiapine and olanzapine.

During the treatment of multisystem atrophy, the acid ceramidase inhibitor can be administered in combination with a medication to raise blood pressure such as fludrocortisone, psyridostigmine, midodrine, and droxidopa; and a Parkinson's disease medication such as carbidopa and/or levodopa. During the treatment of frontotemporal dementia, the acid ceramidase inhibitor can be administered in combination with an antidepressant, a selective serotonin reuptake inhibitor, and an antipsychotic. During the treatment of progressive upranuclear palsy, the acid ceramidase inhibitor can be administered in combination with a Parkinson's disease medication such as carbidopa and/or levodopa. It is understood that other combinations would be known be those skilled in the art.

### V. Kits for Use in Medical Applications

Another aspect of the invention provides a kit for treating a disorder. The kit comprises: i) instructions for treating a medical disorder, such as, cancer (such as melanoma), a lysosomal storage disorder (such as Krabbe disease, Fabry disease, Tay-Sachs disease, Pompe disease, Hunter's syndrome, Niemann Pick disease Types A and B, Gaucher disease), a neurodegenerative disease (such as Alzheimer's disease, Parkinson's disease, Huntington's disease, amyotrophic lateral sclerosis), an inflammatory disorder, and pain; and ii) a compound described herein or related organic compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a composition described herein. The kit may comprise one or more unit dosage forms containing an amount of a compound described herein or related organic compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), that is effective for treating said medical disorder, for example, cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), neurodegenerative disease (such as Alzheimer's disease, Parkinson's disease, Huntington's disease, and amyotrophic lateral sclerosis), inflammatory disorder, and pain.

The description above describes multiple aspects and embodiments of the invention, including substituted benzimidazole carboxamides and related organic compounds, compositions comprising a substituted benzimidazole carboxamides or related organic compounds, methods of using the substituted benzimidazole carboxamides or related organic compounds, and kits. The patent application specifically contemplates all combinations and permutations of the aspects and embodiments. For example, the invention contemplates treating a medical disorder such as Gaucher disease, Parkinson's disease, Lewy body disease, dementia, or multiple system atrophy in a human patient by administering a therapeutically effective amount of a compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a composition comprising such a compound. Further, for example, the invention contemplates a kit for treating a medical disorder such as cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), neurodegenerative disease (such as Alzheimer's disease, Parkinson's disease, Huntington's disease, and amyotrophic lateral sclerosis), inflammatory disorder, and pain and ii) a compound described herein or related organic compound described herein (e.g., a compound of Formula (I), (I-Aa), (I-Ab), (II), (III), (IV), (V), (VI), (I-B) (I-B), (I-C), (I-D), (I-E), (I-F), (I-G), or (I-H)), or a composition comprising such a compound.

In another aspect, the invention provides a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of formula (I), (I-A), (I-Aa), (I-Ab), (II), (III) (IV), (V), (VI), (I-B), (I-C), (I-D), (I-E), (I-F), (I-G), or (I-H)) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of formula (I), (I-A), (I-A a), (I-Ab), (II), (III), (IV), (V), (VI), (I-B), (I-C), (I-D), (I-E), (I-F), (I-G), or (I-H)) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of formula (I), (I-A), (I-Aa), (I-Ab), (II), (III), (IV), (V), (VI), (I-B), (I-C), (I-D), (I-E), (I-F), (I-G), or (I-H)) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### EXAMPLES

The invention now being generally described, will be more readily understood by reference to the following examples, which are included merely for purposes of illustration of certain aspects and embodiments of the present invention, and are not intended to limit the invention. In certain instances, the amount of compound produced by the procedure is stated along with the yield, which may be presented in the format of the procedure produced the title compound (10 mg; 90%) which means that 10 mg of the title compound was obtained and that corresponds to a yield of 90%.

### Preparation of Saturated and Unsaturated N-Heterocyclic Carboxamide Compounds

Saturated or unsaturated N-heterocylic carboxamides and related compounds were prepared based on general procedures described in Part I below. The section of “Methods of preparing compounds” describe these synthetic methods more generally and provide the structures of the intermediates used in the general procedures.

### Part I—General Procedures

### General Procedure A for the Preparation of Saturated and Unsaturated N-heterocyclic Carboxamides

To a solution of saturated or unsaturated N-heterocylic amine (1.0 eq) and triphosgene (0.5-1.0 eq) in DCM (8-20 mL/mmol) at 0° C. or −78° C. was added Et3N (3.0 eq). The reaction mixture was stirred at 0° C. for 10 min-2 h. The corresponding amine IV-A (1.2-3.0 eq) was added at 0° C. or −78° C. and the reaction mixture was stirred at 0° C. or RT for 1 h-4 h. The solution was diluted with DCM, washed with H₂O, brine, dried over Na₂SO₄ and purified by silica gel column chromatography or Prep-HPLC to give a saturated or unsaturated N-heterocyclic carboxamide, which was further triturated with common organic solvents if needed to increase the purity.

### General Procedure B for the Preparation of Saturated and Unsaturated N-heterocyclic Carboxamides

To a solution of a secondary amine (1.0 eq) and Et3N (2.0-5.0 eq) in DCM or CH₃CN (5-20 mL/mmol) was added isocyanate (e.g., (2-isocyanatoethyl)benzene) (1.2-4.0 eq) at 0° C. or at RT. The resulting mixture was stirred at RT or at reflux for 2 h to overnight. The reaction mixture was poured into water and extracted with DCM. The combined organic layers were washed with brine, dried over Na₂SO₄, filtered and concentrated to give a residue which was purified by silica gel column chromatography or Prep-HPLC to give a saturated or unsaturated N-heterocyclic carboxamide which was further triturated with organic solvents if needed to increase the purity.

### General Procedure A: Synthesis of Compounds of Formula VIIa-h.

### Step 1: Synthesis of Compounds of Formula VI-Aa-i.

To a cooled −78° C. solution of HMDS (1.5 eq., 1.0 M in THF) in anhydrous THF (0.1 M) n-BuLi (1.5 eq, 2.5 M in hexane) was added dropwise. The solution was stirred for 20 min, then added dropwise via cannula to a cooled −78° C. solution of the appropriate ketone V-Aa-i (1.0 eq.) in anhydrous THF (0.1 M) under N₂ atmosphere. The reaction mixture was stirred at −78° C. for 2 h, then N-chloro-(2-pyridyl)bis(trifluoromethanesulfonimide) (2.0 eq.) in anhydrous THF (0.1 M) was added dropwise. The reaction mixture was stirred at −78° C. for 2 h and allowed to warm to it. After 1 h, the reaction mixture was diluted with EA, washed with a 10% aq. NaOH solution, brine and dried over Na₂SO₄. After evaporation of the solvent, the residue was purified by flash chromatography (SiO₂) eluting with Cy/EA.

### Step 2: Synthesis of Compounds of Formula VIIa-i.

To a solution of the compound of Step 1 of Formula VI-Aa-i (1.0 eq.) in 1,4-dioxane (0.1 M, previously degassed under N₂ atmosphere) bis(pinacolato)diboron (1.2 eq.), KOAc (2.0 eq.), PdCl₂(dppf)-DCM complex (0.2 eq.) were added. The reaction mixture was stirred at 90° C. for 1 h under N₂ atmosphere. The corresponding boronic ester of Formula VIIa-i was used in situ in the next step.

### Step 3: Synthesis of Compounds of Formula VIIIa-h.

To a mixture of the compound of Step 2 of Formula VIIa-i (1.0 eq.) in 1,4-dioxane (0.2 M, previously degassed under N₂ atmosphere), 5-bromo-2-nitro-phenol (1.1 eq.), Pd catalyst (0.01 eq.), and Na₂CO₃ (2.0 eq., 2M aqueous solution) were added. The reaction mixture was stirred at 90° C. on under N₂ atmosphere. Then, the reaction mixture was cooled to RT, diluted with EA and washed with a saturated aq. NH₄Cl solution, brine and dried over Na₂SO₄. After evaporation of the solvent, the residue was purified by flash chromatography (SiO₂) eluting with Cy/EA.

As previously mentioned, alternative to procedure A (Step1, Step 2, Step 3), compounds of Formula VIIa-h can be prepared using procedures H and J.

### General Procedure B: Synthesis of Compounds of Formula IXa-h.

Method A: To a suspension of the appropriate unsaturated piperidines of Formula VIIIa-h (1.0 eq.) in MeOH (0.4 M) 10% Pd/C (0.25 eq.) and cyclohexene (30 eq.) were added and the mixture was stirred at reflux for 5 h. The suspension was filtered through a pad of Celite and the filtrate was quickly evaporated under reduced pressure. The residue was used in the next step without further purification.

Method B: A suspension of the appropriate unsaturated piperidines of Formula VIIIa-h (1.0 eq.) in MeOH (0.4 M) was hydrogenated with the H-Cube apparatus using 10% Pd/C catalyst at 60° C. and full H2 mode. After complete conversion (UPLC/MS analysis monitoring), the solvent was evaporated under reduced pressure. The residue was used in the next step without further purification.

Method C: To a solution of the appropriate unsaturated piperidines of Formula VIIIa-h (1.0 eq.) in THF (0.4 M) and saturated aq. NH₄Cl solution (8.0 eq.), Zn solid (8.0 eq) was added portion wise and the mixture was stirred at RT for 15 min. The suspension was filtered through a pad of Celite and the filtrate was dried over Na₂SO₄. After evaporation of the solvent, the residue was used in the next step without purification.

Method E: To a solution of the appropriate unsaturated piperidines of Formula VIIIa-h (1.0 eq.) in EtOH (0.1 M) 10% Pd/C (0.2 eq) was added, followed by the addition of Et₃SiH (10.0 eq.). The reaction mixture was stirred at RT for 15 min. The mixture was filtered through a pad of Celite. After evaporation of the solvent, the residue was used in the next step without purification.

### General Procedure C: Synthesis of Compounds of Formula Xa-w, XIVa-l, XVIIa-c, XXa, XXIa-d, XXIIa.

To a suspension of the compound of Formula IXa-w, or XIIIa-I, or XVIa-c, or XIXa, XVa-d, or XVIIIa (1.0 eq.) in 1,4-dioxane (0.1 M) HCl (30 eq, 4M in 1,4-dioxane) was added and the reaction mixture was stirred at RT for 2 h. After evaporation of the solvent, the residue was used in the next step without further purification.

### General Procedure D: Synthesis of Compounds of Formula I-A.

Method A: To a stirred solution of the appropriate compound of Formula Xa-w, or XIVa-l, or XVIIa-c, or XXa, or XXIa-d, or XXIIa, or XXVa-n, or XXVIIa-c (1.0 eq.) and Et3N (4.0 eq.) in anhydrous CH₃CN (0.2 M) the appropriate isocyanate of Formula III-A (1.1 eq.) was added. The reaction mixture was diluted DCM, washed with brine and dried over Na₂SO₄. After evaporation of the solvent, the residue was purified by column chromatography (SiO₂), eluting with Cy/EA or DCM/MeOH.

Method B: To a stirred solution of triphosgene (0.33 eq.) in dry DCM (0.2 M), a solution of the appropriate amine of Formula IV-A (1.0 eq.) and anhydrous Et3N (or DIPEA, 2.0 eq.) in anhydrous DCM (0.2 M) were added at −15° C. The resulting mixture was stirred at RT for 30 min under N₂ atmosphere and then added to a solution of the appropriate compound of Formula Xa-w, or XIVa-l, or XVIIa-c, or XXa, or XXIa-d, or XXIIa, or XXVa-n, or XXVIIa-c (1.0 eq.) and anhydrous Et₃N (1.0 eq.) in anhydrous DCM (0.2 M). The reaction mixture was stirred under N₂ atmosphere at RT for 30 min and then diluted with DCM, washed with saturated aq. NH₄Cl solution, brine and dried over Na₂SO₄. After evaporation of the solvent, the residue was purified by column chromatography (SiO₂), eluting with Cy/EA or DCM/MeOH.

Method C: To a stirred solution of the appropriate compound of Formula Xa-w, or XIVa-l, or XVIIa-c, or XXa, or XXIa-d, or XXIIa, or XXVa-n, or XXVIIa-c (1.0 eq.) in THF and CH₃CN (1:1, 0.1M), Et₃N (or DIPEA, or pyridine, 1.2 eq.) was added, followed by the addition of phenylchloroformate (or p-nitrophenylchloroformate, or CDI, 1.1 eq.). The reaction was stirred at RT overnight, then diluted with DCM, washed with H₂O, brine and dried over Na₂SO₄. After evaporation of the solvent, the residue was taken up in THF (0.1 M) and added dropwise to a solution of the appropriate amine of Formula IV-A (1.0 eq.) and Et₃N (or DIPEA, or pyridine, 1.0 eq.). The reaction mixture was stirred at RT for 2 h and then diluted with DCM, washed with a saturated aq. NH₄Cl solution, brine and dried over Na₂SO₄. After evaporation of the solvent, the residue was purified by column chromatography (SiO₂), eluting with Cy/EA or DCM/MeOH.

### General Procedure E: Synthesis of Compounds of Formula XIIIa-e.

To a solution of the appropriate amine of Formula XIIa-c (1.0 eq.), the appropriate aryl halide (1.2 eq.), K₃PO₄ (2.0 eq.), DMEDA (or DMCD, 0.2 eq.) in 1,4-dioxane (0.2 M, previously degassed under N₂ atmosphere) CuI (0.1 eq.) was added under N₂ atmosphere. The reaction mixture was stirred at reflux for 48 h. Then, cooled to RT, diluted with EA and washed with a saturated aqueous NaHCO₃ solution, brine and dried over Na₂SO₄. After evaporation of the solvent, the residue was purified by flash chromatography (SiO₂) eluting with Cy/EA.

### General Procedure F: Synthesis of Compounds of Formula XHIIa-e.

To a solution of the appropriate amine of Formula XIIa-c (1.0 eq.) in CH₃CN (0.2 M) DIPEA (1.3 eq.) and the appropriate aryl halide (1.3 eq.) were added. The reaction mixture was stirred at 70° C. overnight, cooled to RT, diluted with DCM, washed with a saturated aq. NH₄Cl solution, brine and dried over Na₂SO₄. After evaporation of the solvent, the residue was purified by column chromatography (SiO₂), eluting with Cy/EA, Cy/MTBE or DCM/MeOH.

### General Procedure G: Synthesis of Compounds of Formula XIIIa-e.

To a solution of the appropriate amine of Formula XIIa-c (1.0 eq.) in anhydrous toluene (0.1 M, degassed under N₂ atmosphere) the appropriate aryl bromide (1.1 eq), Pd₂(dba)₃ (0.01 eq), (±)-BINAP (0.02 eq) and tBuOK (1.5 eq.) were added. The reaction mixture was stirred at reflux for 8 h. Then, the reaction mixture was cooled to RT, diluted with EA and washed with saturated aq. NH₄Cl solution, brine and dried over Na₂SO₄. After evaporation of the solvent, the residue was purified by flash chromatography (SiO₂) eluting with Cy/EA.

### General Procedure H: Synthesis of Compounds of Formula XIa-h and XXIVa-n.

Method A, via Grignard reagent preparation: To a solution of Mg turnings (3.6 eq.) in anhydrous THF (2.0 M) a solution of the appropriate aryl bromide (3.0 eq.) in anhydrous THF (1.0 M) was added under argon atmosphere and I₂ (1-2 granules) were added to initiate the reaction. The Grignard reagent was ready for use without further purification when the Mg was consumed. A solution of appropriate ketone V-Aa-i (2.4 eq.) or appropriate N-Boc lactam XXIIIa-e (2.0-5.0 eq), in anhydrous THF (1.0 M) was then added dropwise at −40° C. After 1 h, the reaction mixture was quenched with saturated aq. NH₄Cl solution, extracted with EA, washed with brine, dried over Na₂SO, concentrated and the residue was purified by column chromatography (SiO₂) eluting with Cy/EA.

Method B, via organolithium reagent preparation: To a cooled −78° C. solution of the appropriate aryl bromide (1.1 eq.) in anhydrous THF (1.0 M) n-BuLi (1.0 eq., 2.5 M in hexanes) was added dropwise under argon atmosphere for 30 min. A solution of appropriate ketone V-Aa-i (2.4 eq.) or appropriate N-Boc lactam XXIIIa-e (2.0-5.0 eq), in anhydrous THF (1.0 M) was then added dropwise at −40° C. After 1 h, the reaction mixture was quenched with saturated aqueous NH₄Cl solution and extracted with EA. The organic phase was washed with brine and dried over Na₂SO₄. After evaporation of the solvent, the residue was purified by column chromatography (SiO₂), eluting with Cy/EA.

### General Procedure I: Synthesis of Compounds of Formula IXi-w XIIIf-l, XVIa-c and XIX a.

Method A: To a solution of appropriate ketone or aldehyde (1.0 eq.) in MeOH (0.2 M), Et₃N (1.0 eq.), NaOAc (1.6 eq.), glacial AcOH (1.6 eq.), the appropriate amine (1.1 eq.), and NaBH(AcO)₃ (1.6 eq.) were added. The mixture was stirred at RT overnight under N₂ atmosphere and then diluted with EA, washed with saturated aq. NaHCO₃ solution, brine and dried over Na₂SO₄. After evaporation of the solvent, the residue was purified by IST ISOLUTE SPE column SCX to afford compounds of Formula IXi-w and XIIIf.

Method B: A mixture of the appropriate ketone (1.0 eq.) and amine (1.5 eq.) was stirred in neat Ti(iPrO)₄ (2.0 eq.) for about 1-4 h at RT then a solution of NaBH₃CN (1.5 eq.) in anhydrous MeOH (0.15 M) was added. The mixture was stirred at RT for 4 h under N₂ atmosphere. H₂O was added, and the thick white suspension was filtered through a celite pad. The filtrate was concentrated under vacuo and the residue then was dissolved with EA, washed with saturated aq. NaHCO₃ solution, brine and dried over Na₂SO₄. After evaporation of the solvent, the residue was purified by column chromatography (SiO₂) to afford compound of Formula XVIa-c and XIX a.

Method C: To a solution of appropriate piperazine XIId-h (1.0 eq.) in THF:MeOH (1:1, 0.2 M) cyclopropanone trimethylsilyl acetal (1.2 eq.), glacial AcOH (10.0 eq.) and NaBH₃CN (1.5 eq.) were added. The mixture was stirred at 70° C. for 4 h under N₂ atmosphere and then diluted with EA, washed with a saturated aq. NaHCO₃ solution, brine and dried over Na₂SO₄. After evaporation of the solvent, the residue of compounds of Formula XIIIg-l was used in the next step without further purification.

### General Procedure J: Synthesis of Compounds of Formula VIIa-h.

To a stirred solution of the appropriate compound of Formula XIa-h (1.0 eq.) in anhydrous toluene (0.1 M) Burgess reagent (1.5 eq.) was added. The reaction mixture was stirred at 90° C. for 1 h. The solvent was removed under vacuo and the residue was then dissolved with EA, washed with H₂O and dried over Na₂SO₄. After evaporation of the solvent, the residue was purified by column chromatography (SiO₂), eluting with Cy/EA or used in the next step without further purification, as indicated in each case.

### General Procedure K: Synthesis of Compounds of Formula XXVa-n.

To a solution of appropriate N-Boc amino ketone of formula XXIVa-n (1.0 eq.) in anhydrous DCE (0.1M), TFA (10.0 eq.) was added. The reaction mixture was stirred at RT for 1 h (UPLC/MS analysis monitoring). Then, TFA was removed under vacuo and the resulting crude was solubilized in DCE (0.1M) and NaBH(OAc)₃, or NaBH₃CN (3.0 eq) was added. The reaction mixture was stirred at RT for 0.5-2 h. After completion of the reaction, the excess of reductive reagent was quenched with MeOH. After evaporation of the solvent, the residue was employed directly in the next step, or purified by flash chromatography (SiO₂) eluting with DCM/MeOH, or purified by IST ISOLUTE SPE column SCX.

### Part II—Preparation of Specific Saturated and Unsaturated N-Heterocyclic Compounds

Exemplary procedures for preparing specific saturated and unsaturated N-heterocyclic compounds are provided below.

### 1-2, 2-Dimethyl-1, 2, 3, 4-tetrahydroquinoline (Intermediate A)

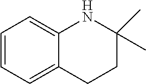

A mixture of aniline (10 g, 107.5 mmol), 3-chloro-3-methylbut-1-yne (14.3 g, 139.7 mmol), Cu (6.8 g, 107.5 mmol) and CuCl (10.6 g, 107.5 mmol) in toluene (140 mL) was stirred at 110° C. under N₂ for 24 hrs. Then the mixture was filtered and concentrated in vacuo to give a residue which was purified by silica gel column chromatography (PE:EA=10:1) to afford 2,2-dimethyl-1,2-dihydroquinoline (400 mg, 2%) as a brown oil. LC-MS m/z: 160.3 [M+H]⁺. HPLC Purity (214 nm): 50%; t_{R}=1.01 min.

A solution of 2, 2-dimethyl-1,2-dihydroquinoline (400 mg, 2.5 mmol) and PtO₂ (50 mg) in MeOH (100 mL) was stirred at RT under H₂ for 2 hrs. Then the mixture was filtered and concentrated in vacuo to give a residue which was purified by silica gel column chromatography (PE:EA=32:1) to afford intermediate A (390 mg, 96%) as a yellow oil. LC-MS m/z: 162.3 [M+H]⁺. HPLC Purity (214 nm): 69%; t_{R}=0.81 min.

### 2,2-Dimethyl-7-(1-methyl-1,2,3,6-tetrahydropyridin-4-yl)-1,2-dihydroquinoline (Intermediate C) and 2,2-Dimethyl-5-(1-methyl-1,2,3,6-tetrahydropyridin-4-yl)-1,2-dihydroquinoline (Intermediate D)

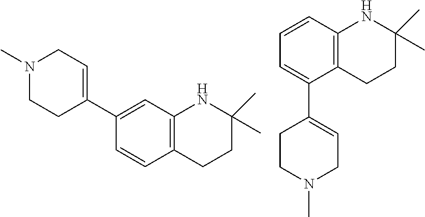

A mixture of 3-bromoaniline (1.71 g, 10 mmol), 3-chloro-3-methylbut-1-yne (1.02 g, mmol), Cu (640 mg, 10 mmol) and CuCl (990 mg, 10 mmol) in toluene (30 mL) was stirred at 110° C. under N₂ for 24 h. Then the mixture was filtered and concentrated in vacuo to give a residue which was purified by silica gel column chromatography (PE:EA=10:1) to give a mixture of 7-bromo-2,2-dimethyl-1,2-dihydroquinoline and 5-bromo-2,2-dimethyl-1,2-dihydro quinoline (660 mg, 28%) as a yellow oil. LC-MS m/z: 238.3 [M+H]⁺. Purity (214 nm): 71%; t_{R}=2.22 min.

A mixture of 7-bromo-2,2-dimethyl-1,2-dihydroquinoline and 5-bromo-2,2-dimethyl-1,2-dihydroquinoline (550 mg, 2.32 mmol), 1-methyl-4-(4,4,5,5-tetramethyl-1,3,2-dioxaborolan-2-yl)-1,2,3,6-tetrahydropyridine (621 mg, 2.78 mmol), Pd(PPh₃)₄ (268 mg, 0.232 mmol) and K₂CO₃ (960 mg, 6.96 mmol) in dioxane (30 mL) and H₂O (10 mL) was heated at 95° C. under N₂ for 16 h. The mixture was concentrated in vacuo to give a residue which was purified by silica gel column chromatography (PE:EA=10:1) to give intermediate C (200 mg, 28%) as a yellow oil (LC-MS m/z: 255.1 [M+H]⁺. HPLC Purity (214 nm): 86%; t_{R}=2.12 min) and intermediate D (300 mg, 43%) as a yellow oil. LC-MS m/z: 255.1 [M+H]⁺. HPLC Purity (214 nm): 61%; t_{R}=2.26 min.

### Example 1: 2,2-Dimethyl-7-(1-methylpiperidin-4-yl)-N-phenethyl-3,4-dihydroquinoline-1(2H)-carboxamide

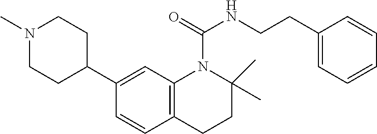 [SMILES: CN1CCC(c2ccc3c(c2)N(C(=O)NCCc2ccccc2)C(C)(C)CC3)CC1]

A solution of 2,2-dimethyl-7-(1-methyl-1,2,3,6-tetrahydropyridin-4-yl)-1,2-dihydroquinoline (254 mg, 1 mmol), PtO₂ (50 mg, 0.2 mmol) in MeOH (10 mL) was stirred at RT for 1 h and filtered. The filtrate was concentrated to afford 2,2-dimethyl-7-(1-methylpiperidin-4-yl)-1,2,3,4-tetrahydroquinoline (210 mg, 81%) as a yellow solid. LC-MS m/z: 259.0 [M+H]⁺. HPLC Purity (214 nm): 89%; t_{R}=2.28 min.

Following general procedure B, 2,2-dimethyl-7-(1-methylpiperidin-4-yl)-1,2,3,4-tetrahydroquinoline (103 mg, 0.4 mmol) and (2-isocyanatoethyl)benzene (120 mg, 0.8 mmol) afforded the title compound (20 mg, 15%) as a white solid. ¹H NMR (400 MHz, CDCl₃) δ 7.29-7.26 (m, 2H), 7.24-7.19 (m, 3H), 7.03 (d, J=7.6 Hz, 1H), 6.88-6.78 (m, 2H), 5.05 (t, J=5.7 Hz, 1H), 3.58-3.53 (m, 4H), 2.92-2.74 (m, 7H), 2.59 (t, J=6.0 Hz, 2H), 2.50-2.38 (m, 3H), 1.88 (d, J=11.9 Hz, 2H), 1.75-1.69 (m, 2H), 1.52 (s, 6H). LC-MS m/z: 406.1 [M+H]⁺. HPLC Purity (214 nm): 97.2%; t_{R}=7.21 min.

### Example 2: 2,2-Dimethyl-5-(1-methylpiperidin-4-yl)-N-phenethyl-3,4-dihydroquinoline-1(2H)-carboxamide

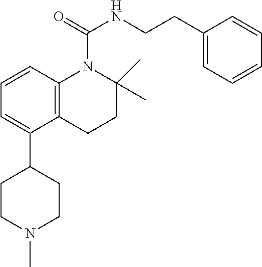 [SMILES: CN1CCC(c2cccc3c2CCC(C)(C)N3C(=O)NCCc2ccccc2)CC1]

A solution of 2,2-dimethyl-5-(1-methyl-1,2,3,6-tetrahydropyridin-4-yl)-1,2-dihydroquinoline (254 mg, 1 mmol), PtO₂ (50 mg, 0.2 mmol) in MeOH (10 mL) was stirred at RT for 1 h and filtered. The filtrate was concentrated to afford 2,2-dimethyl-5-(1-methylpiperidin-4-yl)-1,2,3,4-tetrahydroquinoline (220 mg, 85%) as a yellow solid. LC-MS m/z: 259.1 [M+H]⁺. HPLC Purity (214 nm): 91%; t_{R}=2.21 min

Following general procedure B, 2,2-dimethyl-5-(1-methylpiperidin-4-yl)-1,2,3,4-tetrahydroquinoline (206 mg, 0.8 mmol) and (2-isocyanatoethyl)benzene (235 mg, 1.6 mmol) afforded the title compound (71 mg, 22%) as a white solid. ¹H NMR (400 MHz, CDCl₃) δ 7.30-7.27 (m, 1H), 7.25 (s, 1H), 7.23-7.15 (m, 3H), 7.01-6.84 (m, 2H), 6.79 (d, J=7.6 Hz, 1H), 4.91 (t, J=5.6 Hz, 1H), 3.47 (dt, J=13.1, 6.8 Hz, 2H), 3.10 (brd, J=11.3 Hz, 2H), 2.81 (t, J=7.0 Hz, 2H), 2.79-2.69 (m, 1H), 2.61-2.50 (m, 2H), 2.42 (s, 3H), 2.29-2.18 (m, 2H), 1.99-1.88 (m, 2H), 1.76 (brd, J=13.3 Hz, 2H), 1.68 (dd, J=11.9, 6.1 Hz, 2H), 1.54 (s, 6H). LC-MS m/z: 406.2 [M+H]⁺. HPLC Purity (214 nm): 100%; t_{R}=6.94 min.

### Example 3: 7-(1-Methylpiperidin-4-yl)-N-phenethyl-3,4-dihydroquinoline-1(2H)-carboxamide

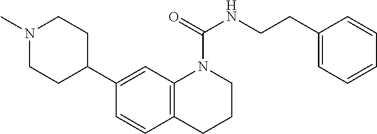 [SMILES: CN1CCC(c2ccc3c(c2)N(C(=O)NCCc2ccccc2)CCC3)CC1]

A mixture of 7-bromoquinoline (200 mg, 0.959 mmol), 1-methyl-4-(4,4,5,5-tetra-methyl-1,3,2-dioxaborolan-2-yl)-1,2,3,6-tetrahydropyridine (214 mg, 0.959 mmol), Na₂CO₃ (254 mg, 2.399 mmol) and Pd(PPh₃)₄ (111 mg, 0.096 mmol) in 1,4-dioxane/H₂O (7.5 mL, 4/1) was stirred at 80° C. under N₂ for 5 h. The reaction mixture was cooled, concentrated and purified by silica gel column chromatography (DCM:MeOH=5:1) to give 7-(1-methyl-1,2,3,6-tetrahydropyridin-4-yl)quinoline (180 mg, 84%) as a yellow solid. LC-MS m/z: 225.0 [M+H]⁺. Purity (214 nm): 97%; t_{R}=1.78 min.

To a solution of 7-(1-methyl-1,2,3,6-tetrahydropyridin-4-yl)quinoline (170 mg, 0.758 mmol) in EtOH (12 mL) was added PtO₂ (35 mg) and the mixture was stirred at RT under H2 for 8 h. The mixture was filtered, concentrated and purified by silica gel column chromatography (DCM:MeOH=10:1) to give 7-(1-methylpiperidin-4-yl)-1,2,3,4-tetrahydroquinoline (80 mg, 52%) as a colorless semisolid. LC-MS m/z: 231.1 [M+H]⁺. Purity (214 nm): 89.7%; t_{R}=1.84 min.

Following general procedure B, 7-(1-methylpiperidin-4-yl)-1,2,3,4-tetrahydroquinoline (70 mg, 0.30 mmol) and (2-isocyanatoethyl)benzene (67 mg, 0.46 mmol) afforded the title compound (40 mg, 34.9%) as a yellow solid. ¹H NMR (400 MHz, DMSO-d₆) δ 7.30 (t, J=7.4 Hz, 2H), 7.20 (dd, J=13.3, 6.0 Hz, 4H), 7.00 (d, J=7.8 Hz, 1H), 6.79 (d, J=7.8 Hz, 1H), 6.66 (t, J=5.3 Hz, 1H), 3.52 (t, J=5.9 Hz, 2H), 3.33 (dd, J=13.1, 6.7 Hz, 2H), 2.99 (d, J=11.3 Hz, 2H), 2.78 (t, J=7.2 Hz, 2H), 2.63 (t, J=6.5 Hz, 2H), 2.43-2.28 (m, 4H), 2.20 (t, J=10.5 Hz, 2H), 1.84-1.52 (m, 6H). LC-MS m/z: 378.4 [M+H]⁺. HPLC Purity (214 nm): >99%; t_{R}=5.62 min.

### Examples 4 and 5: 2,2-Dimethyl-N-phenethyl-7-(pyridin-2-yl)-3,4-dihydroquinoline-1(2H)-carboxamide and 2,2-Dimethyl-N-phenethyl-5-(pyridin-2-yl)-3,4-dihydroquinoline-1(2H)-carboxamide

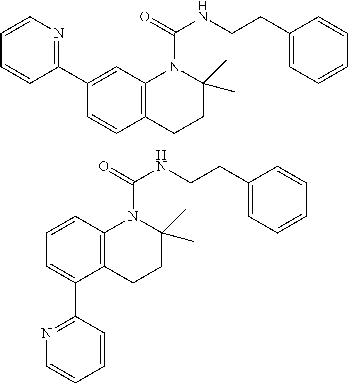

A mixture of 7-bromo-2,2-dimethyl-1,2-dihydroquinoline and 5-bromo-2,2-dimethyl-1,2-dihydroquinoline (238 mg, 1.0 mmol), 2-(tributylstannyl)pyridine (441 mg, 1.2 mmol), Pd(dppf)Cl₂.DCM (120 mg, 0.2 mmol) in 1,4-dioxane (10 mL) was stirred at 100° C. for 12 h under N₂. The reaction was cooled and concentrated in vacuo to give a residue which was purified by silica gel column chromatography (DCM/MeOH=10/1) to afford a mixture of 2,2-dimethyl-7-(pyridin-2-yl)-1,2-dihydroquinoline and 2,2-dimethyl-5-(pyridin-2-yl)-1,2-dihydroquinoline (240 mg, crude) as yellow solids. LC-MS m/z: 237.3 [M+H]⁺. HPLC Purity (214 nm): 79%; t_{R}=0.75 min.

A solution of 2,2-dimethyl-7-(pyridin-2-yl)-1,2-dihydroquinoline and 2,2-dimethyl-5-(pyridin-2-yl)-1,2-dihydroquinoline (236 mg, 1.0 mmol), PtO₂ (50 mg, 0.2 mmol) in MeOH (10 mL) was stirred at RT for 1 h and filtered. The filtrate was concentrated to afford the mixture of 2,2-dimethyl-7-(pyridin-2-yl)-1,2,3,4-tetrahydroquinoline and 2,2-dimethyl-5-(pyridin-2-yl)-1,2,3,4-tetrahydroquinoline (240 mg, crude) as a yellow solid. LC-MS m/z: 239.1 [M+H]⁺. HPLC Purity (214 nm): 70%; t_{R}=1.54 min.

Following general procedure B, 2,2-dimethyl-7-(pyridin-2-yl)-1,2,3,4-tetrahydroquinoline and 2,2-dimethyl-5-(pyridin-2-yl)-1,2,3,4-tetrahydroquinoline (143 mg, 0.6 mmol) and (2-isocyanatoethyl)benzene (132 mg, 0.9 mmol) afforded 2,2-dimethyl-N-phenethyl-7-(pyridin-2-yl)-3,4-dihydroquinoline-1(2H)-carboxamide (32.3 mg, 13.9%) and 2,2-dimethyl-N-phenethyl-5-(pyridin-2-yl)-3,4-dihydroquinoline-1(2H)-carboxamide (4.4 mg, 1.8%) as white solids.

### 2,2-dimethyl-N-phenethyl-7-(pyridin-2-yl)-3,4-dihydroquinoline-1(2H)-carboxamide

¹H NMR (400 MHz, CDCl₃) δ 8.68 (d, J=4.8 Hz, 1H), 7.74 (ddd, J=15.4, 8.1, 1.6 Hz, 2H), 7.63-7.53 (m, 2H), 7.28-7.11 (m, 7H), 5.11 (s, 1H), 3.57-3.50 (m, H), 2.87 (t, J=7.1 Hz, 2H), 2.68-2.60 (m, 2H), 1.81-1.77 (m, 2H), 1.60 (s, 6H). LC-MS m/z: 386.1 [M+H]⁺. HPLC Purity (214 nm): 1000%; t_{R}=7.20 min.

### 2,2-dimethyl-N-phenethyl-5-(pyridin-2-yl)-3,4-dihydroquinoline-1(2H)-carboxamide

¹H NMR (400 MHz, CDCl₃) δ 8.72 (dd, J=4.8, 1.2 Hz, 1H), 7.77 (dt, J=7.6, 1.6 Hz, 1H), 7.43 (d, J=7.6, 1H), 7.33-7.28 (m, 3H), 7.27-7.19 (m, 3H), 6.97-7.11 (m, 3H), 4.94 (bs 1H), 3.54 (dd, J=13.2, 7.0 Hz, 2H), 2.85 (t, J=7.2 Hz, 2H), 2.66-2.60 (m, 2H), 1.64-1.59 (m, 2H), 1.59 (s, 6H). LC-MS m/z: 386.1 [M+H]⁺. HPLC Purity (214 nm): 94.43%; t_{R}=6.97 min.

### Example 6: 2,2-Dimethyl-N-phenethyl-3,4-dihydroquinoline-1(2H)-carboxamide

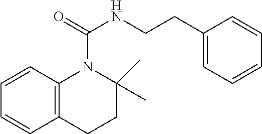 [SMILES: CC1(C)CCc2ccccc2N1C(=O)NCCc1ccccc1]

Following general procedure B, 2,2-dimethyl-1,2,3,4-tetrahydroquinoline (150 mg, 0.9 mmol) and (2-isocyanatoethyl)benzene (685 mg, 4.7 mmol) in CH₃CN afforded the title compound (34.3 mg, 12%) as a white solid. ¹H NMR (400 MHz, CDCl₃) δ 7.29-7.25 (m, 2H), 7.22-7.16 (m, 3H), 7.03 (d, J=7.6 Hz, 1H), 6.97-6.92 (m, 1H), 6.88-6.84 (m, 2H), 4.98 (bs, 1H), 3.51 (q, J=6.4 Hz, 2H), 2.83 (t, J=7 Hz, 2H), 2.58-2.53 (m, 2H), 1.72-1.69 (m, 2H), 1.54 (s, 6H). LC-MS m/z: 309.3 [M+H]⁺. HPLC Purity (214 nm): 100%; t_{R}=10.04 min.

### Example 7: 2,2,4-Trimethyl-N-phenethyl-3,4-dihydroquinoxaline-1(2H)-carboxamide

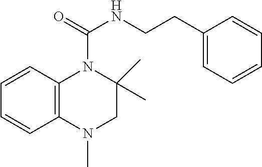 [SMILES: CN1CC(C)(C)N(C(=O)NCCc2ccccc2)c2ccccc21]

To a mixture of 1-fluoro-2-nitrobenzene (3.5 g, 24.8 mmol) in DMF (35 mL) was added methyl 2-amino-2-methylpropanoate (5.7 g, 37.2 mmol) and Cs₂CO₃ (20 g, 62.0 mmol). The mixture was stirred at 100° C. for 24 h. The reaction was concentrated to give a residue which was purified by silica gel column chromatography (PE:EA=7:3) to give methyl 2-methyl-2-(2-nitrophenylamino) propanoate (1.0 g, 16.9%) as a white solid. LC-MS m/z: 239.3 [M+H]⁺. HPLC Purity (254 nm): 56.6%; t_{R}=1.01 min.

To a solution of methyl 2-methyl-2-(2-nitrophenylamino) propanoate (1.2 g, 5.0 mmol) in MeOH (50 mL) was added Pd/C (0.6 g). The mixture was stirred at RT under H2 for 1 h. The mixture was filtered and concentrated to afford 3,3-dimethyl-3,4-dihydroquinoxalin-2(1H)-one (770 mg), which was used directly in the next step. LC-MS m/z: 177.3 [M+H]⁺. HPLC Purity (214 nm): 94.61%; t_{R}=0.71 min.

A solution of 3, 3-dimethyl-3,4-dihydroquinoxalin-2(1H)-one (740 mg, 4.2 mmol) in BH₃-THF (30 mL) was stirred at 60° C. for 1 h followed by the addition of conc. HCl (10 mL). The mixture was stirred at 60° C. for 3 h, and Na₂CO₃ was added to pH=5. The reaction was concentrated to give a residue which was purified by silica gel column chromatography (PE:EA=4:1) to give methyl 2,2-dimethyl-1,2,3,4-tetrahydroquinoxaline (650 mg, 95.4%). LC-MS m/z: 163.1 [M+H]⁺. HPLC Purity (214 nm): 96.65%. t_{R}=1.79 min.

To a mixture of 2, 2-dimethyl-1, 2, 3, 4-tetrahydroquinoxaline (236 mg, 1.46 mmol) in MeOH (10 mL) was added formaldehyde (236.3 g, 2.91 mmol). The mixture was stirred at RT for 30 min, NaCNBH₃ (365.9 g, 5.82 mmol) was added and the mixture was stirred for 2 h. The reaction was concentrated to give a residue which was purified by silica gel column chromatography (PE:EA=6:1) to give 1,3,3-trimethyl-1,2,3,4-tetrahydroquinoxaline (160 mg, 62.5%). LC-MS m/z: 177.2 [M+H]⁺. HPLC Purity (254 nm): 94.9%. t_{R}=1.96 min.

Following general procedure B, 1,3,3-trimethyl-1,2,3,4-tetrahydroquinoxaline (150 mg, 0.85 mmol) and (2-isocyanatoethyl)benzene (376 mg, 2.56 mmol) afforded the title compound (229 mg, 83%) as a white solid. ¹H NMR (400 MHz, CDCl₃) δ 7.30-7.28 (m, 2H), 7.22-7.16 (m, 3H), 6.89 (dd, J=7.6, 1.2 Hz, 1H), 6.80 (dd, J=6.4, 1.2 Hz, 1H), 6.63 (dd, J=7.2, 1.2 Hz, 1H), 6.55 (dd, J=7.6, 1.2 Hz, 1H), 5.09 (bs, 1H), 3.52-3.47 (m, 2H), 2.87 (s, 2H), 2.84 (s, 3H), 2.82-2.80 (m, 2H), 1.50 (s, 6H). LC-MS m/z: 324.1 [M+H]⁺. HPLC Purity (214 nm): 1000%; t_{R}=9.9 min.

### Example 8: 2,2-Dimethyl-N-phenethyl-3,4-dihydro-1,8-naphthyridine-1(2H)-carboxamide

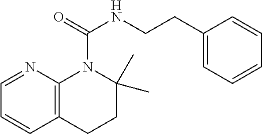 [SMILES: CC1(C)CCc2cccnc2N1C(=O)NCCc1ccccc1]

A suspension of pyridin-3-amine (5.0 g, 53.1 mmol), 3-chloro-3-methylbut-1-yne (10.9 g, 106.3 mmol), Et₃N (10.8 g, 106.3 mmol) and CuCl (5.3 g, 53.1 mmol) in toluene (70 mL) was stirred at 110° C. overnight under N₂. The mixture was cooled, filtered and concentrated to give a residue which was purified by silica gel column chromatography (PE:EA=1:1) to give 2,2-dimethyl-1,2-dihydro-1,5-naphthyridine (300 mg, 3.5%) as a yellow solid. LC-MS m/z: 161.3 [M+H]⁺. HPLC Purity (214 nm): 92%; t_{R}=1.36 min.

To a solution of 2,2-dimethyl-1,2-dihydro-1,5-naphthyridine (300 mg, 1.9 mmol) in MeOH (10 mL) was added PtO₂ (50 mg, 0.2 mmol). The mixture was stirred at RT under an atmospheric pressure of H₂ for 1 h. The mixture was filtered and concentrated to give 2,2-dimethyl-1,2,3,4-tetrahydro-1,5-naphthyridine (250 mg, 81.8%) as a yellow solid. LC-MS m/z: 163.3 [M+H]⁺. HPLC Purity (214 nm): 91%; t_{R}=0.47 min.

Following general procedure B, 2,2-dimethyl-1,2,3,4-tetrahydro-1,5-naphthyridine (150 mg, 0.9 mmol) and (2-isocyanatoethyl)benzene (1.08 g, 7.4 mmol) afforded the title compound (60.3 mg, 21.1%) as a white solid. ¹H NMR (400 MHz, CDCl₃) δ 9.81 (bs, 1H), 8.03 (dd, J=3.6, 1.2 Hz, 1H), 7.33-7.19 (m, 6H), 6.86-6.82 (m, 1H), 3.60 (q, J=6.8 Hz, 2H), 2.91 (t, J=6.8 Hz, 2H), 2.72-2.66 (m, 2H), 1.81-1.75 (m, 2H), 1.66 (s, 6H). LC-MS m/z: 310.2 [M+H]⁺. HPLC Purity (214 nm): 100%; t_{R}=6.26 min.

### Example 9: 2,2-Dimethyl-N-phenethyl-3,4-dihydro-1,5-naphthyridine-1(2H)-carboxamide

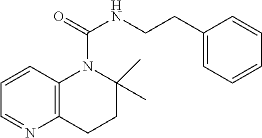 [SMILES: CC1(C)CCc2ncccc2N1C(=O)NCCc1ccccc1]

A suspension of pyridin-3-amine (5.0 g, 53.1 mmol), 3-chloro-3-methylbut-1-yne (10.9 g, 106.3 mmol), Et₃N (10.8 g, 106.3 mmol) and CuCl (5.3 g, 53.1 mmol) in toluene (70 mL) was stirred at 110° C. overnight under N₂. The mixture was filtered, concentrated and purified by silica gel column chromatography (PE:EA=1:1) to give 2,2-dimethyl-1,2-dihydro-1,5-naphthyridine (300 mg, 3.5%) as a yellow solid. LC-MS m/z: 161.3 [M+H]⁺. Purity (214 nm): 92%; t_{R}=1.36 min.

To a solution of 2,2-dimethyl-1,2-dihydro-1,5-naphthyridine (300 mg, 1.9 mmol) in MeOH (10 mL) was added PtO₂ (50 mg, 0.2 mmol) and the mixture was stirred at RT under an atmospheric pressure of H2 for 1 h. The mixture was filtered and concentrated to give 2,2-dimethyl-1,2,3,4-tetrahydro-1,5-naphthyridine (250 mg, 81.8%) as a yellow solid. LC-MS m/z: 163.3 [M+H]⁺. Purity (214 nm): 91%; t_{R}=0.47 min.

Following general procedure B, 2,2-dimethyl-1,2,3,4-tetrahydro-1,5-naphthyridine (150 mg, 0.9 mmol) and (2-isocyanatoethyl)benzene (1.08 g, 7.4 mmol) afforded the title compound (60.3 mg, 21.1%) as a white solid. ¹H NMR (400 MHz, CDCl₃) δ 8.03 (dd, J=3.6 Hz, 1.2 Hz, 1H), 7.30 (t, J=7.6 Hz, 2H), 7.25-7.17 (m, 3H), 7.03 (dd, J=6.8 Hz, 1.2 Hz, 1H), 6.86-6.82 (m, 1H), 4.95 (bs, 1H), 3.56 (q, J=6.8 Hz, 2H), 2.88-2.81 (m, 4H), 1.81 (t, J=6.2 Hz, 2H), 1.50 (s, 6H). LC-MS m/z: 310.2 [M+H]⁺. HPLC: Purity (214 nm): 100%; t_{R}=6.26 min.

### Example 10: 2,2-Dimethyl-7-morpholino-N-phenethyl-3,4-dihydroquinoline-1(2H)-carboxamide

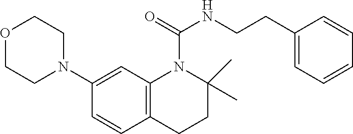 [SMILES: CC1(C)CCc2ccc(N3CCOCC3)cc2N1C(=O)NCCc1ccccc1]

A mixture of 7-bromo-2,2-dimethyl-1,2-dihydroquinoline and 5-bromo-2,2-dimethyl-1,2-dihydroquinoline (1.9 g, 8 mmol), morpholine (1.4 g, 16 mmol), Pd₂(dba)₃ (360 mg, 0.4 mmol), BINAP (480 mg, 0.8 mmol) and t-BuOK (1.8 g, 16 mmol) in toluene (20 mL) was stirred at 90° C. for 12 h under N₂. The reaction was cooled and concentrated in vacuo to give a residue which was purified by silica gel column chromatography (PE/EA=10/1) to give 4-(2,2-dimethyl-1,2-dihydroquinolin-7-yl)morpholine (320 mg, crude) (LC-MS m/z: 245.4 [M+H]⁺. HPLC Purity (214 nm): 90%; t_{R}=0.898 min) and 4-(2,2-dimethyl-1,2-dihydroquinolin-5-yl)morpholine (550 mg, crude) as yellow solids (LC-MS m/z: 245.1 [M+H]+. HPLC Purity (214 nm): 86%; t_{R}=2.005 min).

A solution of 4-(2, 2-dimethyl-1,2-dihydroquinolin-7-yl)morpholine (244 mg, 1.0 mmol), PtO₂ (50 mg, 0.2 mmol) in MeOH (10 mL) was stirred at RT for 1 h and filtered. The filtrate was concentrated to afford 4-(2,2-dimethyl-1,2,3,4-tetrahydroquinolin-7-yl)morpholine (230 mg, 93%) as a yellow solid. LC-MS m/z: 247.3 [M+H]⁺. HPLC Purity (214 nm): 89%; t_{R}=0.74 min.

Following general procedure B, 4-(2,2-dimethyl-1,2,3,4-tetrahydroquinolin-7-yl)morpholine (123 mg, 0.5 mmol) and (2-isocyanatoethyl)benzene (220 mg, 1.5 mmol) afforded the title compound (35.1 mg, 18%) as a white solid. ¹H NMR (400 MHz, CDCl₃) δ 7.31-7.27 (m, 2H), 7.24-7.17 (m, 3H), 6.97 (t, J=11.0 Hz, 1H), 6.58 (d, J=2.4 Hz, 1H), 6.49 (dd, J=8.2, 2.4 Hz, 1H), 5.07 (t, J=5.5 Hz, 1H), 3.93-3.74 (m, 4H), 3.49 (dd, J=13.5, 6.8 Hz, 2H), 3.03-2.99 (m, 4H), 2.84 (t, J=7.2 Hz, 2H), 2.57-2.51 (m, 2H), 1.75-1.70 (m, 2H), 1.53 (s, 6H). LC-MS m/z: 394.2 [M+H]⁺. HPLC Purity (214 nm): 100%; t_{R}=8.02 min.

### Example 11: 2,2-Dimethyl-5-morpholino-N-phenethyl-3,4-dihydroquinoline-1(2H)-carboxamide

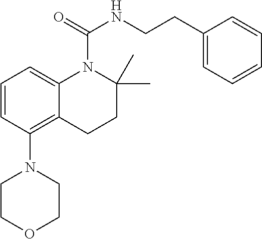 [SMILES: CC1(C)CCc2c(N3CCOCC3)cccc2N1C(=O)NCCc1ccccc1]

A solution of 4-(2,2-dimethyl-1,2-dihydroquinolin-5-yl)morpholine (244 mg, 1.0 mmol), PtO₂ (50 mg, 0.2 mmol) in MeOH (10 mL) was stirred at RT for 1 h and filtered. The filtrate was concentrated to afford 4-(2,2-dimethyl-1,2,3,4-tetrahydroquinolin-5-yl)morpholine (230 mg, 93%) as a yellow solid. LC-MS m/z: 247.3 [M+H]⁺. HPLC Purity (214 nm): 89%; t_{R}=0.74 min.

Following general procedure B, 4-(2,2-dimethyl-1,2,3,4-tetrahydroquinolin-5-yl)morpholine (123 mg, 0.5 mmol) and (2-isocyanatoethyl)benzene (220 mg, 1.5 mmol) afforded the title compound (35.1 mg, 18%) as a white solid. ¹H NMR (400 MHz, CDCl₃) δ 7.32-7.26 (m, 2H), 7.25-.17 (m, 3H), 6.95 (t, J=8.0 Hz, 1H), 6.69 (d, J=7.6 Hz, 1H), 6.63 (d, J=8.0 Hz, 1H), 4.89 (t, J=5.7 Hz, 1H), 3.89-3.84 (m, 4H), 3.49 (dd, J=13.2, 7.2 Hz, 2H), 2.95-2.91 (m, 4H), 2.84 (t, J=7.1 Hz, 2H), 2.61-2.56 (m, 2H), 1.68-1.65 (m, 2H), 1.59 (s, 6H). LC-MS m/z: 394.2 [M+H]⁺. HPLC Purity (214 nm): 97.68%; t_{R}=9.58 min.

### Example 12: 2,2-Dimethyl-7-(1-methyl-1H-imidazol-2-yl)-N-phenethyl-3,4-dihydroquinoline-1(2H)-carboxamide

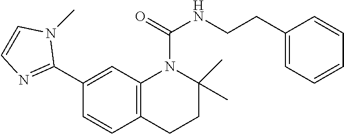 [SMILES: Cn1ccnc1-c1ccc2c(c1)N(C(=O)NCCc1ccccc1)C(C)(C)CC2]

A mixture of 7-bromo-2,2-dimethyl-1,2-dihydroquinoline and 5-bromo-2,2-dimethyl-1,2-dihydroquinoline (1.4 g, 6.0 mmol), 4,4,4′,4′,5,5,5′,5′-octamethyl-2,2′-bi(1,3,2-dioxaborolane) (1.9 g, 7.8 mmol), Pd(dppf)Cl₂.DCM (350 mg, 0.5 mmol), KOAc (1.2 g, 18 mmol) in 1,4-dioxane (20 mL) was stirred at 100° C. for 12 h under N₂. The reaction was cooled and concentrated in vacuo to give a residue which was purified by silica gel column chromatography (PE/EA=10/1) to give a mixture of 2,2-dimethyl-7-(4,4,5,5-tetramethyl-1,3,2-dioxaborolan-2-yl)-1,2-dihydroquinoline and 2,2-dimethyl-5-(4,4,5,5-tetramethyl-1,3,2-dioxaborolan-2-yl)-1,2-dihydroquinoline (1.1 g, 65%) as yellow solids. LC-MS m/z: 286.3 [M+H]⁺. HPLC Purity (214 nm): 81%; t_{R}=1.20 min.

A mixture of 2,2-dimethyl-7-(4,4,5,5-tetramethyl-1,3,2-dioxaborolan-2-yl)-1,2-dihydroquinoline and 2,2-dimethyl-5-(4,4,5,5-tetramethyl-1,3,2-dioxaborolan-2-yl)-1,2-dihydroquinoline (1.1 g, 3.8 mmol), 2-bromo-1-methyl-1H-imidazole (784 mg, 4.9 mmol), Pd(dppf)Cl₂.DCM (120 mg, 0.2 mmol), Na₂CO₃ (760 mg, 7.6 mmol) in 1,4-dioxane/H₂O (20 mL, 2/1) was stirred at 100° C. for 12 h under N₂. The reaction was cooled and concentrated in vacuo to give a residue which was purified by silica gel column chromatography (DCM/MeOH=10/1) to give 2,2-dimethyl-7-(1-methyl-1H-imidazol-2-yl)-1,2-dihydroquinoline (370 mg, crude) (LC-MS m/z: 240.3 [M+H]⁺. HPLC Purity (214 nm): 65%; t_{R}=0.710 min) and 2,2-dimethyl-5-(1-methyl-1H-imidazol-2-yl)-1,2-dihydroquinoline (260 mg, crude) as yellow solids (LC-MS m/z: 240.3 [M+H]⁺. HPLC Purity (214 nm): 70%; t_{R}=0.61 min).

A solution of 2,2-dimethyl-7-(1-methyl-1H-imidazol-2-yl)-1,2-dihydroquinoline (239 mg, 1.0 mmol), PtO₂ (50 mg, 0.2 mmol) in MeOH (10 mL) was stirred at RT for 1 h and filtered. The filtrate was concentrated to afford 2,2-dimethyl-7-(1-methyl-1H-imidazol-2-yl)-1,2,3,4-tetrahydroquinoline (250 mg, crude) as a yellow solid. LC-MS m/z: 242.4 [M+H]⁺. HPLC Purity (214 nm): 83%; t_{R}=0.70 min.

Following general procedure B, 2,2-dimethyl-7-(1-methyl-1H-imidazol-2-yl)-1,2,3,4-tetrahydroquinoline (258 mg, 1 mmol) and (2-isocyanatoethyl)benzene (441 mg, 3 mmol) afforded the title compound (45.7 mg, 12%) as a white solid. ¹H NMR (400 MHz, CDCl₃) δ 7.29-7.13 (m, 8H), 7.07 (d, J=7.7 Hz, 1H), 6.98 (s, 1H), 5.39 (bs, 1H), 3.77 (s, 3H), 3.54 (dd, J=13.3, 6.7 Hz, 2H), 2.86 (t, J=7.2 Hz, 2H), 2.71-2.66 (m, 2H), 1.80-1.75 (m, 2H), 1.54 (s, 6H). LC-MS m/z: 389.1 [M+H]⁺. HPLC Purity (214 nm): 95.77%; t_{R}=6.81 min.

### Example 13: 2,2-Dimethyl-5-(1-methyl-1H-imidazol-2-yl)-N-phenethyl-3,4-dihydroquinoline-1(2H)-carboxamide

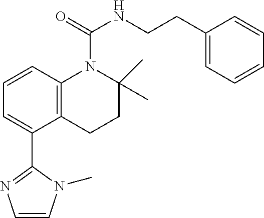 [SMILES: Cn1ccnc1-c1cccc2c1CCC(C)(C)N2C(=O)NCCc1ccccc1]

A solution of 2,2-dimethyl-5-(1-methyl-1H-imidazol-2-yl)-1,2-dihydroquinoline (320 mg, 1.2 mmol), PtO₂ (50 mg, 0.2 mmol) in MeOH (10 mL) was stirred at RT for 1 h and filtered. The filtrate was concentrated to afford 2,2-dimethyl-5-(1-methyl-1H-imidazol-2-yl)-1,2,3,4-tetrahydroquinoline (260 mg, crude) as yellow solid. LC-MS m/z: 242.4 [M+H]⁺. HPLC Purity (214 nm): 90%; t_{R}=0.61 min.

Following general procedure B, 2,2-dimethyl-5-(1-methyl-1H-imidazol-2-yl)-1,2,3,4-tetrahydroquinoline (258 mg, 1 mmol) and (2-isocyanatoethyl)benzene (441 mg, 3 mmol) afforded the title compound (71.8 mg, 18%) as a white solid. ¹H NMR (400 MHz, CDCl₃) δ 7.31-7.27 (m, 2H), 7.25-7.19 (m, 4H), 7.04-6.91 (m, 4H), 5.08 (bs, 1H), 3.57-3.53 (m, 2H), 3.56 (s, 3H), 2.87 (t, J=7.0 Hz, 2H), 2.44-2.39 (m, 2H), 1.67-1.62 (m, 2H), 1.55 (s, 6H). LC-MS m/z: 389.1 [M+H]⁺. HPLC Purity (214 nm): 97.5%; t_{R}=6.67 min.

### Example 14: 8, 8-Dimethyl-N-phenethyl-5,6-dihydro-1,7-naphthyridine-7(8H)-carboxamide

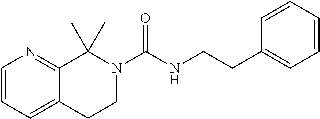 [SMILES: CC1(C)c2ncccc2CCN1C(=O)NCCc1ccccc1]

To a solution of 6,7-dihydro-1,7-naphthyridin-8(5H)-one (352 mg, 2.38 mmol) in DMF (6 mL) was added NaH (143 mg, 3.57 mmol) at 0° C. and the mixture was stirred for 30 min. (Bromomethyl)benzene (485 mg, 2.85 mmol) was added and the mixture was stirred at RT for 2 h. The reaction was quenched with H₂O (30 mL), extracted with EA (3×30 mL) and the combined organic layers were concentrated. The residue was purified by silica gel column chromatography (DCM:MeOH=15:1) to afford 7-benzyl-6,7-dihydro-1,7-naphthyridin-8(5H)-one (450 mg, 80%) as a yellow oil. LC-MS m/z: 239.3 [M+H]⁺. HPLC Purity (254 nm): >99%; t_{R}=0.77 min.

To a solution of 7-benzyl-6,7-dihydro-1,7-naphthyridin-8(5H)-one (450 mg, 1.9 mmol) in THF (6 mL) was added ZrCl₄ (573 mg, 2.46 mmol) at −10° C. and the mixture was stirred for 30 min. MeMgBr (3N, 5.05 mL) was added and the mixture was stirred at RT for 2 h and then concentrated. The residue was purified by silica gel column chromatography (DCM:MeOH=15:1) to afford 7-benzyl-8,8-dimethyl-5,6,7,8-tetrahydro-1,7-naphthyridine (85 mg, 18%) as a yellow solid. LC-MS m/z: 253.0 [M+H]⁺. HPLC Purity (254 nm): >58%; t_{R}=2.28 min.

A solution of 7-benzyl-8, 8-dimethyl-5,6,7,8-tetrahydro-1,7-naphthyridine (85 mg, 0.34 mmol) and Pd/C (10 mg) in MeOH (5 mL) was stirred at RT under H2 for 1 hour. The reaction was filtered, washed with MeOH (5 mL) and concentrated. The residue was purified by silica gel column chromatography (DCM:MeOH=10:1) to afford 8,8-dimethyl-5,6,7,8-tetrahydro-1,7-naphthyridine (20 mg, 37%) as a yellow oil. LC-MS m/z: 163.2 [M+H]⁺. Purity (254 nm): >88%; t_{R}=1.28 min.

Following general procedure B, 8,8-dimethyl-5,6,7,8-tetrahydro-1,7-naphthyridine (20 mg, 0.12 mmol) and (2-isocyanatoethyl)benzene (36 mg, 0.25 mmol) afforded the title compound (12.3 mg, 32.3%) as a white solid. ¹H NMR (400 MHz, CDCl₃) δ 8.48 (dd, J=4.6, 1.6 Hz, 1H), 7.40-7.18 (m, 6H), 7.04 (dt, J=19.1, 9.5 Hz, 1H), 4.51 (bs, 1H), 3.53 (q, J=6.8 Hz, 2H), 3.49-3.46 (m, 2H), 2.88 (t, J=6.8 Hz, 2H), 2.80-2.78 (m, 2H), 1.78 (s, 6H). LC-MS m/z: 310.2 [M+H]⁺. HPLC Purity (214 nm): >99%; t_{R}=1.53 min.

### Example 15: 1,1-Dimethyl-N-phenethyl-3,4-dihydroisoquinoline-2(1H)-carboxamide

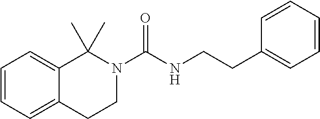 [SMILES: CC1(C)c2ccccc2CCN1C(=O)NCCc1ccccc1]

A solution of 2-(2-bromophenyl)ethanamine (1.0 g, 5 mmol), trifluoroacetic anhydride (1.3 g, 6 mmol) and Et₃N (1.5 g 15 mmol) in DCM (11 mL) was stirred overnight at RT. The mixture was concentrated and purified by column chromatography (PE/EA=3:1) to give N-(2-bromophenethyl)-2,2,2-trifluoroacetamide (1.3 g, 88%) as a white solid. LC-MS m/z: 296 [M+H]⁺. HPLC Purity (214 nm): 98%; Rt=2.03 min.

A mixture of N-(2-bromophenethyl)-2,2,2-trifluoroacetamide (1.3 g, 4.4 mmol), 4,4,5,5-tetramethyl-2-(prop-1-en-2-yl)-1,3,2-dioxaborolane (889 mg, 5.3 mmol), K₂CO₃ (1.4 g, 13.2 mmol) and Pd(dppf)Cl₂. (322 mg, 0.44 mmol) in 1,4-dioxane/H₂O (25 mL, 4/1) was stirred at 110° C. under N₂ for 3 hrs. The reaction was cooled and concentrated under vacuo to give a residue, which was purified by silica gel column chromatography (PE/EA=3:1) to give 2, 2, 2-trifluoro-N-(2-(prop-1-en-2-yl)phenethyl)acetamide (1.0 g, 88%) as a white solid. LC-MS m/z: 258 [M+H]⁺. HPLC Purity (214 nm): 75%; t_{R}=2.10 min.

To a solution of 2,2,2-trifluoro-N-(2-(prop-1-en-2-yl)phenethyl)acetamide (800 mg, 3.1 mmol) in CH₃Cl (50 mL) was added trifluoromethanesulfonic acid (520 mg) and the mixture was stirred at RT under H2 overnight. The reaction was cooled and concentrated under vacuo to give a residue, which was purified by silica gel column chromatography (PE/EA=3:1) to afford 1-(1,1-dimethyl-3,4-dihydroisoquinolin-2(1H)-yl)-2,2,2-trifluoroethanone (200 mg, 25%) as a white solid. LC-MS m/z: 258 [M+H]⁺. HPLC Purity (214 nm): 75%; t_{R}=1.50 min.

To a solution of 1-(1,1-dimethyl-3,4-dihydroisoquinolin-2(1H)-yl)-2,2,2-trifluoroethanone (200 mg, 0.78 mmol) in MeOH (50 mL) was added 5N NaOH (2 ml) and the mixture was stirred at RT overnight. The reaction was cooled and concentrated in vacuo to give a residue, which was purified by silica gel column chromatography (PE/EA=3:1) to give 1,1-dimethyl-1,2,3,4-tetrahydroisoquinoline (100 mg, 25%) as a white solid. LC-MS m/z: 162 [M+H]⁺. HPLC Purity (214 nm): 90%; t_{R}=0.37 min.

Following general procedure B, 1,1-dimethyl-1,2,3,4-tetrahydroisoquinoline (100 mg, 0.062 mmol) and (2-isocyanatoethyl)benzene (410 mg 2.8 mmol) afforded the title compound (13 mg, 6.8%) as a white solid. ¹H NMR (400 MHz, CDCl₃) δ 7.39-7.17 (m, 7H), 7.16-7.01 (m, 2H), 4.40 (bs, 1H), 3.50 (dd, J=12.6, 6.7 Hz, 2H), 3.43-3.32 (m, 2H), 2.86 (t, J=6.8 Hz, 2H), 2.80 (dt, J=10.8, 6.0 Hz, 2H), 1.80 (s, 6H). LC-MS m/z: 309 [M+H]⁺. HPLC Purity (214 nm): 99%; t_{R}=9.47 min.

### Example 16: 3,3-Dimethyl-N-phenethyl-2H-benzo[b][1,4]oxazine-4(3H)-carboxamide

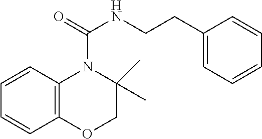 [SMILES: CC1(C)COc2ccccc2N1C(=O)NCCc1ccccc1]

To a mixture of 2-aminophenol (5 g, 45.82 mmol) in DCM (15 mL) was added Boc₂O (9 g, 41.24 mmol) and Et₃N (5.1 g, 50.39 mmol) and the mixture was stirred at RT for 24 h. The mixture was filtered and concentrated to afford tert-butyl 2-hydroxyphenylcarbamate (2.1 g, 30%), which was used directly in the next step. LC-MS m/z: 154.3 [M+H]⁺. HPLC Purity (214 nm): 85.9%; t_{R}=0.96 min.

A mixture of tert-butyl 2-hydroxyphenylcarbamate (10.0 g, 47.8 mmol), 3-chloro-2-methylprop-1-ene (6.5 g, 71.7 mmol) and K₂CO₃ (13.2 g, 95.6 mmol) in acetone (80 mL) was stirred at 80° C. for 24 h. The reaction was cooled and concentrated in vacuo to give a residue which was purified by silica gel column chromatography (PE/EA=6/1) to give tert-butyl-2-(2-methylallyloxy)phenylcarbamate (6.4 g, 58%). LC-MS m/z: 208.3 [M−55]⁺. HPLC Purity (254 nm): 100%; t_{R}=1.22 min.

A solution of tert-butyl 2-(2-methylallyloxy)phenylcarbamate (6.3 g, 23.86 mmol) in a dioxane solution of HCl (20 mL) was stirred at RT for 2 h. The mixture was filtered and concentrated to give 2-(2-methylallyloxy)aniline (4.5 g, 94.0%) which was used directly in the next step. LC-MS m/z: 164.4 [M+H]⁺. HPLC Purity (214 nm): 100%; t_{R}=0.77 min.

To a stirred solution of 2-(2-methylallyloxy)aniline (5 g, 25.0 mmol) in 4 N HCl (50 mL) was added NaNO₂ (1.8 g, 26.3 mmol) at 0° C. NaHCO₃ was then added to adjust the pH to 8, and then NaN₃ (1.63 g, 25.0 mmol) was added and the mixture was stirred at 0° C. for 1 h. The reaction mixture was extracted with DCM (50 mL×2) and the combined organic layers were washed with brine (50 mL×1), dried over Na₂SO₄, filtered and concentrated to give a 1-azido-2-(2-methylallyloxy)benzene (4.73 g), which was used directly in the next step.

A solution of 1-azido-2-(2-methylallyloxy)benzene (4.73 g, 25.0 mmol) in toluene (60 mL) was stirred at 100° C. for 24 h. The mixture was concentrated to give 1a-methyl-1a,2-dihydro-1H-azirino[1,2-d]benzo[b][1,4]oxazine (3.5 g) which was used directly in the next step. LC-MS m/z: 162.0 [M+H]⁺. HPLC Purity (214 nm): 78.4%; t_{R}=1.83 min.

To a solution of 1a-methyl-1a,2-dihydro-1H-azirino[1,2-d]benzo[b][1,4]oxazine (1.8 g, 11.17 mmol) in MeOH (50 mL) was added Pd/C (0.9 g). The mixture was stirred at RT under H2 for 1 h and then filtered and concentrated to give a residue which was purified by silica gel column chromatography (PE/EA=6/1) to afford 3,3-dimethyl-3,4-dihydro-2H-benzo[b][1,4]oxazine (1.6 g, 88.8%) as a brown solid. LC-MS m/z: 164.0 [M+H]⁺. HPLC Purity (254 nm): 100%; t_{R}=1.95 min.

Following general procedure B, 3,3-dimethyl-3,4-dihydro-2H-benzo[b][1,4]oxazine (300 mg, 1.83 mmol) and (2-isocyanatoethyl)benzene (946.7 mg, 6.42 mmol) afforded the title compound (488 mg, 86%) as a white solid. ¹H NMR (500 MHz, CDCl₃) δ 7.33-7.29 (m, 2H), 7.26-7.18 (m, 3H), 6.85-6.76 (m, 3H), 6.71-6.66 (m, 1H), 5.25 (bs, 1H), 3.82 (s, 2H), 3.61-3.55 (m, 2H), 2.89 (t, J=6.4 Hz, 2H), 1.42 (s, 6H). LC-MS m/z: 311.4 [M+H]⁺. HPLC Purity (214 nm): 100%; t_{R}=9.62 min.

### Example 17: 8,8-Dimethyl-N-phenethyl-1-oxa-9-azaspiro[5.5]undecane-9-carboxamide

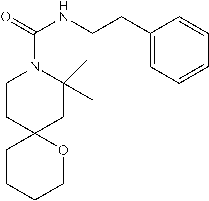 [SMILES: CC1(C)CC2(CCCCO2)CCN1C(=O)NCCc1ccccc1]

To a solution of allylmagnesium bromide (1 M in THF, 6 mL) was added tert-butyl 2,2-dimethyl-4-oxopiperidine-1-carboxylate (600 mg, 2.64 mmol) in THF (3 mL) dropwise at 0° C. and the mixture was stirred at RT for 4 h. The reaction was treated with aq. NH₄Cl (30 mL), extracted with EA (2×30 mL) and concentrated. The residue was purified by silica gel column chromatography (PE:EA=3:1) to afford tert-butyl 4-allyl-4-hydroxy-2,2-dimethylpiperidine-1-carboxylate (550 mg, 77%) as a yellow oil. LC-MS m/z: 170.4 [M−100+H]⁺. HPLC Purity (214 nm): >48%; t_{R}=1.08 min.

To a solution of tert-butyl 4-allyl-4-hydroxy-2, 2-dimethylpiperidine-1-carboxylate (750 mg, 2.79 mmol) in DMF (450 mL) was added NaH (558 mg, 13.9 mmol) at 0° C. and the mixture was stirred for 30 minutes. 3-bromoprop-1-ene (1.67 g, 13.9 mmol) was added and the mixture was stirred at RT for 15 h. The reaction was quenched with water (100 mL), extracted with EA (3×50 mL) and concentrated. The residue was purified by silica gel column chromatography (PE:EA=10:1) to afford tert-butyl 4-allyl-4-(allyloxy)-2,2-dimethylpiperidine-1-carboxylate (650 mg, 75%) as a yellow oil. LC-MS m/z: 210.4 [M−100+H]⁺. HPLC Purity (214 nm): >85%; t_{R}=1.31 min.

To a solution of GRUBB'S catalyst (350 mg, 0.42 mmol) in DCM (450 mL) was added tert-butyl 4-allyl-4-(allyloxy)-2,2-dimethylpiperidine-1-carboxylate (650 mg, 2.1 mmol) in DCM (50 mL) dropwise over 3 h and the mixture was stirred at RT for 15 h. The reaction was concentrated, and the residue was purified by silica gel column chromatography (PE:EA=10:1) to get tert-butyl 8, 8-dimethyl-1-oxa-9-azaspiro[5.5]undec-3-ene-9-carboxylate (500 mg, 85%) as a yellow oil. LC-MS m/z: 182.4 [M−100+H]⁺. HPLC Purity (214 nm): >42%; t_{R}=1.20 min.

A mixture of tert-butyl 8,8-dimethyl-1-oxa-9-azaspiro[5.5]undec-3-ene-9-carboxylate (500 mg, 1.78 mmol) and Pd/C (100 mg) in MeOH (15 mL) was stirred at RT under H2 for 1 h. The reaction was filtered, washed with MeOH (15 mL) and concentrated to give tert-butyl 8, 8-dimethyl-1-oxa-9-azaspiro[5.5]undecane-9-carboxylate (500 mg, 99%) as a yellow oil. LC-MS m/z: 184.4 [M−100+H]⁺. HPLC Purity (214 nm): >68%; t_{R}=1.24 min.

To a solution of tert-butyl 8, 8-dimethyl-1-oxa-9-azaspiro[5.5]undecane-9-carboxylate (450 mg, 1.59 mmol) in DCM (3 mL) was added HCl (4M in dioxane, 3 mL) and the mixture was stirred at RT for 15 h. The reaction mixture was diluted with DCM (15 mL), NaHCO₃ (100 mg) was added and the solution was stirred at RT for 1 hour. The reaction was filtered, washed with DCM (10 mL) and concentrated to give 8,8-dimethyl-1-oxa-9-azaspiro[5.5]undecane (230 mg, 79%) as a yellow oil. LC-MS m/z: 184.3 [M+H]⁺. HPLC Purity (254 nm): >50%; t_{R}=0.58 min.

Following general procedure B, 8,8-dimethyl-1-oxa-9-azaspiro[5.5]undecane (190 mg, 1.04 mmol) and (2-isocyanatoethyl)benzene (763 mg, 5.19 mmol) afforded the title compound (53.3 mg, 15.6%) as a white solid. ¹H NMR (400 MHz, CDCl₃) δ 7.36-7.29 (m, 2H), 7.24-7.17 (m, 3H), 4.39 (bs, 1H), 3.68-3.60 (m, 2H), 3.53-3.42 (m, 2H), 3.29 (ddd, J=13.6, 10.9, 2.9 Hz, 1H), 3.12 (ddd, J=12.6, 5.3, 4.1 Hz, 1H), 2.82 (t, J=6.8 Hz, 2H), 1.92-1.84 (m, 2H), 1.67-1.40 (m, 8H), 1.42 (s, 6H). LC-MS m/z: 331.1 [M+H]⁺. HPLC Purity (214 nm): >99%; t_{R}=2.11 min.

### Example 18: 2,2-Dimethyl-N-(2-phenoxyethyl)-3,4-dihydroquinoline-1(2)-carboxamide

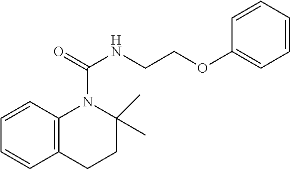 [SMILES: CC1(C)CCc2ccccc2N1C(=O)NCCOc1ccccc1]

Following general procedure B, 2,2-dimethyl-1,2,3,4-tetrahydroquinoline (150 mg, 0.9 mmol) and (2-isocyanatoethoxy)benzene (759 mg, 4.7 mmol) afforded the title compound (13 mg, 4%) as a colorless oil. ¹H NMR (400 MHz, CDCl₃) δ 7.31-7.28 (m, 2H), 7.08 (d, J=7.6 Hz, 2H), 7.02-6.97 (m, 2H), 6.96-6.89 (m, 1H), 6.87 (d, J=8.0 Hz, 2H), 5.45 (bs, 1H), 4.07 (t, J=4.8 Hz, 2H), 3.63 (q, J=5.2 Hz, 2H), 2.60-2.57 (m, 2H), 1.75-1.71 (m, 2H), 1.56 (s, 3H), 1.55 (s, 3H). LC-MS m/z: 325.2 [M+H]⁺. HPLC Purity (214 nm): 95%; t_{R}=10.02 min.

### Example 19: 4-Methoxy-2,2-dimethyl-N-phenethyl-3,4-dihydroquinoline-1(2)-carboxamide

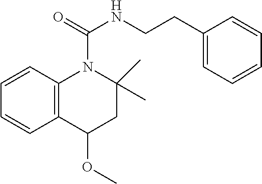 [SMILES: COC1CC(C)(C)N(C(=O)NCCc2ccccc2)c2ccccc21]

A mixture of 2-iodoaniline (5 g, 22.8 mmol), CuI (434 mg, 2.3 mmol), Pd(PPh₃)₂Cl₂ (842 mg, 1.2 mmol) and Et₃N (46 g, 456 mmol) in ACN (60 mL) was stirred at 50° C. for 2 h under N₂. The mixture was then filtered and concentrated in vacuo to give a residue which was purified by silica gel column chromatography (PE:EA=1:1) to afford 4-(2-aminophenyl)-2-methylbut-3-yn-2-ol (3.5 g, 88%) as a brown oil. LC-MS m/z: 158.1 [M+H]⁺. HPLC Purity (214 nm): 73%; t_{R}=1.75 min.

A solution of 4-(2-aminophenyl)-2-methylbut-3-yn-2-ol (3.5 g, 20 mmol) in HCl (50 mL, 0.1 M) was stirred at 120° C. for 3 h. Then the mixture was concentrated in vacuo to give a residue which was purified by silica gel column chromatography (DCM/MeOH=100:1) to give 2,2-dimethyl-2,3-dihydroquinolin-4(1H)-one (2.5 g, 71%) as a yellow solid. LC-MS m/z: 176.4 [M+H]⁺. HPLC Purity (214 nm): 64%; t_{R}=0.84 min.

A solution of 2,2-dimethyl-2,3-dihydroquinolin-4(1H)-one (2.5 g, 14.3 mmol) and (bromomethyl)benzene (3.7 g, 21.4 mmol) in DIEA (30 mL) was stirred at 120° C. for 48 h. Then the mixture was concentrated in vacuo to give a residue which was purified by silica gel column chromatography (PE/EA=10:1) to give 1-benzyl-2,2-dimethyl-2,3-dihydroquinolin-4(1H)-one (3 g, 79%) as a yellow solid. LC-MS m/z: 266.4 [M+H]⁺. HPLC Purity (214 nm): 96%; t_{R}=1.12 min.

A solution of 1-benzyl-2,2-dimethyl-2,3-dihydroquinolin-4(1H)-one (2.8 g, 10.6 mmol) and LiAlH₄ (600 mg, 15.8 mmol) in THF (30 mL) was stirred at RT for 1 h. Then the mixture was filtered and concentrated in vacuo to give 1-benzyl-2,2-dimethyl-1,2,3,4-tetrahydroquinolin-4-ol (2.5 g, 89%) as a yellow oil. LC-MS m/z: 268.3 [M+H]₊. HPLC Purity (214 nm): 96%; t_{R}=1.11 min.

A solution of 1-benzyl-2,2-dimethyl-1,2,3,4-tetrahydroquinolin-4-ol (2.0 g, 7.5 mmol) and NaH (270 mg, 11.2 mmol) in DMF (20 mL) was stirred at RT for 1 h and then MeI (5.3 g, 37.4 mmol) was added and the resulting mixture was stirred at RT overnight. The mixture was concentrated in vacuo to give a residue which was purified by silica gel column chromatography (PE/EA=3:1) to afford 1-benzyl-4-methoxy-2,2-dimethyl-1,2,3,4-tetrahydroquinoline (1.9 g, 90%) as a yellow solid. LC-MS m/z: 282.3 [M+H]⁺. HPLC Purity (214 nm): 97%; t_{R}=1.28 min.

A solution of 1-benzyl-4-methoxy-2,2-dimethyl-1,2,3,4-tetrahydroquinoline (1.9 g, 6.75 mmol) and Pd/C (200 mg) in MeOH (300 mL) was stirred at RT for 24 h. Then the mixture was filtered and concentrated in vacuo to give a residue which was purified by silica gel column chromatography (DCM/MeOH=100:1) to afford 4-methoxy-2,2-dimethyl-1,2,3,4-tetrahydroquinoline (1.1 g, 77%) as a brown solid. LC-MS m/z: 192.3 [M+H]⁺. HPLC Purity (214 nm): 90%; t_{R}=0.91 min.

Following general procedure B, 4-methoxy-2,2-dimethyl-1,2,3,4-tetrahydroquinoline (200 mg, 1.1 mmol) and (2-isocyanatoethyl)benzene (923 mg, 6.3 mmol) afforded the title compound (260.2 mg, 74%) as a white solid. ¹H NMR (400 MHz, CDCl₃) δ 7.33 (dd, J=6.0 Hz, 0.8 Hz, 1H), 7.30-7.27 (m, 2H), 7.23-7.21 (m, 1H), 7.20-7.16 (m, 2H), 7.03-6.94 (m, 2H), 6.87 (dd, J=6.8 Hz, 1.6 Hz, 1H), 4.97 (bs, 1H), 4.19 (dd, J=6.0 Hz, 3.6 Hz, 1H), 3.57-3.53 (m, 1H), 3.51 (s, 3H), 3.49-3.45 (m, 1H), 2.86-2.81 (m, 2H), 2.13 (dd, J=9.2 Hz, 3.6 Hz, 1H), 1.65-1.59 (m, 1H), 1.60 (s, 3H), 1.58 (s, 3H). LC-MS m/z: 307.2 [M+H]⁺. HPLC Purity (214 nm): 96%; t_{R}=10.99 min.

### Example 20: 3,3-Dimethyl-8-(1-methylpiperidin-4-yl)-N-phenethyl-3,4-dihydroisoquinoline-2(1H)-carboxamide

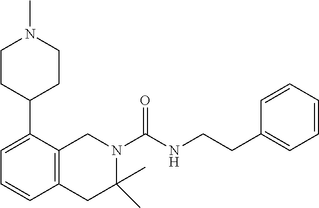 [SMILES: CN1CCC(c2cccc3c2CN(C(=O)NCCc2ccccc2)C(C)(C)C3)CC1]

To a solution of methyl isobutyrate (2.45 g, 24.0 mmol) in dry THF (30 mL) was added LiHMDS (1.0 M in THF) (40 mL, 40.0 mmol) at 0° C. and the resulting reaction mixture was stirred for 20 min at 0° C. Then a solution of 1-bromo-3-(bromomethyl)benzene (5.0 g, 20.0 mmol) in dry THF (20 mL) was added and the mixture was stirred at RT for 2 h. The reaction mixture was quenched with H₂O (50 mL) and extracted with EA (100 mL×2). The combined organic layers were washed with brine (50 mL×1), dried over Na₂SO₄, filtered and concentrated to give a residue which was purified by silica gel column chromatography (PE:EA=9:1) to give methyl 3-(3-bromophenyl)-2,2-dimethylpropanoate (4.5 g, 78%) as a clear oil. LC-MS m/z: 271.0, 273.0 [M+H]⁺. HPLC Purity (214 nm): 88%; t_{R}=2.24 min.

To a solution of methyl 3-(3-bromophenyl)-2,2-dimethylpropanoate (4.5 g, 16.6 mmol) in MeOH (30 mL) was added a solution of NaOH (3.3 g, 82.5 mmol) in H₂O (5 mL). The reaction mixture was stirred at 65° C. for 16 h and then the reaction mixture was concentrated and redissolved in H₂O (20 mL) followed by the addition of 6N HCl solution at 0° C. to adjust the pH to 5. The precipitate was filtered and 3-(3-bromophenyl)-2,2-dimethylpropanoic acid (4.2 g, 98.5%) was collected as a yellow solid. LC-MS m/z: 210.9, 212.9 [M-46]+. HPLC Purity (214 nm): 86%; t_{R}=2.01 min.

To a stirred solution of 3-(3-bromophenyl)-2,2-dimethylpropanoic acid (2.0 g, 7.81 mmol) in toluene were added TEA (1.57 g, 15.6 mmol) and DPPA (3.22 g, 11.7 mmol) and the solution was stirred at 100° C. for 2 h, then MeOH (1.0 g, 31.24 mmol) was added. The reaction was stirred at 100° C. for an additional 16 h and quenched with H₂O (100 mL) and extracted with EA (150 mL×2). The combined organic layers were washed with brine (50 mL×1), dried over Na₂SO₄, filtered and concentrated to give a residue which was purified by silica gel column chromatography (PE:EA=20:1) to give methyl (1-(3-bromophenyl)-2-methylpropan-2-yl)carbamate (1.6 g, 72%) as a clear oil. LC-MS m/z: 286.0, 288.0 [M+H]+. HPLC Purity (214 nm): 88%; t_{R}=2.13 min.

To a solution of methyl (1-(3-bromophenyl)-2-methylpropan-2-yl)carbamate (1.45 g, 5.09 mmol) in AcOH/H₂SO₄ (3:1; 15 mL) at 0° C. was slowly added paraformaldehyde (0.23 g, 7.63 mmol). After 16 h of stirring at RT, the reaction mixture was quenched with H₂O (100 mL) and extracted with EA (150 mL×2). The combined organic layers were washed with saturated NaHCO₃ solution (30 mL), H₂O (30 mL), brine (30 mL), dried over Na₂SO₄, filtered and concentrated to give methyl 8-bromo-3,3-dimethyl-3,4-dihydroisoquinoline-2(1H)-carboxylate and methyl 6-bromo-3,3-dimethyl-3,4-dihydroisoquinoline-2(1H)-carboxylate (1.2 g, 80%) as a mixture. LC-MS m/z: 298.0, 300.0 [M+H]⁺. HPLC Purity (214 nm): 80%; t_{R}=2.20 min.

A mixture of methyl 8-bromo-3,3-dimethyl-3,4-dihydroisoquinoline-2(1H)-carboxylate and methyl 6-bromo-3,3-dimethyl-3,4-dihydroisoquinoline-2(1H)-carboxylate (1.4 g, 4.71 mmol), 1-methyl-4-(4,4,5,5-tetramethyl-1,3,2-dioxaborolan-2-yl)-1,2,3,6-tetrahydropyridine (1.37 g, 6.12 mmol), Na₂CO₃ (1.0 g, 9.42 mmol) and Pd[(tBu)₃P]₂ (241 mg, 0.47 mmol) in 1,4-dioxane/H₂O (15 mL, 3/1) was stirred at 100° C. under N₂ for 2 h. The reaction was cooled and concentrated in vacuo to give a residue which was purified by silica gel column chromatography (DCM:MeOH=9:1) to give a mixture of methyl 3,3-dimethyl-8-(1-methyl-1,2,3,6-tetrahydropyridin-4-yl)-3,4-dihydroisoquinoline-2(1H)-carboxylate and methyl 3,3-dimethyl-6-(1-methyl-1,2,3,6-tetrahydropyridin-4-yl)-3,4-dihydroisoquinoline-2(1H)-carboxylate (1.8 g, 90%) as a brown oil. LC-MS m/z: 315.4 [M+H]⁺. HPLC Purity (214 nm): 30.85%, 69.15%; t_{R}=0.79 min, 0.73 min.

To a solution of methyl 3,3-dimethyl-8-(1-methyl-1,2,3,6-tetrahydropyridin-4-yl)-3,4-dihydroisoquinoline-2(1H)-carboxylate and methyl 3,3-dimethyl-6-(1-methyl-1,2,3,6-tetrahydropyridin-4-yl)-3,4-dihydroisoquinoline-2(1H)-carboxylate (1.2 g, 3.82 mmol) in CH₃OH (100.0 mL) was added PtO₂ (600 mg). The mixture was stirred at RT under H₂ for 2 days. The mixture was filtered and concentrated. The residue was purified by Prep-HPLC (MeCN/TFA) to afford methyl 3,3-dimethyl-8-(1-methylpiperidin-4-yl)-3,4-dihydroisoquinoline-2(1H)-carboxylate (310 mg, 22%) and methyl 3,3-dimethyl-6-(1-methylpiperidin-4-yl)-3,4-dihydroisoquinoline-2(1H)-carboxylate (630 mg, 45%) as white solids. LC-MS m/z: 317.4 [M+H]⁺. HPLC Purity (214 nm): 99%; t_{R}=0.86 min. LC-MS m/z: 317.4 [M+H]⁺. HPLC Purity (214 nm): 97%; t_{R}=0.80 min.

To a solution of methyl 3,3-dimethyl-8-(1-methylpiperidin-4-yl)-3,4-dihydroisoquinoline-2(1H)-carboxylate (310 mg, 0.98 mmol)) in ethylene glycol (6 mL) was added 50% aqueous KOH solution (3 mL) and the reaction mixture was heated at 150° C. for 2 days. The reaction mixture was cooled, diluted with H₂O (6 mL), extracted with DCM (100 mL×2). The combined organic layers were washed with H₂O (30 mL), brine (30 mL), dried over Na₂SO₄ and concentrated to give 3,3-dimethyl-8-(1-methylpiperidin-4-yl)-1,2,3,4-tetrahydroisoquinoline (200 mg, 79.3%) as a white solid. LC-MS m/z: 259.4 [M+H]⁺. HPLC Purity (214 nm): 92%; t_{R}=0.33 min.

Following general procedure B, 3,3-dimethyl-8-(1-methylpiperidin-4-yl)-1,2,3,4-tetrahydroisoquinoline (170 mg, 0.66 mmol) and (2-isocyanatoethyl)benzene (194 mg, 1.32 mmol) afforded the title compound (107 mg, 40%) as a white solid. ¹H NMR (400 MHz, CDCl₃) δ 7.33-7.30 (m, 2H), 7.25-7.16 (m, 5H), 7.00 (dd, J=6.8, 1.6 Hz, 1H), 4.47 (s, 2H), 4.36 (t, J=5.2 Hz, 1H), 3.55 (q, J=6.4 Hz, 2H), 2.97 (d, J=11.2 Hz, 2H), 2.88 (t, J=6.8 Hz, 2H), 2.73 (s, 2H), 2.67-2.65 (m, 1H), 2.33 (s, 3H), 2.10 (td, J=11.6 Hz, 2.8 Hz, 2H), 1.82-1.71 (m, 4H), 1.29 (s, 6H). LC-MS m/z: 406.1 [M+H]⁺. HPLC Purity (214 nm): 95%; t_{R}=5.70 min.

### Example 21: 3,3-Dimethyl-6-(1-methylpiperidin-4-yl)-N-phenethyl-3,4-dihydroisoquinoline-2(1H)-carboxamide

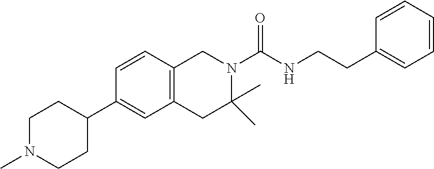 [SMILES: CN1CCC(c2ccc3c(c2)CC(C)(C)N(C(=O)NCCc2ccccc2)C3)CC1]

To a solution of methyl 3,3-dimethyl-6-(1-methylpiperidin-4-yl)-3,4-dihydroisoquinoline-2(1H)-carboxylate (900 mg, 2.58 mmol)) in ethylene glycol (10 mL) was added 50% aqueous KOH solution (5 mL) and the reaction mixture was heated at 150° C. for 3 days. The reaction mixture was cooled, diluted with H₂O (6 mL), extracted with DCM (100 mL×2). The combined organic layers were washed with H₂O (50 mL), brine (30 mL), dried over Na₂SO₄, filtered and concentrated to give methyl 3,3-dimethyl-6-(1-methylpiperidin-4-yl)-3,4-dihydroisoquinoline-2(1H)-carboxylate (495 mg, 74.0%/a). LC-MS m/z: 259.4 [M+H]⁺. HPLC Purity (214 nm): 88%; t_{R}=0.18 min.

Following general procedure B, methyl 3,3-dimethyl-6-(1-methylpiperidin-4-yl)-3,4-dihydroisoquinoline-2(1H)-carboxylate (200 mg, 0.78 mmol) and (2-isocyanatoethyl)benzene (171 mg, 1.16 mmol) afforded the title compound (69.5 mg, 34.9%) as a white solid. ¹H NMR (400 MHz, CDCl₃) δ 7.33-7.30 (m, 2H), 7.25-7.21 (m, 3H), 7.07-7.00 (m, 3H), 4.30 (t, J=5.2 Hz, 1H), 4.24 (s, 2H), 3.51 (q, J=6.8 Hz, 2H), 3.22 (d, J=12.0 Hz, 2H), 2.85 (t, J=6.8 Hz, 2H), 2.71 (s, 2H), 2.55-2.51 (m, 1H), 2.48 (s, 3H), 2.32 (td, J=13.6 Hz, 1.2 Hz, 2H), 2.04-1.86 (m, 4H), 1.36 (s, 6H). LC-MS m/z: 406.1 [M+H]+. HPLC Purity (214 nm): 98%; t_{R}=5.65 min.

### Example 22: 3,3-Dimethyl-N-phenethyl-3,4-dihydroisoquinoline-2(1H)-carboxamide

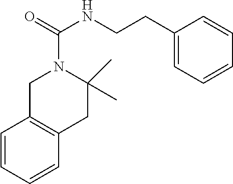 [SMILES: CC1(C)Cc2ccccc2CN1C(=O)NCCc1ccccc1]

To a solution of (bromomethyl)benzene (1.00 g, 9.80 mmol) in THF (3.0 mL) was added LiHMDS in THF dropwise at 0° C. and the solution was stirred for 1 h followed by the slow addition of a solution of methyl isobutyrate dissolved in THF (3.0 mL). The mixture was stirred overnight at RT and then extracted with EA and concentrated. The combined organic phase was purified by silica gel column chromatography (EA/PE=1/4) to give methyl 2,2-dimethyl-3-phenylpropanoate (0.6 g, 52%) as colorless oil. LC-MS m/z: 193.3 [M+H]⁺. HPLC Purity (214 nm): 78%; t_{R}=1.09 min.

To a solution of methyl 2, 2-dimethyl-3-phenylpropanoate (3.5 g, 18.2 mmol) in MeOH (20 ml) was added NaOH (3.63 g, 91.2 mmol) and the mixture was stirred overnight at 65° C. The reaction was cooled and extracted with EA/H₂O and the concentrated organic phase was purified by a flash column (EA:PE=1:10) to give 2,2-dimethyl-3-phenylpropanoic acid (1.8 g, 55%), which was used directly in the next step. LC-MS m/z: 179.2 [M+H]⁺. t_{R}=1.10 min.

Crude 2, 2-dimethyl-3-phenylpropanoic acid (1.20 g, 6.73 mmol) was added to a mixture of DPPA (2.20 g, 8.08 mmol) and TEA (2.0 mL) in toluene (15 mL) and stirred under N₂ for 1 h at 80° C. MeOH (2.0 mL) was then added and the mixture was stirred for another 2 h. The reaction was concentrated and purified by a flash column (EA:PE=1:5) to give methyl 2-methyl-1-phenylpropan-2-ylcarbamate (700 mg, 50.2%) as a colorless oil. LC-MS m/z: 208.1 [M+H]⁺; HPLC Purity (214 nm): 100%; t_{R}=1.80 min.

To a mixture AcOH and H₂SO₄ (8 mL, 3:1) was added methyl 2-methyl-1-phenylpropan-2-ylcarbamate (0.50 g, 0.25 mmol) and (CH₂O)ₙ (0.15 g, 0.50 mmol) and stirred at room temperature for 1 h. The completed reaction was extracted with EA/H₂O (5/10 mL) and NaCO₃/H₂O solution (5/10 mL) to give a combined organic phase, which was later concentrated under vacuum to give crude methyl 3,3-dimethyl-3,4-dihydroisoquinoline-2(1H)-carboxylate (500 mg, 92%). LC-MS m/z: 220.1 [M+H]⁺. HPLC Purity (214 nm): 100%; t_{R}=2.09 min.

To a solution of Et₂OH (25.0 mL) and H₂O (10.0 mL) was added methyl 3,3-dimethyl-3,4-dihydroisoquinoline-2(1H)-carboxylate (3.20 g, 14.61 mmol) and KOH (2.46 g, 43.84 mmol) and the solution was stirred overnight at 135° C. The mixture was purified by silica gel column chromatography (EA:PE/1:5) to give 3,3-dimethyl-1,2,3,4-tetrahydroisoquinoline (210 mg, 8.9%). LC-MS m/z: 162.3 [M+H]⁺ HPLC Purity (214 nm): 95%; t_{R}=1.21 min.

Following general procedure B, 3,3-dimethyl-1,2,3,4-tetrahydroisoquinoline (100 mg, 0.6 mmol) and (2-isocyanatoethyl)benzene (456 mg, 3.1 mmol) afforded the title compound (12.5 mg, 6.5%) as a white solid. ¹H NMR (400 MHz, CDCl₃) δ 7.34-7.30 (m, 2H), 7.26-7.16 (m, 5H), 7.15 (d, J=6.0 Hz, 1H), 7.09 (d, J=6.8 Hz, 1H), 4.30 (bs, 1H), 4.28 (s, 2H), 3.52 (q, J=6.0 Hz, 2H), 2.87 (t, J=6.8 Hz, 2H), 2.75 (s, 2H), 1.37 (s, 6H). LC-MS m/z: 309.1 [M+H]⁺. HPLC Purity (214 nm): 100%; t_{R}=9.43 min.

### Example 23: 2,2-Dimethyl-6-(1-methylpiperidin-4-yl)-N-phenethyl-3,4-dihydroquinoline-1(2H)-carboxamide

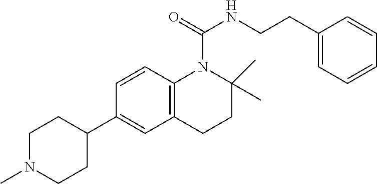 [SMILES: CN1CCC(c2ccc3c(c2)CCC(C)(C)N3C(=O)NCCc2ccccc2)CC1]

A mixture of 4-bromoaniline (3.4 g, 20.0 mmol), 3-chloro-3-methylbut-1-yne (2.6 g, 26.0 mmol), Cu (1.2 g, 20 mmol), CuCl (2.0 g, 20.0 mmol) in toluene (50 mL) was stirred at 110° C. for 12 h under N₂. The reaction was cooled and concentrated in vacuo to give a residue which was purified by silica gel column chromatography (PE/EA=20/1) to give 6-bromo-2,2-dimethyl-1,2-dihydroquinoline (600 mg, crude) as a yellow solid. LC-MS m/z: 238.1 [M+H]⁺. HPLC Purity (214 nm): 79%; t_{R}=1.17 min.

A mixture of 6-bromo-2,2-dimethyl-1,2-dihydroquinoline (600 mg, 2.5 mmol), 1-methyl-4-(4,4,5,5-tetramethyl-1,3,2-dioxaborolan-2-yl)-1,2,3,6-tetrahydropyridine 780 mg, 3.5 mmol), Pd(dppf)Cl₂.DCM (120 mg, 0.2 mmol), Na₂CO₃ (795 mg, 7.5 mmol) in 1,4-dioxane/H₂O (20 mL, 2/1) was stirred at 100° C. for 12 h under N₂. The reaction was cooled and concentrated in vacuo to give a residue which was purified by silica gel column chromatography (DCM/MeOH=10/1) to give 2,2-dimethyl-6-(1-methyl-1,2,3,6-tetrahydropyridin-4-yl)-1,2-dihydroquinoline (320 mg, crude) as a yellow solid. LC-MS m/z: 255.4 [M+H]⁺. HPLC Purity (214 nm): 81%; t_{R}=0.74 min.

A solution of crude 2,2-dimethyl-6-(1-methyl-1,2,3,6-tetrahydropyridin-4-yl)-1,2-dihydroquinoline (320 mg, 1.2 mmol), PtO₂ (50 mg, 0.2 mmol) in MeOH (10 mL) was stirred at RT for 1 h and filtered. The filtrate was concentrated to afford 2,2-dimethyl-6-(1-methylpiperidin-4-yl)-1,2,3,4-tetrahydroquinoline (260 mg, 84%) as a yellow solid. LC-MS m/z: 259.4 [M+H]⁺. HPLC Purity (214 nm): 79%; t_{R}=0.53 min.

Following general procedure B, 2,2-dimethyl-6-(1-methylpiperidin-4-yl)-1,2,3,4-tetrahydroquinoline (258 mg, 1 mmol) and (2-isocyanatoethyl)benzene (441 mg, 3 mmol) afforded the title compound (22.3 mg, 6%) as a white solid. ¹H NMR (400 MHz, CDCl₃) δ 7.30-7.18 (m, 5H), 6.92 (s, 1H), 6.82 (s, 2H), 4.97 (bs 1H), 3.56-3.49 (m, 2H), 3.18 (d, J=11.4 Hz, 2H), 2.83 (t, J=7.0 Hz, 2H), 2.59-2.44 (m, 7H), 2.37-2.28 (m, 2H), 2.03-1.84 (m, 3H), 1.76-1.68 (m, 2H), 1.54 (s, 6H). LC-MS m/z: 406.2 [M+H]⁺. HPLC Purity (214 nm): 100%; t_{R}=6.95 min.

### Example 24: N-Butyl-2,2-dimethyl-5-(1-methylpiperidin-4-yl)-3,4-dihydroquinoline-1(2H)-carboxamide

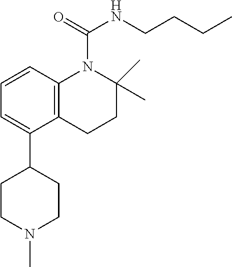 [SMILES: CCCCNC(=O)N1c2cccc(C3CCN(C)CC3)c2CCC1(C)C]

A solution of 2,2-dimethyl-5-(1-methyl-1,2,3,6-tetrahydropyridin-4-yl)-1,2-dihydroquinoline (700 mg, 2.7 mmol), PtO₂ (125 mg, 0.5 mmol) in MeOH (16 mL) was stirred at RT for 3 h under H₂ and filtered. The filtrate was concentrated to afford 2,2-dimethyl-5-(1-methylpiperidin-4-yl)-1,2,3,4-tetrahydroquinoline (600 mg, 84.3%) as a yellow solid. LC-MS m/z: 259.1 [M+H]⁺. HPLC Purity (214 nm): 98%; t_{R}=1.93 min.

Following general procedure B, 2,2-dimethyl-5-(1-methylpiperidin-4-yl)-1,2,3,4-tetrahydroquinoline (200 mg, 0.8 mmol) and 1-isocyanatobutane (788 mg, 7.8 mmol) afforded the title compound (30 mg, 10.5%) as a white solid. ¹H NMR (400 MHz, CDCl₃) δ 7.08 (t, J=7.6 Hz, 1H), 6.99-6.87 (m, 2H), 4.87 (bs, 1H), 3.25-3.18 (m, 2H), 3.12 (d, J=10.8 Hz, 2H), 2.80-2.71 (m, 1H), 2.65-2.59 (m, 2H), 2.43 (s, 3H), 2.21 (t, J=11.2 Hz, 2H), 1.94-1.88 (m, 2H), 1.84-1.74 (m, 2H), 1.74-1.68 (m, 2H), 1.56 (s, 6H), 1.51-1.41 (m, 2H), 1.37-1.28 (m, 2H), 0.93 (t, J=7.2 Hz, 3H). LC-MS m/z: 358.4 [M+H]⁺. HPLC Purity (214 nm): 100%; t_{R}=5.13 min.

### Example 25: 2, 2-Dimethyl-N-phenethylpiperidine-1-carboxamide

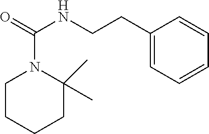 [SMILES: CC1(C)CCCCN1C(=O)NCCc1ccccc1]

Following general procedure B, 2,2-dimethylpiperidine (100 mg, 0.067 mmol) and (2-isocyanatoethyl)benzene (118 mg 0.8 mmol) afforded the title compound (89 mg, 51%) as a colorless oil. ¹H NMR (400 MHz, CDCl₃) δ 7.35-7.17 (m, 5H), 4.41 (bs, 1H), 3.45 (dd, J=12.6, 6.7 Hz, 2H), 3.12 (t, J=5.5 Hz, 2H), 2.82 (t, J=6.8 Hz, 2H), 1.63-1.42 (m, 6H), 1.38 (s, 6H). LC-MS m/z: 261 [M+H]⁺. HPLC Purity (214 nm): 96%; t_{R}=8.35 min.

### Example 26: 2,2-Dimethyl-4-(4-methylpiperazin-1-yl)-N-phenethylpiperidine-1-carboxamide

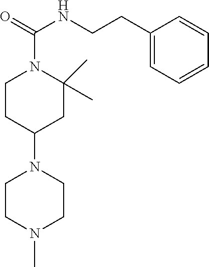 [SMILES: CN1CCN(C2CCN(C(=O)NCCc3ccccc3)C(C)(C)C2)CC1]

A solution of 2, 2-dimethylpiperidin-4-one (400 mg, 3.14 mmol), CbzCl (640 mg 3.77 mmol) and NaH (150 mg 6.28 mmol) in THF (11 mL) was stirred for at RT for 2 h. The mixture was concentrated and purified by column chromatography (DCM/MEOH=10:1) to give benzyl 2,2-dimethyl-4-oxopiperidine-1-carboxylate as a brown oil (600 mg, 74%). LC-MS m/z: 261 [M+H]⁺. HPLC Purity (214 nm): 78%; t_{R}=1.93 min.

A mixture of benzyl 2,2-dimethyl-4-oxopiperidine-1-carboxylate (600 mg, 2.3 mmol) and 1-methylpiperazine (275 mg, 2.7 mmol) in MeOH (10 ml) was stirred at RT for 30 min followed by the addition of NaBH₃CN (159 mg, 2.5 mmol). The mixture was stirred at RT for 2 days, concentrated and purified by column chromatography (DCM/MeOH=1:1) to give benzyl 2,2-dimethyl-4-(4-methylpiperazin-1-yl)piperidine-1-carboxylate as a brown oil (376 mg, 47%). LC-MS m/z: 346 [M+H]⁺. HPLC Purity (214 nm): 100%; t_{R}=1.93 min.

To a solution of benzyl 2, 2-dimethyl-4-(4-methylpiperazin-1-yl) piperidine-1-carboxylate (376 mg, 1.09 mmol) in MeOH (20 mL) was added Pd/C (300 mg) and the mixture was stirred at RT under H₂ overnight. The mixture was filtered and concentrated to afford 1-(2,2-dimethylpiperidin-4-yl)-4-methylpiperazine (280 mg, 96%) as a yellow oil. LC-MS m/z: 216 [M+H]⁺. HPLC Purity (214 nm): 90%; t_{R}=1.45 min.

Following general procedure B, 1-(2,2-dimethylpiperidin-4-yl)-4-methylpiperazine (100 mg, 0.47 mmol) and phenethyl isocyanate (97 mg 0.65 mmol) afforded the title compound (15 mg, 9%) as a white solid. ¹H NMR (400 MHz, CDCl₃) δ 7.34-7.22 (m, 2H), 7.23-7.17 (m, 3H), 4.41 (bs, 1H), 3.47-3.32 (m, 3H), 3.06-2.98 (m, 1H), 3.12 (t, J=5.5 Hz, 2H), 2.61-2.40 (m, 8H), 2.29 (s, 3H), 1.87-1.83 (m, 1H), 1.68-1.39 (m, 4H), 1.52 (s, 3H), 1.30 (s, 3H). LC-MS m/z: 359 [M+H]⁺. HPLC Purity (214 nm): 95%; t_{R}=4.67 min.

### Example 27: N-Butyl-2,2-dimethyl-3,4-dihydroquinoline-1(2H)-carboxamide

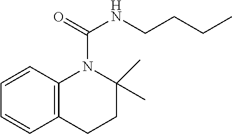 [SMILES: CCCCNC(=O)N1c2ccccc2CCC1(C)C]

Following general procedure B, 2,2-dimethyl-1,2,3,4-tetrahydroquinoline (0.50 g, 3.10 mmol) and 1-isocyanatobutane (1.53 g, 15.50 mmol) afforded the title compound (20.1 mg, 2.5%) as a white solid. ¹H NMR (400 MHz, CDCl₃) δ 7.10-7.03 (m, 3H), 6.92-6.88 (m, 1H), 4.95 (bs, 1H), 3.23 (dd, J=13.2, 7.2 Hz, 2H), 2.60 (t, J=6.0 Hz, 2H), 1.74 (t, J=2.8 Hz, 2H), 1.48 (s, 6H), 1.46-1.42 (m, 2H), 1.32-1.27 (m, 2H), 0.91 (t, J=7.6 Hz, 3H). LC-MS m/z: 261.1 [M+H]⁺. HPLC Purity (214 nm): 100%; t_{R}=7.91 min.

### Example 28: 2,2-Dimethyl-4-oxo-N-phenethyl-3,4-dihydroquinoline-1(2H)-carboxamide

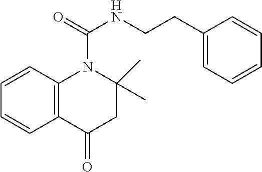 [SMILES: CC1(C)CC(=O)c2ccccc2N1C(=O)NCCc1ccccc1]

Following general procedure B, 2,2-dimethyl-2,3-dihydroquinolin-4 (1H)-one (200 mg, 1.2 mmol), Et₃N (577 mg, 5.7 mmol) and (2-isocyanatoethyl)benzene (840 mg, 5.7 mmol) afforded the title compound (24.2 mg, 7%) as a white solid. ¹H NMR (400 MHz, CDCl₃) δ 7.88 (d, J=7.2 Hz, 1H), 7.35-7.30 (m, 2H), 7.27-7.22 (m, 4H), 6.84 (t, J=7.6 Hz, 1H), 6.67 (d, J=8.8 Hz, 1H), 5.64 (bs, 1H), 3.70 (q, J=6.4 Hz, 2H), 2.96 (t, J=6.8 Hz, 2H), 2.58 (s, 2H), 1.41 (s, 6H). LC-MS m/z: 323.3 [M+H]⁺. HPLC Purity (214 nm): 100%; t_{R}=8.97 min.

### Example 29: 2,2-Dimethyl-5-(4-methylpiperazin-1-yl)-N-phenethyl-3,4-dihydroquinoline-1(2H)-carboxamide

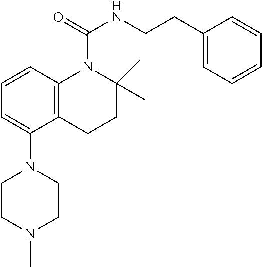 [SMILES: CN1CCN(c2cccc3c2CCC(C)(C)N3C(=O)NCCc2ccccc2)CC1]

To a solution of 3-bromoaniline (17 g, 100 mmol) in toluene (200 mL) were added 3-chloro-3-methylbut-1-yne (13.3 g, 130 mmol), Cu (6.4 g, 100 mmol) and CuCl (9.8 g, 100 mmol) and the mixture was stirred at 120° C. for 16 h. The mixture was cooled, filtered and purified by silica gel column chromatography (PE/EA=20/1) to give crude 5-bromo-2,2-dimethyl-1,2-dihydroquinoline (2.7 g) as a yellow solid. LC-MS m/z: 238.1 [M+H]⁺. HPLC Purity (254 nm): 15.81%; t_{R}=1.33 min.

A mixture of crude 5-bromo-2,2-dimethyl-1,2-dihydroquinoline (500 mg, 2.1 mmol), 1-methylpiperazine (420 mg, 4.2 mmol), Pd₂(dba)₃ (96 mg, 0.11 mmol), BINAP (261 mg, 0.42 mmol) and KTB (512 mg, 4.2 mmol) in Tol (15 mL) was stirred at 90° C. for 16 h. The reaction mixture was cooled and concentrated in vacuo to give a residue which was purified by silica gel column chromatography (PE/EA=20/1) to give 2,2-dimethyl-5-(4-methylpiperazin-1-yl)-1,2-dihydroquinoline (210 mg, 38.9%) as a yellow solid. LC-MS m/z: 258.3 [M+H]⁺. HPLC Purity (254 nm): 78.67%; t_{R}=1.74 min.

A mixture of 2, 2-dimethyl-5-(4-methylpiperazin-1-yl)-1, 2-dihydroquinoline (210 mg, 0.82 mmol) and PtO₂ (36 mg, 0.16 mmol) in MeOH (10 mL) was stirred at RT for 20 h under H₂ and filtered. The filtrate was concentrated to afford 2,2-dimethyl-5-(4-methylpiperazin-1-yl)-1,2,3,4-tetrahydroquinoline (200 mg, 94.2%). LC-MS m/z: 260.1 [M+H]⁺. HPLC Purity (254 nm): 88.01%; t_{R}=1.97 min.

Following general procedure B, 2,2-dimethyl-5-(4-methylpiperazin-I-yl)-1,2,3,4-tetrahydroquinoline (200 mg, 0.77 mmol) and (2-isocyanatoethyl)benzene (1.13 g, 7.7 mmol) and the title compound of (27 mg, 8.6%) as a white solid. ¹H NMR (400 MHz, CDCl₃) δ 7.31-7.26 (m, 2H), 7.23-7.15 (m, 3H), 6.91 (t, J=8.0 Hz, 1H), 6.69-6.61 (m, 2H), 4.89 (bs, 1H), 3.50-3.45 (m, 2H), 2.95 (s, 4H), 2.81 (t, J=7.0 Hz, 2H), 2.67-2.54 (m, 6H), 2.36 (s, 3H), 1.67-1.62 (m, 2H), 1.58 (s, 6H). LC-MS m/z: 407.1 [M+H]⁺. HPLC Purity (214 nm): 95.46%; t_{R}=2.13 min.

### Example 30: 2,2-Dimethyl-6-(4-methylpiperazin-1-yl)-N-phenethyl-3,4-dihydroquinoline-1(2H)-carboxamide

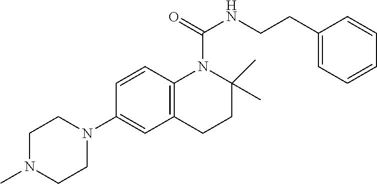 [SMILES: CN1CCN(c2ccc3c(c2)CCC(C)(C)N3C(=O)NCCc2ccccc2)CC1]

A mixture of 4-(4-methylpiperazin-1-yl) aniline (1.9 g, 10.0 mmol), 3-chloro-3-methylbut-1-yne (2.0 g, 20.0 mmol), TEA (2.0 g, 20 mmol) and CuCl (1.0 g, 10.0 mmol) in toluene (30 mL) was stirred at 120° C. for 12 h under N₂. The reaction mixture was cooled and concentrated in vacuo to give a residue which was purified by silica gel column chromatography (DCM/MeOH=20/1) to give 2,2-dimethyl-6-(4-methylpiperazin-1-yl)-1,2-dihydroquinoline (390 mg, crude) as a yellow solid. LC-MS m/z: 258.1 [M+H]⁺. HPLC Purity (214 nm): 9%; t_{R}=1.87 min.

A suspension of 2,2-dimethyl-6-(4-methylpiperazin-1-yl)-1,2-dihydroquinoline (257 mg, 1.0 mmol) and PtO₂ (50 mg, 0.2 mmol) in MeOH (10 mL) was stirred at RT for 3 h under H₂ (1 atm) and filtered. The filtrate was concentrated to afford 2,2-dimethyl-6-(4-methylpiperazin-1-yl)-1,2,3,4-tetrahydroquinoline (260 mg, crude) as a yellow solid. LC-MS m/z: 260.1 [M+H]⁺. HPLC Purity (214 nm): 79%; t_{R}=1.85 min.

Following general procedure B, 2,2-dimethyl-6-(4-methylpiperazin-1-yl)-1,2,3,4-tetrahydroquinoline (259 mg, 1 mmol) and (2-isocyanatoethyl)benzene (735 mg, 5.0 mmol) the title compound (30.6 mg, 7.5%) as a yellow solid. ¹H NMR (400 MHz, CDCl₃) δ 7.33-7.28 (m, 2H), 7.22-7.14 (m, 3H), 6.80 (d, J=8.7 Hz, 1H), 6.64 (d, J=2.8 Hz, 1H), 6.53 (dd, J=8.8, 2.8 Hz, 1H), 4.88 (t, J=5.5 Hz, 1H), 3.52-3.47 (m, 2H), 3.19-3.14 (m, 4H), 2.80 (t, J=7.0 Hz, 2H), 2.63-2.55 (m, 4H), 2.53-2.45 (m, 2H), 2.36 (s, 3H), 1.72-1.67 (m, 2H), 1.55 (s, 6H). LC-MS m/z: 407.2 [M+H]⁺. HPLC Purity (214 nm): 98.41%; t_{R}=6.74 min.

### Example 31: N-Butyl-8,8-dimethyl-1-oxa-9-azaspiro[5.5]undecane-9-carboxamide

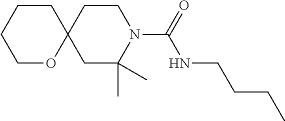 [SMILES: CCCCNC(=O)N1CCC2(CCCCO2)CC1(C)C]

To a solution of allylmagnesium bromide (1 M in THF, 15 mL) in THE (7 mL) was added tert-butyl 2,2-dimethyl-4-oxopiperidine-1-carboxylate (1.5 g, 6.6 mmol) dropwise at 0° C. and then the mixture was stirred at RT for 4 h. The reaction was quenched with aq. NH₄Cl (50 mL), extracted with EA (2×60 mL) and the combined organic layers were concentrated. The residue was purified by silica gel column chromatography (PE:EA=3:1) to give tert-butyl 4-allyl-4-hydroxy-2, 2-dimethylpiperidine-1-carboxylate (1.5 g, 84%) as a yellow oil. LC-MS m/z: 170.3 [M−100+H]⁺. HPLC Purity (214 nm): >78%; t_{R}=1.81 min.

To a solution of tert-butyl 4-allyl-4-hydroxy-2,2-dimethylpiperidine-1-carboxylate (1.5 g, 5.58 mmol) in DMF (20 mL) was added NaH (1.1 g, 27.9 mmol) at 0° C. The mixture was stirred at 0° C. for 30 min and then 3-bromoprop-1-ene (3.35 g, 27.9 mmol) was added and the mixture was stirred at RT for 15 h. The reaction was quenched with water (80 mL), extracted with EA (2×60 mL) and the combined organic layers were concentrated. The residue was purified by silica gel column chromatography (PE:EA=10:1) to give tert-butyl 4-allyl-4-(allyloxy)-2,2-dimethylpiperidine-1-carboxylate (1.4 g, 81%) as a yellow oil. LC-MS m/z: 210.3 [M−100+H]⁺. HPLC Purity (214 nm): >88%; t_{R}=1.69 min.

To a solution of GRUBB'S catalyst (757 mg, 0.9 mmol) in DCM (700 mL) was added tert-butyl 4-allyl-4-(allyloxy)-2,2-dimethylpiperidine-1-carboxylate (1.4 g, 4.5 mmol) in DCM (100 mL) dropwise over 3 h. The mixture was stirred at RT for 15 h, concentrated and the residue was purified by silica gel column chromatography (PE:EA=10:1) to give tert-butyl 8, 8-dimethyl-1-oxa-9-azaspiro [5.5] undec-3-ene-9-carboxylate (1.0 g, 79%) as a yellow oil. LC-MS m/z: 182.4 [M−100+H]⁺. HPLC Purity (214 nm): >80%; t_{R}=1.38 min.

A mixture of tert-butyl 8,8-dimethyl-1-oxa-9-azaspiro[5.5]undec-3-ene-9-carboxylate (1 g, 3.56 mmol) and Pd/C (150 mg) in MeOH (30 mL) was stirred at RT under H₂ for 15 h. The reaction was filtered, washed with MeOH (5 mL) and concentrated to give tert-butyl 8,8-dimethyl-1-oxa-9-azaspiro[5.5]undecane-9-carboxylate (1 g, 99%) as a yellow oil. LC-MS m/z: 184.4 [M−100+H]⁺. HPLC Purity (214 nm): >74%; t_{R}=1.42 min.

To a solution of tert-butyl 8,8-dimethyl-1-oxa-9-azaspiro[5.5]undecane-9-carboxylate (1 g, 3.5 mmol) in DCM (6 mL) was added HCl (4M in dioxane, 6 mL) and the mixture was stirred at RT for 15 h. The reaction was dissolved in DCM (30 mL), NaHCO₃ (5 g) was added and the resulting mixture was stirred at RT for 1 h. The reaction was filtered, washed with DCM (10 mL) and concentrated to give 8,8-dimethyl-1-oxa-9-azaspiro[5.5]undecane (600 mg, 93%) as a yellow oil. LC-MS m/z: 184.3 [M+H]⁺. HPLC Purity (254 nm): >80%; t_{R}=0.61 min.

Following general procedure B, 8,8-dimethyl-1-oxa-9-aza spiro[5.5]undecane (150 mg, 0.83 mmol) and 1-isocyanatobutane (124 mg, 1.25 mmol) afforded the title compound (69.5 mg, 30.0%) as a white solid. ¹H NMR (400 MHz, CDCl₃) δ 4.37 (s, 1H), 3.71-3.55 (m, 2H), 3.42-3.36 (m, 1H), 3.28-3.19 (m, 3H), 1.95-1.88 (m, 2H), 1.69-1.40 (m, 16H), 1.40-1.24 (m, 2H), 0.92 (t, J=7.3 Hz, 3H). LC-MS m/z: 283.1 [M+H]⁺. HPLC Purity (214 nm): >99%; t_{R}=2.05 min.

### Example 32: N-(2-Methoxyethyl)-2,2-dimethyl-3,4-dihydroquinoline-1(2H)-carboxamide

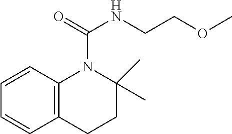 [SMILES: COCCNC(=O)N1c2ccccc2CCC1(C)C]

Following general procedure B, 2,2-dimethyl-1,2,3,4-tetrahydroquinoline (190 mg, 1.2 mmol) and 1-isocyanato-2-methoxyethane (596 mg, 5.9 mmol) afforded the title compound (101.8 mg, 32.9%) as a yellow oil. ¹H NMR (400 MHz, CDCl₃) δ 7.09-7.06 (m, 3H), 6.91-6.89 (m, 1H), 5.32 (bs, 1H), 3.48-3.45 (m, 2H), 3.45-3.39 (m, 2H), 3.32 (s, 3H), 2.63-2.56 (m, 2H), 1.76-1.72 (m, 2H), 1.55 (s, 6H). LC-MS m/z: 263.1 [M+H]⁺. HPLC Purity (214 nm): 96.57%; t_{R}=8.50 min.

### Example 33: N-Butyl-2,2-dimethyl-5-(4-methylpiperazin-1-yl)-3,4-dihydroquinoline-1(2H)-carboxamide

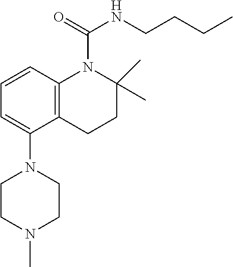 [SMILES: CCCCNC(=O)N1c2cccc(N3CCN(C)CC3)c2CCC1(C)C]

Following general procedure B, 2,2-dimethyl-5-(4-methylpiperazin-1-yl)-1,2,3,4-tetrahydroquinoline (180 mg, 0.69 mmol) and 1-isocyanatobutane (688 mg, 6.95 mmol) afforded the title compound (18.8 mg, 7.6%) as a white solid. ¹H NMR (400 MHz, CDCl₃) δ 7.05 (t, J=8.0 Hz, 1H), 6.82 (d, J=8.1 Hz, 1H), 6.66 (d, J=8.0 Hz, 1H), 4.85 (bs, 1H), 3.23-3.19 (m, 2H), 3.03-2.96 (m, 4H), 2.66-2.55 (m, 6H), 2.37 (s, 3H), 1.81-1.62 (m, 6H), 1.50-1.42 (m, 2H), 1.38-1.28 (m, 2H), 0.90 (t, J=7.3 Hz, 3H). LC-MS m/z: 359.1 [M+H]⁺. HPLC Purity (214 nm): 100%; t_{R}=1.59 min.

### Example 34: 2,2,4-Trimethyl-N-phenethylpiperazine-1-carboxamide

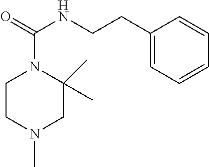 [SMILES: CN1CCN(C(=O)NCCc2ccccc2)C(C)(C)C1]

A solution of tert-butyl 2,2,4-trimethylpiperazine-1-carboxylate (1.00 g, 4.38 mmol) in 4 M dioxane-HCl (10.0 mL) was stirred at RT for 16 h. The solution was filtered and 1,3,3-trimethylpiperazine (0.50 g, 89.2%) was isolated as a white solid. LC-MS m/z: 129.2 [M+H]⁺. HPLC Purity (254 nm): 74%; t_{R}=0.66 min.

Following general procedure B, 1,3,3-trimethylpiperazine (0.10 g, 0.50 mmol) and (2-isocyanatoethyl)benzene (0.09 g, 0.60 mmol) afforded the title compound (68.1 mg, 49.7%) as a white solid. ¹H NMR (400 MHz, CDCl₃) δ 7.32-7.28 (m, 2H), 7.23-7.18 (m, 3H), 4.48 (bs, 1H), 3.46 (dt, J=12.4, 6.4 Hz, 2H), 3.17-3.12 (m, 2H), 2.81 (t, J=6.8 Hz, 2H), 2.37-2.32 (m, 2H), 2.21 (s, 3H), 1.39 (s, 6H). LC-MS m/z: 276.1 [M+H]⁺. HPLC Purity (214 nm): 100%; t_{R}=8.02 min.

### Example 35: 8-Fluoro-2,2-dimethyl-N-phenethyl-3,4-dihydroquinoline-1(2H)-carboxamide

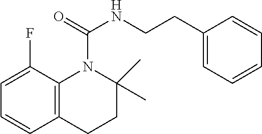 [SMILES: CC1(C)CCc2cccc(F)c2N1C(=O)NCCc1ccccc1]

A mixture of 2-fluoroaniline (5 g, 45 mmol), 3-chloro-3-methylbut-1-yne (9.2 g, 90 mmol), Et₃N (9.1 g, 90 mmol) and CuCl (4.5 g, 45 mmol) in toluene (70 mL) was stirred at 110° C. under N₂ overnight. Then the mixture was filtered and concentrated in vacuo to give a residue which was purified by silica gel column chromatography (PE:EA=10:1) to afford 8-fluoro-2,2-dimethyl-1,2-dihydroquinoline (300 mg, 3.8%) as a yellow solid. LC-MS m/z: 178.3 [M+H]⁺. HPLC Purity (214 nm): 93%; t_{R}=1.18 min.

A suspension of 8-fluoro-2,2-dimethyl-1,2-dihydroquinoline (300 mg, 1.7 mmol) and PtO₂ (50 mg) in MeOH (50 mL) was stirred at RT under H₂ for 2 h. Then the mixture was filtered and concentrated in vacuo to give a residue which was purified by silica gel column chromatography (PE:EA=10:1) to give 8-fluoro-2,2-dimethyl-1,2,3,4-tetrahydroquinoline (300 mg, 98.8%) as a yellow solid. LC-MS m/z: 180.2 [M+H]⁺. HPLC Purity (214 nm): 97%; t_{R}=1.30 min.

Following general procedure B, 8-fluoro-2,2-dimethyl-1,2,3,4-tetrahydroquinoline (150 mg, 0.8 mmol) and (2-isocyanatoethyl)benzene (740 mg, 5.0 mmol) afforded the title compound (38.1 mg, 13.9%) as a white solid. ¹H NMR (400 MHz, CDCl₃) δ 7.25-7.22 (m, 2H), 7.19-7.13 (m, 3H), 6.94-6.83 (m, 3H), 4.72 (bs, 1H), 3.48 (q, J=6 Hz, 2H), 2.81 (t, J=7.6 Hz, 2H), 2.58-2.54 (m, 2H), 1.74-1.70 (m, 2H), 1.57 (d, J=2 Hz, 6H). LC-MS m/z: 327.1 [M+H]⁺. HPLC Purity (214 nm): 100%; t_{R}=9.68 min.

### Example 36: 4-Methoxy-2,2-dimethyl-N-phenethylpiperidine-1-carboxamide

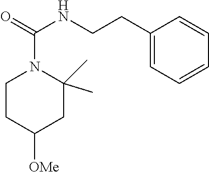 [SMILES: COC1CCN(C(=O)NCCc2ccccc2)C(C)(C)C1]

A mixture of tert-butyl 4-hydroxy-2,2-dimethylpiperidine-1-carboxylate (800 mg, 3.5 mmol and NaH (168 mg, 7 mmol) in DMF (20 mL) was stirred at RT for 0.5 h. Then CH₃I (993 mg, 7 mmol) was added into the reaction mixture was stirred at 80° C. overnight. The mixture was quenched with water (50 mL), extracted with EA (50 mL×3), dried over Na₂SO₄, filtered and concentrated. The residue was purified by silica gel column chromatography (DCM/MeOH=50:1) to give 4-methoxy-2,2-dimethylpiperidine (200 mg, 40%) as a white solid. LC-MS m/z: 144 [M+H]⁺. HPLC Purity (214 nm): 50%; t_{R}=2.08 min.

Following general procedure C, 4-methoxy-2,2-dimethylpiperidine (200 mg, 1.4 mmol) and 3-phenylpropan-1-amine (247 mg 1.68 mmol) afforded the title compound (30 mg, 7.3%) as a white oil. ¹H NMR (400 MHz, CDCl₃) δ 7.35-7.16 (m, 5H), 4.48 (s, 1H), 3.46-3.30 (m, 7H), 3.07-3.00 (m, 1H), 2.81 (t, J=6.8 Hz, 2H), 2.05-1.97 (m, 1H), 1.75 (dd, J=13.2, 2.8 Hz, 2H), 1.53-1.43 (m, 5H), 1.32 (s, 3H). LC-MS m/z: 291 [M+H]⁺. HPLC Purity (214 nm): 99%; t_{R}=7.55 min.

### Example 37: N-Butyl-2,2,4-trimethylpiperazine-1-carboxamide

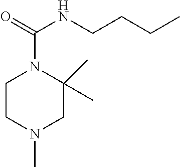 [SMILES: CCCCNC(=O)N1CCN(C)CC1(C)C]

Following general procedure B, 1,3,3-trimethylpiperazine (0.10 g, 0.50 mmol) and 1-isocyanatobutane (0.06 g, 0.60 mmol) afforded the title compound (109.3 mg, 96.7%) as a white solid. ¹H NMR (400 MHz, CDCl₃) δ 4.47 (bs, 1H), 3.25-3.15 (m, 4H), 2.39 (t, J=5.2 Hz, 2H), 2.23 (s, 3H), 2.13 (s, 2H), 1.49-1.46 (m, 2H), 1.45 (s, 6H), 1.38-1.31 (m, 2H), 0.92 (t, J=7.2 Hz, 3H). LC-MS m/z: 228.1 [M+H]⁺. HPLC Purity (214 nm): 100%; t_{R}=7.38 min.

### Example 38: N-Butyl-1,2′,2′-trimethyl-[3,4′-bipipridine]-1′-carboxamide

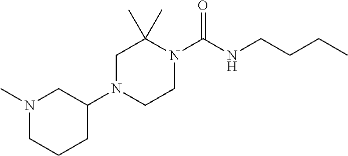 [SMILES: CCCCNC(=O)N1CCN(C2CCCN(C)C2)CC1(C)C]

To a solution of tert-butyl 3-oxopiperidine-1-carboxylate (1.99 g, 10 mmol) in MeOH (20 mL) were added 2,2-dimethylpiperazine (1.14 g, 10 mmol) and 3 drops of AcOH. The mixture was stirred at RT for 30 min and then NaBH₃CN (1.89 g, 30 mmol) was added and the mixture was stirred at RT for 16 h. The reaction mixture was quenched with H₂O (5 mL) extracted with EA (30 mL) and the residue was purified by silica gel column chromatography (DCM/MeOH=20/1) to give tert-butyl 3-(3,3-dimethylpiperazin-1-yl)piperidine-1-carboxylate (1.7 g, crude) as a yellow oil. LC-MS m/z: 298.1 [M+H]⁺. HPLC Purity (214 nm): 19%; t_{R}=1.52 min.

To a solution of tert-butyl 3-(3,3-dimethylpiperazin-1-yl)piperidine-1-carboxylate (1.49 g, 5 mmol) in THF (10 mL) was added LAH (50 mL, 1N in THF) at 0° C. and the mixture was stirred at RT for 24 h and then filtered. The filtrate was concentrated to afford 3,3-dimethyl-1-(1-methylpiperidin-3-yl) piperazine (900 mg, crude) as a yellow oil. LC-MS m/z: 212.2 [M+H]⁺. HPLC Purity (214 nm): 29%; t_{R}=1.59 min.

Following general procedure B, 3,3-dimethyl-1-(1-methyl piperidin-3-yl)piperazine (211 mg, 1 mmol) and 1-isocyanatobutane (400 mg, 4.0 mmol) afforded the title compound (24.1 mg, 11%) as a yellow solid. ¹H NMR (400 MHz, CDCl₃) δ 4.49 (bs, 1H), 3.26-3.15 (m, 4H), 2.92 (d, J=10.4 Hz, 1H), 2.76 (d, J=11.0 Hz, 1H), 2.61 (t, J=5.2 Hz, 2H), 2.52-2.41 (m, 1H), 2.30-2.25 (m, 5H), 1.88-1.70 (m, 4H), 1.61-1.53 (m, 1H), 1.52-1.42 (m, 2H), 1.38 (d, J=9.3 Hz, 6H), 1.39-1.27 (m, 2H), 1.19-1.08 (m, 1H), 0.92 (t, J=7.3 Hz, 3H). LC-MS m/z: 311.4 [M+H]⁺. HPLC Purity (214 nm): 100%; t_{R}=3.53 min.

### Example 39: N-Butyl-2,2-dimethyl-4-((1-methylpiperidin-4-yl)oxy)piperidine-1-carboxamide

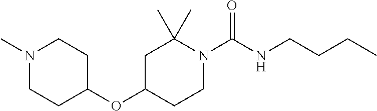 [SMILES: CCCCNC(=O)N1CCC(OC2CCN(C)CC2)CC1(C)C]

To a mixture of t-butyl 4-hydroxy-2,2-dimethylpiperidine-1-carboxylate (660 mg, 2.9 mmol) in DMSO (15 mL) was added NaH (350 mg, 8.7 mmol) at 0° C. The mixture was stirred at RT for 1 h and then 4-chloropyridine hydrochloride (435 mg, 2.9 mmol) was added and the mixture was then stirred at RT for 16 h. The reaction mixture was quenched with water, extracted with EA (×3), concentrated and purified by silica gel column chromatography (DCM/MeOH=10/1) to give t-butyl 2,2-dimethyl-4-(pyridin-4-yloxy)piperidine-1-carboxylate (750 mg, 85%) as a yellow oil. LC-MS m/z: 307.0 [M+H]⁺. Purity (214 nm): 74.72%; t_{R}=1.73 min.

To a solution of t-butyl 2,2-dimethyl-4-(pyridin-4-yloxy)piperidine-1-carboxylate (750 mg, 2.5 mmol) in DCM (20 mL) was added MeI (1.76 g, 12.6 mmol) and the mixture was stirred at RT for 1 h and then concentrated in vacuo to give 4-(1-(t-butoxycarbonyl)-2,2-dimethylpiperidin-4-yloxy)-1-methylpyridinium iodide (1.2 g crude) as a yellow oil. The crude product was used directly in the next step.

A suspension of 4-(1-(t-butoxycarbonyl)-2,2-dimethylpiperidin-4-yloxy)-1-methylpyridinium iodide (1.2 g, 3.7 mmol) and PtO₂ (170 mg, 0.2 mmol) in MeOH (20 mL) was stirred at 50° C. for 16 h under H₂ and then filtered. The filtrate was purified by silica gel column chromatography (DCM/MeOH=20/1) to give t-butyl 2,2-dimethyl-4-(1-methylpiperidin-4-yloxy)piperidine-1-carboxylate (970 mg, 81%) as a yellow oil. LC-MS m/z: 327.4[M+H]⁺. Purity (214 nm): 78.69%; t_{R}=0.89 min.

To a solution of t-butyl 2,2-dimethyl-4-(1-methylpiperidin-4-yloxy)piperidine-1-carboxylate (970 mg, 3.0 mmol) in DCM (10 mL) was added HCl-Dioxane (10 ml). The mixture was stirred at RT for 3 h and then concentrated to give 2,2-dimethyl-4-(1-methylpiperidin-4-yloxy)piperidine (935 mg, crude) as a yellow oil. The crude product was used directly in the next step.

Following general procedure B, 2,2-dimethyl-4-(1-methylpiperidin-4-yloxy)piperidine (150 mg, 0.67 mmol) and 1-isocyanatobutane (332 mg, 3.35 mmol) afforded the title compound (9.7 mg, 4.5%) as a yellow solid. ¹H NMR (400 MHz, CDCl₃) δ 4.48 (s, 1H), 3.68-3.58 (m, 2H), 3.46 (dd, J=12.3, 6.6 Hz, 1H), 3.21-3.07 (m, 3H), 2.94 (d, J=8.1 Hz, 4H), 2.57 (s, 3H), 2.20-1.91 (m, 3H), 1.75-1.66 (m, 1H), 1.64-1.41 (m, 7H), 1.38-1.27 (m, 5H), 0.93 (t, J=7.3 Hz, 3H). LC-MS m/z: 326.2 [M+H]⁺. HPLC Purity (214 nm): 97.05%; t_{R}=7.47 min.

### Example 40: 2,2-Dimethyl-N-(3-phenylpropyl)piperidine-1-carboxamide

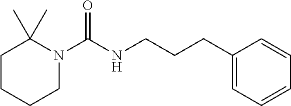 [SMILES: CC1(C)CCCCN1C(=O)NCCCc1ccccc1]

Following general procedure A, 2,2-dimethylpiperidine (75 mg, 0.5 mmol) and 3-phenylpropan-1-amine (202 mg, 1.5 mmol) afforded the title compound (8.4 mg, 6.1%) as a white solid. ¹H NMR (400 MHz, CDCl₃) δ 7.36-7.26 (m, 2H), 7.18 (dd, J=9.9, 4.2 Hz, 3H), 4.30 (d, J=59.5 Hz, 1H), 3.36-3.14 (m, 2H), 3.06 (dt, J=11.6, 5.4 Hz, 2H), 2.74-2.47 (m, 2H), 1.95-1.77 (m, 2H), 1.55 (ddd, J=10.5, 8.0, 4.6 Hz, 4H), 1.47 (dd, J=12.2, 7.2 Hz, 2H), 1.39 (s, 6H). LC-MS m/z: 275.3 [M+H]⁺. HPLC Purity (214 nm): 96.34%; t_{R}=9.57 min.

### Example 41: N-iso-Pentyl-2,2-dimethyl-4-phenoxypiperidine-1-carboxamide

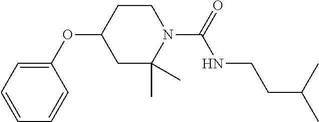 [SMILES: CC(C)CCNC(=O)N1CCC(Oc2ccccc2)CC1(C)C]

To a solution of tert-butyl 2,2-dimethyl-4-oxopiperidine-1-carboxylate (2 g, 8.81 mmol) in MeOH (10 mL) was added NaBH₄ (1 g, 26.4 mmol) and the mixture was stirred at RT for 16 h. The reaction mixture was quenched with H₂O (5 mL) extracted with EA (30 mL) and the residue was purified by silica gel column chromatography (PE/EA=1/3) to give tert-butyl 4-hydroxy-2,2-dimethylpiperidine-1-carboxylate (1.6 g, 79.3%) as an oil. LC-MS m/z: 174.1 [M+H]⁺. HPLC Purity (254 nm): 81.95%; t_{R}=1.89 min.

A mixture of DIAD (657 mg, 3.25 mmol) and PPh₃ (852 mg, 3.25 mmol) in THF (20 mL) was stirred at RT for 10 min and then tert-butyl 4-hydroxy-2,2-dimethylpiperidine-1-carboxylate (500 mg, 2.15 mmol) and phenol (306 mg, 3.25 mmol) were added. The reaction mixture was stirred at RT for 16 h and purified by silica gel column chromatography (DCM/MeOH=50/1) to give tert-butyl 2,2-dimethyl-4-phenoxypiperidine-1-carboxylate (350 mg, crude) as a yellow solid. LC-MS m/z: 206.2 [M+H]⁺. HPLC Purity (214 nm): 17.00%; t_{R}=2.42 min.

A mixture of tert-butyl 2,2-dimethyl-4-phenoxypiperidine-1-carboxylate (350 mg, 1.15 mmol) and HCl-dioxane (4 M, 2 ml) in DCM (5 mL) was stirred at RT for 4 h and then concentrated. The residue was re-dissolved in in DCM (5 ml), Na₂CO₃ (4 g) was added and stirred for several min and then filtered. The filtrate was concentrated to afford 2,2-dimethyl-4-phenoxypiperidine (200 mg, 85.1%). LC-MS m/z: 206.2 [M+H]⁺. HPLC Purity (214 nm): 95.63%; t_{R}=1.72 min.

Following general procedure A, 2,2-dimethyl-4-phenoxypiperidine (200 mg, 0.97 mmol) and 4-methylpentan-1-amine (844 mg, 9.7 mmol) afforded the title compound (56.7 mg, 18.4%) as a white solid. ¹H NMR (400 MHz, CDCl₃) δ 7.33-7.26 (m, 2H), 6.95 (t, J=7.3 Hz, 1H), 6.88 (d, J=8.1 Hz, 2H), 4.55-4.46 (m, 1H), 4.40 (bs, 1H), 3.55-3.48 (m, 1H), 3.28-3.16 (m, 3H), 2.20-2.12 (m, 1H), 1.94-1.88 (m, 1H), 1.79-1.71 (m, 2H), 1.67-1.58 (m, 1H), 1.54 (s, 3H), 1.46 (s, 3H), 1.47-1.32 (m, 2H), 0.94 (d, J=6.6 Hz, 6H). LC-MS m/z: 319.2 [M+H]⁺. HPLC Purity (214 nm): 94.83%; t_{R}=9.94 min.

### Example 42: N-iso-Pentyl-2,2-dimethyl-4-phenylpiperazine-1-carboxamide

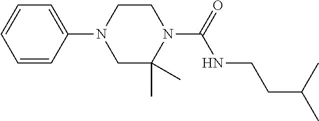 [SMILES: CC(C)CCNC(=O)N1CCN(c2ccccc2)CC1(C)C]

A mixture of bromobenzene (550 mg, 4.8 mmol), 2,2-dimethylpiperazine (630 mg, 4 mmol), Pd₂(dba)₃ (180 mg, 0.2 mmol), BINAP (250 mg, 0.4 mmol) and t-BuOK (1000 g, 8 mmol) in toluene (10 mL) was stirred at 90° C. for 16 h under N₂. The reaction was cooled and concentrated in vacuo. The residue was purified by silica gel column chromatography (DCM/MeOH=10/1) to give 3,3-dimethyl-1-phenylpiperazine (550 mg, 72%) as a bronze solid. LC-MS m/z: 191.2 [M+H]⁺. HPLC Purity (254 nm): 96.14%; t_{R}=1.51 min.

Following general procedure A, 3,3-dimethyl-1-phenylpiperazine (190 mg, 1.0 mmol) and 1-methyl-1H-imidazole (410 mg, 5.0 mmol) and 3-methylbutan-1-amine (175 mg, 2.0 mmol) afforded the title compound (150 mg, 49.5%) as a clear oil. ¹H NMR (400 MHz, CDCl₃) δ 7.28-7.25 (m, 2H), 6.80-6.76 (m, 3H), 4.32 (s, 1H), 3.61 (dd, J=7.1, 4.0 Hz, 2H), 3.38-3.35 (m, 2H), 3.26-3.23 (m, 2H), 3.21 (s, 2H), 1.69-1.61 (m, 1H), 1.49 (s, 6H), 1.47-1.40 (m, 2H), 0.93 (d, J=6.6 Hz, 6H). LC-MS m/z: 304.2 [M+H]⁺. HPLC Purity (214 nm): 99.12%; t_{R}=9.55 min.

### Example 43: N-(4-Cyclopropylbutyl)-8,8-dimethyl-1-oxa-9-azaspiro[5.5]undecane-9-carboxamide

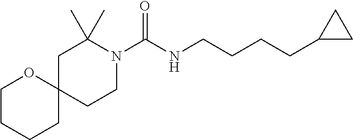 [SMILES: CC1(C)CC2(CCCCO2)CCN1C(=O)NCCCCC1CC1]

Following general procedure A, 8,8-dimethyl-1-oxa-9-azaspiro[5.5]undecane (110 mg, 0.62 mmol) and 4-cyclopropylbutan-1-amine (140 mg, 1.24 mmol) afforded the title compound (38.6 mg, 20%) as a white solid. ¹H NMR (400 MHz, CDCl₃) δ 4.39 (s, 1H), 3.72-3.55 (m, 2H), 3.43-3.29 (m, 1H), 3.26-3.12 (m, 3H), 1.90 (d, J=14.2 Hz, 2H), 1.72-1.59 (m, 2H), 1.61-1.41 (m, 16H), 1.21 (dd, J=14.4, 7.1 Hz, 2H), 0.71-0.52 (m, 1H), 0.44-0.30 (m, 2H), 0.05-0.08 (m, 2H). LC-MS m/z: 323.1 [M+H]⁺. HPLC Purity (214 nm): >99%; t_{R}=9.82 min6

### Example 44: N-(Hex-5-en-1-yl)-8,8-dimethyl-1-oxa-9-azaspiro[5.5]undecane-9-carboxamide

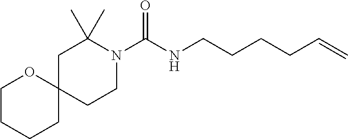 [SMILES: C=CCCCCNC(=O)N1CCC2(CCCCO2)CC1(C)C]

Following general procedure A, 8,8-dimethyl-1-oxa-9-azaspiro[5.5]undecane (110 mg, 0.62 mmol) and hex-5-en-1-amine (140 mg, 1.24 mmol) afforded the title compound (10.3 mg, 5.4%) as a yellow oil. ¹H NMR (400 MHz, CDCl₃) δ 5.80 (ddt, J=16.9, 10.3, 6.6 Hz, 1H), 5.06-4.89 (m, 2H), 4.38 (s, 1H), 3.71-3.55 (m, 2H), 3.35 (dd, J=17.3, 6.3 Hz, 1H), 3.20 (dt, J=12.6, 5.5 Hz, 3H), 2.07 (d, J=14.2, 7.1 Hz, 2H), 1.89 (d, J=13.6 Hz, 2H), 1.65-1.42 (m, 18H). LC-MS m/z: 309.1 [M+H]⁺. HPLC Purity (214 nm): >99%; t_{R}=9.41 min.

### Example 45: N-Phenethyl-6-azaspiro[4.5]decane-6-carboxamide

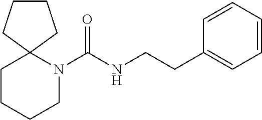 [SMILES: O=C(NCCc1ccccc1)N1CCCCC12CCCC2]

Following general procedure B, 6-azaspiro[4.5]decane hydrogen chloride (139 mg, 0.8 mmol) and (2-isocyanatoethyl)benzene (352 mg, 2.4 mmol) afforded the title compound (59.8 mg, 26%) as a yellow solid. ¹H NMR (400 MHz, CDCl₃) δ 7.70-7.56 (m, 2H), 7.35-7.20 (m, 3H), 4.46 (s, 1H), 3.55-3.45 (m, 2H), 3.29-3.22 (m, 2H), 2.82 (t, J=6.8 Hz, 2H), 1.98-1.82 (m, 4H), 1.81-1.72 (m, 2H), 1.70-1.32 (m, 8H). LC-MS m/z: 287.3 [M+H]⁺. HPLC Purity (214 nm): 96.35%; t_{R}=9.04 min.

### Example 46: N-Phenethyl-5-azaspiro 3.5 nonane-5-carboxamide

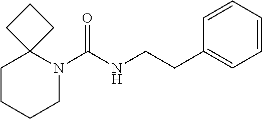 [SMILES: O=C(NCCc1ccccc1)N1CCCCC12CCC2]

To a solution of t-butyl 8-oxo-5-azaspiro[3.5]nonane-5-carboxylate (400 mg, 1.67 mmol) in DCM (3 mL) was added HCl (1 mL, 4M in dioxane). The mixture was stirred at RT for 15 h. The reaction mixture was concentrated to give crude 5-azaspiro[3.5]nonan-8-one (240 mg, 82%) as a yellow oil. LC-MS m/z: 140.0 [M+H]⁺.

A mixture of crude 5-azaspiro[3.5]nonan-8-one (240 mg, 1.37 mmol) and NH₂NH₂.H₂O (206 mg, 4.11 mmol) was stirred at 60° C. for 1 h and then the mixture was added to a solution of KOH (767 mg, 13.7 mmol) in triethylene glycol (3 mL) and H₂O (3 mL). The mixture was stirred at 220° C. for 2 h and then the reaction mixture was concentrated, treated with H₂O (15 mL) and extracted with DCM (2×15 mL). The combined organic extracts were concentrated to give 5-azaspiro[3.5]nonane (75 mg, 44%) as a yellow oil. LC-MS m/z: 126.1 [M+H]⁺.

Following general procedure B, 5-azaspiro[3.5]nonane (75 mg, 0.6 mmol) and (2-isocyanatoethyl)benzene (88 mg, 0.6 mmol) afforded the title compound (1.9 mg, 1.2%) as a white solid. ¹H NMR (400 MHz, CDCl₃) δ 7.38-7.30 (m, 2H), 7.28-7.20 (m, 3H), 4.28 (s, 1H), 3.51 (dd, J=12.5, 6.7 Hz, 2H), 3.24-3.18 (m, 2H), 2.83 (t, J=6.8 Hz, 2H), 2.20-2.11 (m, 2H), 2.01-1.94 (m, 2H), 1.74-1.61 (m, 6H), 1.34 (s, 2H). LC-MS m/z: 273.2 [M+H]⁺. HPLC Purity (214 nm): 96.96%; t_{R}=9.12 min.

### Examples 47 and 48: 5-cyano-2,2-dimethyl-N-phenethyl-3,4-dihydroquinoline-1(2H)-carboxamide and 7-cyano-2,2-dimethyl-N-phenethyl-3,4-dihydroquinoline-1(2H)-carboxamide

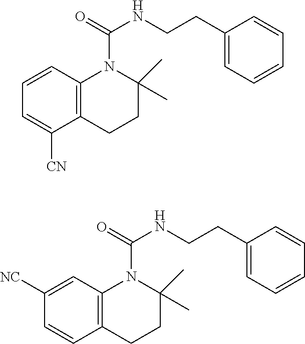 [SMILES: CC1(C)CCc2c(C#N)cccc2N1C(=O)NCCc1ccccc1 ; CC1(C)CCc2ccc(C#N)cc2N1C(=O)NCCc1ccccc1]

A suspension of 3-aminobenzonitrile (3.0 g, 25.4 mmol), 3-chloro-3-methylbut-1-yne (3.4 g, 33.0 mmol), Cu (1.6 g, 25.4 mmol) and CuCl (2.5 g, 25.4 mmol) in toluene (30 mL) was stirred at 110° C. for 15 h. The reaction mixture was concentrated, treated with H₂O (50 mL) and extracted with DCM (2×50 mL). The combined organic layers were concentrated, and the residue was purified by silica gel column chromatography (PE:EA=10:1) to give a mixture of 2,2-dimethyl-1,2-dihydroquinoline-5-carbonitrile and 2,2-dimethyl-1,2-dihydroquinoline-7-carbonitrile (1.6 g, 34%) as yellow solids. LC-MS m/z: 185.1 [M+H]⁺.

A suspension of 2,2-dimethyl-1,2-dihydroquinoline-5-carbonitrile and 2,2-dimethyl-1,2-dihydroquinoline-7-carbonitrile (0.8 g, 4.3 mmol) and Pd/C (150 mg) in MeOH (20 mL) was stirred at RT under H₂ for 15 h. The reaction mixture was filtered, and the filter cake was washed with MeOH (5 mL). The filtrate was concentrated to give a mixture of 2,2-dimethyl-1,2,3,4-tetrahydroquinoline-5-carbonitrile and 2,2-dimethyl-1,2,3,4-tetrahydroquinoline-7-carbonitrile (800 mg, 99%) as a yellow oil. LC-MS m/z: 186.2 [M+H]⁺.

Following general procedure B, a mixture of 2,2-dimethyl-1,2,3,4-tetrahydroquinoline-5-carbonitrile and 2,2-dimethyl-1,2,3,4-tetrahydroquinoline-7-carbonitrile (800 mg, 4.3 mmol) and (2-isocyanatoethyl)benzene (3.2 g, 21.5 mmol) afforded 5-cyano-2,2-dimethyl-N-phenethyl-3,4-dihydroisoquinoline-1(2H)-carboxamide (149.4 mg, 10.4%) and 7-cyano-2,2-dimethyl-N-phenethyl-3,4-dihydroisoquinoline-1(2H)-carboxamide (40.4 mg, 2.8%) as white solids.

### 5-cyano-2,2-dimethyl-N-phenethyl-3,4-dihydroisoquinoline-1(2H)-carboxamide

¹H NMR (400 MHz, CDCl₃) δ 7.33-7.21 (m, 3H), 7.18 (d, J=7.1 Hz, 2H), 7.13 (d, J=7.4 Hz, 1H), 6.94 (dt, J=16.5, 8.2 Hz, 2H), 5.02 (s, 1H), 3.57 (dd, J=12.6, 6.4 Hz, 2H), 2.93-2.79 (m, 4H), 1.77 (t, J=6.3 Hz, 2H), 1.46 (s, 6H). LC-MS m/z: 334.1 [M+H]⁺. HPLC Purity (214 nm): 100%; t_{R}=9.37 min.

### 7-cyano-2,2-dimethyl-N-phenethyl-3,4-dihydroisoquinoline-1(2H)-carboxamide

¹H NMR (400 MHz, CDCl₃) δ 7.32 (t, J=7.3 Hz, 2H), 7.27-7.20 (m, 3H), 7.13 (s, 1H), 7.10 (s, 2H), 5.00 (s, 1H), 3.57 (q, J=6.7 Hz, 2H), 2.88 (t, J=6.8 Hz, 2H), 2.66 (t, J=6.4 Hz, 2H), 1.73 (t, J=6.4 Hz, 2H), 1.47 (s, 6H). LC-MS m/z: 334.3 [M+H]⁺. HPLC Purity (214 nm): 100%; t_{R}=9.38 min.

### Example 49: 2,2-Dimethyl-5-(1-methylazetidin-3-yl)-N-phenethyl-3,4-dihydroisoquinoline-1(2H)-carboxamide

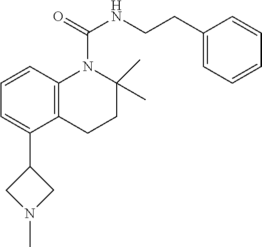 [SMILES: CN1CC(c2cccc3c2CCC(C)(C)N3C(=O)NCCc2ccccc2)C1]

A mixture of 4-methylbenzenesulfonohydrazide (18.6 g, 100 mmol) and t-butyl 3-oxoazetidine-1-carboxylate (17.2 g, 100 mmol) in toluene (300 mL) was stirred at 110° C. for 2 h under N₂ and then filtered to give t-butyl 3-(2-tosylhydrazineylidene)azetidine-1-carboxylate (30 g, crude) as a white solid. LC-MS m/z: 284.1 [M−55]⁺. Purity (214 nm): 92.4%; t_{R}=1.74 min.

To a solution of t-butyl 3-(2-tosylhydrazineylidene)azetidine-1-carboxylate (20 g, 60 mmol) in dioxane (300 mL) were added (3-nitrophenyl)boronic acid (20.5 g, 90 mmol) and Cs₂CO₃ (29.3 g, 90 mmol). The mixture was stirred at 110° C. for 40 h and purified by silica gel column chromatography (PE:EA=10:1) to give t-butyl 3-(3-nitrophenyl)azetidine-1-carboxylate (7.0 g, 39%) as a yellow oil. LC-MS m/z: 284.0 [M−55]⁺. Purity (214 nm): 90.7%; t_{R}=1.59 min.

A suspension of t-butyl 3-(3-nitrophenyl)azetidine-1-carboxylate (3.5 g, 12.6 mmol) and Pd/C (50 mg, 0.2 mmol) in MeOH (10 mL) was stirred at RT for 1 h and filtered. The filtrate was concentrated to afford crude t-butyl 3-(3-aminophenyl)azetidine-1-carboxylate (2.9 g) as a yellow solid which was used directly in the next step. LC-MS m/z: 193.1 [M+H]⁺. Purity (214 nm): 69%; t_{R}=1.98 min.

A mixture of t-butyl 3-(3-aminophenyl)azetidine-1-carboxylate (2.48 g, 10.0 mmol), 3-chloro-3-methylbut-1-yne (1.2 g, 12.0 mmol), Cu (640 mg, 10 mmol) and CuCl (1.0 g, 10.0 mmol) in toluene (30 mL) was stirred at 110° C. for 4 h under N₂. The reaction mixture was cooled and concentrated in vacuo to give a residue which was purified by silica gel column chromatography (PE:EA=6:1) to afford crude t-butyl 3-(2,2-dimethyl-1,2-dihydroquinolin-5-yl)azetidine-1-carboxylate (1.0 g) as a yellow solid. LC-MS m/z: 315.1 [M+H]⁺. Purity (214 nm): 59%; t_{R}=2.22 min.

To a solution of t-butyl 3-(2,2-dimethyl-1,2-dihydroquinolin-5-yl)azetidine-1-carboxylate (942 mg, 3.0 mmol) in THF (4 mL) was added LAH (9 mL, 9.0 mmol). The mixture was stirred at RT for 4 h and quenched with Na₂SO₄.10H₂O (3.0 g). The mixture was filtered, and the filtrate was purified by silica gel column chromatography (DCM:MeOH=1:1) to afford 2,2-dimethyl-5-(1-methylazetidin-3-yl)-1,2-dihydroquinoline (170 mg, 24.7%) and 2,2-dimethyl-7-(1-methylazetidin-3-yl)-1,2-dihydroquinoline (210 mg, 30.1%) as yellow solids. LC-MS m/z: 229.2 [M+H]⁺. Purity (214 nm): 76%; t_{R}=1.54 min.

A suspension of 2,2-dimethyl-5-(1-methylazetidin-3-yl)-1,2-dihydroquinoline (160 mg, 0.7 mmol) and PtO₂ (50 mg, 0.2 mmol) in MeOH (10 mL) was stirred at RT for 1 h and filtered. The filtrate was concentrated to afford crude 2,2-dimethyl-5-(1-methylazetidin-3-yl)-1,2,3,4-tetrahydroquinoline (130 mg) as a yellow solid. LC-MS m/z: 231.2 [M+H]⁺. Purity (214 nm): 92%; t_{R}=1.53 min.

Following general procedure B, 2,2-dimethyl-5-(1-methylazetidin-3-yl)-1,2,3,4-tetrahydroquinoline (115 mg, 0.5 mmol) and (2-isocyanatoethyl)benzene (220 mg, 1.5 mmol) afforded the title compound (27.2 mg, 15.8%) as a white solid. ¹H NMR (400 MHz, CDCl₃) δ 7.29 (t, J=6.5 Hz, 2H), 7.20 (dd, J=19.2, 7.2 Hz, 3H), 6.97 (t, J=7.8 Hz, 1H), 6.78 (d, J=8.1 Hz, 1H), 6.73 (d, J=7.5 Hz, 1H), 4.97 (s, 1H), 4.20 (s, 4H), 3.51 (dd, J=12.6, 6.4 Hz, 3H), 2.84 (t, J=6.9 Hz, 2H), 2.80-2.65 (m, 3H), 2.44-2.29 (m, 2H), 1.72-1.58 (m, 2H), 1.49 (s, 6H). LC-MS m/z: 378.1 [M+H]⁺. HPLC Purity (214 nm): 1000%; t_{R}=6.73 min.

### Example 50: 2,2-Dimethyl-7-(1-methylazetidin-3-yl)-N-phenethyl-3,4-dihydroisoquinoline-1(2H)-carboxamide

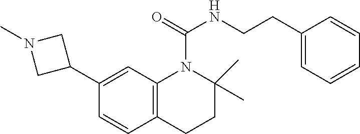 [SMILES: CN1CC(c2ccc3c(c2)N(C(=O)NCCc2ccccc2)C(C)(C)CC3)C1]

A suspension of 2,2-dimethyl-7-(1-methylazetidin-3-yl)-1,2-dihydroquinoline (228 mg, 1.0 mmol) and PtO₂ (50 mg, 0.2 mmol) in MeOH (10 mL) was stirred at RT for 1 h and filtered. The filtrate was concentrated to afford crude 2,2-dimethyl-7-(1-methylazetidin-3-yl)-1,2,3,4-tetrahydroquinoline (160 mg) as a yellow solid. LC-MS m/z: 239.1 [M+H]⁺. Purity (214 nm): 70%; t_{R}=1.54 min.

Following general procedure B, 2,2-dimethyl-7-(1-methylazetidin-3-yl)-1,2,3,4-tetrahydroquinoline (143 mg, 0.6 mmol) and (2-isocyanatoethyl)benzene (132 mg, 0.9 mmol) afforded the title compound (9.7 mg, 10.8%) as a white solid. ¹H NMR (400 MHz, CDCl₃) δ 7.29 (dd, J=12.1, 4.7 Hz, 2H), 7.23 (t, J=7.3 Hz, 1H), 7.17 (d, J=6.9 Hz, 2H), 7.03 (t, J=9.1 Hz, 1H), 6.78 (d, J=6.5 Hz, 2H), 5.09 (t, J=5.7 Hz, 1H), 4.11 (t, J=8.4 Hz, 2H), 3.77 (dt, J=15.6, 7.8 Hz, 1H), 3.52 (dd, J=13.2, 6.6 Hz, 4H), 2.85 (t, J=7.0 Hz, 2H), 2.66-2.50 (m, 5H), 1.84-1.64 (m, 2H), 1.51 (s, 6H). LC-MS m/z: 378.1 [M+H]⁺. HPLC Purity (214 nm): 100%; t_{R}=6.99 min.

### Examples 51 and 52: 2,2-Dimethyl-5-(oxetan-3-yl)-N-phenethyl-3,4-dihydroisoquinoline-1(2H)-carboxamide and 2,2-dimethyl-7-(oxetan-3-yl)-N-phenethyl-3,4-dihydroisoquinoline-1(2H)-carboxamide

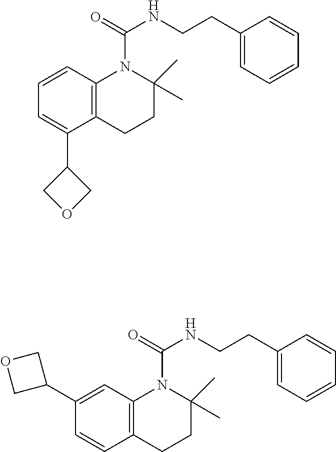 [SMILES: CC1(C)CCc2c(C3COC3)cccc2N1C(=O)NCCc1ccccc1 ; CC1(C)CCc2ccc(C3COC3)cc2N1C(=O)NCCc1ccccc1]

A mixture of 4-methylbenzenesulfonohydrazide (18.6 g, 100 mmol) and oxetan-3-one (7.2 g, 100 mmol) in toluene (300 mL) was stirred at 110° C. for 2 h under N₂ and then filtered to give 4-methyl-N-(oxetan-3-ylidene)benzenesulfonohydrazide (16.5 g, 68.8%) as a white solid. LC-MS m/z: 241.1 [M+H]⁺. Purity (214 nm): 95.4%; t_{R}=1.67 min.

To a solution of 4-methyl-N-(oxetan-3-ylidene)benzenesulfonohydrazide (24 g, 100 mmol) in dioxane (300 mL) were added (3-nitrophenyl)boronic acid (16.7 g, 100 mmol) and Cs₂CO₃ (48.9 g, 150 mmol). The mixture was stirred at 110° C. for 40 hrs and purified by silica gel column chromatography (PE:EA=10:1) to give crude 3-(3-nitrophenyl)oxetane (1.1 g) as a yellow oil. LC-MS m/z: 180.1 [M+H]⁺. Purity (214 nm): 85.2%; t_{R}=1.09 min.

A suspension of 3-(3-nitrophenyl)oxetane (1.1 g, 6.1 mmol) and Pd/C (50 mg, 0.2 mmol) in MeOH (10 mL) was stirred at RT for 1 h and filtered. The filtrate was concentrated to afford 3-(oxetan-3-yl)aniline (800 mg, crude) as a yellow solid. LC-MS m/z: 150.1 [M+H]⁺. Purity (214 nm): 86.3%; t_{R}=1.25 min.

A mixture of 3-(oxetan-3-yl)aniline (745 mg, 5.0 mmol), 3-chloro-3-methylbut-1-yne (714 mg, 7.0 mmol), Cu (320 mg, 5.0 mmol) and CuCl (500 mg, 5.0 mmol) in toluene (10 mL) was stirred at 110° C. for 4 h under N₂. The reaction mixture was cooled and concentrated in vacuo to give a residue which was purified by silica gel column chromatography (PE:EA=6:1) to afford a mixture of 2,2-dimethyl-5-(oxetan-3-yl)-1,2-dihydroquinoline and 2,2-dimethyl-7-(oxetan-3-yl)-1,2,3,4-tetrahydroquinoline (390 mg, crude) as a yellow solid. LC-MS m/z: 216.1 [M+H]⁺. Purity (214 nm): 86.1%; t_{R}=1.25 min.

A suspension of 2,2-dimethyl-5-(oxetan-3-yl)-1,2-dihydroquinoline and 2,2-dimethyl-7-(oxetan-3-yl)-1,2-dihydroquinoline (330 mg, 2.0 mmol), PtO₂ (50 mg, 0.2 mmol) in MeOH (10 mL) was stirred at RT for 1 h and filtered. The filtrate was concentrated to afford a crude mixture of 2,2-dimethyl-5-(oxetan-3-yl)-1,2,3,4-tetrahydro-quinoline and 2,2-dimethyl-7-(oxetan-3-yl)-1,2-dihydroquinoline (260 mg) as yellow solids. LC-MS m/z: 218.2 [M+H]⁺. Purity (214 nm): 76%; t_{R}=2.07 min.

Following general procedure B, a mixture of 2,2-dimethyl-5-(oxetan-3-yl)-1,2,3,4-tetrahydroquinoline and 2,2-dimethyl-7-(oxetan-3-yl)-1,2-dihydroquinoline (217 mg, 1.0 mmol) and (2-isocyanatoethyl)benzene (735 mg, 5.0 mmol) afforded 2,2-dimethyl-5-(oxetan-3-yl)-N-phenethyl-3,4-dihydroisoquinoline-1(21)-carboxamide (5.8 mg, 1.6%) and 2,2-dimethyl-7-(oxetan-3-yl)-N-phenethyl-3,4-dihydroisoquinoline-1(2H)-carboxamide (6.3 mg, 1.7%) as white solids.

### 2,2-dimethyl-5-(oxetan-3-yl)-N-phenethyl-3,4-dihydroisoquinoline-1(2H)-carboxamide

¹H NMR (400 MHz, CDCl₃) δ 7.33-7.27 (m, 2H), 7.22 (d, J=7.2 Hz, 1H), 7.17 (d, J=7.0 Hz, 2H), 7.00 (t, J=7.8 Hz, 1H), 6.94 (d, J=7.2 Hz, 1H), 6.79 (d, J=7.8 Hz, 1H), 5.02 (dd, J=8.5, 5.8 Hz, 2H), 4.93 (d, J=5.4 Hz, 1H), 4.83 (dd, J=7.4, 5.9 Hz, 2H), 4.59-4.36 (m, 1H), 3.50 (dd, J=13.0, 6.7 Hz, 2H), 2.87 (dt, J=13.9, 7.5 Hz, 2H), 2.44-2.17 (m, 2H), 1.65 (d, J=5.7 Hz, 2H), 1.51 (s, 6H). HPLC Purity (214 nm): 96.13%; t_{R}=9.17 min.

### 2,2-dimethyl-7-(oxetan-3-yl)-N-phenethyl-3,4-dihydroisoquinoline-1(2H)-carboxamide

¹H NMR (400 MHz, CDCl₃) δ 7.32-7.26 (m, 2H), 7.21 (t, J=7.3 Hz, 1H), 7.17 (t, J=7.5 Hz, 2H), 7.06 (d, J=7.7 Hz, 1H), 7.00-6.94 (m, 1H), 6.91 (d, J=1.5 Hz, 1H), 5.08-4.88 (m, 3H), 4.75-4.55 (m, 2H), 3.99 (dt, J=15.0, 7.4 Hz, 1H), 3.52 (dd, J=13.1, 6.8 Hz, 2H), 2.84 (t, J=7.0 Hz, 2H), 2.66-2.49 (m, 2H), 1.8-1.68 (m, 2H), 1.53 (s, 6H). LC-MS m/z: 365.1 [M+H]⁺. HPLC Purity (214 nm): 95.97%; t_{R}=9.37 min.

### Example 53: 3,3-Dimethyl-N-phenethylmorpholine-4-carboxamide

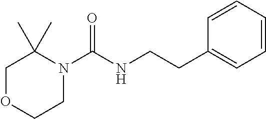 [SMILES: CC1(C)COCCN1C(=O)NCCc1ccccc1]

Following general procedure B, 3,3-dimethylmorpholine (100 mg, 0.87 mmol) and (2-isocyanatoethyl)benzene (153 mg, 1.04 mmol) afforded the title compound (63.2 mg, 27.7%) as a colorless oil. ¹H NMR (400 MHz, CDCl₃) δ 7.31 (t, J=7.3 Hz, 2H), 7.26-7.14 (m, 3H), 4.49 (s, 1H), 3.78-3.64 (m, 2H), 3.48 (dd, J=12.5, 6.8 Hz, 2H), 3.30 (s, 2H), 3.17-3.05 (m, 2H), 2.83 (t, J=6.8 Hz, 2H), 1.33 (s, 6H). LC-MS m/z: 263.2 [M+H]⁺. HPLC Purity (214 nm): >99%; t_{R}=7.74 min.

### Example 54: 2,2-Dimethyl-N-phenethylpyrrolidine-1-carboxamide

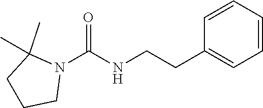 [SMILES: CC1(C)CCCN1C(=O)NCCc1ccccc1]

Following general procedure B, 2,2-dimethylpyrrolidine (270 mg, 2 mmol) and (2-isocyanatoethyl)benzene (352 mg 2.4 mmol) afforded the title compound (71 mg, 14%) as a white solid. ¹H NMR (400 MHz, CDCl₃) δ 7.36-7.24 (m, 2H), 7.22-7.18 (m, 3H), 4.07 (s, 1H), 3.47 (dd, J=12.7, 6.8 Hz, 2H), 3.18 (t, J=6.6 Hz, 2H), 2.82 (t, J=6.8 Hz, 2H), 1.88-1.70 (m, 4H), 1.41 (s, 6H). LC-MS m/z: 247.1 [M+H]⁺. HPLC Purity (214 nm): 99%; t_{R}=7.86 min.

### Example 55: 2,2-Dimethyl-N-phenethyl-4-phenylpipridine-1-carboxamide

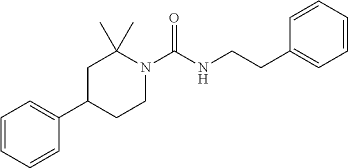 [SMILES: CC1(C)CC(c2ccccc2)CCN1C(=O)NCCc1ccccc1]

Following general procedure B, 2,2-dimethyl-4-phenylpiperidine (100 mg, 0.53 mmol) and (2-isocyanatoethyl)benzene (93 mg, 0.63 mmol) afforded the title compound (58.5 mg, 33%) as a white solid. ¹H NMR (400 MHz, CDCl₃) δ 7.37-7.27 (m, 4H), 7.27-7.20 (m, 6H), 4.53 (s, 1H), 3.56-3.38 (m, 3H), 3.16-3.05 (m, 1H), 2.87-2.77 (m, 3H), 1.95-1.88 (m, 1H), 1.77-1.55 (m, 3H), 1.53 (s, 3H), 1.38 (s, 3H). LC-MS m/z: 337.1 [M+H]⁺. HPLC Purity (214 nm): 98.27%; t_{R}=9.85 min.

### Example 56: N-(3-Phenylpropyl)-6-azaspiro[4.5]decane-6-carboxamide

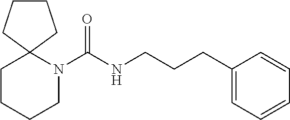 [SMILES: O=C(NCCCc1ccccc1)N1CCCCC12CCCC2]

Following general procedure A, 6-azaspiro[4.5]decane hydrochloride (120 mg, 0.69 mmol), 3-phenylpropan-1-amine (465 mg, 3.5 mmol) and triphosgene (465 mg, 3.5 mmol) afforded the title compound (78.5 mg, 38.2%) as a clear oil. ¹H NMR (400 MHz, CDCl₃) δ 7.34-7.26 (m, 2H), 7.23-7.18 (m, 3H), 4.45 (s, 1H), 3.32-3.24 (m, 4H), 2.65 (t, J=7.6 Hz, 2H), 1.96-1.48 (m, 16H). LC-MS m/z: 301.1 [M+H]⁺. HPLC Purity (214 nm): 100.0%; t_{R}=9.68 min.

### Example 57: N-(3-Phenylpropyl)-1-azaspiro[4.4]nonane-1-carboxamide

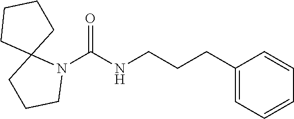 [SMILES: O=C(NCCCc1ccccc1)N1CCCC12CCCC2]

To a solution of nitrocyclopentane (2.5 g, 21.715 mmol) and phenyltrimethylammonium hydroxide (73 mg, 0.434 mmol) in dioxane (1.5 mL) was added methyl acrylate (1.87 g, 21.72 mmol) at 70° C. The mixture was stirred at 70° C. for 3 h. The mixture was cooled and diluted with EA (100 mL), washed with HCl (1 N, 40 mL), water (30 mL×2), Na₂CO₃ (a.q., 40 mL) and dried over Na₂SO₄ and concentrated to afford methyl 3-(1-nitrocyclopentyl)propanoate (3.3 g, 75%) as a light-yellow oil. ¹H NMR (400 MHz, CDCl₃) δ 3.69 (s, 3H), 2.65-2.48 (m, 2H), 2.40-2.25 (m, 4H), 1.88-1.69 (m, 6H).

To a solution of methyl 3-(1-nitrocyclopentyl)propanoate (4.0 g, 19.88 mmol) in EtOH (80 mL) was added Pd/C (10%) (500 mg). The mixture was stirred at 50° C. for 48 h. The mixture was filtered and concentrated to give 1-azaspiro[4.4]nonan-2-one (1.1 g, 39.8%) as a gray solid. ¹H NMR (400 MHz, MeOD-d₄) δ 2.31-2.20 (m, 2H), 1.97-1.78 (m, 2H), 1.76-1.54 (m, 8H).

To a solution of 1-azaspiro[4.4]nonan-2-one (500 mg, 3.59 mmol) in THF (10 mL) was added LAH (1 M in THF) (15 mL, 15.0 mmol). The mixture was stirred at 65° C. for 13 h and then quenched with sodium sulfate decahydrate and filtrated. The filtrate was concentrated to give crude 1-azaspiro[4.4]nonane (600 mg) as a light brown oil. LC-MS m/z: 126.2 [M+H]⁺. Purity (214 nm): 87%; t_{R}=0.96 min.

Following general procedure B, 1-azaspiro[4.4]nonane (400 mg, 3.19 mmol) and (3-isocyanatopropyl)benzene (1.5 g, 9.58 mmol) afforded the title compound (256 mg, 25%) as a colorless oil. ¹H NMR (400 MHz, CDCl₃) δ 7.28-7.24 (m, 2H), 7.22-7.14 (m, 3H), 4.02 (s, 1H), 3.27 (dd, J=12.9, 6.9 Hz, 2H), 3.16 (t, J=6.5 Hz, 2H), 2.71-2.59 (m, 2H), 2.42-2.26 (m, 2H), 1.93-1.73 (m, 8H), 1.56-1.45 (m, 2H), 1.44-1.35 (m, 2H). LC-MS m/z: 287.2 [M+H]⁺. HPLC Purity (214 nm): 99%; t_{R}=9.56 min.

### Example 58: N-(3-(4-Fluorophenyl)propyl)-6-azaspiro[4.5]decane-6-carboxamide

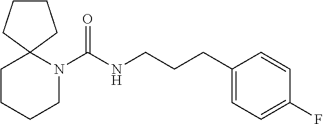 [SMILES: O=C(NCCCc1ccc(F)cc1)N1CCCCC12CCCC2]

To a solution of BTC (387 mg, 1.30 mmol) in DCM (8 mL) was added a solution of 3-(4-fluorophenyl)propan-1-amine (500 mg, 3.26 mmol) and 1,8-bis(dimethylamino)naphthalene (1.4 g, 6.53 mmol) in DCM (4 mL) and the mixture was stirred at RT for 2 h. The mixture was washed with HCl (1 N, 6 mL) twice, dried over Na₂SO₄ and concentrated to give 1-fluoro-4-(3-isocyanatopropyl)benzene (500 mg, crude) as a light orange oil. LC-MS m/z: 212.2 [M+H]⁺. Purity (214 nm): 90%; t_{R}=1.24 min.

Following general procedure B, 6-azaspiro[4.5]decane hydrochloride (150 mg, 0.85 mmol) and 1-fluoro-4-(3-isocyanatopropyl)benzene (500 mg, 2.56 mmol) afforded the title compound (137.7 mg, 51%) as a white solid. ¹H NMR (400 MHz, CDCl₃) δ 7.06 (dd, J=8.4, 5.6 Hz, 2H), 6.89 (t, J=8.7 Hz, 2H), 4.37 (s, 1H), 3.21-3.11 (m, 4H), 2.55 (t, J=7.7 Hz, 2H), 1.88-1.79 (m, 4H), 1.78-1.64 (m, 4H), 1.62-1.36 (m, 8H). LC-MS m/z: 319.2 [M+H]⁺. HPLC Purity (214 nm): 99%; t_{R}=9.80 min.

### Example 59: N-(3-(2,4-difluorophenyl)propyl)-6-azaspiro[4.5]decane-6-carboxamide

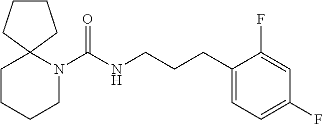 [SMILES: O=C(NCCCc1ccc(F)cc1F)N1CCCCC12CCCC2]

To a solution of BTC (347 mg, 1.17 mmol) in DCM (10 mL) was added a solution of 3-(2,4-difluorophenyl)propan-1-amine (500 mg, 3.26 mmol) and 1,8-bis(dimethylamino)naphthaene (1.4 g, 6.53 mmol) in DCM (5 mL) and the mixture was stirred at RT for 2 h. The reaction mixture was washed with HCl (1 N, 6 mL) twice, dried over Na₂SO₄ and concentrated to give 2,4-difluoro-1-(3-isocyanatopropyl)benzene (505 mg, crude) as a light brown oil. LC-MS m/z: 230.2 [M+H]⁺. Purity (214 nm): 54.72%; t_{R}=1.26 min.

Following general procedure B, 6-azaspiro[4.5]decane hydrochloride (150 mg, 0.85 mmol) and 2,4-difluoro-1-(3-isocyanatopropyl)benzene (500 mg, 2.56 mmol) afforded the title compound (183 mg, 64%) as a white solid. ¹H NMR (400 MHz, CDCl₃) δ 7.14 (dd, J=15.1, 8.4 Hz, 1H), 6.85-6.67 (m, 2H), 4.51 (s, 1H), 3.44-3.29 (m, 2H), 3.23 (q, J=7.2 Hz, 2H), 2.64 (t, J=7.6 Hz, 2H), 1.99-1.87 (m, 4H), 1.85-1.72 (m, 4H), 1.69-1.50 (m, 8H). LC-MS m/z: 337.0 [M+H]⁺. HPLC Purity (214 nm): 99%; t_{R}=9.92 min.

### Example 60: N-(3-Phenylpropyl)-9-oxa-6-azaspiro[4.5]decane-6-carboxamide

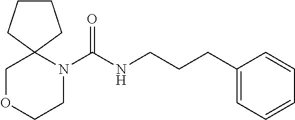 [SMILES: O=C(NCCCc1ccccc1)N1CCOCC12CCCC2]

To a solution of 1-aminocyclopentanecarboxylic acid (4 g, 31.0 mmol) in THF (50 mL) was added LAH (62 mL, 62.0 mmol) at 0° C. and the mixture was stirred at 0° C. for 1 h. The reaction mixture was treated with Na₂SO₄.10H₂O (50 g) and stirred at RT for another 1 h. The mixture was filtered, washed with THF (10 mL) and the filtrate was concentrated to give (1-aminocyclopentyl)methanol (3.17 g, 89%) as a colorless oil. LC-MS m/z: 116.1 [M+H]⁺; t_{R}=0.43 min.

A mixture of (1-aminocyclopentyl)methanol (3.17 g, 27.6 mmol), TEA (4.18 g, 41.3 mmol) and 2-bromoacetyl chloride (5.16 g, 33.1 mmol) in DCM (30 mL) was stirred at 0° C. for 2 h. The reaction mixture was concentrated, and the residue was purified by silica gel column chromatography (DCM:MeOH=50:1) to give 2-bromo-N-(1-(hydroxymethyl)cyclopentyl) acetamide (3.5 g, 54%) as a yellow oil. LC-MS m/z: 236.0 [M+H]⁺. Purity (254 nm): >60%; t_{R}=1.29 min.

To a solution of 2-bromo-N-(1-(hydroxymethyl)cyclopentyl)acetamide (3.5 g, 14.9 mmol) in THF (20 mL) was added NaH (655 mg, 16.3 mmol) at 0° C. The reaction mixture was stirred at RT for 15 h and then quenched with H₂O (100 mL) and extracted with EA (2×50 mL). The combined organic layers were concentrated, and the residue was purified by silica gel column chromatography (DCM:MeOH=50:1) to give 9-oxa-6-azaspiro[4.5]decan-7-one (2 g, 87%) as a yellow solid. LC-MS m/z: 156.2 [M+H]⁺. Purity (254 nm): >59%; t_{R}=0.59 min.

To a solution of 9-oxa-6-azaspiro[4.5]decan-7-one (500 mg, 3.22 mmol) in THF (3 mL) was added LAH (9.7 mL, 9.67 mmol). The mixture was stirred at 60° C. for 2 h. The reaction was cooled to RT and Na₂SO₄.10H₂O (5 g) was added and the mixture was stirred at RT for 30 min, filtered, washed with EA (10 mL) and concentrated. The residue was purified by silica gel column chromatography (DCM:MeOH=15:1) to give 9-oxa-6-azaspiro[4.5]decane (300 mg, 66%) as a yellow oil. LC-MS m/z: 142.1 [M+H]⁺; t_{R}=1.23 min.

A solution of 3-phenylpropan-1-amine (115 mg, 0.85 mmol) and bis(trichloromethyl) carbonate (252 mg, 0.85 mmol) in toluene (3 mL) was stirred at 120° C. for 2 h. Then the reaction mixture was cooled to 60° C. and 9-oxa-6-azaspiro[4.5]decane (100 mg, 0.71 mmol) was added followed by TEA (143 mg, 1.42 mmol). The resulting reaction mixture was stirred at 60° C. for another 2 h. The reaction mixture was concentrated, and the residue was purified by silica gel column chromatography (DCM:MeOH=50:1) to give the crude product which was purified by Prep-HPLC (FA) to afford the title compound (35.2 mg, 16.4%) as a white solid. ¹H NMR (400 MHz, CDCl₃) δ 7.35-7.29 (m, 2H), 7.24-7.17 (m, 3H), 4.38 (s, 1H), 3.71 (t, J=5.2 Hz, 2H), 3.34 (s, 2H), 3.30 (q, J=6.8 Hz, 2H), 3.26 (dd, J=12.6, 7.0 Hz, 2H), 3.17-3.12 (m, 2H), 2.67 (t, J=7.2 Hz, 2H), 2.11-1.97 (m, 2H), 1.97-1.80 (m, 4H), 1.69-1.61 (m, 2H), 1.56-1.44 (m, 2H). LC-MS m/z: 303.1 [M+H]⁺. HPLC Purity (214 nm): >99%; t_{R}=8.97 min.

### Examples 61 and 62: 4,4-Difluoro-2,2-dimethyl-N-(3-phenylpropyl)piperidine-1-carboxamide and 4-fluoro-2,2-dimethyl-N-(3-phenylpropyl)-3,6-dihydropyridine-1(2H)-carboxamide

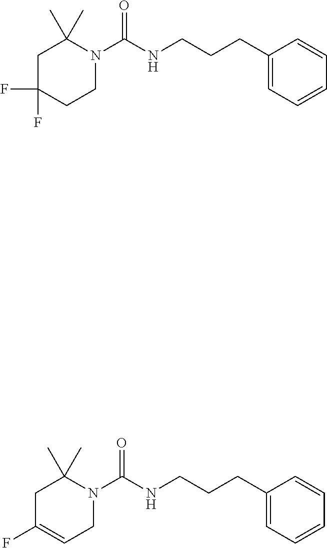 [SMILES: CC1(C)CC(F)(F)CCN1C(=O)NCCCc1ccccc1 ; CC1(C)CC(F)=CCN1C(=O)NCCCc1ccccc1]

To a mixture of I-butyl 2,2-dimethyl-4-oxopiperdine-1-carboxylate (500 mg, 2.2 mmol) in DCM (6 mL) was added DAST (10 ml). The mixture was stirred at RT for 40 h and then washed with aq Na₂CO₃ and concentrated in vacuo to give a residue which was purified by silica gel column chromatography (DCM/MeOH=20/1) affording t-butyl 4,4-difluoro-2,2-dimethylpiperidine-1-carboxylate (566 mg) as a yellow oil. LC-MS m/z: 194.2 [M+H]⁺. Purity (214 nm): 15.8%; t_{R}=1.51 min.

To a solution of dioxane/HCl (10 ml) was added tert-butyl 4,4-difluoro-2,2-dimethylpiperidine-1-carboxylate (254 mg, 1 mmol) and the mixture was stirred at RT for 2 h. The mixture was concentrated under vacuum to give the crude 4,4-difluoro-2,2-dimethylpiperdine (160 mg) as a yellow oil. LC-MS m/z: 150.1 [M+H]⁺. Purity (214 nm): 32.98%; t_{R}=1.52 min.

Following general procedure B, 4,4-difluoro-2,2-dimethylpiperidine (160 mg, 1 mmol) and (3-isocyanatopropyl)benzene (771 mg, 5.0 mmol) afforded the titled compounds of 4,4-difluoro-2,2-dimethyl-N-(3-phenylpropyl)piperidine-1-carboxamide (78.1 mg, 29%) and 4-fluoro-2,2-dimethyl-N-(3-phenylpropyl)-3,6-dihydropyridine-1(2H)-carboxamide (13.1 mg, 4.8%) as white solids.

### 4,4-difluoro-2,2-dimethyl-N-(3-phenylpropyl)piperidine-1-carboxamide

¹H NMR (400 MHz, CDCl₃) δ 7.32-7.27 (m, 2H), 7.24-7.18 (m, 3H), 4.40 (s, 1H), 3.25 (ddd, J=14.0, 10.3, 6.5 Hz, 4H), 2.67 (t, J=7.5 Hz, 2H), 2.07-1.98 (m, 2H), 1.96 (s, 1H), 1.93-1.82 (m, 3H), 1.44 (s, 6H). LC-MS m/z: 311.0 [M+H]⁺. HPLC Purity (214 nm): 97.76%; t_{R}=9.11 min.

### 4-fluoro-2,2-dimethyl-N-(3-phenylpropyl)-3,6-dihydropyridine-1(2H)-carboxamide

¹H NMR (400 MHz, CDCl₃) δ 7.32-7.27 (m, 2H), 7.23-7.16 (m, 3H), 5.00 (dd, J=17.2, 1.2 Hz, 1H), 4.30 (s, 1H), 3.33-3.24 (m, 4H), 2.68 (t, J=7.5 Hz, 2H), 2.30-2.25 (m, 2H), 1.92-1.86 (m, 2H), 1.47 (d, J=0.9 Hz, 6H). LC-MS m/z: 291.0 [M+H]⁺ HPLC Purity (214 nm): 97.31%; t_{R}=9.15 min.

### Example 63: 1-(Bicyclo[2.2.2]octan-1-yl)-1-methyl-3-(3-phenylpropyl)urea

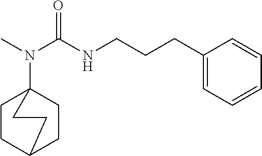 [SMILES: CN(C(=O)NCCCc1ccccc1)C12CCC(CC1)CC2]

To a solution of bicyclo[2.2.2]octane-1-carboxylic acid (0.6 g, 3.9 mmol) and TEA (1.17 g, 11.7 mmol) in 10 mL toluene was added DPPA (1.29 g, 4.7 mmol) and the mixture was stirred at RT for 2 h under N₂. Then the mixture was heated to 80° C. and stirred at RT for 16 h. The mixture was concentrated, and the residue was purified by silica gel column chromatography (PE/EA=15/1) to give benzyl bicyclo[2.2.2]octan-1-ylcarbamate (500 mg, 49.5%) as a colorless oil. LC-MS m/z: 260.1 [M+H]⁺.

To a solution of benzyl bicyclo[2.2.2]octan-1-ylcarbamate (0.5 g, 1.92 mmol) in THF (10 mL) was added LAH (220 mg, 5.77 mmol) at 0° C. and the mixture was stirred at RT for 2 h. Then Na₂SO₄.10H₂O (1.0 g) was added to the reaction mixture and the mixture was stirred for 10 min, filtered and concentrated to give N-methylbicyclo[2.2.2]octan-1-amine crude (140 mg, 52.4%) as a colorless oil. LC-MS m/z: 140.1 [M+H]⁺.

Following general procedure B, N-methylbicyclo[2.2.2]octan-1-amine (140 mg, 1.0 mmol) and (3-isocyanatopropyl)benzene (161 mg, 1.0 mmol) afforded the title compound (90.0 mg, 30.1%) as a white solid. ¹H NMR (400 MHz, CDCl₃) δ 7.29-7.24 (m, 2H), 7.21-7.15 (m, 3H), 4.19 (s, 1H), 3.24-3.19 (m, 2H), 2.67 (s, 3H), 2.66 (t, J=8.0 Hz, 2H), 1.96-1.87 (m, 6H), 1.85-1.79 (m, 2H), 1.79-1.72 (m, 1H), 1.67-1.61 (m, 7H), 1.53-1.50 (m, 1H). LC-MS m/z: 301.1 [M+H]⁺. HPLC Purity (214 nm): 95%; t_{R}=10.26 min.

### Example 64: 1-Methyl-3-(3-phenylpropyl)-1-(quinuclidin-3-yl)urea

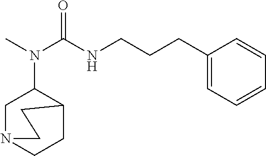 [SMILES: CN(C(=O)NCCCc1ccccc1)C1CN2CCC1CC2]

To a solution of quinuclidin-3-amine (1.0 g, 5.0 mmol) in DMF (10 mL) was added NaH (0.3 g, 7.5 mmol) and the mixture was stirred at 0° C. for 1 h. Then benzyl carbonochloridate (1.02 g, 6.0 mmol) was added and the mixture was stirred at RT for 16 h. Water (20 mL) was added and the mixture was extracted with EA (50 mL×3). The combined organic layers were concentrated to give crude benzyl quinuclidin-3-ylcarbamate (1.16 g, 56.3%) as a white solid. LC-MS m/z: 261.1 [M+H]⁺.

To a solution of benzyl quinuclidin-3-ylcarbamate (0.26 g, 1.0 mmol) in THF (10 mL) was added LAH (190 mg, 5.0 mmol) at 0° C. and the mixture was stirred at RT for 2 h. Then Na₂SO₄.10H₂O (1.0 g) was added and the mixture was stirred for 10 min. The mixture was filtered, and the filtrate was concentrated to give N-methylquinuclidin-3-amine (140 mg, 100%) as a colorless oil. LC-MS m/z: 141.1 [M+H]⁺.

Following general procedure B, N-methylquinuclidin-3-amine (140 mg, 1.0 mmol) and (3-isocyanatopropyl)benzene (161 mg, 1.0 mmol) afforded the title compound (50.0 mg, 30.1%) as a colorless oil. ¹H NMR (400 MHz, CDCl₃) δ 7.32-7.27 (m, 2H), 7.22-7.17 (m, 3H), 4.47-4.43 (m, 2H), 3.51-3.06 (m, 7H), 2.77 (s, 3H), 2.68 (t, J=7.6 Hz, 2H), 2.20-2.17 (m, 1H), 2.09-1.73 (m, 6H). LC-MS m/z: 302.3 [M+H]⁺. HPLC Purity (214 nm): 98%; t_{R}=7.13 min.

### Example 65: N-benzyl-2,2-dimethyl-7-(1-methylazetidin-3-yl)-3,4-dihydroquinoline-1(2H)-carboxamide

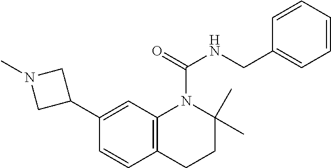 [SMILES: CN1CC(c2ccc3c(c2)N(C(=O)NCc2ccccc2)C(C)(C)CC3)C1]

Following general procedure B, 2,2-dimethyl-5-(1-methylazetidin-3-yl)-1,2,3,4-tetrahydroquinoline (115 mg, 0.5 mmol) and (isocyanatomethyl)benzene (220 mg, 1.5 mmol) afforded the title compound (21.9 mg, 15.8%) as a white solid. ¹H NMR (400 MHz, CDCl₃) δ 7.47-7.28 (m, 5H), 7.03 (d, J=7.5 Hz, 1H), 6.74 (d, J=7.8 Hz, 2H), 5.31 (t, J=5.4 Hz, 1H), 4.38 (d, J=6.0 Hz, 2H), 4.04 (t, J=8.5 Hz, 2H), 3.80-3.71 (m, 1H), 3.32 (t, J=8.6 Hz, 2H), 2.65-2.58 (m, 2H), 2.57 (s, 3H), 1.72 (dd, J=16.9, 11.0 Hz, 2H), 1.56 (s, 6H). LC-MS m/z: 364.2 [M+H]⁺. HPLC Purity (214 nm): 100%; t_{R}=6.96 min.

### Example 66: N-Benzyl-2,2-dimethyl-5-(1-methylazetidin-3-yl)-3,4-dihydroquinoline-1(2H)-carboxamide

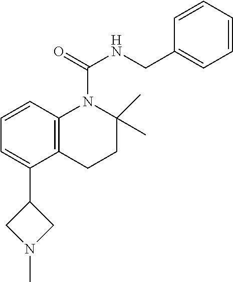 [SMILES: CN1CC(c2cccc3c2CCC(C)(C)N3C(=O)NCc2ccccc2)C1]

Following general procedure B, 2,2-dimethyl-7-(1-methylazetidin-3-yl)-1,2,3,4-tetrahydroquinoline (143 mg, 0.6 mmol) and (isocyanatomethyl)benzene (132 mg, 0.9 mmol). afforded the title compound (31.0 mg, 10.8%) as a white solid. ¹H NMR (400 MHz, CDCl₃) δ 7.38-7.31 (m, 4H), 7.31-7.26 (m, 2H), 7.07 (t, J=7.9 Hz, 1H), 6.95 (d, J=8.1 Hz, 1H), 6.74 (d, J=7.6 Hz, 1H), 5.20 (t, J=5.5 Hz, 1H), 4.47-4.40 (m, 4H), 4.34-4.20 (m, 1H), 3.74-3.56 (m, 2H), 2.73 (s, 3H), 2.46-2.39 (m, 2H), 1.75-1.68 (m, 2H), 1.53 (s, 6H). LC-MS m/z: 364.2 [M+H]⁺. HPLC Purity (214 nm): 100%; t_{R}=6.75 min.

### Example 67: 2,2-Dimethyl-N-phenethylindoline-1-carboxamide

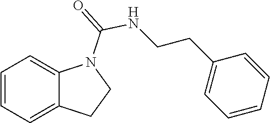 [SMILES: O=C(NCCc1ccccc1)N1CCc2ccccc21]

To a solution of 1-acetylindolin-3-one (750 mg, 4.3 mmol) in DMF (10 mL) was added NaH (514 mg, 12.8 mmol). The mixture was stirred at RT for 0.5 h, then MeI (6.1 g, 5.7 mmol) was added and the mixture was stirred at 80° C. for 15 h. The reaction mixture was quenched with H₂O (100 mL) and extracted with EA (2×100 mL). The combined organic layers were concentrated, and the residue was purified by silica gel column chromatography (PE:EA=9:1) to give 1-acetyl-2,2-dimethylindolin-3-one (230 mg, 26%) as a yellow solid. LC-MS m/z: 204.2 [M+H]⁺. HPLC Purity (254 nm): >96%; t_{R}=1.14 min.

A mixture of 1-acetyl-2,2-dimethylindolin-3-one (230 mg, 1.13 mmol) and NaOH (4 mL, 2 N) in EtOH (4 mL) was stirred at 100° C. for 1 h and then the reaction mixture was concentrated, and the residue was washed with H₂O (20 mL). The reaction mixture was extracted with EA (2×20 mL) and the combined organic layers were concentrated to give 2,2-dimethylindolin-3-one (160 mg, 88%) as a yellow oil. LC-MS m/z: 162.3 [M+H]⁺. HPLC Purity (254 nm): >99%; t_{R}=1.04 min.

To a solution of 2,2-dimethylindolin-3-one (160 mg, 1.0 mmol) and AlCl₃ (133 mg, 1.0 mmol) in THF (2 mL) was added LAH (2 mL, 2 mmol) at 0° C. and the mixture was stirred at RT for 2 h. Then the reaction mixture was quenched with Na₂SO₄.10H₂O and stirred at RT for 0.5 h. The mixture was filtered and washed with EA (10 mL), concentrated and purified by silica gel column chromatography (PE:EA=9:1) to give 2,2-dimethylindoline (85 mg, 58%) as a yellow oil. LC-MS m/z: 148.1 [M+H]⁺. HPLC Purity (214 nm): >90%; t_{R}=1.21 min.

Following general procedure B, 2,2-dimethylindoline (85 mg, 0.58 mmol) and (2-isocyanatoethyl)benzene (170 mg, 1.16 mmol) afforded the title compound (65.8 mg, 38.7%) as a white solid. ¹H NMR (400 MHz, CDCl₃) δ 7.37-7.33 (m, 2H), 7.29-7.24 (m, 3H), 7.09 (d, J=7.3 Hz, 1H), 6.99 (t, J=7.7 Hz, 1H), 6.91 (d, J=8.0 Hz, 1H), 6.83 (t, J=7.3 Hz, 1H), 4.91 (s, 1H), 3.64 (dd, J=12.5, 6.7 Hz, 2H), 2.93 (t, 1=6.8 Hz, 2H), 2.89 (s, 2H), 1.52 (s, 6H). LC-MS m/z: 295.0 [M+H]⁺. HPLC Purity (214 nm): >99%; t_{R}=10.12 min.

### Example 68: 8-Methoxy-2,2-dimethyl-N-phenethyl-3,4-dihydroquinoline-1(2H)-carboxamide

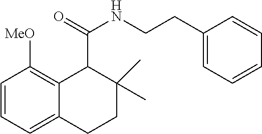 [SMILES: COc1cccc2c1C(C(=O)NCCc1ccccc1)C(C)(C)CC2]

A mixture of 2-methoxyaniline (1 g, 8.1 mmol), 3-chloro-3-methylbut-1-yne (1.24 g, 12.2 mmol), Cu (520 mg, 8.1 mmol) and CuCl (805 mg, 8.1 mmol) in toluene (10 mL) was stirred at 120° C. for 3 h. The reaction mixture was concentrated and purified by silica gel column chromatography (PE:EA=4:1) to give 8-methoxy-2,2-dimethyl-1,2-dihydroquinoline (270 mg, 18%) as a yellow oil. LC-MS m/z: 190.2 [M+H]⁺. Purity (214 nm): >84%; t_{R}=1.41 min.

A mixture of 8-methoxy-2,2-dimethyl-1,2-dihydroquinoline (270 mg, 1.43 mmol) and Pd/C (80 mg) in MeOH (10 mL) was stirred at RT under H₂ for 15 h. Then the reaction mixture was filtered and washed with MeOH (10 mL). The filtrate was concentrated to give 8-methoxy-2,2-dimethyl-1,2,3,4-tetrahydroquinoline (250 mg, 92%) as a yellow oil. LC-MS m/z: 192.3 [M+H]⁺. Purity (214 nm): >90%; t_{R}=1.06 min.

Following general procedure B, 8-methoxy-2,2-dimethyl-1,2,3,4-tetrahydroquinoline (200 mg, 1.05 mmol) and (2-isocyanatoethyl)benzene (185 mg, 1.26 mmol) afforded the title compound (77.4 mg, 21.9%) as a white solid. ¹H NMR (400 MHz, CDCl₃) δ 7.28-7.15 (m, 3H), 7.10 (d, J=6.9 Hz, 2H), 6.95 (t, J=7.8 Hz, 1H), 6.72 (d, J=7.3 Hz, 1H), 6.67 (d, J=8.3 Hz, 1H), 4.69 (s, 1H), 3.70 (s, 3H), 3.48-3.33 (m, 2H), 2.75 (t, J=7.3 Hz, 2H), 2.60-2.48 (m, 2H), 1.76-1.63 (m, 2H), 1.55 (s, 6H). LC-MS m/z: 339.0 [M+H]⁺. HPLC Purity (214 nm): >99%; t_{R}=9.90 min.

### Example 69: 2,2-Dimethyl-7-(1′-methyl-[1,3′-biazetidin]-3-yl)-N-phenethyl-3,4-dihydroquinoline-1(2H)-carboxamide

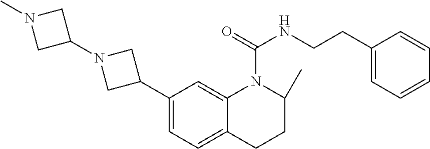 [SMILES: CC1CCc2ccc(C3CN(C4CN(C)C4)C3)cc2N1C(=O)NCCc1ccccc1]

To a solution of 7-(azetidin-3-yl)-2,2-dimethyl-1,2,3,4-tetrahydroquinoline (250 mg, 1.2 mmol) in MeOH (15 mL) was added 1-methylazetidin-3-one (153 mg, 1.8 mmol). The mixture was stirred at RT for 2 h and then NaBH₃CN (375 mg, 6 mmol) was added at 0° C. and the solution was stirred at 50° C. for 16 h. The reaction mixture was concentrated and purified by silica gel column chromatography (DCM/MeOH=1/1) to give 2,2-dimethyl-7-(1′-methyl-1,3′-biazetidin-3-yl)-1,2,3,4-tetrahydroquinoline (120 mg, 36.4%) as a yellow oil. LC-MS m/z: 286.3 [M+H]⁺. HPLC Purity (254 nm): 91.54%; t_{R}=1.78 min.

Following general procedure B, 2,2-dimethyl-7-(1′-methyl-1,3′-biazetidin-3-yl)-1,2,3,4-tetrahydroquinoline (120 mg, 0.42 mmol) and (2-isocyanatoethyl)benzene (310 mg, 2.1 mmol) afforded the title compound (4.1 mg, 2.3%) as a white solid. ¹H NMR (400 MHz, CDCl₃) δ 8.63 (s, 1H), 7.32-7.18 (m, 5H), 6.96 (d, J=7.7 Hz, 1H), 6.49 (d, J=7.6 Hz, 1H), 6.36 (s, 1H), 4.71 (s, 1H), 3.81-3.52 (m, 7H), 3.30-3.20 (m, 5H), 2.96-2.89 (m, 2H), 2.73-2.63 (m, 4H), 1.70 (t, J=6.6 Hz, 2H), 1.22 (s, 6H). LC-MS m/z: 433.1 [M+H]⁺. HPLC Purity (214 nm): 100.0%; t_{R}=5.33 min.

### Example 70: 7-(1-Acetylazetidin-3-yl)-2,2-dimethyl-N-phenethyl-3,4-dihydroquinoline-1(2H)-carboxamide

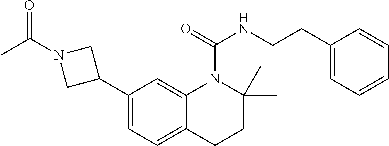 [SMILES: CC(=O)N1CC(c2ccc3c(c2)N(C(=O)NCCc2ccccc2)C(C)(C)CC3)C1]

To a solution of 3-(3-nitrophenyl)azetidine (929 g, 5.2 mmol) in DCM (15 mL) was added TEA (1.6 g, 15.6 mmol) and acetyl chloride (612 mg, 7.8 mmol). The mixture was stirred at RT overnight and then washed with water and concentrated under vacuum to give a residue which was purified by FCC (PE:EA=2:1) to give 1-(3-(3-nitrophenyl)azetidin-1-yl)ethan-1-one (746 mg, 65.2%) as a yellow oil. LC-MS m/z: 221.2 [M+H]⁺. HPLC Purity (254 nm): 60.5%; t_{R}=1.05 min.

To a solution of 1-(3-(3-nitrophenyl)azetidin-1-yl)ethan-1-one (696 g, 3 mmol) in MeOH (15 mL) was added Pd/C (70 mg). The mixture was stirred at RT for 1 h under H₂ and then the mixture was filtered and concentrated to give 1-(3-(3-aminophenyl)azetidin-1-yl)ethan-1-one (564 mg, 95.6%) as a green oil. LC-MS m/z: 191.1 [M+H]⁺. HPLC Purity (214 nm): 65.2%: t_{R}=1.42 min.

To a solution of 1-(3-(3-aminophenyl)azetidin-1-yl)ethan-1-one (564 g, 3 mmol) in toluene (15 mL) was added Cu (189 mg, 3 mmol), CuCl (294 mg, 3 mmol) and 3-chloro-3-methylbut-1-yne (454 mg, 4.5 mmol). The mixture was stirred at 120° C. for 3 h under N₂. The mixture was filtered, concentrated and purified by silica gel column chromatography to give 1-(3-(2,2-dimethyl-1,2-dihydroquinolin-7-yl)azetidin-1-yl)ethan-1-one (176 mg, 22.9%) as a green oil. LC-MS m/z: 257.1 [M+H]⁺. Purity (214 nm): 40.14%; t_{R}=1.25 min.

To a solution of 1-(3-(2,2-dimethyl-1,2-dihydroquinolin-7-yl)azetidin-1-yl)ethan-1-one (176 mg, 0.7 mmol) in MeOH (10 mL) was added PtO₂ (454 mg, 4.5 mmol). The mixture was stirred at RT for 2 h. The mixture was filtered and concentrated to give 1-(3-(2,2-dimethyl-1,2,3,4-tetrahydroquinolin-7-yl)-azetidin-1-yl)ethan-1-one (150 mg, 83.3%) as a green oil. LC-MS m/z: 259.3 [M+H]⁺. HPLC Purity (214 nm): 61.21%; t_{R}=1.03 min.

Following general procedure B, 1-(3-(2,2-dimethyl-1,2,3,4-tetrahydroquinolin-7-yl)azetidin-1-yl)-ethan-1-one (150 mg, 0.58 mmol) and (2-isocyanatoethyl)benzene (427 mg, 2.9 mmol) afforded the title compound (5.7 mg, 2.4%) as a white solid. ¹H NMR (400 MHz, CDCl₃) δ 7.33-7.28 (m, 2H), 7.21 (d, J=7.3 Hz, 1H), 7.17 (d, J=6.8 Hz, 2H), 7.05 (d, J=7.7 Hz, 1H), 6.88-6.81 (m, 2H), 5.03 (s, 1H), 4.42 (t, J=8.6 Hz, 1H), 4.33 (t, J=9.4 Hz, 1H), 4.10-3.98 (m, 1H), 3.94 (dd, J=9.8, 6.3 Hz, 1H), 3.63-3.50 (m, 3H), 2.84 (t, J=6.9 Hz, 2H), 2.65-2.58 (m, 2H), 1.89 (s, 3H), 1.77-1.73 (m, 2H), 1.52 (s, 6H). LC-MS m/z: 406.1 [M+H]⁺. HPLC Purity (214 nm): =100%; t_{R}=8.67 min.

### Example 71: 1-(Bicyclo[2.2.2]octan-1-yl)-1-methyl-3-(3-(1-(trifluoromethyl)cyclopropyl) propyl)urea

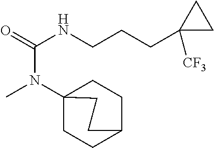 [SMILES: CN(C(=O)NCCCC1(C(F)(F)F)CC1)C12CCC(CC1)CC2]

A solution of 3-(1-(trifluoromethyl)cyclopropyl)propan-1-amine (600 mg, 3.6 mmol) and N1,N1,N8,N8-tetramethylnaphthalene-1,8-diamine (1.5 g, 7.2 mmol) in DCM (2 mL) was added dropwise to a stirred solution of BTC (323 mg, 1.08 mmol) in DCM (4 mL) at 0° C. and the mixture was stirred at RT for 15 min. After dilution with DCM (30 mL), the mixture was washed with 1N HCl (2×10 mL) and H₂O (6 mL). The organic layer was dried over Na₂SO₄, filtered and concentrated to give 1-(3-isocyanatopropyl)-1-(trifluoromethyl)cyclopropane (700 mg, >99%, purity: 34%) as a colorless oil. LC-MS m/z: 194.1 [M+H]⁺ t_{R}=1.88 min.

Following general procedure B, N-methylbicyclo[2.2.2]octan-1-amine (50 mg, 0.36 mmol) and 1-(3-isocyanatopropyl)-1-(trifluoromethyl)cyclopropane (140 mg, 0.72 mmol) afforded the title compound (33.2 mg, 27.78%) as a white oil. ¹H NMR (400 MHz, CDCl₃) δ 4.24 (s, 1H), 3.15 (dd, J=12.8, 6.4 Hz, 2H), 2.76 (s, 3H), 1.95 (dd, J=9.6, 6.2 Hz, 6H), 1.69 (s, 2H), 1.66-1.61 (m, 8H), 1.54-1.51 (m, 1H), 0.93 (t, J=5.8 Hz, 2H), 0.56 (s, 2H). LC-MS m/z: 333.2 [M+H]⁺. HPLC Purity (214 nm): 100%; t_{R}=10.07 min.

### Examples 72 and 73: 4,4-Difluoro-2,2-dimethyl-N-(3-(1-(trifluoromethyl)cyclopropyl)propyl)piperidine-1-carboxamide and 4-fluoro-2,2-Dimethyl-N-(3-(1-(trifluoromethyl)cyclopropyl)propyl)-3,6-dihydropyridine-1(2H)-carboxamide

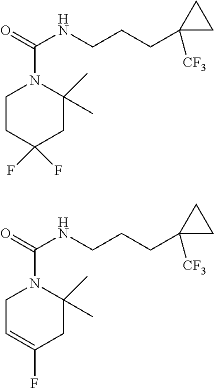 [SMILES: CC1(C)CC(F)(F)CCN1C(=O)NCCCC1(C(F)(F)F)CC1 ; CC1(C)CC(F)=CCN1C(=O)NCCCC1(C(F)(F)F)CC1]

To a solution of tert-butyl 2, 2-dimethyl-4-oxopiperidine-1-carboxylate (500 mg, 2.2 mmol) in DCM (5 mL) was added DAST (7.1 g, 44 mmol) and the reaction mixture was stirred at RT for 40 h. The mixture was washed with saturated aq. Na₂CO₃ solution and extracted with EA. The organic layer was concentrated and purified by FCC (DCM/MeOH=50/1) to give tert-butyl 4,4-difluoro-2,2-dimethylpiperidine-1-carboxylate (292 mg, 53%) as a yellow oil. LC-MS m/z: 194 [M−56+H]⁺. Purity (214 nm): 70.88%; t_{R}=1.58 min.

To a solution of tert-butyl 4, 4-difluoro-2,2-dimethylpiperidine-1-carboxylate (292 g, 1.17 mmol) in DCM (3 mL) was added 1,4-dioxane HCl (10 ml) and the reaction mixture was stirred at RT for 2 h. The mixture was concentrated to afford 4,4-difluoro-2,2-dimethylpiperidine (250 mg, 93%) as a yellow oil. LC-MS m/z: 150.1 [M+H]⁺. Purity (214 nm): 65.16%; t_{R}=1.56 min.

Following general procedure B, 4, 4-difluoro-2,2-dimethylpiperidine (250 mg, 1.68 mmol) and 1-(3-isocyanatopropyl)-1-(trifluoromethyl)cyclopropane (648 mg, 3.35 mmol) afforded 4,4-difluoro-2,2-dimethyl-N-(3-(1-(trifluoromethyl)cyclopropyl)propyl)piperidine-1-carboxamide (18.7 mg, 3.2%) and 4-fluoro-2,2-dimethyl-N-(3-(1-(trifluoromethyl)cyclopropyl)propyl)-3,6-dihydropyridine-1(2H)-carboxamide (4.2 mg, 0.7%) both as white solids.

### 4,4-difluoro-2,2-dimethyl-N-(3-(1-(trifluoromethyl)cyclopropyl)propyl) piperidine-1-carboxamide

¹H NMR (400 MHz, CDCl₃) δ 4.49 (s, 1H), 3.45-3.35 (m, 2H), 3.17 (dd, J=12.7, 6.9 Hz, 2H), 2.13-2.01 (m, 2H), 1.95 (t, J=14.8 Hz, 2H), 1.71-1.60 (m, 2H), 1.60-1.52 (m, 2H), 1.46 (s, 6H), 0.98-0.88 (m, 2H), 0.59 (d, J=22.0 Hz, 2H). LC-MS m/z: 343.1 [M+H]⁺. HPLC Purity (214 nm): 100%; t_{R}=9.21 min.

### 4-fluoro-2,2-dimethyl-N-(3-(1-(trifluoromethyl)cyclopropyl)propyl)-3,6-dihydropyridine-1(2H)-carboxamide

¹H NMR (400 MHz, CDCl₃) δ 5.01 (d, J=15.9 Hz, 1H), 4.39 (s, 1H), 3.47 (td, J=5.6, 2.1 Hz, 2H), 3.18 (dd, J=12.7, 6.8 Hz, 2H), 2.40-2.24 (m, 2H), 1.70-1.61 (m, 2H), 1.61-1.56 (m, 2H), 1.50 (d, J=0.8 Hz, 6H), 0.95 (t, J=5.8 Hz, 2H), 0.57 (s, 2H). LC-MS m/z: 323.1 [M+H]⁺. HPLC Purity (214 nm): 100%; t_{R}=9.25 min.

### Example 74: 2,2-Dimethyl-4-phenyl-N-(4-phenylbutyl)piperidine-1-carboxamide tert-Butyl 4-hydroxy-2,2-dimethyl-4-phenylpiperidine-1-carboxylate (XIa)

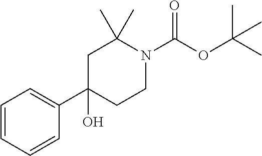 [SMILES: CC(C)(C)OC(=O)N1CCC(O)(c2ccccc2)CC1(C)C]

Following general procedure H (method A), V-Aa (0.200 g, 0.88 mmol) and PhMgBr (1.0 mL, 2.82 mmol, 2.8 M solution in 2-MeTHF) afforded XIa as a colorless oil (0.228 g, 85%). ¹H NMR (400 MHz, DMSO-d₆) δ 7.52-7.47 (m, 2H), 7.35-7.27 (m, 2H), 7.24-7.18 (m, 1H), 4.92 (s, 1H), 3.82 (dt, J=13.2, 4.2 Hz, 1H), 3.41-3.34 (m, 1H), 2.06-1.93 (m, 1H), 1.80 (d, J=14.2 Hz, 1H), 1.70 (ddd, J=13.5, 4.1, 2.6 Hz, 1H), 1.61 (dd, J=14.2, 2.5 Hz, 1H), 1.45 (s, 3H), 1.41 (s, 12H). UPLC/MS (method A): Rt 2.40 min. MS (ES) C₁₈H₂₇NO₃ requires 305, found 306 [M+H]⁺.

### tert-Butyl 6,6-dimethyl-4-phenyl-2,5-dihydropyridine-I-carboxylate (VIIIa)

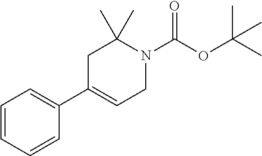 [SMILES: CC(C)(C)OC(=O)N1CC=C(c2ccccc2)CC1(C)C]

Following general procedure J, XIa (0.228 g, 0.75 mmol) and Burgess reagent (0.268 g, 1.13 mmol) afforded VIIa which was used in the next step without further purification. UPLC/MS (method B): Rt 2.15 min. MS (ES) C₁₈H₂₅NO₂ requires 287, found 288 [M+H]⁺.

### tert-Butyl 2,2-dimethyl-4-phenylpiperidine-1-carboxylate (IXa)

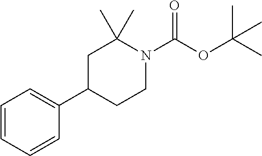 [SMILES: CC(C)(C)OC(=O)N1CCC(c2ccccc2)CC1(C)C]

Following general procedure B (Method E), VIIIa (0.103 g, 0.36 mmol) afforded IXa as a colorless oil (0.085 g, 82%). ¹H NMR (400 MHz, DMSO-d₆) δ 7.33-7.22 (m, 4H), 7.21-7.16 (m, 1H), 3.85 (dt, J=13.5, 4.8 Hz, 1H), 3.13 (ddd, J=13.5, 10.6, 3.7 Hz, 1H), 2.89-2.78 (m, 1H), 1.91-1.80 (m, 1H), 1.69 (t, J=13.2 Hz, 1H), 1.61-1.51 (m, 2H), 1.49 (s, 3H), 1.41 (s, 9H), 1.32 (s, 3H). UPLC/MS (method B): Rt 2.18 min. MS (ES) C₁₈H₂₇NO₂ requires 289, found 290 [M+H]⁺.

### 2,2-Dimethyl-4-phenylpiperidine hydrochloride (Xa)

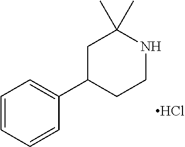

Following general procedure C, IXa (0.085 g, 0.29 mmol) afforded Xa which was used in the next step without further purification. UPLC/MS (method A): Rt 1.35 min. MS (ES) C₁₃H₁₉N requires 189, found 190 [M+H]⁺.

### 2,2-Dimethyl-4-phenyl-N-(4-phenylbutyl)piperidine-1-carboxamide

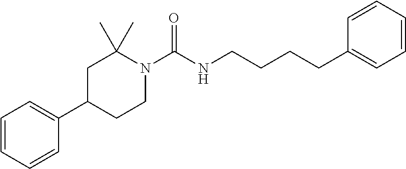 [SMILES: CC1(C)CC(c2ccccc2)CCN1C(=O)NCCCCc1ccccc1]

Following general procedure D (method A), Xa (0.066 g, 0.29 mmol) and 4-phenylbutyl isocyanate (0.056 g, 0.32 mmol) afforded the titled compound of example 78 as a colorless oil (0.044 g, 42%). ¹H NMR (400 MHz, DMSO-d₆) δ 7.37-7.09 (m, 10H), 6.46 (t, J=5.5 Hz, 1H), 3.60 (dt, J=12.9, 4.0 Hz, 1H), 3.07-2.92 (m, 3H), 2.78 (tt, J=12.1, 3.9 Hz, 1H), 2.57 (t, J=7.6 Hz, 2H), 1.86-1.76 (m, 1H), 1.65-1.48 (m, 5H), 1.45 (s, 3H), 1.44-1.35 (m, 2H), 1.31 (s, 3H). UPLC/MS (method B): Rt 1.90 min. MS (ES) C₂₄H₃₂N₂O requires 364, found 365 [M+H]⁺.

### Example 75: N-(2-Benzyloxyethyl)-2,2-dimethyl-4-phenylpiperidine-1-carboxamide

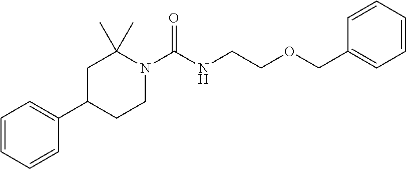 [SMILES: CC1(C)CC(c2ccccc2)CCN1C(=O)NCCOCc1ccccc1]

Following general procedure D (method B), Xa (0.025 g, 0.11 mmol), Et₃N (0.022 g, 0.22 mmol) and 2-benzyloxyethylamine (0.017 g, 0.11 mmol) afforded the title compound as a colorless oil (0.034 g, 83%). ¹H NMR (400 MHz, DMSO-d₆) δ 7.38-7.16 (m, 10H), 6.52 (t, J=5.6 Hz, 1H), 4.48 (s, 2H), 3.62 (dt, J=13.0, 4.1 Hz, 1H), 3.43 (t, J=6.2 Hz, 2H), 3.26-3.12 (m, 2H), 3.01 (ddd, J=12.9, 11.7, 3.1 Hz, 1H), 2.79 (tt, J=12.1, 4.0 Hz, 1H), 1.86-1.76 (m, 1H), 1.65-1.48 (m, 3H), 1.46 (s, 3H), 1.31 (s, 3H). UPLC/MS (method A): Rt 2.56 min. MS (ES) C₂₃H₃₀N₂O₂ requires 366, found 367 [M+H]⁺.

### Example 76: 2,2-Dimethyl-N-pentyl-4-phenylpiperidine-1-carboxamide

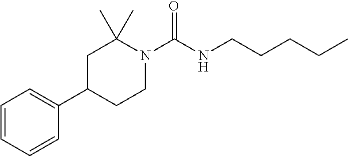 [SMILES: CCCCCNC(=O)N1CCC(c2ccccc2)CC1(C)C]

Following general procedure D (method A), Xa (0.040 g, 0.18 mmol) and n-pentyl isocyanate (0.022 g, 0.19 mmol) afforded the title compound as a colorless oil (0.043 g, 79%). ¹H NMR (400 MHz, DMSO-d₆) δ 7.34-7.12 (m, 5H), 6.43 (t, J=5.5 Hz, 1H), 3.61 (dt, J=12.9, 4.0 Hz, 1H), 3.05-2.89 (m, 3H), 2.78 (tt, J=12.1, 4.0 Hz, 1H), 1.86-1.76 (m, 1H), 1.65-1.48 (m, 3H), 1.45 (s, 3H), 1.43-1.34 (m, 2H), 1.31 (s, 3H), 1.30-1.16 (m, 4H), 0.86 (t, J=7.0 Hz, 3H). UPLC/MS (method B): Rt 1.69 min. MS (ES) C₁₉H₃₀N₂O requires 302, found 303 [M+H]⁺.

### Example 77: N-(2-Ethoxyethyl)-2,2-dimethyl-4-phenylpiperidine-1-carboxamide

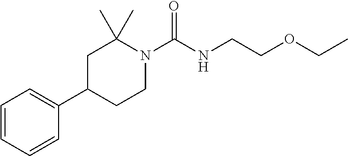 [SMILES: CCOCCNC(=O)N1CCC(c2ccccc2)CC1(C)C]

Following general procedure D (method B), Xa (0.050 g, 0.24 mmol), Et₃N (0.048 g, 0.48 mmol) and 2-ethoxyethylamine (0.021 g, 0.24 mmol) afforded the title compound as a white solid (0.031 g, 42%). ¹H NMR (400 MHz, DMSO-d₆) δ 7.34-7.15 (m, 5H), 6.47 (t, J=5.5 Hz, 1H), 3.61 (dt, J=12.9, 4.1 Hz, 1H), 3.42 (q, J=7.0 Hz, 2H), 3.38-3.33 (m, 2H), 3.12 (q, J=6.3 Hz, 2H), 3.01 (ddd, J=12.9, 11.7, 3.1 Hz, 1H), 2.79 (tt, J=12.1, 4.0 Hz, 1H), 1.86-1.76 (m, 1H), 1.66-1.48 (m, 3H), 1.46 (s, 3H), 1.31 (s, 3H), 1.10 (t, J=7.0 Hz, 3H). UPLC/MS (method A): Rt 2.28 min MS (ES) C₁₈H₂₈N₂O₂ requires 304, found 305 [M+H]⁺.

### Example 78: 2,2-Dimethyl-4-phenyl-N-tetrahydropyran-4-yl-piperidine-1-carboxamide

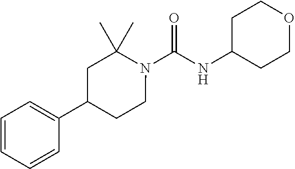 [SMILES: CC1(C)CC(c2ccccc2)CCN1C(=O)NC1CCOCC1]

Following general procedure D (method B), Xa (0.025 g, 0.11 mmol), Et₃N (0.022 g, 0.22 mmol) and 4-aminotetrahydropyran (0.011 g, 0.11 mmol) afforded the title compound as a colorless oil (0.034 g, 97%). ¹H NMR (400 MHz, DMSO-d₆) δ 7.34-7.15 (m, 5H), 6.34 (d, J=7.5 Hz, 1H), 3.82 (dt, J=11.6, 3.4 Hz, 2H), 3.69-3.51 (m, 2H), 3.35-3.24 (m, 2H), 3.00 (td, J=12.4, 3.0 Hz, 1H), 2.79 (tt, J=12.2, 4.0 Hz, 1H), 1.86-1.76 (m, 1H), 1.74-1.48 (m, 5H), 1.45 (s, 3H), 1.44-1.33 (m, 2H), 1.32 (s, 3H). UPLC/MS (method A): Rt 2.16 min. MS (ES) C₁₉H₂₈N₂O₂ requires 316, found 317 [M+H]⁺.

### Example 79: 2,2-Dimethyl-N-(4-phenylbutyl)-4-(2-pyridyl)piperidine-1-carboxamide tert-Butyl 4-hydroxy-2,2-dimethyl-4-(2-pyridyl)piperidine-1-carboxylate (XIb)

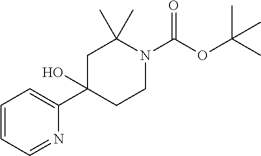 [SMILES: CC(C)(C)OC(=O)N1CCC(O)(c2ccccn2)CC1(C)C]

Following general procedure H (method B), V-Aa (0.72 g, 3.18 mmol) and 2-bromo-pyridine (0.5 g, 3.18 mmol) afforded XIb as a yellow oil (0.50 g, 51%). ¹H NMR (400 MHz, CDCl₃) δ 8.54 (d, J=4.9 Hz, 1H), 7.75 (td, J=7.8, 1.7 Hz, 1H), 7.39 (d, J=8.0 Hz, 1H), 4.10-3.95 (m, 1H), 3.90 (t, J=6.0 Hz, 1H), 3.57 (ddd, J=13.9, 11.5, 3.0 Hz, 1H), 2.57 (s, 1H), 2.44 (t, J=6.0 Hz, 3H), 2.08 (ddd, J=13.5, 11.5, 4.3 Hz, 3H), 1.85-1.75 (m, 9H). UPLC/MS (method A): Rt 2.22 min. MS (ES) C₁₇H₂₆N₂O₃ requires 306, found 307 [M+H]⁺.

### tert-Butyl 6,6-dimethyl-4-(2-pyridyl)-2,3-dihydropyridine-1-carboxylate (VIIb)

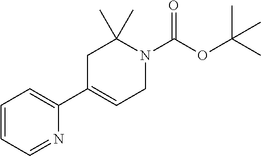 [SMILES: CC(C)(C)OC(=O)N1CC=C(c2ccccn2)CC1(C)C]

Following general procedure J, XIb (0.45 g, 1.47 mmol) and Burgess reagent (0.525 g, 2.2 mmol) afforded VIIb (0.250 g, 59%). ¹H NMR (400 MHz, CDCl₃) δ 8.58 (ddd, J=0.9, 1.8, 4.8 Hz, 1H), 7.71-7.63 (m, 1H), 7.38 (d, J=8.0 Hz, 1H), 7.15 (ddt, J=1.5, 4.8, 7.5 Hz, 1H), 6.35 (t, J=1.4 Hz, 1H), 4.24-4.01 (m, 1H), 3.66 (t, J=5.5 Hz, 1H), 2.76-2.68 (m, 1H), 2.67-2.53 (m, 1H), 1.51 (s, 6H), 1.49 (s, 9H). UPLC/MS (method A): Rt 2.45 min. MS (ES) C₁₇H₂₄N₂O₂ requires 288, found 289 [M+H]⁺.

### tert-Butyl 2,2-dimethyl-4-(2-pyridyl)piperidine-1-carboxylate (IXb)

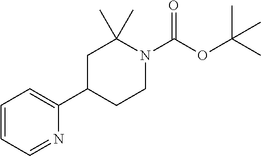 [SMILES: CC(C)(C)OC(=O)N1CCC(c2ccccn2)CC1(C)C]

Following general procedure B (method E), VIIIb (0.05 g, 0.17 mmol) afforded IXb which was used in the next step without further purification. UPLC/MS (method A): Rt 2.38 min. MS (ES) C₁₇H₂₆N₂O₂ requires 290, found 291 [M+H]⁺.

### 2-(2,2-Dimethyl-4-piperidyl)pyridine hydrochloride (Xb)

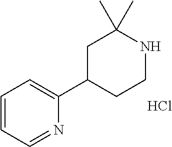

Following general procedure C, IXb (0.05 g, 0.17 mmol) afforded Xb as a white solid (0.037 g, 96%). UPLC/MS (method A): Rt 1.05 min. MS (ES) C₁₂H₁₈N₂ requires 190, found 191 [M+H]⁺.

### 2,2-Dimethyl-N-(4-phenylbutyl)-4-(2-pyridyl)piperidine-1-carboxamide

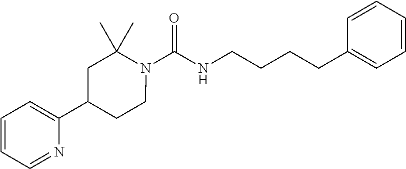 [SMILES: CC1(C)CC(c2ccccn2)CCN1C(=O)NCCCCc1ccccc1]

Following general procedure D (method A), Xb (0.050 g, 0.23 mmol) and 4-phenylbutyl isocyanate (0.080 g, 0.46 mmol) afforded the title compound as a transparent oil (0.02 g, 24%). ¹H NMR (400 MHz, CDCl₃) δ 8.62-8.40 (m, 1H), 7.62 (td, J=1.8, 7.7 Hz, 1H), 7.37-7.23 (m, 2H), 7.23-7.07 (m, 5H), 4.49 (t, J=5.0 Hz, 1H), 3.54 (dt, J=4.4, 12.9 Hz, 1H), 3.34-3.14 (m, 3H), 3.02 (ddt, J=4.1, 8.3, 12.6 Hz, 1H), 2.64 (t, J=7.5 Hz, 2H), 2.08-1.98 (m, 1H), 1.92-1.61 (m, 5H), 1.60-1.48 (m, 5H), 1.42 (s, 3H). UPLC/MS (method A): Rt 2.32 min. MS (ES) C₂₃H₃₁N₃O requires 365, found 366 [M+H]⁺.

### Example 80: 2-Methyl-5-phenyl-N-(4-phenylbutyl)piperidine-1-carboxamide tert-Butyl 5-hydroxy-2-methyl-5-phenyl-piperidine-1-carboxylate (XIf)

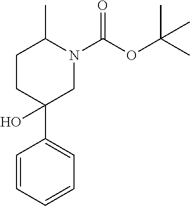 [SMILES: CC1CCC(O)(c2ccccc2)CN1C(=O)OC(C)(C)C]

Following general procedure H (method A), V-Ag (0.150 g, 0.70 mmol) and PhMgBr (0.800 mL, 2.25 mmol, 2.8 M solution in 2-MeTHF) afforded XIf as a colorless oil (0.113 g, 55%). ¹H NMR (400 MHz, CDCl₃) δ 7.59-7.55 (m, 2H), 7.41-7.31 (m, 2H), 7.31-7.27 (m, 1H), 4.50 (dd, J=13.6, 2.4 Hz, 1H), 4.31-4.22 (m, 1H), 3.08 (d, J=13.6 Hz, 1H), 2.24-2.15 (m, 1H), 2.08-1.97 (m, 1H), 1.96-1.87 (m, 1H), 1.50-1.44 (m, 11H), 1.21 (d, J=6.9 Hz, 3H). UPLC/MS (method A): Rt 2.20 min. MS (ES) C₁₇H₂₅NO₃ requires 291, found 292 [M+H]⁺.

### tert-Butyl 2-methyl-5-phenyl-3,6-dihydro-2H-pyridine-1-carboxylate (VIIIf)

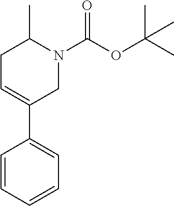 [SMILES: CC1CC=C(c2ccccc2)CN1C(=O)OC(C)(C)C]

Following general procedure J, XIf (0.067 g, 0.23 mmol) and Burgess reagent (0.082 g, 0.35 mmol) afforded VIIIf as a colorless oil (0.055 g, 88%). ¹H NMR (400 MHz, CDCl₃) δ 7.42-7.37 (m, 2H), 7.37-7.31 (m, 2H), 7.30-7.26 (m, 1H), 6.14-6.10 (m, 1H), 4.71-4.47 (m, 2H), 3.85 (d, J=18.0 Hz, 1H), 2.61 (ddd, J=17.7, 6.4, 3.2 Hz, 1H), 2.11-2.00 (m, 1H), 1.50 (s, 9H), 1.15 (d, J=6.8 Hz, 3H). UPLC/MS (method B): Rt 1.89 min. MS (ES) C₁₇H₂₃NO₂ requires 273, found 274 [M+H]⁺.

### tert-Butyl 2-methyl-5-phenylpiperidine-1-carboxylate (IXf)

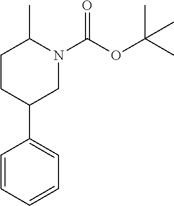 [SMILES: CC1CCC(c2ccccc2)CN1C(=O)OC(C)(C)C]

Following general procedure B (method E), VIIIf (0.071 g, 0.26 mmol) afforded IXf as a colorless oil (0.043 g, 60%). ¹H NMR (400 MHz, CDCl₃) δ 7.36-7.28 (m, 4H), 7.24-7.16 (m, 1H), 4.34-4.20 (m, 2H), 3.34 (dd, J=14.0, 4.5 Hz, 1H), 3.03-2.94 (m, 1H), 2.16-2.04 (m, 1H), 1.87-1.71 (m, 2H), 1.46 (s, 9H), 1.35-1.25 (m, 1H), 1.21 (d, J=6.8, 3H). UPLC/MS (method B): Rt 1.85 min (major diastereomer)/1.91 min (minor diastereomer) MS (ES) C₁₇H₂₅NO₂ requires 275, found 276 [M+H]⁺.

### 2-Methyl-5-phenylpiperidine hydrochloride (Xf)

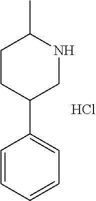

Following general procedure C, IXf (0.071 g, 0.26 mmol) afforded Xf which was used in the next step without further purification. UPLC/MS (method A): Rt 1.29 min. MS (ES) C₁₂H₁₇N requires 175, found 176 [M+H]⁺.

### 2-Methyl-5-phenyl-N-(4-phenylbutyl)piperidine-1-carboxamide

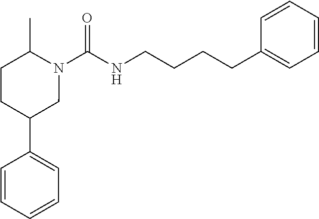 [SMILES: CC1CCC(c2ccccc2)CN1C(=O)NCCCCc1ccccc1]

Following general procedure D (method A), Xf (0.033 g, 0.156 mmol) and 4-phenylbutyl isocyanate (0.030 g, 0.172 mmol) afforded the title compound as a colorless oil (70:30 diastereomeric mixture, 0.043 g, 79%). ¹H NMR (400 MHz, DMSO-d₆) δ 7.36-7.11 (m, 10H), 6.45-6.30 (m, 1H), 4.16-4.04 (m, 2H), 3.22-3.10 (m, 2H), 3.09-2.98 (m, 1H), 2.98-2.91 (m, 1H), 2.62-2.54 (m, 2H), 1.74-1.63 (m, 2H), 1.63-1.49 (m, 4H), 1.49-1.37 (m, 2H), 1.11 (t, J=6.6 Hz, 3H). UPLC/MS (method A): Rt 2.59 min (major diastereoisomer)/2.63 (minor diastereoisomer) MS (ES) C₂₃H₃₀N₂O requires 350, found 351 [M+H]⁺.

### Example 81: 2-Methyl-3-phenyl-N-(4-phenylbutyl)piperidine-1-carboxamide tert-Butyl 3-hydroxy-2-methyl-3-phenylpiperidine-1-carboxylate (XIg)

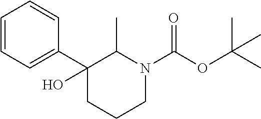 [SMILES: CC1N(C(=O)OC(C)(C)C)CCCC1(O)c1ccccc1]

Following general procedure H (method A), V-Ah (0.150 g, 0.70 mmol) and PhMgBr (0.8 mL, 2.25 mmol, 2.8 M) afforded XIg as a colorless oil (0.160 g, 78%). ¹H NMR (400 MHz, CDCl₃) δ 7.63-7.56 (m, 2H), 7.37-7.31 (m, 2H), 7.30-7.24 (m, 1H), 4.79 (q, J=6.9 Hz, 1H), 3.90-3.81 (m, 1H), 2.95 (td, J=12.9, 3.6 Hz, 1H), 2.17-2.07 (m, 1H), 2.00 (td, J=12.9, 4.3 Hz, 1H), 1.81-1.79 (m, 1H), 1.60-1.53 (m, 1H), 1.47 (s, 9H), 1.35-1.25 (m, 4H). UPLC/MS (method A): Rt 2.20 min. MS (ES) C₁₇H₂₅NO₃ requires 291, found 292 [M+H]⁺.

### tert-Butyl 6-methyl-5-phenyl-3,6-dihydro-2H-pyridine-1-carboxylate (VIIIg)

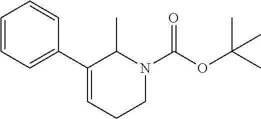 [SMILES: CC1C(c2ccccc2)=CCCN1C(=O)OC(C)(C)C]

Following general procedure J, XIe (0.067 g, 0.23 mmol) and Burgess reagent (0.082 g, 0.35 mmol afforded VIIg which was used in the next step without further purification. UPLC/MS (method B): Rt 1.88 min. MS (ES) C₁₇H₂₃NO₂ requires 273, found 274 [M+H]⁺.

### tert-Butyl 2-methyl-3-phenylpiperidine-1-carboxylate (IXg)

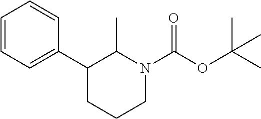 [SMILES: CC1C(c2ccccc2)CCCN1C(=O)OC(C)(C)C]

Following general procedure B (method E), VIIg (0.082 g, 0.30 mmol) afforded IXg as a colorless oil (70:30 mixture of two diastereoisomers, 0.077 g, 93/). ¹H NMR (400 MHz, CDCl₃) δ 7.38-7.27 (m, 2H), 7.25-7.15 (m, 3H), 4.69-4.36 (m, 1H), 4.17-3.87 (m, 1H), 3.04-2.93 (m, 1H), 2.9-2.79 (m, 1H), 1.99 (qd, J=13.2, 4.0 Hz, 1H), 1.8-1.72 (m, 2H), 1.50-1.46 (m, 1H), 0.81 (d, J=6.9 Hz, 2H). UPLC/MS (method B): Rt 1.86 min (minor diastereoisomer)/1.93 min (major diastereoisomer) MS (ES) C₁₇H₂₅NO₂ requires 275, found 276 [M+H]⁺.

### 2-Methyl-3-phenylpiperidine hydrochloride (Xg)

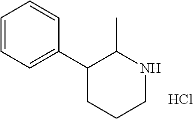

Following general procedure C, IXg (0.077 g, 0.28 mmol) afforded Xg which was used in the next step without further purification. UPLC/MS (method B): Rt 0.46 min. MS (ES) C₁₂H₁₇N requires 175, found 176 [M+H]⁺.

### 2-Methyl-3-phenyl-N-(4-phenylbutyl)piperidine-1-carboxamide

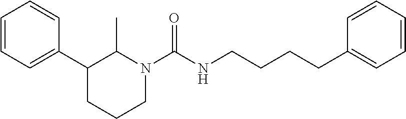 [SMILES: CC1C(c2ccccc2)CCCN1C(=O)NCCCCc1ccccc1]

Following general procedure D (method A), Xg (0.059 g, 0.28 mmol) and 4-phenylbutyl isocyanate (0.053 g, 0.31 mmol) afforded the title compound as a white powder (70:30 mixture of two diastereomers, 0.034 g, 35%). ¹H NMR (400 MHz, DMSO-d₆) δ 7.37-7.13 (m, 10H), 6.46 (t, J=5.5 Hz, 1H), 4.46-4.29 (m, 1H), 3.89-3.76 (m, 1H), 3.16-2.99 (m, 2H), 2.92-2.81 (m, 1H), 2.80-2.69 (m, 1H), 2.64-2.54 (m, 2H), 2.06-1.89 (m, 1H), 1.80-1.60 (m, 1H), 1.60-1.49 (m, 2H), 1.49-1.31 (m, 4H), 0.69 (d, J=6.8 Hz, 3H). UPLC/MS (method A): Rt 2.57 min (minor diastereoisomer)/2.61 (major diastereoisomer) MS (ES) C₂₃H₃₀N₂O requires 350, found 351 [M+H]⁺.

### Example 82: endo- and exo-N-Pentyl-3-phenyl-8-azabicyclo[3.2.1]octane-8-carboxamide

### tert-Butyl 3-hydroxy-3-phenyl-8-azabicyclo[3.2.1]octane-8-carboxylate (XIh)

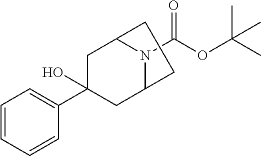 [SMILES: CC(C)(C)OC(=O)N1C2CCC1CC(O)(c1ccccc1)C2]

Following general procedure H, V-Ai (0.225 g, 1.00 mmol) and PhMgBr (0.58 g, 1.14 mL, 3.20 mmol, 2.8 M solution in 2-MeTHF) afforded XIh as a colorless oil (0.192 g, 63%). ¹H NMR (400 MHz, CDCl₃) δ 7.42-7.36 (m, 2H), 7.32 (td, J=7.8, 2.3 Hz, 2H), 7.25-7.18 (m, 1H), 4.41-4.20 (m, 2H), 2.59-2.47 (m, 1H), 2.31-2.20 (m, 2H), 2.02-1.91 (m, 2H), 1.89-1.73 (m, 2H), 1.63-1.55 (m, 1H), 1.50 (s, 9H). UPLC/MS (method A): Rt 2.32 min. MS (ES) C₁₈H₂₅NO₃ requires 303, found 304 [M+H]⁺.

### tert-Butyl 3-phenyl-8-azabicyclo[3.2.1]oct-2-ene-8-carboxylate (VIIIh)

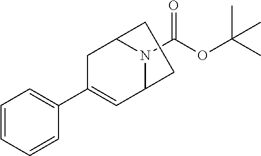 [SMILES: CC(C)(C)OC(=O)N1C2C=C(c3ccccc3)CC1CC2]

Following general procedure J, XIh (0.192 g, 0.63 mmol) and Burgess reagent (0.227 g, 0.95 mmol) afforded VIIIh as a white solid (0.099 g, 55%). ¹H NMR (400 MHz, CDCl₃) δ 7.39-7.27 (m, 4H), 7.26-7.19 (m, 1H), 6.43 (s, 1H), 4.67-4.32 (m, 2H), 3.27-2.95 (m, 1H), 2.32-2.12 (m, 2H), 2.07-1.86 (m, 2H), 1.76-1.63 (m, 1H), 1.45 (s, 9H). UPLC/MS (method B): Rt 1.80 min. MS (ES) C₁₈H₂₃NO₂ requires 285, found 286 [M+H]⁺.

### endo- and exo-tert-Butyl 3-phenyl-8-azabicyclo[3.2.1]octane-8-carboxylate (IXh)

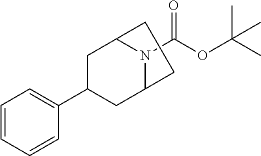 [SMILES: CC(C)(C)OC(=O)N1C2CCC1CC(c1ccccc1)C2]

Following general procedure B (method E), VIIIh (0.099 g, 0.35 mmol) afforded IXh as a colorless oil (50:50 mixture of two diastereoisomers, 0.092 g, 91%). ¹H NMR (400 MHz, CDCl₃) δ 7.32-7.26 (m, 2H), 7.25-7.16 (m, 3H), 4.46-4.14 (m, 2H), 3.08 (tt, J=11.9, 5.4 Hz, 0.5H), 2.65 (tt, J=10.4, 7.1 Hz, 0.5H), 2.59-2.38 (m, 1H), 2.12-1.91 (m, 3H), 1.87-1.76 (m, 1H), 1.75-1.69 (m, 1H), 1.64-1.57 (m, 2H), 1.50 (s, 9H). UPLC/MS (method B): Rt 1.87 min. MS (ES) C₁₈H₂₅NO₂ requires 287, found 288 [M+H]⁺.

### endo- and exo-3-Phenyl-8-azabicyclo[3.2.1]octane hydrochloride (Xh)

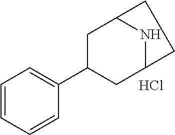 [SMILES: c1ccc(C2CC3CCC(C2)N3)cc1 ; Cl]

Following general procedure C, IXh (0.092 g, 0.32 mmol) afforded Xh which was used in the next step without further purification. UPLC/MS (method A): Rt 1.38 min. MS (ES) C₁₃H₁₇N requires 187, found 188 [M+H]⁺.

### endo- and exo-N-Pentyl-3-phenyl-8-azabicyclo[3.2.1]octane-8-carboxamide

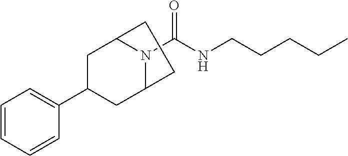 [SMILES: CCCCCNC(=O)N1C2CCC1CC(c1ccccc1)C2]

Following general procedure D (method A), Xh (0.071 g, 0.32 mmol) and n-pentyl isocyanate (0.040 g, 0.35 mmol) afforded the title compound as a colorless oil (55:45 exo/endo mixture, 0.060 g, 63%). ¹H NMR (400 MHz, CDCl₃) δ 7.31-7.26 (m, 2H, endo and exo), 7.24-7.15 (m, 3H, endo and exo), 4.50-4.35 (m, 1H, endo and exo), 4.29-4.18 (m, 2H, endo and exo), 3.28 (dtd, J=7.9, 5.8, 2.5 Hz, 2H, endo and exo), 3.09 (tt, J=11.8, 5.3 Hz, 0.55H, exo), 2.69 (tt, J=10.3, 7.2 Hz, 0.45H, endo), 2.50 (dt, J=14.7, 7.6 Hz, 0.90H, endo), 2.12-2.00 (m, 2H, endo and exo), 1.93 (td, J=13.0, 3.0 Hz, 1.10H, exo), 1.88-1.79 (m, 1.10H, exo), 1.75-1.67 (m, 1.10H, exo), 1.67-1.63 (m, 0.9H, endo), 1.61-1.48 (m, 2.9H, endo and exo), 1.39-1.26 (m, 4H, endo and exo), 0.91 (t, J=7.1 Hz, 3H, endo and exo). UPLC/MS (method A): Rt 2.39 min MS (ES) C₁₉H₂₈N₂O requires 300, found 301 [M+H]⁺.

### Example 83: 2,2-Dimethyl-N-(4-phenylbutyl)-4-(1-piperidyl)piperidine-1-carboxamide tert-Butyl 2,2-dimethyl-4-(1-piperidyl)piperidine-1-carboxylate (IXi)

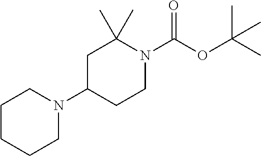 [SMILES: CC(C)(C)OC(=O)N1CCC(N2CCCCC2)CC1(C)C]

Following general procedure I, V-Aa (0.420 g, 1.85 mmol) and piperidine (0.628 g, 7.38 mmol) afforded IXi as an oil (0.309 g, 56%). ¹H NMR (400 MHz, CDCl₃) δ 3.65 (ddd, J=13.7, 7.4, 4.5 Hz, 1H), 3.31 (ddd, J=13.7, 8.2, 4.3 Hz, 1H), 2.69-2.56 (m, 1H), 2.56-2.39 (m, 4H), 1.95-1.84 (m, 1H), 1.68 (t, J=12.7 Hz, 1H), 1.64-1.55 (m, 5H), 1.55-1.42 (m, 3H), 1.51 (s, 3H), 1.47 (s, 9H), 1.34 (s, 3H). UPLC/MS (method A): Rt 1.65 min. MS (ES) C₁₇H₃₂N₂O₂ requires 296, found 297 [M+H]⁺.

### 2,2-Dimethyl-4-(1-piperidyl)piperidine dihydrochloride (Xi)

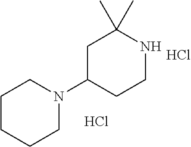

Following general procedure C, IXi (0.434 g, 1.46 mmol) afforded Xi as a white solid (0.392 g, quant.). UPLC/MS (method A): Rt 0.56 min. MS (ES) C₁₂H₂₄N₂ requires 196, found 197 [M+H]⁺.

### 2,2-Dimethyl-N-(4-phenylbutyl)-4-(1-piperidyl)piperidine-1-carboxamide

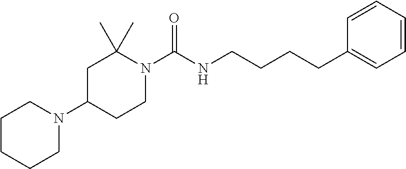 [SMILES: CC1(C)CC(N2CCCCC2)CCN1C(=O)NCCCCc1ccccc1]

Following general procedure D (method A), Xi (0.08 g, 0.32 mmol) and 4-phenylbutyl isocyanate (0.05 g, 0.37 mmol) afforded the title compound as a white solid (0.025 g, 31%). ¹H NMR (400 MHz, CDCl₃) δ 7.35-7.26 (m, 2H), 7.24-7.15 (m, 3H), 4.56 (t, J=4.9 Hz, 1H), 3.54 (dt, J=13.4, 4.8 Hz, 1H), 3.28-3.12 (m, 4H), 3.11-2.72 (m, 4H), 2.65 (t, J=7.5 Hz, 2H), 2.31-2.20 (m, 1H), 2.13-1.92 (m, 3H), 1.89-1.80 (m, 3H), 1.73-1.61 (m, 4H), 1.60-1.50 (m, 6H), 1.37 (s, 3H). UPLC/MS (method A): Rt 1.84 min. MS (ES) C₂₃H₃₇N₃O requires 371, found 372 [M+H]⁺.

### Example 84: 2,2-Dimethyl-N-pentyl-4-(1-piperidyl)piperidine-1-carboxamide

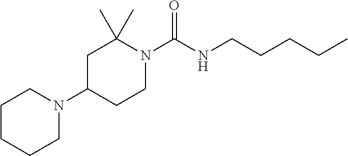 [SMILES: CCCCCNC(=O)N1CCC(N2CCCCC2)CC1(C)C]

Following general procedure D (method A), Xi (0.050 g, 0.215 mmol) and pentyl isocyanate (0.027 g, 0.236 mmol) afforded the title compound as an oil (0.023 g, 34%). ¹H NMR (400 MHz, CDCl₃) δ 4.62-4.49 (bs, 1H), 3.58 (dt, J=13.5, 4.8 Hz, 1H), 3.27-3.10 (m, 4H), 2.85-2.57 (m, 2H), 2.40-2.28 (m, 2H), 2.08-1.78 (m, 6H), 1.56 (s, 3H), 1.57-1.46 (m, 4H), 1.41-1.25 (m, 6H), 1.38 (s, 3H), 0.92 (t, J=7.0 Hz, 3H). UPLC/MS (method A): Rt 1.57 min. MS (ES), C₁₈H₃₅N₃O requires 309, found 310 [M+H]⁺.

### Example 85: N-Heptyl-2,2-dimethyl-4-(1-piperidyl)piperidine-1-carboxamide

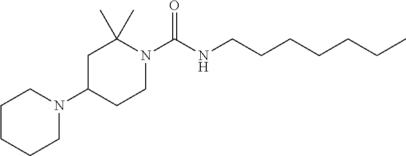 [SMILES: CCCCCCCNC(=O)N1CCC(N2CCCCC2)CC1(C)C]

Following general procedure D (method A), (0.050 g, 0.215 mmol) and heptyl isocyanate (0.033 g, 0.236 mmol) afforded the title compound as a white solid (0.038 g, 53%). ¹H NMR (400 MHz, CDCl₃) δ 4.61 (t, J=5.3 Hz, 1H), 3.59 (dt, J=13.6, 4.8 Hz, 1H), 3.30-3.03 (m, 4H), 2.99-2.53 (m, 2H), 2.49-2.23 (m, 2H), 2.21-1.83 (m, 6H), 1.82-1.59 (m, 3H), 1.56 (s, 3H), 1.55-1.43 (m, 3H), 1.38 (s, 3H), 1.36-1.20 (m, 8H), 0.97-0.83 (m, 3H). UPLC/MS (method A): Rt 1.94 min. MS (ES), C₂₀H₃₉N₃O requires 337, found 338 [M+H]⁺.

### Example 86: 4-(4,4-Difluoro-1-piperidyl)-2,2-dimethyl-N-(4-phenylbutyl)piperidine-1-carboxamide

### tert-Butyl 4-(4,4-difluoro-1-piperidyl)-2,2-dimethyl-piperidine-1-carboxylate (IXj)

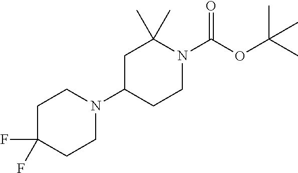 [SMILES: CC(C)(C)OC(=O)N1CCC(N2CCC(F)(F)CC2)CC1(C)C]

Following general procedure I (method A), V-Aa (0.100 g, 0.440 mmol) and 4,4-difluoropiperidine (0.041 g, 0.440 mmol) afforded IXj as an oil (0.080 g, 55%). ¹H NMR (400 MHz, CDCl₃) δ 3.70 (ddd, J=13.7, 7.1, 4.5 Hz, 1H), 3.30 (ddd, J=13.7, 8.6, 4.2 Hz, 1H), 2.79-2.69 (m, 1H), 2.69-2.59 (m, 4H), 2.08-1.94 (m, 4H), 1.94-1.83 (m, 1H), 1.71-1.61 (m, 1H), 1.57-1.46 (m, 2H), 1.53 (s, 3H), 1.48 (s, 9H), 1.34 (s, 3H). UPLC/MS (method A): Rt 2.42 min. MS (ES) C₁₇H₃₀F₂N₂O₂, requires 332, found 333 [M+H]⁺.

### 1-(2,2-Dimethyl-4-piperidyl)-4,4-difluoro-piperidine hydrochloride (Xj)

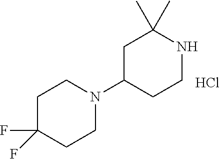 [SMILES: CC1(C)CC(N2CCC(F)(F)CC2)CCN1 ; Cl]

Following general procedure C, IXj (0.037 g, 0.111 mmol) afforded Xj as a white solid (0.340 g, quant.). UPLC/MS (method A): Rt 1.05 min. MS (ES) C₁₂H₂₂F₂N₂, requires 232, found 233 [M+H]⁺.

### 4-(4,4-Difluoro-1-piperidyl)-2,2-dimethyl-N-(4-phenylbutyl)piperidine-1-carboxamide

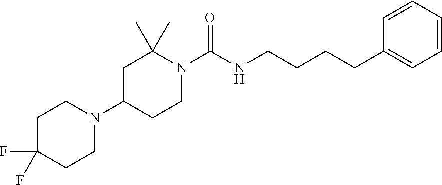 [SMILES: CC1(C)CC(N2CCC(F)(F)CC2)CCN1C(=O)NCCCCc1ccccc1]

Following general procedure D (Method A), Xj (0.030 g, 0.111 mmol) and 4-phenylbutyl isocyanate (21 g, 0.120 mmol) afforded the title compound as a white solid (0.018 g, 40%). ¹H NMR (400 MHz, CDCl₃) δ 7.35-7.25 (m, 2H), 7.25-7.15 (m, 3H), 4.42 (t, J=5.2 Hz, 1H), 3.44 (dt, J=13.1, 4.8 Hz, 1H), 3.22 (td, J=7.1, 5.5 Hz, 2H), 3.10 (ddd, J=13.1, 10.7, 3.5 Hz, 1H), 2.73-2.61 (m, 6H), 2.00 (tt, J=13.1, 5.6 Hz, 4H), 1.92-1.82 (m, 1H), 1.73-1.62 (m, 2H), 1.60-1.43 (m, 6H), 1.55 (s, 3H), 1.35 (s, 3H). UPLC/MS (method A): Rt 2.29 min. MS (ES) C₂₃H₃₅F₂N₃O requires 407, found 408 [M+H]⁺.

### Example 87: 2,2-Dimethyl-4-(4-methylpiperazin-1-yl)-N-(4-phenylbutyl)piperidine-1-carboxamide tert-Butyl 2,2-dimethyl-4-(4-methylpiperazin-1-yl)piperidine-1-carboxylate (IXk)

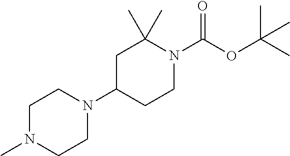 [SMILES: CN1CCN(C2CCN(C(=O)OC(C)(C)C)C(C)(C)C2)CC1]

Following general procedure I, V-Aa (100 mg, 0.440 mmol) and 1-methylpiperazine (0.044 g, 0.440 mmol) afforded IXk as an oil (0.107 g, 76%). ¹H NMR (400 MHz, CDCl₃) δ 3.69 (ddd, J=13.7, 7.2, 4.5 Hz, 1H), 3.29 (ddd, J=13.7, 8.6, 4.3 Hz, 1H), 2.66-2.54 (m, 5H), 2.48 (m, 3H), 2.31 (s, 3H), 1.97-1.85 (m, 1H), 1.70-1.57 (m, 3H), 1.53-1.45 (m, 1H), 1.52 (s, 3H), 1.48 (s, 9H), 1.34 (s, 3H). UPLC/MS (method A): Rt 1.56 min. MS (ES) C₁₇H₃₃N₃O₂, requires 311, found 312 [M+H]⁺.

### 1-(2,2-Dimethyl-4-piperidyl)-4-methylpiperazine trihydrochloride (Xk)

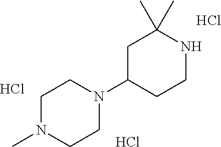

Following general procedure C, IXk (0.107 g, 0.344 mmol) afforded Xk as a white solid (0.109 g, quant.). UPLC/MS (method A): Rt 0.57 min. MS (ES) C₁₂H₂₅N₃, requires 211, found 212 [M+H]⁺.

### 2,2-Dimethyl-4-(4-methylpiperazin-1-yl)-N-(4-phenylbutyl)piperidine-1-carboxamide dihydrochloride

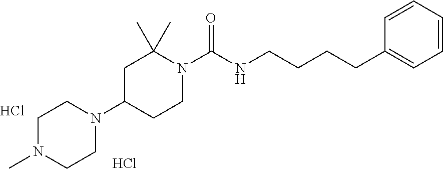 [SMILES: Cl ; CN1CCN(C2CCN(C(=O)NCCCCc3ccccc3)C(C)(C)C2)CC1 ; Cl]

Following general procedure D (Method A), Xk (0.085 g, 0.344 mmol) and 4-phenylbutyl isocyanate (0.066 g, 0.378 mmol) afforded the free base of the title compound as a white solid (0.102 g, 77%). The free base of the title compound was dissolved in DCM (0.1 M) and HCl (0.080 mL, 37% aqueous solution, 10 eq.) was added. The solvent was removed under vacuo to afford the title compound hydrochloride salt as a white solid. ¹H NMR (400 MHz, DMSO-d₆) δ 12.16 (bs, 1H), 12.02 (bs, 1H), 7.33-7.23 (m, 2H), 7.23-7.09 (m, 3H), 6.61 (bs, 1H), 3.85-3.55 (m, 5H), 3.55-3.27 (m, 5H), 3.03-2.90 (m, 3H), 2.88-2.75 (m, 3H), 2.57 (t, J=7.6 Hz, 2H), 2.12-2.08 (m, 1H), 1.93-1.90 (m, 1H), 1.77-1.63 (m, 2H), 1.60-1.47 (m, 2H), 1.45 (s, 3H), 1.44-1.34 (m, 2H), 1.26 (s, 3H). UPLC/MS (method A): Rt 1.72 min. MS (ES) C₂₃H₃₈N₄O requires 386, found 387 [M+H]⁺.

### Example 88: 2,2-Dimethyl-4-(N-methylanilino)-N-(4-phenylbutyl)piperidine-1-carboxamide hydrochloride tert-Butyl 2,2-dimethyl-4-(N-methylanilino)piperidine-1-carboxylate (IXI)

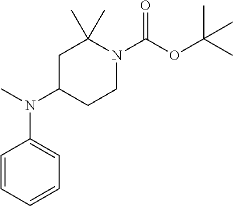 [SMILES: CN(c1ccccc1)C1CCN(C(=O)OC(C)(C)C)C(C)(C)C1]

To a solution of V-Aa (0.1 g, 0.440 mmol, 1.0 eq.) in MeOH, aniline (0.041 g, 0.440 mmol 1.0 eq.), AcOH (0.026 g, 0.440 mmol) and NaBH(OAc)₃ (0.186 g, 0.880 mmol, 2.0 eq.) were added. The mixture was stirred at RT under N₂ atmosphere for 1 h and then, 37% aq. solution of formaldehyde (0.026 g, 0.024 mL, 0.88 mmol), AcOH (0.026 g, 0.025 mL, 0.440 mmol) and NaBH(OAc)₃ (0.186 g, 0.880 mmol) were added. The mixture was stirred at RT for 1 h and then quenched with MeOH, diluted with EA, washed with saturated aq. NaHCO₃ solution, brine and dried over Na₂SO₄. After concentration, the residue was purified by column chromatography (SiO₂), eluting with Cy/EA (99:1) to afford IXI as an oil (77 mg, 55%). ¹H NMR (400 MHz, CDCl₃) δ 7.25-7.20 (m, 2H), 6.80 (m, 2H), 6.73 (m, 1H), 4.02-3.90 (m, 1H), 3.86 (m, 1H), 3.31 (m, 1H), 2.77 (s, 3H), 1.92 (m, 1H), 1.79 (t, J=12.9 Hz, 1H), 1.72 (m, 1H), 1.53 (s, 3H), 1.52-1.49 (m, 1H), 1.47 (s, 9H), 1.40 (s, 3H). UPLC/MS (method B): Rt 2.14 min. MS (ES) C₁₉H₃₀N₂O₂ requires 318, found 319 [M+H]⁺.

### N,2,2-Trimethyl-N-phenylpiperidin-4-amine hydrochloride (XI)

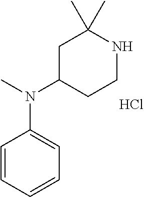 [SMILES: CN(c1ccccc1)C1CCNC(C)(C)C1 ; Cl]

Following general procedure C, IXI (0.074 g, 0.232 mmol) afforded XI as a white solid (0.059 g, quant.). UPLC/MS (method B): Rt 1.49 min. MS (ES) C₁₄H₂₂N₂ requires 218, found 219 [M+H]⁺.

### 2,2-Dimethyl-4-(N-methylanilino)-N-(4-phenylbutyl)piperidine-1-carboxamide hydrochloride

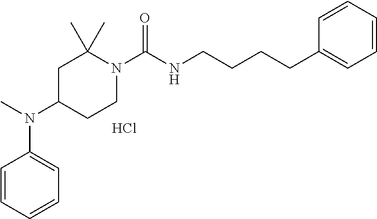 [SMILES: CN(c1ccccc1)C1CCN(C(=O)NCCCCc2ccccc2)C(C)(C)C1 ; Cl]

Following general procedure D (method A), XI (0.059 g, 0.232 mmol) and 4-phenylbutyl isocyanate (0.045 g, 0.255 mmol) afforded the title compound as a white solid (0.074 g, 81%). The title compound was dissolved in DCM (0.1 M) and HCl (0.056 mL, 37% aqueous solution, 10 eq.) was added. The solvent was removed under vacuo to afford the hydrochloride salt of the title compound as a white solid. ¹H-NMR (400 MHz, DMSO-d₆) δ 7.31-7.24 (m, 2H), 7.22-7.14 (m, 5H), 6.83-6.74 (m, 2H), 6.67-6.58 (m, 1H), 6.45 (t, J=5.5 Hz, 1H), 3.96-3.80 (m, 1H), 3.57 (dt, J=13.1, 4.3 Hz, 11H), 3.13-2.92 (m, 3H), 2.69 (s, 3H), 2.57 (t, J=7.6 Hz, 2H), 1.78-1.47 (m, 5H), 1.46-1.27 (m, 3H), 1.43 (s, 3H), 1.32 (s, 3H). UPLC/MS (method B): Rt 1.91 min. MS (ES) C₂₅H₃₅N₃O requires 393, found 394 [M+H]⁺.

### Example 89: 2,2-Dimethyl-4-phenoxy-N-(4-phenylbutyl)piperidine-1-carboxamide tert-Butyl 4-hydroxy-2,2-dimethylpiperidine-1-carboxylate

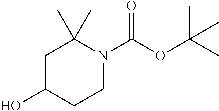 [SMILES: CC(C)(C)OC(=O)N1CCC(O)CC1(C)C]

To a stirred solution of compound V-Aa (0.200 g, 0.88 mmol, 1.0 eq.) in MeOH (4 mL, 0.2 M), NaBH₄ (0.050 g, 1.32 mmol, 1.5 eq.) was added portion wise at 0° C. The mixture was stirred at RT for 30 min under N₂ atmosphere and then diluted with EA, washed with saturated aq. NH₄Cl solution, brine, dried over Na₂SO₄ and concentrated to afford tert-butyl 4-hydroxy-2,2-dimethylpiperidine-1-carboxylate which was used in the next step without further purification. ¹H NMR (400 MHz, DMSO-d₆) δ 4.63 (bs, 1H), 3.77-3.60 (m, 2H), 3.07 (ddd, J=13.6, 10.1, 3.6 Hz, 1H), 1.88-1.78 (m, 1H), 1.64 (ddd, J=13.2, 4.6, 1.7 Hz, 1H), 1.43 (s, 3H), 1.38 (s, 10H), 1.32-1.24 (m, 1H), 1.22 (s, 3H). UPLC/MS (method A): Rt 1.80 min. MS (ES) C₁₂H₂₃NO₃ requires 229, found 230 [M+H]⁺.

### tert-Butyl 2,2-dimethyl-4-methylsulfonyloxypiperidine-1-carboxylate

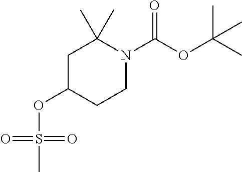 [SMILES: CC(C)(C)OC(=O)N1CCC(OS(C)(=O)=O)CC1(C)C]

To a stirred solution of tert-butyl 4-hydroxy-2,2-dimethyl-piperidine-1-carboxylate (0.130 g, 0.57 mmol) and Et₃N (0.115 g, 0.159 mL, 1.14 mmol) in DCM 0.2 M (3 mL, 0.2 M), MsCl (0.097 g, 0.066 mL, 0.85 mmol) was added. The mixture was stirred at RT for 2 h under N₂, diluted with EA, washed with saturated aq. NH₄Cl solution, brine, dried over Na₂SO₄ and concentrated to afford tert-butyl 2,2-dimethyl-4-methylsulfonyloxypiperidine-1-carboxylate which was used in the next step without further purification. ¹H NMR (400 MHz, CDCl₃) δ 4.93 (ddt, J=9.6, 7.7, 5.5 Hz, 1H), 3.79 (ddd, J=14.1, 7.1, 4.2 Hz, 1H), 3.32 (ddd, J=14.1, 9.2, 3.7 Hz, 1H), 3.02 (s, 3H), 2.24-2.13 (m, 1H), 1.99-1.77 (m, 3H), 1.53 (s, 3H), 1.46 (s, 9H), 1.36 (s, 3H). UPLC/MS (method A): Rt 2.18 min. MS (ES) C₁₃H₂₅NO₅S requires 307, found 308 [M+H]⁺.

### tert-Butyl 2,2-dimethyl-4-phenoxypiperidine-1-carboxylate

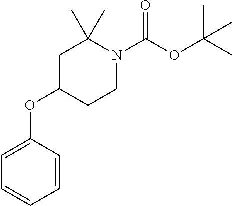 [SMILES: CC(C)(C)OC(=O)N1CCC(Oc2ccccc2)CC1(C)C]

To a stirred solution of tert-butyl 2,2-dimethyl-4-methylsulfonyloxy-piperidine-1-carboxylate (0.175 g, 0.57 mmol) in DMF (3.0 mL, 0.2 M), Cs₂CO₃ (0.371 g, 1.14 mmol) and phenol (0.081 g, 0.86 mmol) were added. The mixture was stirred at 90° C. on under N₂ atmosphere and then diluted with EA, washed with a saturated aq. NaHCO₃ solution, 5% aq. solution of LiCl, brine and dried over Na₂SO₄. After concentration, the residue was purified by column chromatography (SiO₂), eluting with Cy/EA (85:15) to afford tert-Butyl 2,2-dimethyl-4-phenoxy-piperidine-1-carboxylate as a colorless oil (0.67 g, 39%). ¹H NMR (400 MHz, CDCl₃) δ 7.31-7.27 (m, 2H), 6.94 (td, J=7.4, 1.1 Hz, 1H), 6.87 (dt, J=7.8, 1.1 Hz, 2H), 4.59-4.51 (m, 1H), 3.78-3.67 (m, 1H), 3.47-3.41 (m, 1H), 2.22-2.12 (m, 1H), 1.96-1.81 (m, 2H), 1.80-1.68 (m, 1H), 1.52 (s, 3H), 1.48 (s, 9H), 1.46 (s, 3H). UPLC/MS (method B): Rt 1.99 min. MS (ES) C₁₈H₂₇NO₃ requires 305, found 306 [M+H]⁺.

### 2,2-Dimethyl-4-phenoxypiperidine hydrochloride

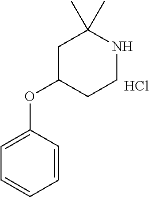

Following general procedure C, tert-butyl 2,2-dimethyl-4-phenoxypiperidine-1-carboxylate (0.067 g, 0.22 mmol) afforded 2,2-dimethyl-4-phenoxypiperidine hydrochloride which was used in the next step without further purification. UPLC/MS (method A): Rt 1.40 min. MS (ES) C₁₃H₁₉NO requires 205, found 206 [M+H]⁺.

### 2,2-Dimethyl-4-phenoxy-N-(4-phenylbutyl)piperidine-1-carboxamide

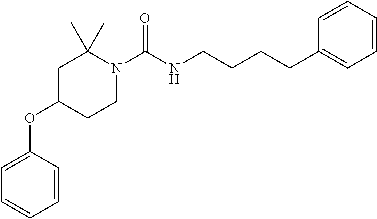 [SMILES: CC1(C)CC(Oc2ccccc2)CCN1C(=O)NCCCCc1ccccc1]

Following general procedure D, (method A) using 2,2-dimethyl-4-phenoxy-piperidine hydrochloride (0.053 g, 0.22 mmol) and 4-phenylbutyl isocyanate (0.050 g, 0.286 mmol) afforded the title compound as a white solid (0.022 g, 26%). ¹H NMR (400 MHz, DMSO-d₆) δ 7.30-7.24 (m, 4H), 7.22-7.13 (m, 3H), 6.97-6.87 (m, 3H), 6.45 (t, J=5.5 Hz, 1H), 4.55 (tt, J=9.2, 4.6 Hz, 1H), 3.56-3.46 (m, 1H), 3.10 (ddd, J=13.4, 10.3, 3.3 Hz, 1H), 3.02-2.95 (m, 2H), 2.57 (t, J=7.6 Hz, 2H), 2.13-2.02 (m, 1H), 1.82 (ddd, J=13.1, 4.4, 1.8 Hz, 1H), 1.60-1.48 (m, 4H), 1.46-1.37 (m, 5H), 1.33 (s, 3H). UPLC/MS (method B): Rt 1.76 min. MS (ES) C₂₄H₃₂N₂O₂ requires 380, found 381 [M+H]⁺.

### Example 90: 2,2-Dimethyl-N-(4-phenylbutyl)piperidine-1-carboxamide carboxamide

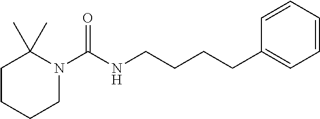 [SMILES: CC1(C)CCCCN1C(=O)NCCCCc1ccccc1]

Following general procedure D (method A), 2,2-dimethylpiperidine (0.055 g, 0.486 mmol) and 4-phenylbutyl isocyanate (0.094 g, 0.534 mmol) afforded the title compound as a colorless oil (0.110 g, 78%). ¹H NMR (400 MHz, DMSO-d₆) δ 7.32-7.23 (m, 2H), 7.23-7.09 (m, 3H), 6.32 (t, J=5.4 Hz, 1H), 3.21-3.07 (m, 2H), 2.97 (td, J=7.0, 5.5 Hz, 2H), 2.57 (t, J=7.6 Hz, 2H), 1.59-1.42 (m, 6H), 1.42-1.33 (m, 4H), 1.30 (s, 6H). UPLC/MS (method A): Rt 2.50 min. MS (ES) C₁₈H₂₈N₂O requires 288, found 289 [M+H]⁺.

### Example 91: 2,2-Dimethyl-N-(4-phenylbutyl)-4-(2-phenyl-1-piperidyl)piperidine-1-carboxamide tert-Butyl 2,2-dimethyl-4-(2-phenyl-1-piperidyl)piperidine-1-carboxylate (IXm)

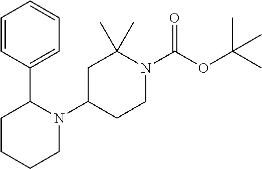 [SMILES: CC(C)(C)OC(=O)N1CCC(N2CCCCC2c2ccccc2)CC1(C)C]

Following general procedure I (method B), V-Aa (0.150 g, 0.66 mmol) and 2-phenylpiperidine (0.138 g, 0.858 mmol) afforded IXm as a white solid (0.070 g, 28%). UPLC/MS (method A): Rt 2.24 min. MS (ES) C₂₃H₃₆N₂O₂ requires 372, found 373 [M+H]⁺.

### 2,2-Dimethyl-4-(2-phenyl-1-piperidyl)piperidine dihydrochloride (Xm)

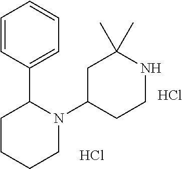 [SMILES: CC1(C)CC(N2CCCCC2c2ccccc2)CCN1 ; Cl ; Cl]

Following general procedure C, IXm (0.070 g, 0.188 mmol) afforded Xm which was used in the next step without further purification. UPLC/MS (method A): Rt 1.18 min. MS (ES) C₁₈H₂₈N₂ require 272, found 273 [M+H]⁺.

### 2,2-Dimethyl-N-(4-phenylbutyl)-4-(2-phenyl-1-piperidyl)piperidine-1-carboxamide

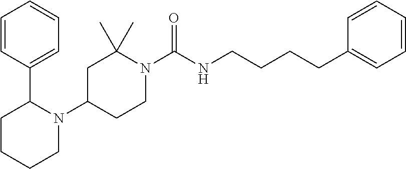 [SMILES: CC1(C)CC(N2CCCCC2c2ccccc2)CCN1C(=O)NCCCCc1ccccc1]

Following general procedure D (Method A), Xm (0.065 g, 0.188 mmol) and 4-phenylbutyl isocyanate (0.036 g, 0.207 mmol) afforded the title compound as a yellowish oil (6:4 mixture of two diastereoisomers, 0.037 g, 45%) ¹H NMR (400 MHz, DMSO-d₆) δ 7.57-6.78 (m, 10H), 6.39-6.03 (m, 1H), 3.43-3.36 (m, 1H), 3.32-3.26 (m, 1H), 2.97-2.81 (m, 3H), 2.68-2.53 (m, 3H), 2.50-2.39 (m, 1H), 2.28-2.18 (m, 1H), 1.92-1.18 (m, 16H), 1.17-1.01 (m, 1H), 0.81 (s, 2.0H), 0.71 (s, 1H). UPLC/MS (method A): Rt 2.22 min. MS (ES) C₂₉H₄₁N₃O requires 447, found 448 [M+H]⁺.

### Example 92: 2,2-Dimethyl-N-(4-phenylbutyl)-4-(3-phenyl-1-piperidyl)piperidine-1-carboxamide tert-Butyl 2,2-dimethyl-4-(3-phenyl-1-piperidyl)piperidine-1-carboxylate (IXn)

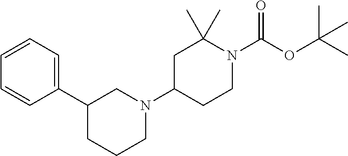 [SMILES: CC(C)(C)OC(=O)N1CCC(N2CCCC(c3ccccc3)C2)CC1(C)C]

Following general procedure I (method B), V-Aa (0.150 g, 0.66 mmol) and 3-phenylpiperidine (0.138 g, 0.858 mmol) afforded IXn as a white solid (0.211 g, 86%). ¹H NMR (400 MHz, CDCl₃) δ 7.44-7.29 (m, 3H), 7.30-7.23 (m, 3H), 3.96-3.76 (m, 1H), 3.50 (d, J=11.5 Hz, 1H), 3.47-3.11 (m, 4H), 2.94-2.58 (m, 2H), 2.39-2.02 (m, 4H), 1.95-1.83 (m, 2H), 1.82-1.60 (m, 2H), 1.58 (d, J=2.1 Hz, 3H), 1.47 (s, 9H), 1.38 (d, J=1.7 Hz, 3H). UPLC/MS (method A): Rt 2.13 min. MS (ES) C₂₃H₃₆N₂O₂ requires 372, found 373 [M+H]⁺.

### 2,2-Dimethyl-4-(3-phenyl-1-piperidyl)piperidine dihydrochloride (Xn)

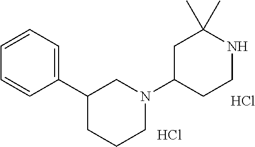 [SMILES: CC1(C)CC(N2CCCC(c3ccccc3)C2)CCN1 ; Cl ; Cl]

Following general procedure C, IXn (0.130 g, 0.35 mmol) afforded Xn which was used in the next step without further purification. ¹H NMR (400 MHz, DMSO-d₆) δ 11.05 (bs, 1H), 9.37 (d, J=8.8 Hz, 1H), 9.27 (d, J=9.1 Hz, 1H), 7.45-7.24 (m, 5H), 3.77-3.61 (m, 1H), 3.53-3.43 (m, 2H), 3.29-3.21 (m, 3H), 3.19-2.92 (m, 3H), 2.39-2.17 (m, 2H), 2.16-1.81 (m, 5H), 1.80-1.66 (m, 1H), 1.41 (s, 3H), 1.31 (d, J=2.9 Hz, 3H). UPLC/MS (method A): Rt 1.21 min. MS (ES) C₁₈H₂₈N₂ require 272, found 273 [M+H]⁺.

### 2,2-Dimethyl-N-(4-phenylbutyl)-4-(3-phenyl-1-piperidyl)piperidine-1-carboxamide

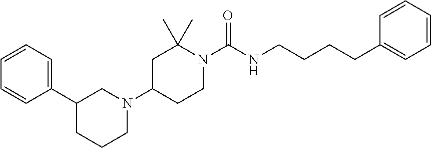 [SMILES: CC1(C)CC(N2CCCC(c3ccccc3)C2)CCN1C(=O)NCCCCc1ccccc1]

Following general procedure D (method A), Xn (0.065 g, 0.188 mmol) and 4-phenylbutyl isocyanate (0.036 g, 0.207 mmol) afforded the title compound as a yellowish oil (1:1 mixture of two diastereoisomers, 0.044 g, 52%). ¹H NMR (400 MHz, DMSO-d₆) δ 7.40-7.21 (m, 6H), 7.22-7.08 (m, 4H), 6.43-6.23 (m, 1H), 3.54-3.40 (m, 1H), 3.09-2.77 (m, 5H), 2.75-2.62 (m, 1H), 2.61-2.52 (m, 3H), 2.25-2.06 (m, 2H), 1.86-1.66 (m, 3H), 1.63-1.29 (m, 12H), 1.22 (s, 3H). UPLC/MS (method A): Rt 2.23 min. MS (ES) C₂₉H₄N₃O requires 447, found 448 [M+H]⁺.

### Example 93: 2,2-Dimethyl-N-(4-phenylbutyl)-4-(4-phenyl-1-piperidyl)piperidine-1-carboxamidetert-Butyl 2,2-dimethyl-4-(4-phenyl-1-piperidyl)piperidine-1-carboxylate (IXo)

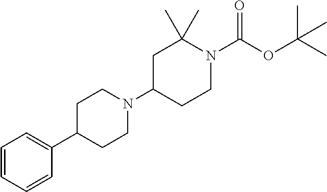 [SMILES: CC(C)(C)OC(=O)N1CCC(N2CCC(c3ccccc3)CC2)CC1(C)C]

Following general procedure H (method B), V-Aa (0.120 g, 0.528 mmol) and 4-phenylpiperidine (0.094 g, 0.581 mmol) afforded IXo as a white solid (0.155 g, 79%). ¹H NMR (400 MHz, CDCl₃) δ 7.38-7.29 (m, 2H), 7.28-7.21 (m, 3H), 3.83-3.68 (m, 1H), 3.42-3.31 (m, 1H), 3.30-3.09 (m, 2H), 3.00-2.82 (m, 1H), 2.67-2.37 (m, 3H), 2.13-1.85 (m, 5H), 1.84-1.61 (m, 3H), 1.56 (s, 3H), 1.49 (s, 9H), 1.39 (s, 3H). UPLC/MS (method A): Rt 2.10 min. MS (ES) C₂₃H₃₆N₂O₂ requires 372, found 373 [M+H]⁺.

### 2,2-Dimethyl-4-(4-phenyl-1-piperidyl)piperidine dihydrochloride (Xo)

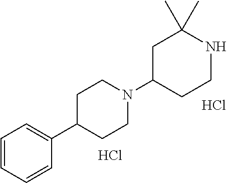 [SMILES: CC1(C)CC(N2CCC(c3ccccc3)CC2)CCN1 ; Cl ; Cl]

Following general procedure C, IXo (0.152 g, 0.408 mmol) afforded Xo which was used in the next step without further purification. ¹H NMR (400 MHz, DMSO-d₆) δ 11.02 (bs, 1H), 9.35 (bs, 2H), 7.40-7.33 (m, 2H), 7.30-7.19 (m, 3H), 3.65 (t, J=11.6 Hz, 1H), 3.57-3.46 (m, 2H), 3.32-3.27 (m, 1H), 3.21-2.97 (m, 3H), 2.86 (t, J=12.4 Hz, 1H), 2.32-2.10 (m, 4H), 2.08-1.85 (m, 4H), 1.43 (s, 3H), 1.34 (s, 3H). UPLC/MS (method A): Rt 1.15 min. MS (ES) C₁₈H₂₈N₂ require 272, found 273 [M+H]⁺.

### 2,2-Dimethyl-N-(4-phenylbutyl)-4-(4-phenyl-1-piperidyl)piperidine-1-carboxamide

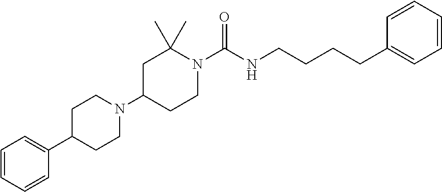 [SMILES: CC1(C)CC(N2CCC(c3ccccc3)CC2)CCN1C(=O)NCCCCc1ccccc1]

Following general procedure D (Method A), Xo (0.060 g, 0.174 mmol) and 4-phenylbutyl isocyanate (0.033 g, 0.191 mmol) afforded the title compound as a colorless oil (0.057 g, 73%). ¹H NMR (400 MHz, DMSO-d₆) δ 7.44-7.01 (m, 10H), 6.37 (t, J=5.4 Hz, 1H), 3.49 (dt, J=12.7, 4.5 Hz, 1H), 3.18-2.83 (m, 5H), 2.63-2.54 (m, 2H), 2.50-2.38 (m, 2H), 2.30-2.11 (m, 2H), 1.90-1.69 (m, 3H), 1.68-1.47 (m, 5H), 1.48-1.29 (m, 7H), 1.24 (s, 3H). UPLC/MS (method A): Rt 2.25 min. MS (ES) C₂₉H₄₁N₃O requires 447, found 448 [M+H]⁺.

### Example 94: 2-Methyl-N-(4-phenylbutyl)-4-(1-piperidyl)piperidine-1-carboxamide tert-Butyl 2-methyl-4-(1-piperidyl)piperidine-1-carboxylate (IXp)

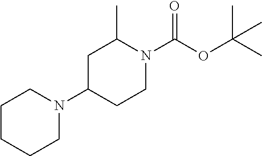 [SMILES: CC1CC(N2CCCCC2)CCN1C(=O)OC(C)(C)C]

Following general procedure I (method B), V-Ab (0.16 g, 0.75 mmol) afforded IXp as a colorless oil (4:1 mixture of two diastereoisomers, 0.210 g, quantitative). ¹H NMR (400 MHz, CDCl₃) δ 4.16-3.57 (m, 2H), 3.19-3.09 (m, 1H), 2.92-2.59 (m, 5H), 2.18-1.87 (m, 2H), 1.86-1.74 (m, 4H), 1.73-1.61 (m, 1H), 1.59-1.53 (m, 2H), 1.48 (s, 9H), 1.25 (d, J=6.3 Hz, 2.4H), 1.19 (d, J=7.0 Hz, 0.6H). UPLC/MS (method A): Rt 1.52 min. MS (ES) C₁₆H₃₀N₂O₂ requires 282, found 283 [M+H]⁺.

### 2-Methyl-4-(1-piperidyl)piperidine;dihydrochloride (Xp)

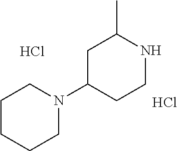

Following general procedure C, IXp (0.210 g, 0.74 mmol) afforded Xp which was used in the next step without further purification.

### 2-Methyl-N-(4-phenylbutyl)-4-(1-piperidyl)piperidine-1-carboxamide

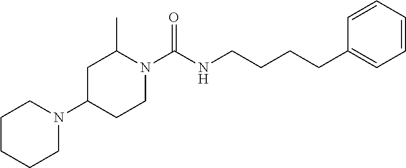 [SMILES: CC1CC(N2CCCCC2)CCN1C(=O)NCCCCc1ccccc1]

Following general procedure D (Method A), Xp (0.080 g, 0.31 mmol) and 4-phenylbutyl isocyanate (0.060 g, 0.34 mmol) afforded the title compound as a colorless oil (9:1 mixture of two diastereoisomers, 0.049 g, 45%). ¹H NMR (400 MHz, DMSO) δ 7.31-7.23 (m, 2H), 7.24-7.10 (m, 3H), 6.33 (t, J=5.4 Hz, 0.1H), 6.19 (t, J=5.5 Hz, 0.9H), 4.40-4.27 (m, 0.1H), 3.96-3.66 (m, 0.9H), 3.62-3.49 (m, 1H), 3.23-2.84 (m, 3H), 2.57 (t, J=6.5 Hz, 2H), 2.45-2.20 (m, 5H), 1.76-1.32 (m, 14H), 1.12 (d, J=6.7 Hz, 2.7H), 1.03 (d, J=6.7 Hz, 0.3H). UPLC/MS (method A): Rt 1.74 min. MS (ES) C₂₂H₃₅N₃O requires 357, found 358 [M+H]⁺.

### Example 95: 2-Methyl-N-pentyl-4-(1-piperidyl)piperidine-1-carboxamide

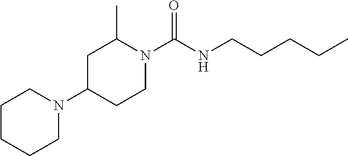 [SMILES: CCCCCNC(=O)N1CCC(N2CCCCC2)CC1C]

Following general procedure D (method A), Xp (0.080 g, 0.313 mmol) and pentyl isocyanate (0.039 g, 0.344 mmol) afforded the title compound (0.050 g, 55%) as a transparent oil. ¹H NMR (400 MHz, DMSO-d₆) δ 6.15 (t, J=5.5 Hz, 1H), 3.87-3.75 (m, 1H), 3.61-3.52 (m, 1H), 3.05-2.90 (m, 3H), 2.46-2.23 (m, 5H), 1.74-1.66 (m, 1H), 1.66-1.54 (m, 2H), 1.52-1.42 (m, 5H), 1.42-1.32 (m, 4H), 1.32-1.16 (m, 4H), 1.13 (d, J=6.4 Hz, 3H), 0.86 (t, J=7.0 Hz, 3H). UPLC/MS (method A): Rt 1.43 min. MS (ES), C₁₇H₃₃N₃O requires 295, found 296 [M+H]⁺.

### Example 96: (2R)-Methyl-N-pentyl-4-(1-piperidyl)piperidine-1-carboxamide hydrochloride salt tert-Butyl-(2R)-methyl-4-(1-piperidyl)piperidine-1-carboxylate (IXq)

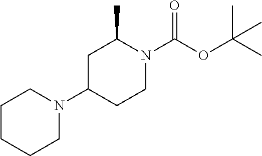 [SMILES: C[C@@H]1CC(N2CCCCC2)CCN1C(=O)OC(C)(C)C]

Following general procedure I (Method B), using V-Ac (0.16 g, 0.75 mmol) and piperidine (0.096 g, 1.125 mmol) afforded IXq as a colorless oil (85:15 mixture of two diastereoisomers, 0.195 g, 91%). ¹H NMR (400 MHz, CDCl₃) δ 4.16-3.57 (m, 2H), 3.19-3.09 (m, 1H), 2.92-2.59 (m, 5H), 2.18-1.87 (m, 2H), 1.86-1.74 (m, 4H), 1.73-1.61 (m, 1H), 1.59-1.53 (m, 2H), 1.48 (s, 9H), 1.25 (d, J=6.5 Hz, 2.4H), 1.19 (d, J=6.5 Hz, 0.6H). UPLC/MS (method A): Rt 1.49 min. MS (ES) C₁₆H₃₀N₂O₂ requires 282, found 283 [M+H]⁺.

### (2R)-2-Methyl-4-(1-piperidyl)piperidine dihydrochloride (Xq)

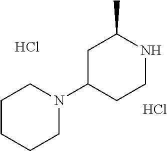

Following general procedure C, IXq (0.195 g, 0.69 mmol) afforded Xq which was used in the next step without further purification.

### (2R)-Methyl-N-pentyl-4-(1-piperidyl)piperidine-1-carboxamide hydrochloride salt

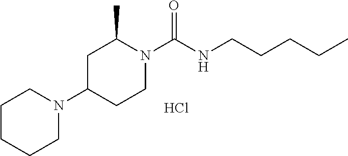 [SMILES: CCCCCNC(=O)N1CCC(N2CCCCC2)C[C@H]1C ; Cl]

Following general procedure D (Method A), Xq (0.085 g, 0.38 mmol) and pentylisocyanate (0.047 g, 0.42 mmol) afforded the free base of the title compound of as a colorless oil (0.027 g, 27%). The free base of the title compound (0.024 g, 0.081 mmol) was dissolved in Et₂O (0.1 M) and HCl (0.06 mL, 4M in dioxane, 10 eq.) was added. The solvent was concentrated, and the residue was triturated with Et₂O to afford the title compound hydrochloride salt as a white solid (0.025 g, 92%). ¹H NMR (400 MHz, DMSO-d₆) δ 10.48 (bs, 1H), 6.37 (bs, 1H), 3.88-3.70 (m, 1H), 3.70-3.56 (m, 1H), 3.37 (dd, J=31.2, 12.0 Hz, 2H), 3.26-2.73 (m, 6H), 2.20-1.95 (m, 2H), 1.91-1.49 (m, 7H), 1.46-1.30 (m, 3H), 1.31-1.18 (m, 4H), 1.13 (d, J=6.2 Hz, 3H), 0.86 (t, J=7.0 Hz, 3H). UPLC/MS (method A): Rt 1.45 min. MS (ES) C₁₇H₃₃N₃O requires 295, found 296 [M+H]⁺.

### Example 97: (2S)-Methyl-N-pentyl-4-(1-piperidyl)piperidine-1-carboxamide tert-Butyl (2S)-methyl-4-(1-piperidyl)piperidine-1-carboxylate (IXr)

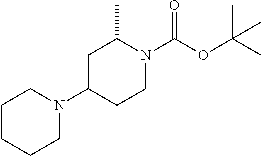 [SMILES: C[C@H]1CC(N2CCCCC2)CCN1C(=O)OC(C)(C)C]

Following general procedure I (Method B), V-Ad (0.16 g, 0.75 mmol) and piperidine (0.096 g, 1.125 mmol) afforded IXr as a colorless oil (4:1 mixture of two diastereoisomers, 0.185 g, 87%). ¹H NMR (400 MHz, CDCl₃) δ 4.16-3.57 (m, 2H), 3.19-3.09 (m, 1H), 2.92-2.59 (m, 5H), 2.18-1.87 (m, 2H), 1.86-1.74 (m, 4H), 1.73-1.61 (m, 1H), 1.59-1.53 (m, 2H), 1.48 (s, 9H), 1.25 (d, J=6.6 Hz, 2.4H), 1.19 (d, J=6.6 Hz, 0.6H). UPLC/MS (method A): Rt 1.52 min. MS (ES) C₁₆H₃₀N₂O₂ requires 282, found 283 [M+H]⁺.

### (2S)-Methyl-4-(1-piperidyl)piperidine dihydrochloride (Xr)

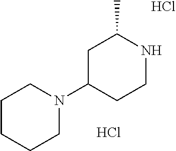

Following general procedure C, IXr (0.185 g, 0.66 mmol) afforded Xr which was used in the next step without further purification.

### (2S)-Methyl-N-pentyl-4-(1-piperidyl)piperidine-1-carboxamide

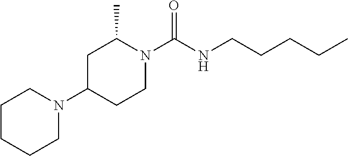 [SMILES: CCCCCNC(=O)N1CCC(N2CCCCC2)C[C@@H]1C]

Following general procedure D (Method A), Xr (0.070 g, 031 mmol) and pentylisocyanate (0.039 g, 0.34 mmol) afforded the title compound as a yellowish oil (4:1 mixture of two diastereoisomers, 0.034 g, 37%). ¹H NMR (400 MHz, DMSO-d₆) δ 6.47 (t, J=5.4 Hz, 0.2H), 6.33 (t, J=5.5 Hz, 0.8H), 4.49-4.37 (m, 0.2H), 3.98-3.86 (m, 0.2H), 3.84-3.72 (m, 0.8H), 3.69-3.59 (m, 0.8H), 3.26-2.63 (m, 8H), 2.24-1.82 (m, 2H), 1.82-1.62 (m, 5H), 1.62-1.42 (m, 3H), 1.42-1.35 (m, 2H), 1.32-1.17 (m, 4H), 1.13 (d, J=6.5 Hz, 2.4H), 1.06 (d, J=6.5 Hz, 0.6H), 0.86 (t, J=7.1 Hz, 3H). UPLC/MS (method A): Rt 1.42 min. MS (ES) C₁₇H₃₃N₃O requires 295, found 296 [M+H]⁺.

### Example 98: 2,6-Dimethyl-N-pentyl-4-(1-piperidyl)piperidine-1-carboxamide hydrochloride tert-Butyl 2,6-dimethyl-4-(1-piperidyl)piperidine-1-carboxylate (IXs)

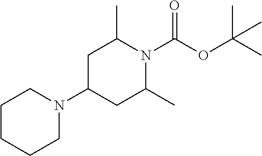 [SMILES: CC1CC(N2CCCCC2)CC(C)N1C(=O)OC(C)(C)C]

Following general procedure I (method B), V-Ae (0.100 g, 0.44 mmol) and piperidine (0.056 g, 0.66 mmol) afforded IXs which was used in the next step without further purification. UPLC/MS (method A): Rt 1.64 min. MS (ES) C₁₇H₃₂N₂O₂ requires 296, found 297 [M+H]⁺.

### 2,6-Dimethyl-4-(1-piperidyl)piperidine hydrochloride (Xs)

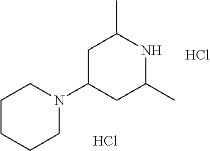

Following general procedure C, IXs (0.072 g, 0.24 mmol) afforded Xs which was used in the next step without further purification. UPLC/MS (method A): Rt 0.47 min. MS (ES) C₁₂H₂₄N₂ requires 196, found 197 [M+H]⁺.

### 2,6-Dimethyl-N-pentyl-4-(1-piperidyl)piperidine-1-carboxamide hydrochloride

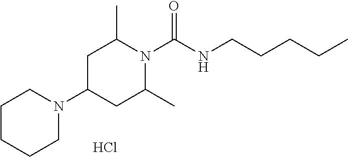 [SMILES: CCCCCNC(=O)N1C(C)CC(N2CCCCC2)CC1C ; Cl]

Following general procedure D (method A), Xs (0.071 g, 0.24 mmol) and n-pentyl isocyanate (0.030 g, 0.26 mmol) afforded the free base of the title compound as a colorless oil (mixture of two diastereoisomers 6:4, 0.033 g, 44%). ¹H NMR (400 MHz, DMSO-d₆) δ 6.51 (t, J=5.6 Hz, 0.6H), 6.31 (t, J=5.6 Hz, 0.4H), 4.07-3.97 (m, 0.4H), 3.82 (h, J=6.8 Hz, 0.6H), 3.44-3.33 (m, 0.4H), 3.10-2.84 (m, 2H), 2.60-2.50 (m, 0.6H), 2.46-2.35 (m, 4H), 2.34-2.22 (m, 0.4H), 1.87 (dt, J=12.4, 6.0 Hz, 0.6H), 1.77 (dt, J=13.3, 4.6 Hz, 0.4H), 1.69-1.56 (m, 1H), 1.52-1.18 (m, 12.6H), 1.16 (d, J=6.1 Hz, 1.2H), 1.12 (d, J=6.6 Hz, 3.6H), 1.02 (d, J=6.7 Hz, 1.2H), 0.85 (t, J=7.0 Hz, 3H). UPLC/MS (method A): Rt 1.54 min MS (ES) C₁₈H₃₅N₃O requires 309, found 310 [M+H]⁺.

### Example 99: 2,2-Dimethyl-N-(4-phenylbutyl)-5-(1-piperidyl)piperidine-1-carboxamide tert-Butyl 2,2-dimethyl-5-(1-piperidyl)piperidine-1-carboxylate (IXt)

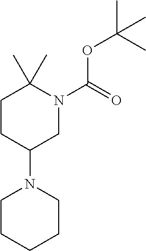 [SMILES: CC(C)(C)OC(=O)N1CC(N2CCCCC2)CCC1(C)C]

Following general procedure H (Method C), tert-butyl 2,2-dimethyl-5-oxo-piperidine-1-carboxylate (0.050 g, 0.22 mmol) afforded IXt as a colorless oil (0.044 g, 68%). %). ¹H NMR (400 MHz, CDCl₃) δ 3.70 (dd, J=14.1, 5.3 Hz, 1H), 3.39 (dd, J=14.1, 7.1 Hz, 1H), 2.78-2.56 (m, 5H), 1.9-1.82 (m, 1H), 1.70-1.55 (m, 5H), 1.52-1.47 (m, 4H), 1.47 (s, 9H), 1.38 (s, 6H). UPLC/MS (method A): Rt 1.72 min. MS (ES) C₁₇H₃₂N₂O₂ requires 296, found 297 [M+H]⁺.

### 2,2-Dimethyl-5-(1-piperidyl)piperidine hydrochloride (Xt)

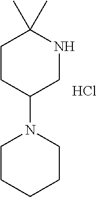

Following general procedure C, IXt (0.044 g, 0.15 mmol) afforded Xt which was used in the next step without further purification. UPLC/MS (method A): Rt 0.76 min. MS (ES) C₁₂H₂₄N₂ requires 196, found 197 [M+H]⁺.

### 2,2-Dimethyl-N-(4-phenylbutyl)-5-(1-piperidyl)piperidine-1-carboxamide

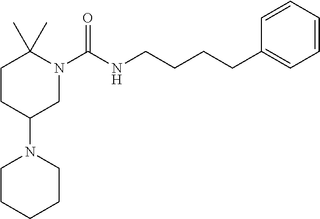 [SMILES: CC1(C)CCC(N2CCCCC2)CN1C(=O)NCCCCc1ccccc1]

Following general procedure D (method A), Xt (0.035 g, 0.15 mmol) and 4-phenylbutyl isocyanate (0.029 g, 0.028 ml, 0.165 mmol) afforded the title compound as a yellow oil (0.048 g, 86%). ¹H NMR (400 MHz, DMSO-d₆) δ 7.29-7.22 (m, 2H), 7.20-7.13 (m, 3H), 6.37 (t, J=5.5 Hz, 1H), 3.42-3.36 (m, 1H), 3.05-2.90 (m, 2H), 2.90-2.81 (m, 1H), 2.56 (t, J=7.6 Hz, 2H), 2.53-2.35 (m, 3H), 1.69-1.58 (m, 1H), 1.58-1.35 (m, 15H), 1.34 (s, 3H), 1.22 (s, 3H). UPLC/MS (method A): Rt 1.93 min. MS (ES) C₂₃H₃₇N₃O requires 371, found 372 [M+H]⁺.

### Example 100: 2-Methyl-N-(4-phenylbutyl)-5-(1-piperidyl)piperidine-1-carboxamide tert-Butyl 2-methyl-5-(1-piperidyl)piperidine-1-carboxylate (IXu)

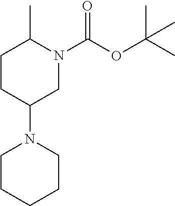 [SMILES: CC1CCC(N2CCCCC2)CN1C(=O)OC(C)(C)C]

Following general procedure I, V-Ag (0.150 g, 0.70 mmol) and piperidine (0.238 g, 2.8 mmol) afforded IXu as an oil (72 mg, 36%). ¹H NMR (400 MHz, CDCl₃) δ 4.14 (h, J=6.5 Hz, 1H), 4.05 (dd, J=14.7, 2.4 Hz, 1H), 2.96 (dd, J=14.7, 4.6 Hz, 1H), 2.59-2.40 (m, 5H), 2.03-1.88 (m, 1H), 1.68 (qdd, J=10.2, 8.0, 3.2 Hz, 2H), 1.63-1.53 (m, 4H), 1.48 (s, 9H), 1.49-1.40 (m, 2H), 1.19-1.28 (m, 4H). UPLC/MS (method A): Rt 1.55 min. MS (ES), C₁₆H₃₀N₂O₂ requires 282, found 283 [M+H]⁺.

### 2-Methyl-5-(1-piperidyl)piperidine dihydrochloride (Xu)

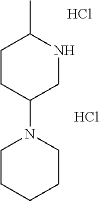

Following general procedure C, IXu (0.073 g, 0.258 mmol) afforded Xu as a white solid (0.067 g, quant.). UPLC/MS (method A): Rt 0.54 min. MS (ES), C₁₁H₂₂N₂ requires 182, found 183 [M+H]⁺.

### 2-Methyl-N-(4-phenylbutyl)-5-(1-piperidyl)piperidine-1-carboxamide

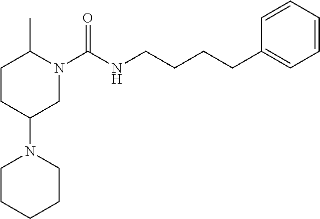 [SMILES: CC1CCC(N2CCCCC2)CN1C(=O)NCCCCc1ccccc1]

Following general procedure D (Method A), Xu (0.073 g, 0.258 mmol) and 4-phenylbutyl isocyanate (0.051 g, 0.293 mmol) afforded the title compound as an oil (0.074 g, 80%). ¹H NMR (400 MHz, DMSO-d₆) δ 7.30-7.24 (m, 2H), δ 7.21-7.13 (m, 3H), 6.45 (bs, 1H), 4.40-4.15 (m, 1H), 3.94 (q, J=6.4 Hz, 1H), 3.21-3.08 (m, 2H), 3.07-2.93 (m, 2H), 2.92-2.71 (m, 2H), 2.57 (t, J=7.5 Hz, 2H), 1.98-1.82 (m, 2H), 1.80-1.59 (m, 6H), 1.58-1.50 (m, 3H), 1.45-1.38 (m, 3H), 1.29-1.12 (m, 2H), 1.05 (d, J=6.3 Hz, 3H). UPLC/MS (method A): Rt 1.84 min. MS (ES), C₂₂H₃₅N₃O requires 357, found 358 [M+H]⁺.

### Example 101: 2-Methyl-N-(4-phenylbutyl)-3-(1-piperidyl)piperidine-1-carboxamide tert-Butyl 2-methyl-3-(1-piperidyl)piperidine-1-carboxylate (IXv)

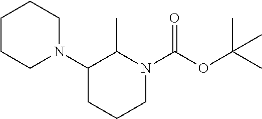 [SMILES: CC1C(N2CCCCC2)CCCN1C(=O)OC(C)(C)C]

Following general procedure I, V-Ah (0.150 g, 0.70 mmol) and piperidine (0.238 g, 2.8 mmol) afforded IXv as an oil (0.075 g, 38%). ¹H NMR (400 MHz, CDCl₃) δ 4.59 (d, J=7.0 Hz, 1H), 4.02-3.78 m, 1H) 2.86-2.68 (m, 1H), 2.56-2.37 (m, 4H), 2.25-2.11 (m, 1H), 1.93-1.81 (m, 1H), 1.75-1.61 (m, 2H), 1.60-1.51 (m, 4H), 1.48 (s, 9H), 1.46-1.34 (m, 3H), 1.08 (d, J=7.0 Hz, 3H). UPLC/MS (method A): Rt 1.55 min. MS (ES), C₁₆H₃₀N₂O₂ requires 282, found 283 [M+H]⁺.

### 2-Methyl-3-(1-piperidyl)piperidine dihydrochloride (Xv)

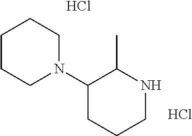

Following general procedure C, IXv (0.075 g, 0.266 mmol) afforded Xv as a white solid (0.067 g, quant.). ¹H NMR (400 MHz, DMSO-d₆) δ 10.81 (bs, 1H), 9.72 (bs, 1H), 9.62 (bs, 1H), 4.19-4.11 (m, 1H), 3.68-3.60 (m, 1H), 3.49 (overlapped H₂O signal, 1H), 3.40-3.33 (m, 1H), 3.09-2.89 (m, 4H), 2.20-2.08 (m, 1H), 2.04-1.58 (m, 8H), 1.53-1.47 (m, 1H), 1.45 (d, J=6.8 Hz, 3H).

### 2-Methyl-N-(4-phenylbutyl)-3-(1-piperidyl)piperidine-1-carboxamide

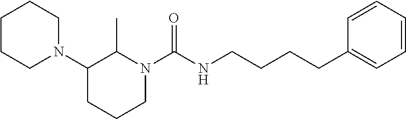 [SMILES: CC1C(N2CCCCC2)CCCN1C(=O)NCCCCc1ccccc1]

Following general procedure D (method A), Xv (0.068 g, 0.266 mmol) and 4-phenylbutyl isocyanate (0.051 g, 0.293 mmol) afforded the title compound as an oil (0.072 g, 76%). ¹H NMR (400 MHz, DMSO-d₆) δ 7.29-7.21 (m, 2H), 7.21-7.11 (m, 3H), 6.42 (bs, 1H), 4.63-4.16 (m, 1H), 3.68 (d, J=13.2 Hz, 1H), 3.03 (qd, J=6.8, 4.0 Hz, 2H), 2.73-2.59 (m, 2H), 2.56 (t, J=7.6 Hz, 2H), 2.48-2.31 (m, 2H), 2.13-1.94 (m, 1H), 1.90-1.71 (m, 1H), 1.68-1.58 (m, 1H), 1.57-1.45 (m, 6H), 1.45-1.33 (m, 5H), 1.32-1.15 (m, 2H), 0.95 (d, J=6.6 Hz, 3H). UPLC/MS (method A): Rt 1.80 min. MS (ES), C₂₂H₃₅N₃O requires 357, found 358 [M+H]⁺.

### Example 102: Exo-N-Pentyl-3-(1-piperidyl)-8-azabicyclo[3.2.1]octane-8-carboxamide tert-Butyl 3-(1-piperidyl)-8-azabicyclo[3.2.1]octane-8-carboxylate (IXw)

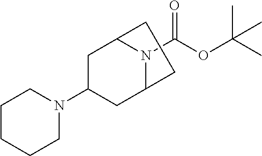 [SMILES: CCC1CC(N2CCCCC2)CC(CC)N1C(=O)OC(C)(C)C]

Following general procedure I (Method B), V-Ai (0.225 g, 1.00 mmol) and piperidine (0.341 g, 4.00 mmol) afforded IXw as a colorless oil (0.177 g, 60%). ¹H NMR (400 MHz, CDCl₃) δ 4.38-4.08 (m, 2H), 2.78 (p, J=9.0 Hz, 1H), 2.45 (t, 1=5.3 Hz, 4H), 2.00-1.85 (m, 2H), 1.80-1.51 (m, 10H), 1.46 (s, 9H), 1.44-1.37 (m, 2H). UPLC/MS (method A): Rt 1.53 min. MS (ES), C₁₇H₃₀N₂O₂ requires 294, found 295 [M+H]⁺.

### 3-(1-Piperidyl)-8-azabicyclo[3.2.1]octane dihydrochloride (Xw)

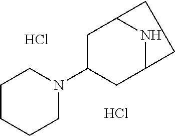 [SMILES: CCC1CC(N2CCCCC2)CC(CC)N1 ; Cl ; Cl]

Following general procedure C, IXw (0.177 g, 0.601 mmol) afforded Xw which was used in the next step without further purification. ¹H NMR (400 MHz, DMSO-d₆) δ 10.84 (bs, 1H), 9.76-9.14 (m, 2H), 4.14-4.03 (m, 2H), 3.71-3.59 (m, 1H), 3.52-3.44 (m, 1H), 2.98-2.79 (m, 2H), 2.31-2.05 (m, 4H), 2.03-1.61 (m, 10H), 1.48-1.31 (m, 1H).

### Exo-N-Pentyl-3-(1-piperidyl)-8-azabicyclo[3.2.1]octane-8-carboxamide

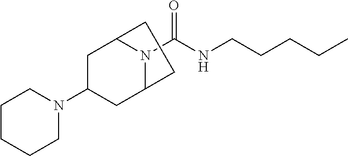 [SMILES: CCCCCNC(=O)N1C(CC)CC(N2CCCCC2)CC1CC]

Following general procedure D (method A), Xw (0.070 g, 0.262 mmol) and pentyl isocyanate (0.036 g, 0.314 mmol) afforded the title compound as a yellow oil (0.030 g, 37%). NMR analysis suggest exo conformation. ¹H NMR (400 MHz, CDCl₃) δ 4.33 (t, J=5.6 Hz, 1H), 4.23-4.14 (m, 2H), 3.22 (td, J=7.2, 5.7 Hz, 2H), 2.77 (tt, J=11.2, 6.1 Hz, 1H), 2.44 (t, J=5.3 Hz, 4H), 2.01-1.90 (m, 2H), 1.77-1.23 (m, 18H), 0.90 (t, J=6.8 Hz, 3H). UPLC/MS (method A): Rt 1.42 min. MS (ES), C₁₈H₃₃N₃O requires 307, found 308 [M+H]⁺.

### Example 103: 2,2-Dimethyl-4-phenyl-N-(4-phenylbutyl)piperazine-1-carboxamide

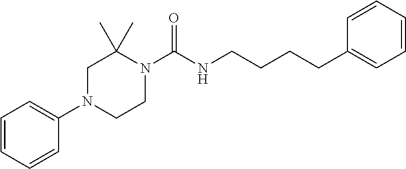 [SMILES: CC1(C)CN(c2ccccc2)CCN1C(=O)NCCCCc1ccccc1]

Following general procedure D (method A), 3,3-dimethyl-1-phenylpiperazine (0.020 g, 0.11 mmol) and 4-phenylbutyl isocyanate (0.022 g, 0.13 mmol) afforded the title compound as a clear oil (0.012 g, 30%). ¹H NMR (400 MHz, CDCl₃) δ 7.33-7.22 (m, 4H), 7.22-7.12 (m, 3H), 6.89-6.64 (m, 3H), 4.33 (bs, 1H), 3.56 (d, J=5.8 Hz, 2H), 3.48-3.30 (m, 2H), 3.30-3.22 (m, 2H), 3.20 (s, 2H), 2.65 (t, J=7.5 Hz, 2H), 1.72-1.63 (m, 2H), 1.61-1.51 (m, 2H), 1.48 (s, 6H). UPLC/MS (method A): Rt 2.64 min. MS (ES) C₂₃H₃₁N₃O requires 365, found 366 [M+H]⁺.

### Example 104: 2,2-Dimethyl-N-(4-phenylbutyl)-4-(2-pyridyl)piperazine-1-carboxamide tert-Butyl 2,2-dimethyl-4-(2-pyridyl)piperazine-1-carboxylate (XIIIb)

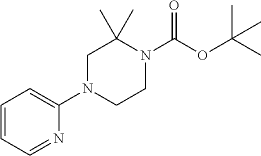 [SMILES: CC(C)(C)OC(=O)N1CCN(c2ccccn2)CC1(C)C]

Following general procedure G, XIIa (0.120 g, 0.56 mmol) and 2-bromopyridine (0.097 g, 0.62 mmol) afforded XIIIb as a yellow solid (0.073 g, 44%). ¹H NMR (400 MHz, CDCl₃) δ 8.17 (ddd, J=5.1, 2.0, 0.9 Hz, 1H), 7.68-7.41 (m, 1H), 6.59 (ddd, J=7.1, 4.9, 0.9 Hz, 1H), 6.45 (dt, J=8.5, 0.9 Hz, 1H), 4.02-3.84 (m, 2H), 3.83 (s, 2H), 3.59-3.36 (m, 2H), 1.52 (s, 9H), 1.43 (s, 6H). UPLC/MS (method A): Rt 2.39 min. MS (ES) C₁₆H₂₅N₃O₂ requires 291, found 292 [M+H]⁺.

### 3,3-Dimethyl-1-(2-pyridyl)piperazine hydrochloride (XIVb)

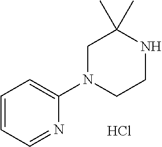

Following general procedure C, XIIIb (0.070 g, 0.240 mmol) afforded XIVb which was used in the next step without further purification. ¹H NMR (400 MHz, DMSO-d₆) δ 9.63 (bs, 2H), 8.10 (dd, J=5.6, 1.8 Hz, 1H), 7.86 (s, 1H), 7.23 (d, J=8.9 Hz, 1H), 6.89 (t, J=6.3 Hz, 1H), 3.88 (t, J=5.4 Hz, 2H), 3.78 (s, 2H), 3.26 (s, 2H), 1.37 (s, 6H). UPLC/MS (method C): Rt 1.92 min. MS (ES) C₁₁H₁₇N₃ requires 191, found 192 [M+H]⁺.

### 2,2-Dimethyl-N-(4-phenylbutyl)-4-(2-pyridyl)piperazine-1-carboxamide

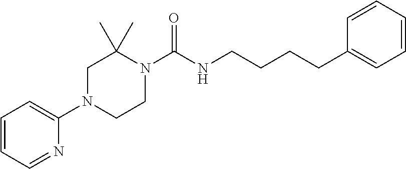 [SMILES: CC1(C)CN(c2ccccn2)CCN1C(=O)NCCCCc1ccccc1]

Following general procedure D (method A), XIVb (0.030 g, 0.114 mmol) and 4-phenylbutyl isocyanate (0.022 g, 0.125 mmol) afforded the title compound as a colorless oil (0.032 g, 76%). ¹H NMR (400 MHz, DMSO-d₆) δ 8.07 (dd, J=5.2, 2.0 Hz, 1H), 7.51 (ddd, J=8.9, 7.1, 2.0 Hz, 1H), 7.36-7.23 (m, 2H), 7.23-7.11 (m, 3H), 6.64 (d, J=8.6 Hz, 1H), 6.57 (dd, J=7.1, 4.9 Hz, 1H), 6.24 (t, J=5.5 Hz, 1H), 3.63 (s, 2H), 3.55 (dd, J=6.7, 4.5 Hz, 2H), 3.45 (dd, J=6.5, 4.6 Hz, 2H), 3.02 (td, J=7.0, 5.4 Hz, 2H), 2.58 (t, J=7.6 Hz, 2H), 1.63-1.47 (m, 2H), 1.49-1.36 (m, 2H), 1.34 (s, 6H). UPLC/MS (method A): Rt 1.76 min. MS (ES) C₂₂H₃₀N₄O requires 366, found 367 [M+H]⁺.

### Example 105: 2,2-Dimethyl-N-(4-phenylbutyl)-4-pyrimidin-5-yl-piperazine-1-carboxamide tert-Butyl 2,2-dimethyl-4-pyrimidin-5-yl-piperazine-1-carboxylate (XIIIc)

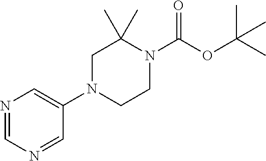 [SMILES: CC(C)(C)OC(=O)N1CCN(c2cncnc2)CC1(C)C]

Following general procedure G, XIIa (0.214 g, 1.0 mmol) and 5-bromopyrimidine (0.170 g, 1.1 mmol) afforded XIIIc as yellow solid (0.090 g, 31%). ¹H NMR (400 MHz, CDCl₃) δ 8.61 (s, 1H), 8.20 (s, 2H), 3.85 (d, J=5.8 Hz, 2H), 3.44 (s, 2H), 3.38 (s, 2H), 1.50 (s, 9H), 1.44 (s, 6H). UPLC/MS (method A): Rt 1.89 min. MS (ES) C₁₅H₂₄N₄O₂ requires 292, found 293 [M+H]⁺.

### 5-(3,3-Dimethylpiperazin-1-yl)pyrimidine hydrochloride (XIVc)

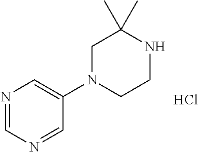

Following general procedure C, XIIIc (0.090 g, 0.31 mmol) afforded XIVc as a white solid (0.07 g, quant.). UPLC/MS (method A): Rt 0.6 min. MS (ES) C₁₀H₁₆N₄ requires 192, found 193 [M+H]⁺.

### 2,2-Dimethyl-N-(4-phenylbutyl)-4-pyrimidin-5-yl-piperazine-1-carboxamide

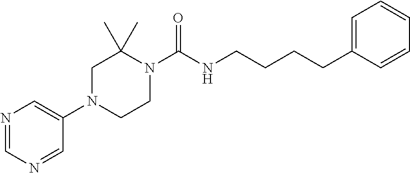 [SMILES: CC1(C)CN(c2cncnc2)CCN1C(=O)NCCCCc1ccccc1]

Following general procedure D (method A), XIVc (0.070 g, 0.31 mmol) and 4-phenylbutyl isocyanate (0.065 g, 0.37 mmol) afforded the title compound as a yellow oil (0.073 g, 64%). ¹H NMR (400 MHz, CDCl₃) δ ¹H NMR (400 MHz, CDCl₃) δ 8.64 (s, 1H), 8.23 (s, 2H), 7.31-7.24 (m, 2H), 7.21-7.15 (m, 3H), 4.32 (s, 1H), 3.69-3.54 (m, 2H), 3.51-3.40 (m, 2H), 3.29 (s, 2H), 3.24 (q, J=7.0 Hz, 2H), 2.64 (t, J=7.5 Hz, 2H), 1.71-1.62 (m, 2H), 1.55 (dt, J=14.7, 6.7 Hz, 2H), 1.48 (s, 6H). UPLC/MS (method A): Rt 1.94 min. MS (ES) C₂₁H₂₉N₅O requires 367, found 368 [M+H]⁺.

### Example 106: 2,2-Dimethyl-3-oxo-4-phenyl-N-(4-phenylbutyl)piperazine-1-carboxamide tert-Butyl 2,2-dimethyl-3-oxo-4-phenylpiperazine-1-carboxylate (XIIId)

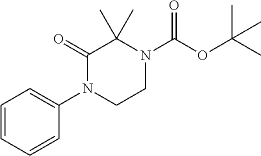 [SMILES: CC(C)(C)OC(=O)N1CCN(c2ccccc2)C(=O)C1(C)C]

Following general procedure E, XIIb (0.500 g, 3.9 mmol) and bromobenzene (0.420 g, 2.64 mmol) afforded XIId as a white solid (0.440 g, 66%). ¹H NMR (400 MHz, CDCl₃) δ 7.44-7.33 (m, 2H), 7.31-7.20 (m, 3H), 4.15-4.08 (m, 2H), 3.85-3.77 (m, 2H), 1.76 (s, 6H), 1.52 (s, 9H). UPLC/MS (method A): Rt 2.22 min. MS (ES) C₁₇H₂₄N₂O₃ requires 304, found 305 [M+H]⁺.

### 3,3-Dimethyl-1-phenylpiperazin-2-one hydrochloride (XIVd)

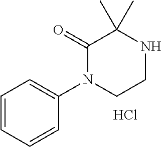

Following general procedure C, XIIId (0.200 g, 0.66 mmol) afforded XIVd as a white solid (0.150 g, 94%). UPLC/MS (method A): Rt 0.96 min. MS (ES) C₁₂H₁₆N₂O requires 204, found 205 [M+H]⁺.

### 2,2-Dimethyl-3-oxo-4-phenyl-N-(4-phenylbutyl)piperazine-1-carboxamide

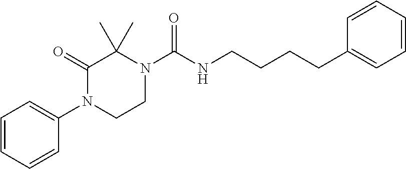 [SMILES: CC1(C)C(=O)N(c2ccccc2)CCN1C(=O)NCCCCc1ccccc1]

Following general procedure D (method A), XIVd (0.03 g, 0.12 mmol) and 4-phenylbutyl isocyanate (0.021 g, 0.12 mmol) afforded the title compound as a clear oil (0.04 g, 81%). ¹H NMR (400 MHz, CDCl₃) δ 7.40 (dd, J=8.4, 7.3 Hz, 2H), 7.32-7.24 (m, 5H), 7.22-7.15 (m, 3H), 4.42 (s, 1H), 3.75 (dd, J=6.4, 3.4 Hz, 2H), 3.67 (dd, J=6.2, 3.3 Hz, 2H), 3.32-3.22 (m, 2H), 2.66 (t, J=7.5 Hz, 2H), 1.79 (s, 6H), 1.75-1.64 (m, 2H), 1.63-1.46 (m, 2H). UPLC/MS (method A): Rt 2.27 min. MS (ES) C₂₃H₂₉N₃O₂ requires 379, found 380 [M+H]⁺.

### Example 107: 2,2-Dimethyl-3-oxo-N-pentyl-4-phenylpiperazine-1-carboxamide

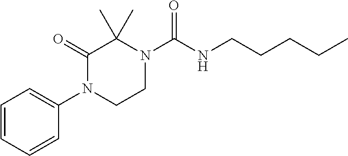 [SMILES: CCCCCNC(=O)N1CCN(c2ccccc2)C(=O)C1(C)C]

Following general procedure D (method A), XIVd (0.04 g, 0.17 mmol) and n-pentyl isocyanate (0.019 g, 0.17 mmol) afforded the title compound as a white solid (0.050 g, 83%). ¹H NMR (400 MHz, DMSO-d₆) δ 7.44-7.37 (m, 2H), 7.35-7.30 (m, 2H), 7.29-7.22 (m, 1H), 6.51 (t, J=5.4 Hz, 1H), 3.75-3.68 (m, 2H), 3.64-3.49 (m, 2H), 3.07-2.95 (m, 2H), 1.64 (s, 6H), 1.49-1.39 (m, 2H), 1.36-1.13 (m, 4H), 0.87 (t, J=7.0 Hz, 3H). UPLC/MS (method A): Rt 2.03 min. MS (ES) C₁₈H₂₇N₃O₂ requires 317, found 318 [M+H]⁺.

### Example 108: N-(2-Benzyloxyethyl)-2,2-dimethyl-3-oxo-4-phenylpiperazine-1-carboxamide

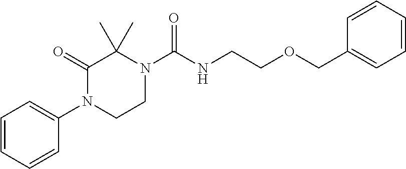 [SMILES: CC1(C)C(=O)N(c2ccccc2)CCN1C(=O)NCCOCc1ccccc1]

Following general procedure D (method B), XIVd (0.040 g, 0.17 mmol) and 2-benzyloxyethanamine (0.060 g, 0.51 mmol) afforded the title compound as a white solid (0.06 g, 93%). ¹H NMR (400 MHz, CDCl₃) δ 7.50-7.11 (m, 10H), 4.91 (d, J=4.9 Hz, 1H), 4.54 (s, 2H), 3.78-3.72 (m, 2H), 3.71-3.65 (m, 2H), 3.64-3.59 (m, 2H), 3.48 (q, J=5.1 Hz, 2H), 1.79 (s, 6H). UPLC/MS (method A): Rt 1.96 min. MS (ES) C₂₂H₂₇N₃O₃ requires 381, found 382 [M+H]⁺.

### Example 109: 4-(4-Fluoro-3-methoxy-phenyl)-2,2-dimethyl-3-oxo-N-(4-phenylbutyl)piperazine-1-carboxamide Benzyl 4-(4-fluoro-3-methoxyphenyl)-2,2-dimethyl-3-oxopiperazine-1-carboxylate (XIIIe)

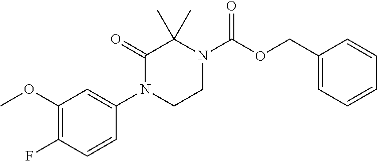 [SMILES: COc1cc(N2CCN(C(=O)OCc3ccccc3)C(C)(C)C2=O)ccc1F]

Following general procedure E, XIIc (0.40 g, 1.52 mmol) and 5-bromo-2-fluoroanisole (0.44 g, 2.13 mmol) afforded XVIIIe as a yellowish oil (0.380 g, 64%). ¹H NMR (400 MHz, CDCl₃) δ 7.52-7.31 (m, 5H), 7.10 (dd, J=10.9, 8.6 Hz, 1H), 6.96 (dd, J=7.6, 2.5 Hz, 1H), 6.81-6.72 (m, 1H), 5.20 (s, 2H), 3.99-3.84 (m, 5H), 3.78-3.65 (m, 2H), 1.80 (s, 6H). UPLC/MS (method A): Rt 2.26 min. MS (ES) C₂₁H₂₃FN₂O₄ require 386, found 387 [M+H]⁺.

### 1-(4-Fluoro-3-methoxyphenyl)-3,3-dimethyl-piperazin-2-one (XIVe)

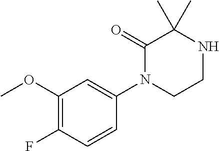 [SMILES: COc1cc(N2CCNC(C)(C)C2=O)ccc1F]

Following general procedure B (Method E), XVIIIe (0.190 g, 0.492 mmol) afforded XIVe which was used in the next step without further purification. UPLC/MS (method A): Rt 1.15 min. MS (ES) C₁₃H₁₇FN₂O₂ require 252, found 253 [M+H]⁺.

### 4-(4-Fluoro-3-methoxy-phenyl)-2,2-dimethyl-3-oxo-N-(4-phenylbutyl)piperazine-1-carboxamide

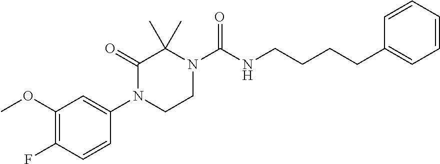 [SMILES: COc1cc(N2CCN(C(=O)NCCCCc3ccccc3)C(C)(C)C2=O)ccc1F]

Following general procedure D (Method A), XIVe (0.057 g, 0.225 mmol) and 4-phenylbutyl isocyanate (0.043 g, 0.25 mmol) afforded the title compound as a colorless oil (0.067 g, 69%). ¹H NMR (400 MHz, DMSO-d₆) δ 7.32-7.12 (m, 7H), 6.94-6.78 (m, 1H), 6.55 (t, J=5.4 Hz, 1H), 3.84 (s, 3H), 3.73-3.63 (m, 2H), 3.64-3.52 (m, 2H), 3.10-2.96 (m, 2H), 2.60 (t, J=7.6 Hz, 2H), 1.65 (s, 6H), 1.64-1.51 (m, 2H), 1.51-1.36 (m, 2H). UPLC/MS (method A): Rt 2.25 min. MS (ES) C₂₄H₃₀FN₃O₃ requires 427, found 428 [M+H]⁺.

### Example 110: 4-Cyclohexyl-2,2-dimethyl-N-(4-phenylbutyl)piperazine-1-carboxamide tert-Butyl 4-cyclohexyl-2,2-dimethylpiperazine-1-carboxylate (XIIIf)

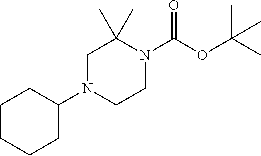 [SMILES: CC(C)(C)OC(=O)N1CCN(C2CCCCC2)CC1(C)C]

Following general procedure I (method B), XIIa (0.16 g, 0.75 mmol) and cyclohexanone (0.095 g; 0.97 mmol) afforded XIIIf as a white solid (0.100 g, 45%). ¹H NMR (400 MHz, CDCl₃) δ 3.49-3.30 (m, 2H), 2.61-2.49 (m, 2H), 2.28 (s, 2H), 2.27-2.15 (m, 1H), 1.93-1.71 (m, 4H), 1.69-1.61 (m, 1H), 1.48 (s, 9H), 1.38 (s, 6H), 1.32-1.11 (m, 5H). UPLC/MS (method A): Rt 2.42 min. MS (ES) C₁₇H₃₂N₂O₂ requires 296, found 297 [M+H]⁺.

### 1-Cyclohexyl-3,3-dimethylpiperazine dihydrochloride (XIVf)

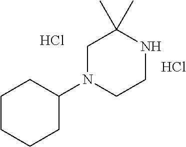

Following general procedure C, XIIIf (0.100 g, 0.34 mmol) afforded XIVf which was used in the next step without further purification. UPLC/MS (method A): Rt 1.48 min. MS (ES) C₁₂H₂₄N₂ requires 196, found 197 [M+H]⁺.

### 4-Cyclohexyl-2,2-dimethyl-N-(4-phenylbutyl)piperazine-1-carboxamide

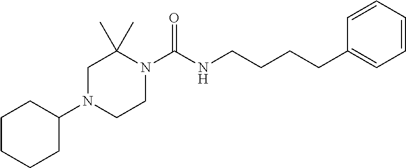 [SMILES: CC1(C)CN(C2CCCCC2)CCN1C(=O)NCCCCc1ccccc1]

Following general procedure D (Method A), XIVf (0.060 g, 0.26 mmol) and 4-phenylbutyl isocyanate (0.051 g, 0.29 mmol) afforded the title compound as a colorless oil (0.054 g, 55%). ¹H NMR (400 MHz, DMSO) δ 7.34-7.22 (m, 2H), 7.22-7.08 (m, 3H), 6.34 (t, J=5.4 Hz, 1H), 3.21-3.08 (m, 2H), 2.99 (dt, J=19.5, 3.4 Hz, 2H), 2.56 (t, J=7.6 Hz, 2H), 2.49-2.44 (m, 2H), 2.23-2.10 (m, 3H), 1.81-1.63 (m, 4H), 1.61-1.48 (m, 3H), 1.46-1.33 (m, 2H), 1.28 (s, 6H), 1.24-1.00 (m, 5H). UPLC/MS (method A): Rt 2.28 min. MS (ES) C₂₃H₃₇N₃O requires 371, found 372 [M+H]⁺.

### Example 111: 4-Cyclopropyl-2,2-dimethyl-N-(4-phenylbutyl)piperazine-1-carboxamide tert-Butyl 4-cyclopropyl-2,2-dimethylpiperazine-1-carboxylate (XIIIg)

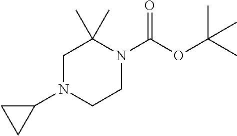 [SMILES: CC(C)(C)OC(=O)N1CCN(C2CC2)CC1(C)C]

Following general procedure I (method C), XIIa (0.12 g, 0.56 mmol) and [(1-ethoxycyclopropyl)oxy]trimethylsilane (0.117 g; 0.67 mmol) afforded XIIIg which was used in the next step without further purification. ¹H NMR (400 MHz, CDCl₃) δ 3.44-3.32 (m, 2H), 2.6-2.52 (m, 2H), 2.35 (s, 2H), 1.59-1.52 (m, 1H), 1.47 (s, 9H), 1.35 (s, 6H), 0.53-0.32 (m, 4H). UPLC/MS (method B): Rt 1.90 min. MS (ES) C₁₄H₂₆N₂O₂ requires 254, found 255 [M+H]⁺.

### 1-Cyclopropyl-3,3-dimethylpiperazine;dihydrochloride (XIVg)

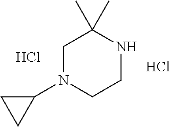

Following general procedure C, XIIIg (0.142 g, 0.56 mmol) afforded XIVg was used in the next step without further purification. UPLC/MS (method A): Rt 1.11 min. MS (ES) C₉H₁₈N₂ requires 154, found 155 [M+H]⁺.

### 4-Cyclopropyl-2,2-dimethyl-N-(4-phenylbutyl)piperazine-1-carboxamide

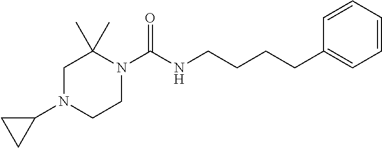 [SMILES: CC1(C)CN(C2CC2)CCN1C(=O)NCCCCc1ccccc1]

Following general procedure D (Method A), XIVg (0.075 g, 0.33 mmol) and 4-phenylbutyl isocyanate (0.064 g, 0.36 mmol) afforded the title compound as a colorless oil (0.058 g, 53%). ¹H NMR (400 MHz, DMSO) δ 7.35-7.23 (m, 2H), 7.23-7.10 (m, 3H), 6.39 (t, J=5.4 Hz, 1H), 3.23-3.10 (m, 2H), 3.02-2.92 (m, 2H), 2.56 (t, J=7.6 Hz, 2H), 2.24 (s, 2H), 1.61-1.47 (m, 3H), 1.47-1.34 (m, 2H), 0.48-0.35 (m, 2H), 0.35-0.23 (m, 2H). UPLC/MS (method A): Rt 2.59 min. MS (ES) C₂₀H₃₁N₃O requires 329, found 330 [M+H]⁺.

### Example 112: 4-Cyclopropyl-2,2-dimethyl-N-pentylpiperazine-1-carboxamide

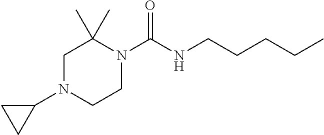 [SMILES: CCCCCNC(=O)N1CCN(C2CC2)CC1(C)C]

Following general procedure D (Method A), XIVg (0.045 g, 0.20 mmol) and pentylisocyanate (0.025 g, 0.22 mmol) afforded the title compound as a white solid (0.034 g, 62%). ¹H NMR (400 MHz, DMSO-d₆) δ 6.36 (t, J=5.4 Hz, 1H), 3.22-3.08 (m, 2H), 2.93 (td, J=7.1, 5.5 Hz, 2H), 2.52-2.49 (m, 2H), 2.24 (s, 2H), 1.63-1.47 (m, 1H), 1.45-1.31 (m, 2H), 1.26 (s, 10H), 0.86 (t, J=7.1 Hz, 3H), 0.46-0.37 (m, 2H), 0.32-0.24 (m, 2H). UPLC/MS (method A): Rt 2.36 min. MS (ES) C₁₈H₂₉N₃O requires 267, found 268 [M+H]⁺.

### Example 113: (2S,5R)-4-Cyclopropyl-2,5-dimethyl-N-(4-phenylbutyl)piperazine-1-carboxamide

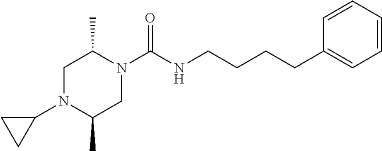 [SMILES: C[C@@H]1CN(C(=O)NCCCCc2ccccc2)[C@@H](C)CN1C1CC1]

Following general procedure D (method A), XIVh (0.050 g, 0.22 mmol) and 4-phenylbutyl isocyanate (0.041 g, 0.24 mmol) afforded the title compound as a colorless oil (0.050 g, 69%). ¹H NMR (400 MHz, CDCl₃) δ 7.31-7.23 (m, 2H), 7.21-7.13 (m, 3H), 4.30 (t, J=5.4 Hz, 1H), 4.02-3.92 (m, 1H), 3.42 (dd, J=12.5, 1.8 Hz, 1H), 3.34-3.19 (m, 2H), 3.14 (dd, J=12.6, 3.7 Hz, 1H), 3.08-2.98 (m, 1H), 2.94 (dd, J=11.7, 4.3 Hz, 1H), 2.64 (t, J=7.5 Hz, 2H), 2.37 (dd, J=11.7, 1.9 Hz, 1H), 1.82 (tt, J=6.5, 3.6 Hz, 1H), 1.72-1.60 (m, 2H), 1.59-1.48 (m, 2H), 1.15 (d, J=6.6 Hz, 3H), 1.04 (d, J=6.5 Hz, 3H), 0.49-0.35 (m, 3H), 0.32-0.21 (m, 1H). UPLC/MS (method A): Rt 2.38 min MS (ES) C₂₀H₃₁N₃O requires 329, found 330 [M+H]⁺.

### Example 114: (2S,5R)-4-cyclopropyl-2,5-dimethyl-N-pentylpiperazine-1-carboxamide tert-Butyl (2S,5R)-4-cyclopropyl-2,5-dimethylpiperazine-1-carboxylate (XIIIh)

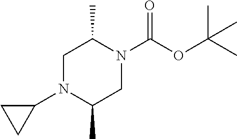 [SMILES: C[C@@H]1CN(C(=O)OC(C)(C)C)[C@@H](C)CN1C1CC1]

Following general procedure I (method C), XIId (0.150 g, 0.70 mmol) and [(1-ethoxycyclopropyl)oxy]trimethylsilane (0.144 g, 0.84 mmol) afforded XIIIh which was used in the next step without further purification. ¹H NMR (400 MHz, CDCl₃) δ 4.19 (t, J=6.1 Hz, 1H), 3.61 (d, J=13.5 Hz, 1H), 3.13 (dd, J=13.1, 3.7 Hz, 1H), 3.07-2.97 (m, 1H), 2.92 (dd, J=11.7, 4.4 Hz, 1H), 2.36 (d, J=11.7 Hz, 1H), 1.87-1.78 (m, 1H), 1.45 (s, 9H), 1.15 (d, J=6.6 Hz, 3H), 1.02 (d, J=6.6 Hz, 3H), 0.51-0.22 (m, 4H). UPLC/MS (method A): Rt 2.63 min. MS (ES) C₁₁H₂₂N₂O₂ requires 254, found 255 [M+H]⁺.

### (2S,5R)-1-Cyclopropyl-2,5-dimethylpiperazine dihydrochloride (XIVh)

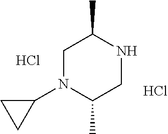

Following general procedure C, XIIIh (0.160 g, 0.63 mmol) afforded XIVh which was used in the next step without further purification. UPLC/MS (method A): Rt 0.90 min. MS (ES) C₉H₁₈N₂ requires 154, found 154 [M+H]⁺.

### (2S,5R)-4-Cyclopropyl-2,5-dimethyl-N-pentylpiperazine-1-carboxamide

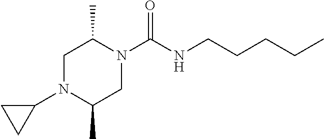 [SMILES: CCCCCNC(=O)N1C[C@@H](C)N(C2CC2)C[C@@H]1C]

Following general procedure D (method A), XIVh (0.050 g, 0.22 mmol) and n-pentyl isocyanate (0.027 g, 0.24 mmol) afforded the title compound as a colorless oil (0.020 g, 34%). ¹H NMR (400 MHz, CDCl₃) δ 4.31 (bs, 1H), 4.05-3.93 (m, 1H), 3.43 (dd, J=12.6, 1.9 Hz, 1H), 3.30-3.11 (m, 3H), 3.08-3.00 (m, 1H), 2.95 (dd, J=11.7, 4.3 Hz, 1H), 2.38 (dd, J=11.7, 1.9 Hz, 1H), 1.83 (tt, J=6.5, 3.6 Hz, 1H), 1.50 (p, J=7.3 Hz, 2H), 1.40-1.22 (m, 4H), 1.16 (d, J=6.7 Hz, 3H), 1.05 (d, J=6.6 Hz, 3H), 0.90 (t, J=6.9 Hz, 3H), 0.48-0.35 (m, 3H), 0.30-0.21 (m, 1H). UPLC/MS (method A): Rt 2.01 min MS (ES) C₁₅H₂₉N₃O requires 267, found 268 [M+H]⁺.

### Example 115: (2S,6R)-4-cyclopropyl-2,6-dimethyl-N-pentylpiperazine-1-carboxamide (2S,6R)-tert-Butyl-4-cyclopropyl-2,6-dimethylpiperazine-1-carboxylate (XIIIi)

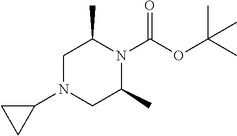 [SMILES: C[C@@H]1CN(C2CC2)C[C@H](C)N1C(=O)OC(C)(C)C]

Following general procedure I (method C), XIIe (0.100 g, 0.47 mmol) and [(1-ethoxycyclopropyl)oxy]trimethylsilane (0.113 g, 0.56 mmol) afforded XIIIi which was used in the next step without further purification. ¹H NMR (400 MHz, CDCl₃) δ 4.11-3.99 (m, 2H), 2.71 (d, J=11.1 Hz, 2H), 2.34 (d, J=11.1 Hz, 2H), 1.65-1.54 (m, 1H), 1.46 (s, 9H), 1.18 (d, J=6.8 Hz, 6H), 0.51-0.29 (m, 4H). UPLC/MS (method A): Rt 2.07 min. MS (ES) C₁₁H₂₂N₂O₂ requires 254, found 255 [M+H]⁺.

### (3S,5R)-1-Cyclopropyl-3,5-dimethylpiperazine dihydrochloride (XIVi)

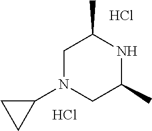

Following general procedure C, XIIIi (0.110 g, 0.43 mmol) afforded XIVi was used in the next step without further purification. UPLC/MS (method A): Rt 0.87 min. MS (ES) C₉H₁₈N₂ requires 154, found 154 [M+H]⁺.

### (2S,6R)-4-Cyclopropyl-2,6-dimethyl-N-pentyl-piperazine-1-carboxamide

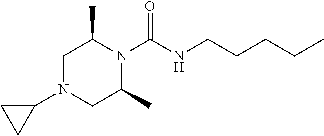 [SMILES: CCCCCNC(=O)N1[C@H](C)CN(C2CC2)C[C@@H]1C]

Following general procedure D (method A), XIVi (0.060 g, 0.26 mmol) and n-pentyl isocyanate (0.036 g, 0.32 mmol) afforded the title compound as a white solid (0.042 g, 60%). ¹H NMR (400 MHz, CDCl₃) δ 4.33 (bs, 1H), 4.02-3.87 (m, 2H), 3.23 (td, J=7.2, 5.4 Hz, 2H), 2.74 (d, J=11.1 Hz, 2H), 2.37 (dd, J=11.3, 4.4 Hz, 2H), 1.68-1.56 (m, 1H), 1.50 (p, J=7.3 Hz, 2H), 1.40-1.25 (m, 4H), 1.21 (d, J=6.8 Hz, 6H), 0.90 (t, J=6.9 Hz, 3H), 0.50-0.29 (m, 4H). UPLC/MS (method A): Rt 2.44 min MS (ES) C₁₅H₂₉N₃O requires 267, found 268 [M+H]⁺.

### Example 116: (2R)-4-Cyclopropyl-2-isopropyl-N-(4-phenylbutyl)piperazine-1-carboxamide tert-Butyl (2R)-4-cyclopropyl-2-isopropyl-piperazine-1-carboxylate (XIIIj)

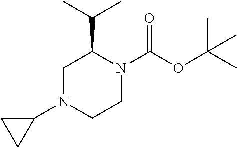 [SMILES: CC(C)[C@@H]1CN(C2CC2)CCN1C(=O)OC(C)(C)C]

Following general procedure I (method C), XIIIg (0.16 g, 0.70 mmol) and [(1-ethoxycyclopropyl)oxy]trimethylsilane (0.147 g, 0.84 mmol) afforded XIIIj as a colorless oil (0.188 g, quantitative). ¹H NMR (400 MHz, CDCl₃) δ 4.12-3.79 (m, 1H), 3.79-3.46 (m, 1H), 3.03 (d, J=11.6 Hz, 1H), 2.95-2.72 (m, 2H), 2.29-2.04 (m, 3H), 1.61-1.51 (m, 2H), 1.48 (s, 9H), 0.91 (d, J=6.6 Hz, 3H), 0.82 (d, J=6.8 Hz, 3H), 0.56-0.34 (m, 3H), 0.37-0.24 (m, 1H). UPLC/MS (method B): Rt 2.02 min. MS (ES) C₁₈H₂₈N₂O₂ requires 268, found 269 [M+H]⁺.

### (3R)-1-Cyclopropyl-3-isopropylpiperazine dihydrochloride (XIVj)

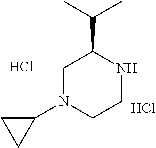

Following general procedure C, XIIIj (0.185 g, 0.69 mmol) afforded XIVj which was used in the next step without further purification. UPLC/MS (method C): Rt 1.77 min. MS (ES) C₁₀H₂₀N₂ requires 168, found 169 [M+H]⁺.

### (2R)-4-Cyclopropyl-2-isopropyl-N-(4-phenylbutyl)piperazine-1-carboxamide

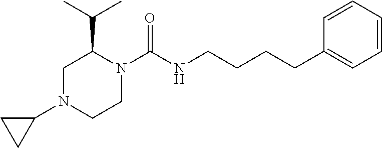 [SMILES: CC(C)[C@@H]1CN(C2CC2)CCN1C(=O)NCCCCc1ccccc1]

Following general procedure D (Method A), XIVj (0.060 g, 0.25 mmol) and 4-phenylbutyl isocyanate (0.048 g, 0.27 mmol) afforded the title compound as a colorless oil (0.045 g, 52%). ¹H NMR (400 MHz, DMSO-d₆) δ 7.31-7.22 (m, 2H), 7.22-7.11 (m, 3H), 6.25 (t, J=5.5 Hz, 1H), 3.78 (d, J=14.0 Hz, 1H), 3.58 (d, J=9.9 Hz, 1H), 3.13-2.96 (m, 2H), 2.94 (d, J=11.4 Hz, 1H), 2.75 (t, J=11.9 Hz, 2H), 2.57 (t, J=7.6 Hz, 2H), 2.18-1.98 (m, 3H), 1.60-1.48 (m, 3H), 1.47-1.34 (m, 2H), 0.85 (d, J=6.5 Hz, 3H), 0.70 (d, J=6.8 Hz, 3H), 0.48-0.35 (m, 2H), 0.39-0.27 (m, 1H), 0.26-0.16 (m, 1H). UPLC/MS (method A): Rt 2.59 min. MS (ES) C₂₁H₃₃N₃O requires 343, found 344 [M+H]⁺.

### Example 117: (2S)-4-Cyclopropyl-2-isopropyl-N-(4-phenylbutyl)piperazine-1-carboxamide

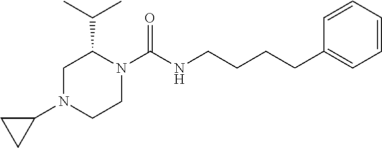 [SMILES: CC(C)[C@H]1CN(C2CC2)CCN1C(=O)NCCCCc1ccccc1]

Following general procedure D (Method A), XIVk (0.060 g, 0.25 mmol) and 4-phenylbutyl isocyanate (0.048 g, 0.27 mmol) afforded the title compound as a colorless oil (0.057 g, 66%). ¹H NMR (400 MHz, DMSO-d₆) δ 7.32-7.22 (m, 2H), 7.23-7.11 (m, 3H), 6.26 (t, J=5.5 Hz, 1H), 3.78 (d, J=13.8 Hz, 1H), 3.58 (d, J=10.0 Hz, 1H), 3.14-2.88 (m, 3H), 2.81-2.65 (m, 2H), 2.57 (t, J=7.6 Hz, 2H), 2.18-1.98 (m, 3H), 1.62-1.49 (m, 3H), 1.47-1.34 (m, 2H), 0.85 (d, J=6.6 Hz, 3H), 0.70 (d, J=6.8 Hz, 3H), 0.46-0.37 (m, 2H), 0.37-0.27 (m, 1H), 0.26-0.14 (m, 1H). UPLC/MS (method A): Rt 2.59 min. MS (ES) C₂₁H₃₃N₃O requires 343, found 344 [M+H]⁺.

### Example 118: (2S)-4-Cyclopropyl-2-isopropyl-N-pentylpiperazine-1-carboxamide tert-Butyl (2S)-4-cyclopropyl-2-isopropylpiperazine-1-carboxylate (XIIIk)

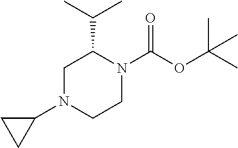 [SMILES: CC(C)[C@H]1CN(C2CC2)CCN1C(=O)OC(C)(C)C]

Following general procedure I (method C), XIIf (0.16 g, 0.70 mmol) and [(1-ethoxycyclopropyl)oxy]trimethylsilane (0.147 g, 0.84 mmol) afforded XIIIk as a colorless oil (0.188 g, quantitative). ¹H NMR (400 MHz, CDCl₃) δ 4.12-3.79 (m, 1H), 3.79-3.46 (m, 1H), 3.03 (d, J=11.6 Hz, 1H), 2.95-2.72 (m, 2H), 2.29-2.04 (m, 3H), 1.61-1.51 (m, 2H), 1.48 (s, 9H), 0.91 (d, J=6.6 Hz, 3H), 0.82 (d, J=6.8 Hz, 3H), 0.56-0.34 (m, 3H), 0.37-0.24 (m, 1H). UPLC/MS (method B): Rt 2.02 min. MS (ES) C₁₈H₂₈N₂O₂ requires 268, found 269 [M+H]⁺.

### (3S)-1-Cyclopropyl-3-isopropylpiperazine dihydrochloride (XIVk)

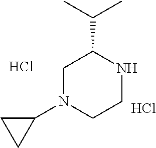

Following general procedure C, XIIIk (0.188 g, 0.70 mmol) afforded XIVk which was used in the next step without further purification. UPLC/MS (method C): Rt 1.77 min. MS (ES) C₁₀H₂₀N₂ requires 168, found 169 [M+H]⁺.

### (2S)-4-Cyclopropyl-2-isopropyl-N-pentylpiperazine-1-carboxamide

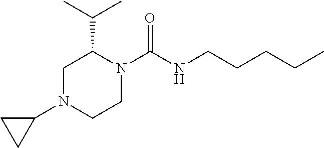 [SMILES: CCCCCNC(=O)N1CCN(C2CC2)C[C@@H]1C(C)C]

Following general procedure D (Method A), XIVk (0.060 g, 0.25 mmol) and pentylisocyanate (0.031 g, 0.27 mmol) afforded the title compound as a colorless oil (0.037 g, 57%). ¹H NMR (400 MHz, DMSO-d₆) δ 6.23 (t, J=5.5 Hz, 1H), 3.78 (d, J=14.3 Hz, 1H), 3.58 (d, J=10.2 Hz, 1H), 3.14-2.85 (m, 3H), 2.83-2.65 (m, 2H), 2.24-1.90 (m, 3H), 1.59-1.48 (m, 1H), 1.45-1.30 (m, 2H), 1.32-1.14 (m, 4H), 0.94-0.78 (m, 6H), 0.70 (d, J=6.8 Hz, 3H), 0.49-0.36 (m, 2H), 0.35-0.28 (m, 1H), 0.25-0.16 (m, 1H). UPLC/MS (method A): Rt 2.35 min. MS (ES) C₁₆H₃₁N₃O requires 281, found 282 [M+H]⁺.

### Example 119: 2,2,5-Trimethyl-4-oxo-N-(4-phenylbutyl)piperidine-1-carboxamide 2,2,5-Trimethylpiperidin-4-one hydrochloride (XVIIa)

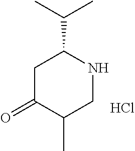

Following general procedure C, XVa (0.012 g, 0.050 mmol) afforded XVIIa as a white solid (0.009 g, quant.). UPLC/MS (method A): Rt 0.52 min. MS (ES), C₈H₁₅NO requires 141, found 142 [M+H]⁺.

### 2,2,5-Trimethyl-4-oxo-N-(4-phenylbutyl)piperidine-1-carboxamide

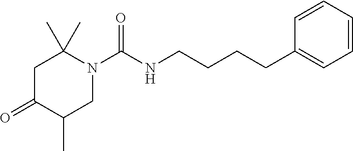 [SMILES: CC1CN(C(=O)NCCCCc2ccccc2)C(C)(C)CC1=O]

Following general procedure D (method A), XVIIa (0.009 g, 0.050 mmol) and 4-phenylbutyl isocyanate (0.010 g, 0.055 mmol) afforded the title compound as a white solid (0.010 g, 62%). ¹H NMR (400 MHz, DMSO-d₆) δ 7.31-7.23 (m, 2H), 7.23-7.12 (m, 3H), 6.34 (t, J=5.5 Hz, 1H), 3.70 (dd, J=13.6, 5.3 Hz, 1H), 3.27 (dd, J=13.6, 9.6 Hz, 1H), 3.09-2.96 (m, 2H), 2.64 (d, J=14.4 Hz, 1H), 2.58 (t, J=7.6 Hz, 2H), 2.47-2.38 (m, 1H), 2.38 (d, J=14.5 Hz, 1H), 1.60-1.52 (m, 2H), 1.46-1.40 (m, 2H), 1.39 (s, 3H), 1.36 (s, 3H), 0.96 (d, J=7.1 Hz, 3H). UPLC/MS (method A): Rt 2.16 min. MS (ES), C₁₀H₂₈N₂O₂ requires 316, found 317 [M+H]⁺.

### Example 120: 2,2,5,5-Tetramethyl-4-oxo-N-(4-phenylbutyl)piperidine-1-carboxamide tert-Butyl 2,2,5,5-tetramethyl-4-oxopiperidine-1-carboxylate (XVb)

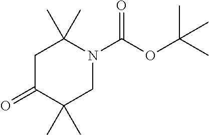 [SMILES: CC(C)(C)OC(=O)N1CC(C)(C)C(=O)CC1(C)C]

To a solution of V-Aa (0.100 g, 0.440 mmol, 1.0 eq.) in anhydrous THF (1.5 mL) a solution of LiHMDS (0.880 mL, 1.0 M in THF) was added dropwise at −78° C. and the reaction mixture was stirred for 1 h. MeI (0.250 g, 1.76 mmol, 0.110 mL, 4.0 eq.) was added at −78° C., the reaction was stirred at −78° C. for 30 min and then at RT for 3 h. The mixture was then quenched with the addition of saturated aq. NH₄Cl solution, extracted with EA, washed with brine, dried over Na₂SO₄, concentrated and purified by column chromatography (SiO₂), eluting with Cy/EA (9:1) to afford XVb as a white solid (0.030 g, 27%). ¹H NMR (400 MHz, CDCl₃) δ 3.65 (s, 2H), 2.55 (s, 2H), 1.50 (s, 9H), 1.48 (s, 6H), 1.10 (s, 6H). UPLC/MS (method A): Rt 2.35 min. MS (ES), C₁₄H₂₅NO₃ requires 255, found 256 [M+H]⁺.

### 2,2,5,5-Tetramethylpiperidin-4-one hydrochloride (XXIb)

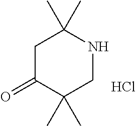

Following general procedure C, XVb (0.024 g, 0.094 mmol) afforded XXIb as a white solid (0.018 g, quant.). ¹H NMR (400 MHz, DMSO-d₆) δ 9.42 (s, 2H), 3.21 (s, 2H), 2.59 (s, 2H), 1.33 (s, 6H), 1.17 (s, 6H).

### 2,2,5,5-Tetramethyl-4-oxo-N-(4-phenylbutyl)piperidine-1-carboxamide

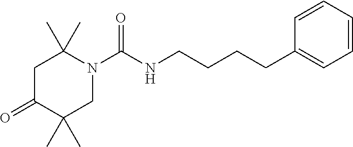 [SMILES: CC1(C)CN(C(=O)NCCCCc2ccccc2)C(C)(C)CC1=O]

Following general procedure D (method A), XXIb (0.018 g, 0.094 mmol) and 4-phenylbutyl isocyanate (18 mg, 0.103 mmol) afforded the title compound as a white solid (0.019 g, 61%). ¹H NMR (400 MHz, DMSO-d₆) δ 7.31-7.22 (m, 2H), 7.22-7.11 (m, 3H), 6.18 (t, J=5.4 Hz, 1H), 3.44 (s, 2H), 3.03 (q, J=6.5 Hz, 2H), 2.61-2.54 (m, 4H), 1.58-1.50 (m, 2H), 1.45-1.36 (m, 2H), 1.39 (s, 6H), 0.98 (s, 6H). UPLC/MS (method A): Rt 2.30 min. MS (ES), C₁₄H₂₅NO₃ requires 330, found 331 [M+H]⁺.

### Example 121: 5-Benzyl-2,2-dimethyl-4-oxo-N-(4-phenylbutyl)piperidine-1-carboxamide tert-Butyl 5-benzyl-2,2-dimethyl-4-oxopiperidine-1-carboxylate (XVc)

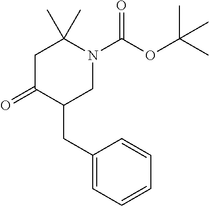 [SMILES: CC(C)(C)OC(=O)N1CC(Cc2ccccc2)C(=O)CC1(C)C]

To a solution of XVIIIa (0.067 g, 0.21 mmol) in EtOH (2 mL) was added 10% Pd/C (0.034 g, 0.032 mmol) under N₂ atmosphere. Then, Et₃SiH (0.33 mL, 2.1 mmol) was added dropwise and the reaction mixture was stirred at RT for 1 h, filtered through a pad of Celite, concentrated and purified by column chromatography (SiO₂), eluting with Cy/EA (85:15) to afford XVc (0.042 g, 62%). ¹H NMR (400 MHz, CDCl₃) δ 7.34-7.27 (m, 2H), 7.27-7.15 (m, 3H), 3.97 (dd, J=14.2, 4.8 Hz, 1H), 3.50 (dd, J=14.2, 8.2 Hz, 1H), 3.15 (dd, J=13.6, 3.6 Hz, 1H), 2.71-2.44 (m, 4H), 1.47 (s, 6H), 1.47 (s, 9H). UPLC/MS (method A): Rt 1.63 min. MS (ES), C₁₉H₂₇NO₃ requires 317, found 318 [M+H]⁺.

### 5-Benzyl-2,2-dimethyl-piperidin-4-one hydrochloride (XXIc)

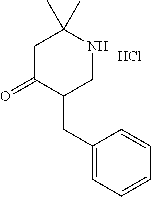 [SMILES: CC1(C)CC(=O)C(Cc2ccccc2)CN1 ; Cl]

Following general procedure C, XVc (0.027 g, 0.085 mmol) afforded XXIc as a white solid (0.021 g, quant.). UPLC/MS (method C): Rt 2.27 min. MS (ES), C₁₄H₁₉NO requires 217, found 218 [M+H]⁺.

### 5-Benzyl-2,2-dimethyl-4-oxo-N-(4-phenylbutyl)piperidine-1-carboxamide

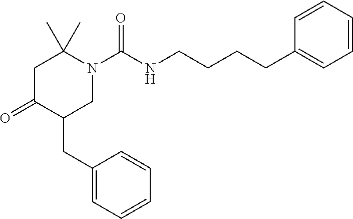 [SMILES: CC1(C)CC(=O)C(Cc2ccccc2)CN1C(=O)NCCCCc1ccccc1]

Following general procedure D (Method A), XXIc (0.021 g, 0.085 mmol) and 4-phenylbutyl isocyanate (0.017 g, 0.094 mmol) afforded the title compound as a white solid (0.023 g, 69%). ¹H NMR (400 MHz, DMSO-d₆) δ 7.30-7.21 (m, 4H), 7.21-7.12 (m, 6H), 6.21 (t, J=5.5 Hz, 1H), 3.60 (dd, J=13.6, 5.1 Hz, 1H), 3.30 (dd, J=13.6, 8.7 Hz, 1H), 3.08-2.86 (m, 3H), 2.70-2.61 (m, 1H), 2.60-2.45 (m, 4H), 1.57-1.45 (m, 2H), 1.41-1.34 (m, 2H), 1.38 (s, 3H), 1.36 (s, 3H). UPLC/MS (method A): Rt 2.57 min. MS (ES), C₂₅H₃₂N₂O₂ requires 392, found 393 [M+H]⁺.

### Example 122: (5E)-5-Benzylidene-2,2-dimethyl-4-oxo-N-(4-phenylbutyl)piperidine-1-carboxamide tert-Butyl (5E)-5-benzylidene-2,2-dimethyl-4-oxopiperidine-1-carboxylate (XVIIIa)

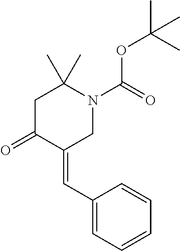 [SMILES: CC(C)(C)OC(=O)N1C/C(=C\c2ccccc2)C(=O)CC1(C)C]

To a solution of V-Aa (0.5 g, 2.2 mmol, 1.0 eq.) and pyrrolidine (0.157 g, 0.181 mL, 2.2 mmol) in DCM (5 mL) benzaldehyde (0.223 g, 2.2 mmol) was added and the reaction mixture was stirred at RT for 48 h. After evaporation of the solvent, the residue was purified by column chromatography (SiO₂), eluting with Cy/EA (9:1) to afford XVIIIa as a white solid (0.612 g, 88%). ¹H NMR (400 MHz, CDCl₃) δ 7.64 (d, J=1.4 Hz, 1H), 7.47-7.36 (m, 5H), 4.80 (d, J=1.4 Hz, 2H), 2.74 (s, 2H), 1.52 (s, 6H), 1.46 (s, 9H). UPLC/MS (method B): Rt 1.53 min. MS (ES), C₁₉H₂₅NO₃ requires 315, found 316 [M+H]⁺.

### (5E)-5-Benzylidene-2,2-dimethyl-piperidin-4-one hydrochloride (XXIIa)

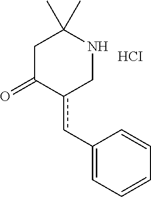

Following general procedure C, XVIIIa (0.018 g, 0.057 mmol) afforded XXIIa as a white solid (0.014 g, quant.). UPLC/MS (method A): Rt 1.08 min. MS (ES), C₁₄H₁₇NO requires 215, found 216 [M+H]⁺.

### (5E)-5-Benzylidene-2,2-dimethyl-4-oxo-N-(4-phenylbutyl)piperidine-1-carboxamide

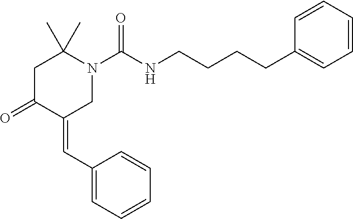 [SMILES: CC1(C)CC(=O)/C(=C/c2ccccc2)CN1C(=O)NCCCCc1ccccc1]

Following general procedure D (Method A), XXIIa (0.014 g, 0.057 mmol) and 4-phenylbutyl isocyanate (0.011 g, 0.063 mmol) afforded the title compound as a white solid (0.012 g, 54%). ¹H NMR (400 MHz, CDCl₃) δ 7.66 (s, 1H), 7.42-7.35 (m, 5H), 7.33-7.29 (m, 2H), 7.24-7.13 (m, 3H), 4.60 (d, J=1.4 Hz, 2H), 4.11 (t, J=4.7 Hz, 1H), 3.19 (td, J=7.0, 5.4 Hz, 2H), 2.75 (s, 2H), 2.61 (t, J=7.6 Hz, 2H), 1.55 (s, 6H), 1.57-1.43 (m, 4H). UPLC/MS (method A): Rt 2.45 min. MS (ES), C₂₅H₃₀N₂O₂ requires 390, found 391 [M+H]⁺.

### Example 123: 2,2-Dimethyl-4-oxo5-phenyl-N-(4-phenylbutyl)piperidine-1-carboxamide 2,2-Dimethyl-5-phenylpiperidin-4-one hydrochloride (XXId)

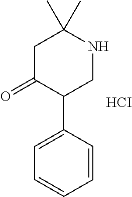

Following general procedure C, XVd (0.020 g, 0.066 mmol) afforded XXId as a white solid (0.016 g, quant.). ¹H NMR (400 MHz, DMSO-d₆) δ 9.69 (s, 2H), 7.41-7.29 (m, 3H), 7.28-7.20 (m, 2H), 4.19 (dd, J=12.7, 6.6 Hz, 1H), 3.69 (dd, J=13.0, 6.7 Hz, 1H), 3.65-3.54 (m, 1H), 3.00 (d, J=14.3 Hz, 1H), 2.50-2.45 (m, 1H), 1.49 (s, 3H), 1.36 (s, 3H). UPLC/MS (method A): Rt 1.17 min. MS (ES), C₁₃H₁₇NO requires 203, found 204 [M+H]⁺.

### 2,2-Dimethyl-4-oxo5-phenyl-N-(4-phenylbutyl)piperidine-1-carboxamide

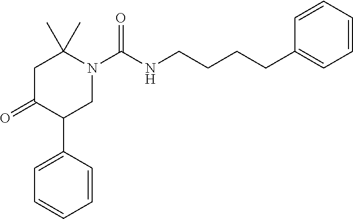 [SMILES: CC1(C)CC(=O)C(c2ccccc2)CN1C(=O)NCCCCc1ccccc1]

Following general procedure D (method A), XXId (0.016 g, 0.066 mmol) and 4-phenylbutyl isocyanate (0.018 g, 0.103 mmol) afforded the title compound as a white solid (0.023 g, 92%). ¹H NMR (400 MHz, DMSO-d₆) δ 7.38-7.21 (m, 7H), 7.21-7.08 (m, 3H), 6.38 (t, J=5.4 Hz, 1H), 3.86-3.79 (m, 2H), 3.70 (dd, J=9.2, 7.0 Hz, 1H), 3.12 (d, J=14.5 Hz, 1H), 3.08-2.93 (m, 2H), 2.57 (t, J=7.6 Hz, 2H), 2.40 (d, J=14.4 Hz, 1H), 1.57-1.50 (m, 2H), 1.47 (s, 3H), 1.47 (s, 3H), 1.44-1.37 (m, 2H). UPLC/MS (method A): Rt 2.30 min. MS (ES), C₂₄H₃₀N₂O₂ requires 378, found 379 [M+H]⁺.

### Example 124: 2,2,5-Trimethyl-N-pentyl-4-(1-piperidyl)piperidine-1-carboxamide tert-Butyl 2,2,5-trimethyl-4-oxopiperidine-1-carboxylate (XVa)

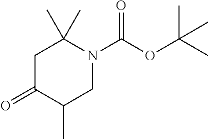 [SMILES: CC1CN(C(=O)OC(C)(C)C)C(C)(C)CC1=O]

To a solution of V-Aa (0.100 g, 0.440 mmol) in anhydrous THF (1.5 mL) a solution of LiHMDS (0.880 g, 0.880 mmol, 1.0 M in THF) was added dropwise at −78° C. and the reaction mixture was stirred for 1 h. MeI (0.110 mL, 1.76 mmol) was added at −78° C., the reaction was stirred at −78° C. for 30 min and then at RT for 3 h. The mixture was quenched with saturated aq. NH₄Cl solution, extracted with EA, washed with brine, dried over Na₂SO₄ concentrated and the residue was purified by column chromatography (SiO₂), eluting with Cy/EA (9:1) to afford XVa as a white solid (0.053 g, 50%). ¹H NMR (400 MHz, CDCl₃) δ 4.14 (dd, J=14.0, 5.0 Hz, 1H), 3.41 (dd, J=14.0, 9.3 Hz, 1H), 2.69 (d, J=14.7 Hz, 1H), 2.49-2.38 (m, 2H), 1.52 (s, 9H), 1.50 (s, 3H), 1.46 (s, 3H), 1.13 (d, J=7.2 Hz, 3H). UPLC/MS (method A): Rt 2.18 min. MS (ES), C₁₃H₂₃NO₃ requires 241, found 242 [M+H]⁺.

### tert-Butyl 2,5-dimethyl-4-(1-piperidyl)piperidine-1-carboxylate (XVIa)

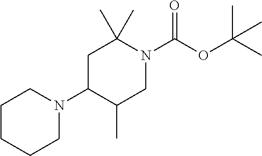 [SMILES: CC1CN(C(=O)OC(C)(C)C)C(C)(C)CC1N1CCCCC1]

Following general procedure H (method B), XVa (0.063 g, 0.198 mmol) and piperidine (0.017 g, 0.198 mmol) afforded XVIa as an oil. UPLC/MS (method A): Rt 1.76 min. MS (ES), C₁₈H₃₄N₂O₂ requires 310, found 311 [M+H]⁺.

### 2,2,5-Trimethyl-4-(1-piperidyl)piperidine dihydrochloride (XVIIa)

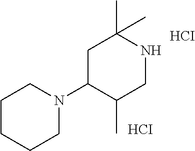

Following general procedure C, XVIa (0.054 g, 0.174 mmol) afforded XVIIa as a white solid (0.049 g, quant.). ¹H NMR (400 MHz, DMSO-d₆) δ 9.58 (bs, 1H), 9.39 (bs, 2H), 3.25-3.04 (m, 3H), 3.03-2.78 (m, 2H), 2.42-2.23 (m, 1H), 2.23-2.04 (m, 2H), 2.00-1.64 (m, 5H), 1.49-1.37 (m, 3H), 1.43 (s, 3H), 1.33 (s, 3H), 1.13 (d, J=6.5 Hz, 3H).

### 2,2,5-Trimethyl-N-pentyl-4-(1-piperidyl)piperidine-1-carboxamide

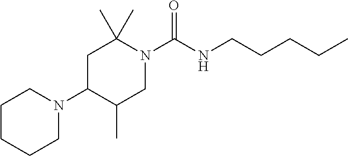 [SMILES: CCCCCNC(=O)N1CC(C)C(N2CCCCC2)CC1(C)C]

Following general procedure D (method A), XVIIa (0.049 g, 0.174 mmol) and pentyl isocyanate (0.022 g, 0.191 mmol) afforded the title compound (0.043 g, 76%). ¹H NMR (400 MHz, DMSO-d₆) δ 6.34 (t, J=5.5 Hz, 1H), 3.44 (dd, J=12.9, 4.3 Hz, 1H), 2.97-2.88 (m, 2H), 2.58 (dd, J=12.9, 9.9 Hz, 1H), 2.51-2.47 (m, 2H), 2.30-2.23 (m, 2H), 2.21-2.11 (m, 1H), 1.70-1.59 (m, 1H), 1.56-1.43 (m, 4H), 1.41 (s, 3H), 1.43-1.30 (m, 6H), 1.30-1.16 (m, 4H), 1.20 (s, 3H), 0.93-0.82 (m, 6H). UPLC/MS (method A): Rt 1.61 min. MS (ES), C₁₉H₃₇N₃O requires 323, found 324 [M+H]⁺.

### Example 125: 5-Benzyl-2,2-dimethyl-N-(4-phenylbutyl)-4-(1-piperidyl)piperidine-1-carboxamide tert-Butyl 5-benzyl-2,2-dimethyl-4-(1-piperidyl)piperidine-1-carboxylate (XVIb)

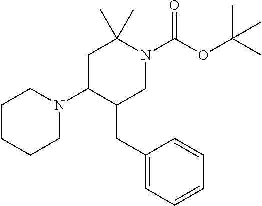 [SMILES: CC(C)(C)OC(=O)N1CC(Cc2ccccc2)C(N2CCCCC2)CC1(C)C]

Following general procedure I (method B), XVc (0.063 g, 0.198 mmol) and piperidine (0.017 g, 0.198 mmol) afforded XVIb as an oil (0.056 g, 73%). UPLC/MS (method A): Rt 1.11 min and 1.15 min. MS (ES), C₂₄H₃₈N₂O₂ requires 386, found 387 [M+H]⁺.

### 5-Benzyl-2,2-dimethyl-4-(1-piperidyl)piperidine dihydrochloride (XVIIb)

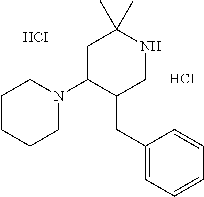

Following general procedure C, XVIb (0.051 g, 0.132 mmol) afforded XVIIb as a white solid (0.047 g, quant.). UPLC/MS (method A): Rt 1.84 min. MS (ES), C₁₉H₃₀N₂ requires 286, found 287 [M+H]⁺.

### Example 125: 5-Benzyl-2,2-dimethyl-N-(4-phenylbutyl)-4-(1-piperidyl)piperidine-1-carboxamide

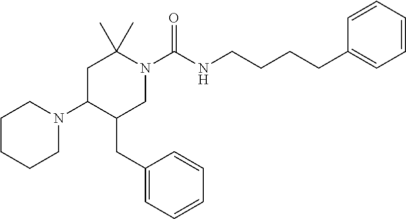 [SMILES: CC1(C)CC(N2CCCCC2)C(Cc2ccccc2)CN1C(=O)NCCCCc1ccccc1]

Following general procedure D (method A), XVIIb (0.047 g, 0.132 mmol) and 4-phenylbutyl isocyanate (0.025 g, 0.145 mmol) afforded the title compound as an oil (0.049 g, 80%). ¹H NMR (400 MHz, DMSO-d₆) δ 7.32-7.05 (m, 10H), 6.18 (t, J=5.5 Hz, 0.6H), 5.67 (t, J=5.5 Hz, 0.4H), 3.32-3.29 (m, 1H), 3.04-2.83 (m, 3H), 2.81-2.72 (m, 1H), 2.71-2.63 (m, 1H), 2.58-2.53 (m, 4H), 2.48-2.43 (m, 1H), 2.34-2.20 (m, 2H), 2.17-2.08 (m, 0.4H), 1.90-1.78 (m, 0.6H), 1.56-1.40 (m, 12H), 1.39-1.33 (m, 3H), 1.18-1.14 (m, 3H). UPLC/MS (method B): Rt 1.14 min. MS (ES), C₃₀H₄₃N₃O requires 461, found 462 [M+H]⁺.

### Example 126: 5-Benzyl-2,2-dimethyl-N-(2-phenylethyl)-4-(1-piperidyl)piperidine-1-carboxamide

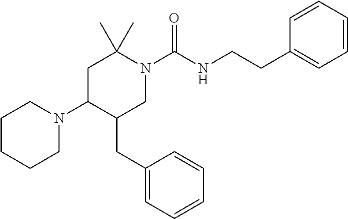 [SMILES: CC1(C)CC(N2CCCCC2)C(Cc2ccccc2)CN1C(=O)NCCc1ccccc1]

Following general procedure D (method A), XVIIb (0.041 g, 0.116 mmol) and phenethyl isocyanate (0.019 g, 0.128 mmol) afforded the title compound (0.042 g, 84%). ¹H NMR (400 MHz, DMSO-d₆) δ 7.32-7.23 (m, 4H), 7.23-7.07 (m, 6H), 6.20 (t, J=5.5 Hz, 0.6H), 5.68 (t, J=5.6 Hz, 0.4H), 3.24-3.06 (m, 3H), 2.90-2.84 (m, 1H), 2.80-2.74 (m, 1H), 2.70-2.62 (m, 3H), 2.58-2.53 (m, 1H), 2.45-2.36 (m, 2H), 2.30-2.20 (m, 2H), 2.16-2.07 (m, 0.4H), 1.82-1.78 (m, 0.6H), 1.57-1.34 (m, 11H), 1.20-1.12 (m, 3H), UPLC/MS (method A): Rt 2.09 min. MS (ES), C₂₈H₃₉N₃O requires 433, found 434. [M+H]⁺.

### Example 127: 5-Benzyl-2,2-dimethyl-N-pentyl-4-(1-piperidyl)piperidine-1-carboxamide

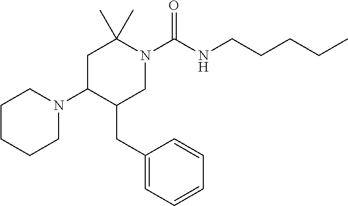 [SMILES: CCCCCNC(=O)N1CC(Cc2ccccc2)C(N2CCCCC2)CC1(C)C]

Following general procedure D (Method A), XVIIb (0.041 g, 0.116 mmol) and pentyl isocyanate (0.019 g, 0.128 mmol) afforded the title compound (0.043 g, 93%). ¹H NMR (400 MHz, DMSO-d₆) δ 7.31-7.23 (m, 2H), 7.23-7.13 (m, 3H), 6.13 (t, J=5.6 Hz, 0.6H), 5.62 (t, J=5.5 Hz, 0.4H), 3.31-3.26 (m, 1H), 3.00-2.82 (m, 3H), 2.80-2.73 (m, 1H), 2.71-2.61 (m, 1H), 2.60-2.53 (m, 3H), 2.48-2.34 (m, 2H), 2.32-2.21 (m, 3H), 2.18-2.08 (m, 0.4H), 1.89-1.77 (m, 0.6H), 1.55-1.21 (m, 14H), 1.20-1.12 (m, 3H), 0.90-0.78 (m, 3H). UPLC/MS (method A): Rt 2.09 min and 2.13. MS (ES), C₂₅H₄₁N₃O requires 399, found 400. [M+H]⁺.

### Example 128: (5E)-5-Benzylidene-2,2-dimethyl-N-(4-phenylbutyl)-4-(1-piperidyl)piperidine-1-carboxamide tert-Butyl (5E)-5-benzylidene-2,2-dimethyl-4-(1-piperidyl)piperidine-1-carboxylate (XIXa)

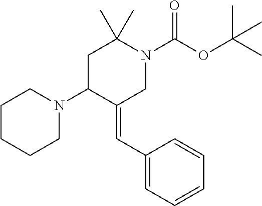 [SMILES: CC(C)(C)OC(=O)N1C/C(=C\c2ccccc2)C(N2CCCCC2)CC1(C)C]

To a solution of XVIIIa (0.100 g, 0.284 mmol) in anhydrous THF (1.0 mL), was added Ti(OEt)₄ (0.146 g, 0.640 mmol) and piperidine (0.146 g, 0.845 mmol) and the reaction mixture was refluxed under Ar for 4 h. The mixture was then allowed to cool to RT and NaBH₃CN (0.054 g, 0.852 mmol) was added and stirring was continued at RT for 72 h. The mixture was quenched with the addition of MeOH, diluted with DCM, washed with saturated aq. NaHCO₃ solution, brine, dried over Na₂SO₄, concentrated and the residue was purified by column chromatography (SiO₂), eluting with DCM/MeOH (95:5) to afford XIXa (0.070 g, 64%). ¹H NMR (400 MHz, CDCl₃) δ 7.35 (t, J=7.6 Hz, 2H), 7.28-7.19 (m, 3H), 6.80 (s, 1H), 5.03 (d, J=15.6 Hz, 1H), 3.69 (dt, J=15.6, 2.1 Hz, 1H), 3.51-3.39 (m, 1H), 2.65 (ddd, J=10.8, 6.7, 3.8 Hz, 2H), 2.46 (s, 2H), 1.88 (t, J=12.8 Hz, 1H), 1.71 (dd, J=13.3, 4.0 Hz, 1H), 1.67-1.53 (m, 4H), 1.51 (s, 3H), 1.50-1.44 (m, 2H), 1.46 (s, 3H), 1.40 (s, 9H). UPLC/MS (method C): Rt 0.89 min. MS (ES), C₂₄H₃₆N₂O₂ requires 384, found 385 [M+H]⁺.

### (5E)-5-Benzylidene-2,2-dimethyl-4-(1-piperidyl)piperidine (XXa)

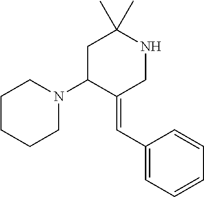 [SMILES: CC1(C)CC(N2CCCCC2)/C(=C/c2ccccc2)CN1]

To a solution of XIXa (0.098 g, 0.255 mmol) in anhydrous DCM (5.9 mL) ZnBr₂ (1.149 g, 5.1 mmol) was added and the reaction mixture was stirred at RT for 16 h. The reaction solution was diluted with DCM, washed with a saturated aq. Na₂CO₃ solution, dried over Na₂SO₄ and concentrated to afford XXa which was used in the next step without further purification. ¹H NMR (400 MHz, CDCl₃) δ 7.36-7.30 (m, 2H), 7.26-7.19 (m, 3H), 6.60 (s, 1H), 3.83 (d, J=15.0 Hz, 1H), 3.51 (dd, J=14.9, 1.5 Hz, 1H), 3.20 (ddd, J=9.8, 4.3, 1.8 Hz, 1H), 2.68 (dt, J=11.5, 4.9 Hz, 2H), 2.50-2.32 (m, 2H), 1.80-1.73 (m, 1H), 1.72-1.54 (m, 5H), 1.53-1.43 (m, 2H), 1.26 (s, 3H), 1.19 (s, 3H). UPLC/MS (method A): Rt 1.93 min. MS (ES), C₁₉H₂₈N₂ requires 284, found 285 [M+H]⁺.

### (5E)-5-Benzylidene-2,2-dimethyl-N-(4-phenylbutyl)-4-(1-piperidyl)piperidine-1-carboxamide

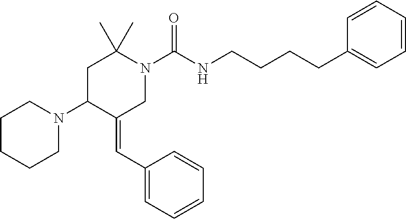 [SMILES: CC1(C)CC(N2CCCCC2)/C(=C/c2ccccc2)CN1C(=O)NCCCCc1ccccc1]

Following general procedure D (Method A), XIXa (0.043 g, 0.15 mmol) and 4-phenylbutyl isocyanate (0.029 g, 0.165 mmol) afforded the title compound (0.048 g, 69%). ¹H NMR (400 MHz, DMSO-d₆) δ 7.34-7.06 (m, 10H), 6.63 (s, 1H), 5.90 (t, J=5.5 Hz, 1H), 4.40 (d, J=14.8 Hz, 1H), 3.77 (J=14.8 Hz, 1H), 3.30 (m, 1H), 3.01 (dq, J=12.9, 6.6 Hz, 1H), 2.88 (dq, J=12.7, 6.6 Hz, 1H), 2.53-2.50 (4H), 2.47-2.38 (m, 2H), 1.87-1.77 (m, 1H), 1.68 (dd, J=13.3, 4.7 Hz, 1H), 1.59-1.40 (m, 8H), 1.42 (s, 3H), 1.38 (s, 3H), 1.37-1.29 (m, 2H). UPLC/MS (method B): Rt 1.10 min. MS (ES), C₃₀H₄₁N₃O requires 459, found 460 [M+H]⁺.

### Example 129: (5E)-5-Benzylidene-2,2-dimethyl-N-(2-phenylethyl)-4-(1-piperidyl)piperidine-1-carboxamide

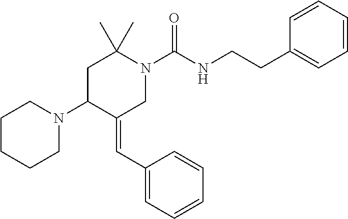 [SMILES: CC1(C)CC(N2CCCCC2)/C(=C/c2ccccc2)CN1C(=O)NCCc1ccccc1]

Following general procedure D (method A), XXa (0.038 g, 0.134 mmol) and phenethyl isocyanate (0.022 g, 0.147 mmol) afforded the title compound (0.025 g, 43%). ¹H NMR (400 MHz, DMSO-d₆) δ 7.37 (t, J=7.5 Hz, 2H), 7.35-7.07 (m, 8H), 6.65 (s, 1H), 5.97 (t, J=5.5 Hz, 1H), 4.43 (d, J=15.2 Hz, 1H), 3.82 (d, J=15.2 Hz, 1H), 3.30 (m, 1H), 3.21-3.05 (m, 2H), 2.63 (t, J=7.5 Hz, 2H), 2.60-2.54 (m, 2H), 2.46-2.36 (m, 2H), 1.79 (t, J=12.6 Hz, 1H), 1.68 (dd, J=13.2, 4.4 Hz, 11H), 1.63-1.46 (m, 4H), 1.40-1.43 (m, 2H), 1.43 (s, 3H), 1.38 (s, 3H). UPLC/MS (method A): Rt 2.06 min. MS (ES), C₂₈H₃₇N₃O requires 431, found 432 [M+H]⁺.

### Example 130: (5E)-5-Benzylidene-2,2-dimethyl-N-pentyl-4-(1-piperidyl)piperidine-1-carboxamide

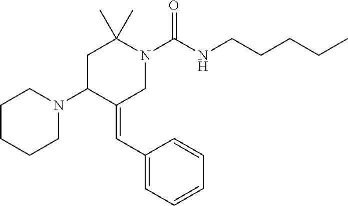 [SMILES: CCCCCNC(=O)N1C/C(=C\c2ccccc2)C(N2CCCCC2)CC1(C)C]

Following general procedure D (method A), XXa (0.028 g, 0.098 mmol) and pentyl isocyanate (0.012 g, 0.108 mmol) afforded the title compound (0.028 g, 31%). ¹H NMR (400 MHz, DMSO-d₆) δ 7.35 (t, J=7.5 Hz, 2H), 7.29-7.19 (m, 3H), 6.64 (s, 1H), 5.85 (t, J=5.4 Hz, 1H), 4.41 (d, J=14.8 Hz, 1H), 3.78 (d, J=14.8 Hz, 1H), 3.29 (m, 1H), 3.00-2.90 (m, 1H), 2.90-2.83 (m, 1H), 2.59-2.49 (m, 2H), 2.46-2.38 (m, 2H), 1.82 (t, J=12.5 Hz, 1H), 1.68 (dd, J=13.3, 4.7 Hz, 1H), 1.60-1.45 (m, 4H), 1.42 (s, 3H), 1.44-1.37 (m, 2H), 1.39 (s, 3H), 1.31 (p, J=7.1 Hz, 2H), 1.22 (p, J=6.7 Hz, 2H), 1.18-1.07 (m, 2H), 0.82 (t, J=7.2 Hz, 3H). UPLC/MS (method A): Rt 2.05 min. MS (ES), C₂₈H₃₇N₃O requires 397, found 398 [M+H]⁺.

### Example 131: 2,2-Dimethyl-N-pentyl-5-phenyl-4-(1-piperidyl)piperidine-1-carboxamide tert-Butyl 2,2-dimethyl-4-oxo-5-phenyl-piperidine-1-carboxylate (XVd)

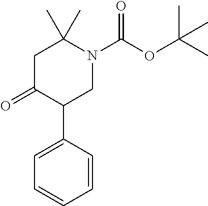 [SMILES: CC(C)(C)OC(=O)N1CC(c2ccccc2)C(=O)CC1(C)C]

To a solution of tBuONa (0.250 g, 1.76 mmol), Pd₂(dba)₃ (0.002 g, 0.0022 mmol), and xantphos (0.003 g, 0.0053 mmol) in anhydrous THF (0.5 mL), bromobenzene (0.058 g, 0.367 mmol) and V-Aa (0.100 g, 0.440 mmol) were sequentially added under argon. The reaction mixture was irradiated at the microwave for 3 h at 100° C. The mixture was then partitioned between H₂O and DCM, extracted with DCM, dried over Na₂SO₄, concentrated and the residue was purified by column chromatography (SiO₂), eluting with Cy/EA (9:1) to afford XVd (0.092 g, 69%). ¹H NMR (400 MHz, CDCl₃) δ 7.38-7.26 (m, 3H), 7.23-7.17 (m, 2H), 4.44 (dd, J=14.1, 5.3 Hz, 1H), 3.85 (dd, J=14.1, 10.4 Hz, 1H), 3.61 (dd, J=10.4, 5.3 Hz, 1H), 3.00 (d, J=14.5 Hz, 1H), 2.52 (d, J=14.5 Hz, 1H), 1.56 (s, 3H), 1.55 (s, 3H), 1.52 (s, 9H). UPLC/MS (method A): Rt 2.48 min. MS (ES), C₁₈H₂₅NO₃ requires 303, found 304 [M+H]⁺.

### tert-Butyl 2,2-dimethyl-5-phenyl-4-(1-piperidyl)piperidine-1-carboxylate (XVIc)

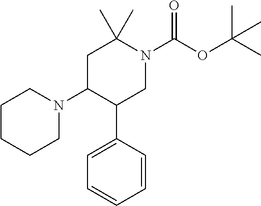 [SMILES: CC(C)(C)OC(=O)N1CC(c2ccccc2)C(N2CCCCC2)CC1(C)C]

Following general procedure I (method B), XVd (0.069 g, 0.227 mmol) and piperidine (0.019 g, 0.227 mmol) afforded XVIc as an oil (0.056 g, 0.145 mmol, 73%). UPLC/MS (method A): Rt 2.21 min. MS (ES), C₂₃H₃₆N₂O₂ requires 372, found 373 [M+H]⁺.

### 2,2-Dimethyl-5-phenyl-4-(1-piperidyl)piperidine dihydrochloride (XVIIc)

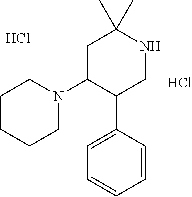 [SMILES: CC1(C)CC(N2CCCCC2)C(c2ccccc2)CN1 ; Cl ; Cl]

Following general procedure C, XVIc (0.024 g, 0.064 mmol) afforded XVIIc as a white solid (0.022 g, quant.). UPLC/MS (method A): Rt 1.18 min. MS (ES), C₁₈H₂₈N₂ requires 272, found 273 [M+H]⁺.

### 2,2-Dimethyl-N-pentyl-5-phenyl-4-(1-piperidyl)piperidine-1-carboxamide

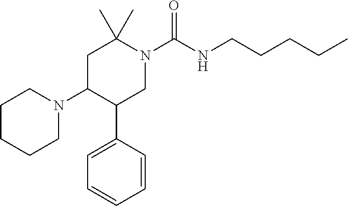 [SMILES: CCCCCNC(=O)N1CC(c2ccccc2)C(N2CCCCC2)CC1(C)C]

Following general procedure D (Method A), XVIIc (0.022 g, 0.064 mmol) and pentyl isocyanate (0.008 g, 0.070 mmol) afforded the title compound as an orange oil (18 mg, 73%). ¹H NMR (400 MHz, DMSO-d₆) δ 7.41-7.10 (m, 5H), 6.43-6.16 (m, 1H), 3.67-3.43 (m, 1H), 3.40-3.24 (m, 2H), 3.03-2.82 (m, 4H), 2.58-2.53 (m, 1H, overlapped with solvent signal), 2.33-2.13 (m, 2H), 1.63-1.52 (m, 2H), 1.49-1.09 (m, 18H), 0.90-0.77 (m, 3H) bsbs. UPLC/MS (method A): Rt 2.06 min. MS (ES), C₂₄H₃₉N₃O requires 385, found 386 [M+H]⁺.

### Example 132: 3,3,5-Trimethyl-N-pentylmorpholine-4-carboxamide

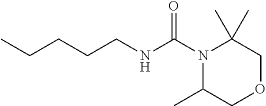 [SMILES: CCCCCNC(=O)N1C(C)COCC1(C)C]

Following general procedure D (method A), 3,3,5-trimethylmorpholine hydrochloride (0.050 g, 0.3 mmol) and n-pentyl isocyanate (0.041 g, 0.36 mmol) afforded the title compound as a colorless oil (0.059 g, 81%). ¹H NMR (400 MHz, CDCl₃) δ 4.73 (bs, 1H), 3.76-3.64 (m, 1H), 3.55 (d, J=4.1 Hz, 1H), 3.52 (dd, J=7.0, 3.2 Hz, 1H), 3.43 (d, J=11.3 Hz, 1H), 3.30-3.15 (m, 3H), 1.51 (p, J=7.3 Hz, 2H), 1.35 (s, 3H), 1.34 (s, 3H), 1.33-1.25 (m, 4H), 1.19 (d, J=6.4 Hz, 3H), 0.90 (t, J=7.0 Hz, 3H). UPLC/MS (method A): Rt 2.03 min. MS (ES) C₁₃H₂₆N₂O₂ requires 242, found 243 [M+H]⁺.

### Example 133: 3,3-Dimethyl-5-phenyl-N-(4-phenylbutyl)morpholine-4-carboxamide tert-Butyl 3,3-dimethyl-5-oxomorpholine-4-carboxylate (XXIIIa)

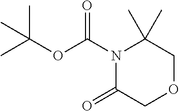 [SMILES: CC(C)(C)OC(=O)N1C(=O)COCC1(C)C]

To a solution of 5,5-dimethylmorpholinone (1.0 g, 7.8 mmol) in anhydrous THF (2.5 mL) nBuLi was added dropwise (3.41 mL, 2.5 M in hexanes) at −78° C. under N₂ atmosphere. After 30 min, a solution of Boc₂O (6.75 g, 30.96 mmol) in anhydrous THF was added at −78° C. The reaction mixture was allowed to warm to RT, diluted with EA, washed with saturated aq. NaHCO₃ solution, brine, dried over Na₂SO₄, concentrated and the residue was purified by column chromatography (SiO₂), eluting with Cy/EA (9:1) to afford XXIIIa as a white solid (1.6 g, 90%). ¹H NMR (400 MHz, CDCl₃) δ 4.20 (s, 2H), 3.58 (s, 2H), 1.54 (s, 9H), 1.44 (s, 6H). UPLC/MS (method A): Rt 1.78 min. MS (ES) C₁₁H₁₉NO₄ requires 229, found 230 [M+H]⁺.

### tert-Butyl N-(1,1-dimethyl-2-phenacyloxyethyl)carbamate (XXIVa)

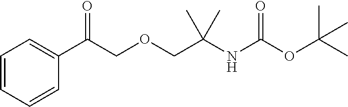 [SMILES: CC(C)(COCC(=O)c1ccccc1)NC(=O)OC(C)(C)C]

Following general procedure H (method A), XXIIIa (0.30 g, 1.31 mmol) and PhMgBr (0.290 g, 1.57 mmol, 2.8 M in THF) afforded compound XXIVa as a transparent oil (0.250 g, 62%). ¹H NMR (400 MHz, CDCl₃) δ 7.94-7.92 (m, 2H), 7.58 (d, J=7.4 Hz, 1H), 7.47 (t, J=7.7 Hz, 2H), 4.78 (s, 2H), 3.52 (s, 2H), 1.44 (s, 9H), 1.33 (s, 6H). UPLC/MS (method A): Rt 2.39 min. MS (ES) C₁₇H₂₅NO₄ requires 307, found 308 [M+H]⁺.

### 3,3-Dimethyl-5-phenylmorpholine (XXVa)

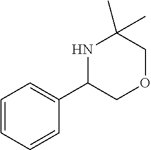

Following general procedure K, XXIVa (0.240 g, 0.78 mmol) and NaBH(OAc)₃ (0.496 g, 2.34 mmol) afforded XXVa. ¹H NMR (400 MHz, CDCl3) δ 7.45-7.37 (m, 2H), 7.35-7.27 (m, 3H), 4.66-4.49 (m, 1H), 4.31 (dd, J=11.1, 3.6 Hz, 1H), 3.94 (dd, J=11.8, 3.7 Hz, 1H), 3.57-3.45 (m, 2H), 1.45 (s, 6H). UPLC/MS (method A): Rt 1.04 min. MS (ES) C₁₂H₁₇NO requires 191, found 192 [M+H]⁺.

### 3,3-Dimethyl-5-phenyl-N-(4-phenylbutyl)morpholine-4-carboxamide

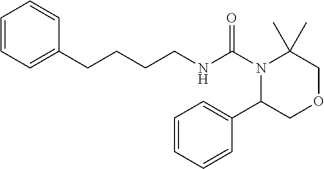 [SMILES: CC1(C)COCC(c2ccccc2)N1C(=O)NCCCCc1ccccc1]

Following general procedure D (method A), XXVa (0.040 g, 0.21 mmol) and 4-phenylbutyl isocyanate (0.018 g, 0.11 mmol) afforded the title compound as a transparent oil (0.013 g, 17%). ¹H NMR (400 MHz, CDCl₃) δ 7.37-7.25 (m, 5H), 7.25-7.14 (m, 3H), 7.09 (d, J=6.9 Hz, 2H), 4.66 (bs, 1H), 4.49 (dd, J=9.2, 4.4 Hz, 1H), 3.97 (dd, J=11.5, 4.5 Hz, 1H), 3.56 (dd, J=11.5, 9.2 Hz, 1H), 3.53-3.43 (m, 2H), 3.16-2.81 (m, 2H), 2.48 (t, J=7.6 Hz, 2H), 1.55-1.52 (m, 2H), 1.46 (s, 3H), 1.40 (s, 3H), 1.39-1.30 (m, 2H), 1.27-1.15 (m, 2H). UPLC/MS (method A): Rt 2.49 min. MS (ES) C₂₃H₃₀N₂O₂ requires 366, found 367 [M+H]⁺.

### Example 134: N-Heptyl-3,3-dimethyl-5-phenylmorpholine-4-carboxamide

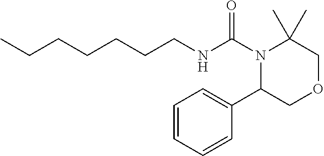 [SMILES: CCCCCCCNC(=O)N1C(c2ccccc2)COCC1(C)C]

Following general procedure D (method A), XXVa (0.040 g, 0.21 mmol) and heptyl isocyanate (0.060 g, 0.24 mmol) afforded the title compound as a transparent oil (0.013 g, 19%). ¹H NMR (400 MHz, CDCl₃) δ 7.39-7.28 (m, 4H), 7.29-7.23 (m, 1H), 4.67 (bs, 1H), 4.50 (dd, J=9.2, 4.4 Hz, 1H), 3.97 (dd, J=11.5, 4.4 Hz, 1H), 3.56 (dd, J=11.5, 9.2 Hz, 1H), 3.53-3.42 (m, 2H), 2.99 (dh, J=18.9, 6.6, 6.1 Hz, 2H), 1.46 (s, 3H), 1.41 (s, 3H), 1.32-0.93 (m, 11H), 0.86 (t, J=7.1 Hz, 3H). UPLC/MS (method A): Rt 2.62 min. MS (ES) C₂₀H32N₂O₂ requires 332, found 333 [M+H]⁺.

### Example 135: N-Cyclohexyl-3,3-dimethyl-5-phenylmorpholine-4-carboxamide

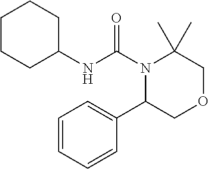 [SMILES: CC1(C)COCC(c2ccccc2)N1C(=O)NC1CCCCC1]

Following general procedure D (method A), XXVa (0.040 g, 0.21 mmol) and cyclohexyl isocyanate (0.157 g, 1.26 mmol) afforded the title compound as a white solid (0.02 g, 30%). ¹H NMR (400 MHz, CDCl₃) δ 7.41-7.23 (m, 5H), 4.71-4.58 (m, 1H), 4.47 (dd, J=9.6, 4.4 Hz, 1H), 3.94 (dd, J=11.5, 4.4 Hz, 1H), 3.52 (dd, J=11.4, 9.8 Hz, 1H), 3.48 (s, 2H), 3.46-3.34 (m, 1H), 1.73-1.58 (m, 2H), 1.49 (d, J=7.9 Hz, 2H), 1.45 (s, 3H), 1.40 (s, 3H), 1.25 (dd, J=24.4, 13.6 Hz, 4H), 0.92-0.68 (m, 2H). UPLC/MS (method A): Rt 2.36 min. MS (ES) C₁₉H₂₈N₂O₂ requires 316, found 317 [M+H]⁺.

### Example 136: 5-(4-Fluorophenyl)-3,3-dimethyl-N-(4-phenylbutyl)morpholine-4-carboxamide

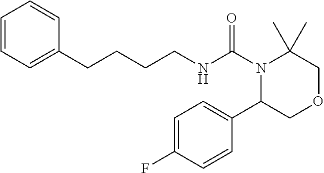 [SMILES: CC1(C)COCC(c2ccc(F)cc2)N1C(=O)NCCCCc1ccccc1]

Following general procedure D (method A), XXVb (0.048 g, 0.23 mmol) and 4-phenylbutyl isocyanate (0.048 g, 0.28 mmol) afforded the title compound as a transparent oil (0.020 g, 17%). ¹H NMR (400 MHz, DMSO-d₆) δ 7.37 (dd, J=8.7, 5.6 Hz, 2H), 7.25 (t, J=7.4 Hz, 2H), 7.19-7.00 (m, 5H), 6.96 (t, J=5.8 Hz, 1H), 4.45 (dd, J=9.2, 4.0 Hz, 1H), 3.75 (dd, J=11.2, 4.0 Hz, 1H), 3.46-3.33 (m, 4H), 2.86 (dp, J=19.7, 6.4 Hz, 2H), 2.44 (t, J=7.6 Hz, 2H), 1.33 (q, J=7.6 Hz, 2H), 1.25-1.15 (m, 7H). UPLC/MS (method A): Rt 2.51 min. MS (ES) C₂₃H₂₉FN₂O₂ requires 384, found 385 [M+H]⁺.

### Example 137: 5-(4-Fluorophenyl)-3,3-dimethyl-N-pentylmorpholine-4-carboxamide tert-Butyl N-[2-[2-(4-fluorophenyl)-2-oxoethoxy]-1,1-dimethylethyl]carbamate (XXIVb)

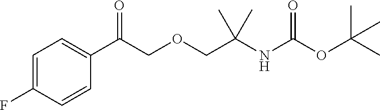 [SMILES: CC(C)(COCC(=O)c1ccc(F)cc1)NC(=O)OC(C)(C)C]

Following general procedure H (method A), XXIIa (0.230 g, 1.0 mmol) and 4-fluorophenylmagnesium bromide (1.0M in THF, 0.330 g, 1.50 mmol) afforded compound XXIVb as transparent oil (0.150 g, 49%). ¹H NMR (400 MHz, CDCl₃) δ 8.01-7.89 (m, 2H), 7.14 (t, J=8.7 Hz, 2H), 4.73 (s, 2H), 3.52 (s, 2H), 1.44 (s, 9H), 1.32 (s, 6H). UPLC/MS (method A): Rt 2.41 min. MS (ES) C₁₇H₂₄NFO₄ requires 325, found 326 [M+H]⁺.

### 5-(4-Fluorocyclohexa-2,4-dien-1-yl)-3,3-dimethylmorpholine 2,2,2-trifluoroacetic acid (XXVb)

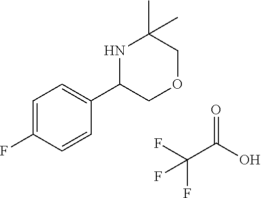

Following general procedure K, XXIVb (0.150 g, 0.46 mmol) afforded XXVb which was used in the next step without further purification. ¹H NMR (600 MHz, DMSO-d₆) δ 7.44 (dd, J=8.5, 5.8 Hz, 2H), 7.12 (t, J=8.9 Hz, 2H), 4.10 (dd, J=10.6, 3.4 Hz, 1H), 3.72 (dd, J=10.6, 3.5 Hz, JH), 3.42 (d, J=10.6 Hz, 1H), 3.13 (d, J=10.6 Hz, 1H), 2.96 (t, J=10.6 Hz, 1H), 1.24 (s, 3H), 0.99 (s, 3H). UPLC/MS (method A): Rt 1.13 min. MS (ES) C₁₂H₁₆FNO requires 209, found 210 [M+H]⁺.

### 5-(4-Fluorophenyl)-3,3-dimethyl-N-pentylmorpholine-4-carboxamide

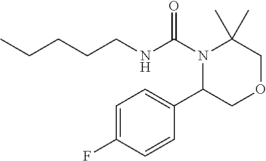 [SMILES: CCCCCNC(=O)N1C(c2ccc(F)cc2)COCC1(C)C]

Following general procedure D (method A), XXVb (0.048 g, 0.23 mmol) and n-pentyl isocyanate (0.031 g, 0.28 mmol) afforded the title compound as white solid (0.040 g, 54%). ¹H NMR (400 MHz, DMSO-d₆) δ 7.43-7.29 (m, 2H), 7.14-7.00 (m, 2H), 6.92 (t, J=5.7 Hz, 1H), 4.44 (dd, J=9.3, 4.0 Hz, 1H), 3.75 (dd, J=11.2, 4.0 Hz, 1H), 3.47-3.33 (m, 3H), 2.82 (qq, J=13.1, 6.7 Hz, 2H), 1.27-1.07 (m, 10H), 1.03-0.91 (m, 2H), 0.77 (t, J=7.3 Hz, 3H). UPLC/MS (method A): Rt 2.34 min. MS (ES) C₁₈H₂₇FN₂O₂ requires 322, found 323 [M+H]⁺. The title compound (104 mg, 0.32 mmol) was subjected to chiral HPLC separation, using a Daicel ChiralPak AD column (250×4.6 mm ID, particle size 10 μM) and as mobile phase a mixture Heptane/2-Propanol (98:2) to afford the two enantiomers.

### Example 138: (5S) or (5R)-5-(4-Fluorophenyl)-3,3-dimethyl-N-pentylmorpholine-4-carboxamide

First eluted enantiomer (16.97 min), 0.022 g. ¹H NMR (400 MHz, DMSO-d₆) δ 7.38 (dd, J=8.6, 5.7 Hz, 2H), 7.08 (t, J=8.9 Hz, 2H), 6.92 (t, J=5.7 Hz, 1H), 4.44 (dd, J=9.3, 4.0 Hz, 1H), 3.75 (dd, J=11.3, 4.0 Hz, 1H), 3.45-3.32 (m, 3H), 2.82 (ddt, J=19.3, 13.1, 6.3 Hz, 2H), 1.23 (s, 3H), 1.21 (s, 3H), 1.19-1.07 (m, 4H), 1.04-0.90 (m, 2H), 0.77 (t, J=7.3 Hz, 3H). UPLC/MS (method A): Rt 2.34 min. MS (ES) C₁₈H₂₇FN₂O₂ requires 322, found 323 [M+H]⁺. [ [symbol] ]²⁷_{D}+12.030 (c 1.0, CHCl₃). 98.2% ee.

### Example 139: (5S) or (5R)-5-(4-fluorophenyl)-3,3-dimethyl-N-pentylmorpholine-4-carboxamide

Second eluted enantiomer (29.45 min), 0.016 g. ¹H NMR (400 MHz, DMSO-d₆) δ 7.38 (dd, J=8.6, 5.7 Hz, 2H), 7.08 (t, J=8.9 Hz, 2H), 6.92 (t, J=5.7 Hz, 1H), 4.44 (dd, J=9.3, 4.0, 1H), 3.75 (dd, J=11.3, 4.0 Hz, 1H), 3.45-3.32 (m, 3H), 2.82 (ddt, J=19.3, 13.1, 6.3 Hz, 2H), 1.23 (s, 3H), 1.21 (s, 3H), 1.19-1.07 (m, 4H), 1.04-0.90 (m, 2H), 0.77 (t, J=7.3 Hz, 3H). UPLC/MS (method A): Rt 2.34 min. MS (ES) C₁₈H₂₇FN₂O₂ requires 322, found 323 [M+H]⁺. [ [symbol] ]²⁷_{D} −32.34° (c 1.0, CHCl₃). 81.7% ee.

### Example 140: 5-(4-Fluorophenyl)-N-isobutyl-3,3-dimethylmorpholine-4-carboxamide

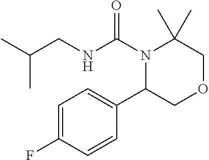 [SMILES: CC(C)CNC(=O)N1C(c2ccc(F)cc2)COCC1(C)C]

Following general procedure D (method B), XXVb (0.096 g, 0.3 mmol) and isobutylamine (0.066 g, 0.9 mmol) afforded the title compound as a colorless oil (0.010 g, 11%). ¹H NMR (400 MHz, CDCl₃) δ 7.33 (dd, J=8.6, 5.4 Hz, 2H), 6.99 (t, J=8.6 Hz, 2H), 4.85 (bs, 1H), 4.51 (dd, J=9.6, 4.3 Hz, 1H), 3.91 (dd, J=11.6, 4.3 Hz, 1H), 3.52-3.40 (m, 3H), 2.84 (td, J=6.4, 2.4 Hz, 2H), 1.50 (m, 1H), 1.43 (s, 3H), 1.35 (s, 3H), 0.69 (dd, J=16.4, 6.7 Hz, 6H). UPLC/MS (method A): Rt 2.21 min. MS (ES) C₁₇H₂₅FN₂O₂ requires 308, found 309 [M+H]⁺.

### Example 141: (3R,5S)-3-(4-Fluorophenyl)-5-methyl-N-pentylmorpholine-4-carboxamide tert-Butyl N-[(1S)-2-[2-(4-fluorophenyl)-2-oxoethoxy]-1-methylethyl]carbamate (XXIVd)

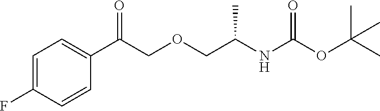 [SMILES: C[C@@H](COCC(=O)c1ccc(F)cc1)NC(=O)OC(C)(C)C]

Following general procedure H, tert-butyl (3S)-3-methyl-5-oxo-morpholine-4-carboxylate (0.430 g, 2.00 mmol) and 4-fluorophenylmagnesium bromide (0.5 M in THF) (0.44 g, 2.20 mmol) afforded XXIVd as a colorless oil (0.407 g, 65%). ¹H NMR (400 MHz, CDCl₃) δ 8.06-7.87 (m, 2H), 7.24-7.11 (m, 2H), 4.95 (s, 1H), 4.73 (d, J=3.7 Hz, 2H), 4.04-3.73 (m, 1H), 3.55 (d, J=4.7 Hz, 2H), 1.46 (s, 9H), 1.22 (d, J=6.7 Hz, 3H). UPLC/MS (method A): Rt 2.22 min. MS (ES) C₁₆H₂₂FNO₄ requires 311, found 312 [M+H]⁺.

### (3R,5S)-3-(4-Fluorophenyl)-5-methyl-morpholine (XXVc)

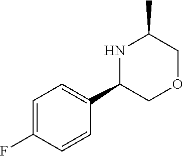

Following general procedure K, XXIVd (0.400 g, 1.28 mmol) afforded XXVc which was used in the next step without further purification. ¹H NMR (400 MHz, DMSO-d₆) δ 7.50-7.40 (m, 2H), 7.21-7.09 (m, 2H), 3.88 (dd, J=10.3, 3.2 Hz, 1H), 3.73-3.65 (m, 2H), 3.16-2.82 (m, 4H), 0.94 (d, J=5.9 Hz, 3H). UPLC/MS (method A): Rt 1.06 min. MS (ES) C₁₁H₁₄FNO requires 195, found 196 [M+H]⁺.

### (3R,5S)-3-(4-Fluorophenyl)-5-methyl-N-pentylmorpholine-4-carboxamide

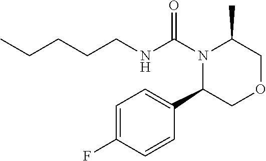 [SMILES: CCCCCNC(=O)N1[C@@H](C)COC[C@H]1c1ccc(F)cc1]

Following general procedure D (Method A), XXVc (0.060 g, 0.31 mmol) and pentylisocyanate (0.039 g, 0.34 mmol) afforded the title compound as a white solid (0.055 g, 57%). ¹H NMR (400 MHz, DMSO-d₆) δ 7.62-7.44 (m, 2H), 7.20-7.05 (m, 2H), 6.49 (t, J=5.4 Hz, 1H), 5.19-5.07 (m, 1H), 4.51 (d, J=11.8 Hz, 1H), 4.04-3.86 (m, 1H), 3.78-3.53 (m, 3H), 3.20-2.99 (m, 2H), 1.57-1.38 (m, 2H), 1.36-1.18 (m, 4H), 0.87 (t, J=7.1 Hz, 3H), 0.78 (d, J=6.9 Hz, 3H). UPLC/MS (method A): Rt 2.21 min. MS (ES) C₁₇H₂₅FN₂O₂ requires 308, found 309 [M+H]⁺. [α]²⁷_{D}=−110° (c 1.0, CHCl₃).

### Example 142: (3S,5R)-3-(4-Fluorophenyl)-5-methyl-N-pentylmorpholine-4-carboxamide tert-Butyl N-[(1R)-2-[2-(4-fluorophenyl)-2-oxoethoxy]-1-methylethyl]carbamate (XXIVc)

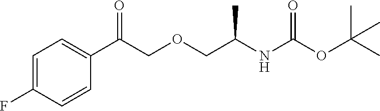 [SMILES: C[C@H](COCC(=O)c1ccc(F)cc1)NC(=O)OC(C)(C)C]

Following general procedure H, tert-butyl (3R)-3-methyl-5-oxo-morpholine-4-carboxylate (0.290 g, 1.35 mmol) and 4-fluorophenylmagnesium bromide (0.5 M in THF) (0.295 g, 1.48 mmol) afforded XXIVc as a colorless oil (0.338 g, 80%). ¹H NMR (400 MHz, CDCl₃) δ 8.08-7.94 (m, 2H), 7.23-7.09 (m, 2H), 4.94 (s, 1H), 4.73 (d, J=3.8 Hz, 2H), 4.06-3.76 (m, 1H), 3.55 (d, J=4.6 Hz, 2H), 1.46 (s, 9H), 1.22 (d, J=6.7 Hz, 3H). UPLC/MS (method A): Rt 2.22 min. MS (ES) C₁₆H₂₂FNO₄ requires 311, found 312 [M+H]⁺.

### (3S,5R)-3-(4-Fluorophenyl)-5-methylmorpholine (XXVd)

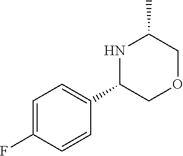

Following general procedure K, XXIVc (0.335 g, 1.08 mmol) afforded XXVd which was used in the next step without further purification. ¹H NMR (400 MHz, CDCl₃) δ 7.50-7.33 (m, 2H), 7.15-6.91 (m, 2H), 4.03 (dd, J=10.4, 3.3 Hz, 1H), 3.83 (dt, J=10.7, 2.7 Hz, 2H), 3.39 (t, J=10.8 Hz, 1H), 3.33-3.10 (m, 2H), 1.04 (d, J=6.2 Hz, 3H). UPLC/MS (method A): Rt 1.06 min. MS (ES) C₁₁H₁₄FNO requires 195, found 196 [M+H]⁺.

### (3S,5R)-3-(4-Fluorophenyl)-5-methyl-N-pentylmorpholine-4-carboxamide

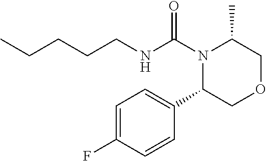 [SMILES: CCCCCNC(=O)N1[C@H](C)COC[C@@H]1c1ccc(F)cc1]

Following general procedure D (Method A), XXVd (0.060 g, 0.31 mmol) and pentylisocyanate (0.039 g, 0.34 mmol) afforded the title compound as a white solid (0.068 g, 71%). ¹H NMR (400 MHz, DMSO-d₆) δ 7.58-7.43 (m, 2H), 7.18-7.07 (m, 2H), 6.49 (t, J=5.5 Hz, 1H), 5.14 (d, J=3.6 Hz, 1H), 4.67-4.16 (m, 1H), 4.00-3.82 (m, 1H), 3.73-3.50 (m, 3H), 3.09 (td, J=7.1, 5.4 Hz, 2H), 1.58-1.36 (m, 2H), 1.35-1.15 (m, 4H), 0.87 (t, J=7.1 Hz, 3H), 0.78 (d, J=6.9 Hz, 3H). UPLC/MS (method A): Rt 2.21 min. MS (ES) C₁₇H₂₅FN₂O₂ requires 308, found 309 [M+H]⁺. [α]²⁷_{D}=+90° (c 1.0, CHCl₃).

### Example 143: N-iso-Butyl-5-(4-methoxyphenyl)-3,3-dimethylmorpholine-4-carboxamide

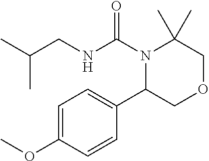 [SMILES: COc1ccc(C2COCC(C)(C)N2C(=O)NCC(C)C)cc1]

Following general procedure D (method B), XXVe (0.158 g, 0.47 mmol) and isobutylamine (0.103 g, 0.41 mmol) afforded the title compound as a colorless oil (0.050 g, 31%). ¹H NMR (400 MHz, CDCl₃) δ 7.31-7.24 (m, 2H), 6.85 (d, J=8.7 Hz, 2H), 4.78 (bs, 1H), 4.45 (dd, J=9.2, 4.4 Hz, 1H), 3.94 (dd, J=11.6, 4.5 Hz, 1H), 3.78 (s, 3H), 3.58-3.52 (m, 1H), 3.51-3.44 (m, 2H), 2.87-2.73 (m, 2H), 1.53-1.45 (m, 1H), 1.48 (s, 3H), 1.43 (s, 3H), 0.67 (dd, J=10.1, 6.7 Hz, 6H). UPLC/MS (method A): Rt 2.13 min. MS (ES) C₁₈H₂₈N₂O₃ requires 320, found 321 [MH]⁺.

### Example 144: N-(Cyclopropylmethyl)-5-(4-methoxyphenyl)-3,3-dimethylmorpholine-4-carboxamide

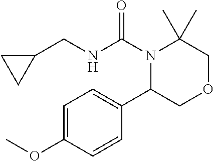 [SMILES: COc1ccc(C2COCC(C)(C)N2C(=O)NCC2CC2)cc1]

Following general procedure D (method A), XXVe (0.070 g, 0.20 mmol) and (isocyanatomethyl)cyclopropane (0.023 g, 0.24 mmol) afford the titled compound as a white solid (0.020 g, 33%). ¹H NMR (400 MHz, CDCl₃) δ 7.33-7.24 (m, 2H), 6.89-6.83 (m, 2H), 4.83 (s, 1H), 4.46 (dd, J=9.3, 4.4 Hz, 1H), 3.93 (dd, J=11.5, 4.4 Hz, 1H), 3.78 (s, 3H), 3.57-3.51 (m, 1H), 3.51-3.42 (m, 2H), 2.86 (ddd, J=9.6, 7.1, 5.5 Hz, 2H), 1.45 (s, 3H), 1.41 (s, 3H), 0.66 (ddt, J=10.3, 7.4, 3.7 Hz, 1H), 0.31 (d, J=7.2 Hz, 2H), 0.21-0.04 (m, 2H). UPLC/MS (method A): Rt 2.01 min. MS (ES) C₁₈H₂₆N₂O₃ requires 318, found 319 [M+H]⁺.

### Example 145: 5-(4-Methoxyphenyl)-3,3-dimethyl-N-(tetrahydropyran-4-ylmethyl)morpholine-4-carboxamide

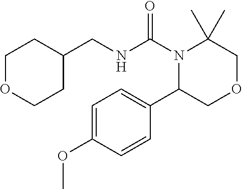 [SMILES: COc1ccc(C2COCC(C)(C)N2C(=O)NCC2CCOCC2)cc1]

Following general procedure D (method B), XXVe (0.100 g, 0.45 mmol) and 4-aminomethyltetrahydropyran (0.156 g, 1.35 mmol) afforded the title compound as a white solid (0.050 g, 31%). ¹H NMR (400 MHz, CDCl₃) δ 7.35-7.15 (m, 2H), 6.91-6.75 (m, 2H), 4.84 (bs, 1H), 4.42 (dd, J=9.5, 4.5 Hz, 1H), 3.93 (dd, J=11.6, 4.5 Hz, 1H), 3.78 (s, 5H), 3.51 (dd, J=11.5, 9.5 Hz, 1H), 3.48 (s, 2H), 3.18 (tt, J=11.6, 2.6 Hz, 2H), 3.03-2.68 (m, 2H), 1.45 (s, 3H), 1.39 (s, 3H), 1.28-1.19 (m, 2H), 1.15-0.91 (m, 3H). UPLC/MS (method A): Rt 1.75 min. MS (ES) C₂₀H₃₀N₂O₄ requires 362, found 363 [M+H]⁺.

### Example 146: N-(2-Cyclopropylethyl)-5-(4-methoxyphenyl)-3,3-dimethylmorpholine-4-carboxamide

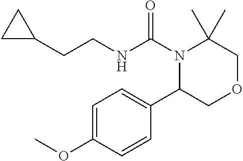 [SMILES: COc1ccc(C2COCC(C)(C)N2C(=O)NCCC2CC2)cc1]

Following general procedure D (method B), XXVe (0.100 g, 0.45 mmol) and 2-cyclopropylethanamine hydrochloride (0.165 g, 1.36 mmol) afforded the title compound as a white solid (0.087 g, 58%). ¹H NMR (400 MHz, CDCl₃) δ 7.30 (d, J=9.2 Hz, 2H), 6.87 (d, J=8.7 Hz, 2H), 4.83 (s, 1H), 4.49 (dd, J=8.8, 4.3 Hz, 1H), 4.05-3.90 (m, 1H), 3.80 (s, 3H), 3.60 (dd, J=11.5, 8.8 Hz, 1H), 3.56-3.43 (m, 2H), 3.14-3.07 (m, 2H), 1.49 (s, 3H), 1.42 (s, 3H), 1.21-1.09 (m, 2H), 0.46-0.27 (m, 3H), 0.08-0.03 (m, 2H). UPLC/MS (method A): Rt 2.14 min. MS (ES), C₁₉H₂₈N₂O₃ requires 332, found 333 [M+H]⁺.

### Example 147: N-iso-Pentyl-5-(4-methoxyphenyl)-3,3-dimethylmorpholine-4-carboxamide

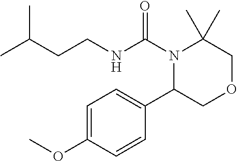 [SMILES: COc1ccc(C2COCC(C)(C)N2C(=O)NCCC(C)C)cc1]

Following general procedure D (method B), XXVe (0.069 g, 0.310 mmol) and isopentylamine (0.082 g, 0.939 mmol) afforded the title compound as a clear oil (0.058 g, 56%). ¹H NMR (400 MHz, CDCl₃) δ 7.29 (d, J=8.7 Hz, 2H), 6.88 (d, J=8.7 Hz, 2H), 4.69 (t, J=4.7 Hz, 1H), 4.46 (dd, J=9.3, 4.4 Hz, 1H), 3.95 (dd, J=11.4, 4.4 Hz, 1H), 3.81 (s, 3H), 3.60-3.44 (m, 3H), 3.11-2.96 (m, 2H), 1.48 (s, 3H), 1.41 (s, 3H), 1.31-1.21 (m, 1H), 1.15-1.03 (m, 2H), 0.78 (dd, J=6.6, 3.5 Hz, 6H). UPLC/MS (method A): Rt 2.27 min. MS (ES), C₁₉H₃₀N₂O₃ requires 334, found 335 [M+H]⁺.

### Example 148: N-[2-(4-Fluorophenyl)ethyl]-5-(4-methoxyphenyl)-3,3-dimethylmorpholine-4-carboxamide

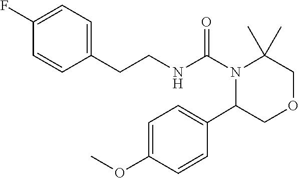 [SMILES: COc1ccc(C2COCC(C)(C)N2C(=O)NCCc2ccc(F)cc2)cc1]

Following general procedure D (method B), XXVe (0.039 g, 0.176 mmol) and 4-fluorophenethylamine (0.074 g, 0.533 mmol) afforded the title compound as a white solid (0.045 g, 66%). ¹H NMR (400 MHz, CDCl₃) δ 7.24-7.16 (m, 2H), 7.03-6.89 (m, 4H), 6.87-6.79 (m, 2H), 4.63 (t, J=5.2 Hz, 1H), 4.43 (dd, J=8.4, 4.4 Hz, 1H), 4.05-3.93 (m, 1H), 3.82 (s, 3H), 3.60 (dd, J=11.5, 8.4 Hz, 1H), 3.56-3.40 (m, 2H), 3.34-3.25 (m, 2H), 2.62-2-54 (m, 2H), 1.48 (s, 3H), 1.37 (s, 3H). UPLC/MS (method A): Rt 2.29 min. MS (ES), C₂₂H₂₇FN₂O₃ requires 386, found 387 [M+H]⁺, 385 [M−H]⁻.

### Example 149: N-(4-Cyclopropylbutyl)-5-(4-methoxyphenyl)-3,3-dimethylmorpholine-4-carboxamide

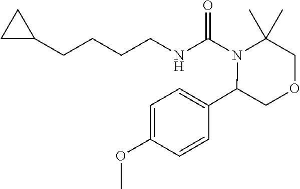 [SMILES: COc1ccc(C2COCC(C)(C)N2C(=O)NCCCCC2CC2)cc1]

Following general procedure D (method B), XXVe (0.100 g, 0.45 mmol) and 4-cyclopropylbutan-1-amine (0.202 g, 0.45 mmol) afforded the title compound as a white solid (0.020 g, 12%). ¹H NMR (400 MHz, CDCl₃) δ 7.31-7.24 (m, 2H), 6.91-6.74 (m, 2H), 4.72 (bs, 1H), 4.44 (dd, J=9.2, 4.4 Hz, 1H), 3.98-3.89 (m, 1H), 3.78 (s, 3H), 3.57-3.43 (m, 3H), 3.09-2.82 (m, 2H), 1.45 (s, 3H), 1.39 (s, 3H), 1.30-0.99 (m, 6H), 0.69-0.46 (m, 1H), 0.44-0.27 (m, 2H), 0.18-0.02 (m, 2H). UPLC/MS (method A): Rt 1.75 min. MS (ES) C₂₁H₃₂N₂O₃ requires 360, found 361 [M+H]⁺.

### Example 150: N-(Cyclohexylmethyl)-5-(4-methoxyphenyl)-3,3-dimethylmorpholine-4-carboxamide

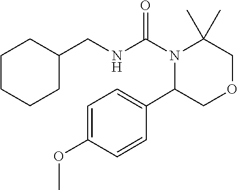 [SMILES: COc1ccc(C2COCC(C)(C)N2C(=O)NCC2CCCCC2)cc1]

Following general procedure D (method B), XXVe (0.060 g, 0.27 mmol) and cyclohexanemethylamine (0.092 g, 0.81 mmol) afforded the title compound as a white solid (0.020 g, 22%). ¹H NMR (400 MHz, CDCl₃) δ 7.28 (d, J=8.7 Hz, 2H), 6.89-6.69 (m, 2H), 4.79 (bs, 1H), 4.42 (dd, J=9.5, 4.4 Hz, 1H), 3.92 (dd, J=11.5, 4.4 Hz, 1H), 3.78 (s, 3H), 3.61-3.34 (m, 3H), 2.84 (t, J=6.2 Hz, 2H), 1.63-1.51 (m, 1H), 1.44 (s, 3H), 1.41-1.19 (m, 7H), 1.18-0.91 (m, 4H), 0.63 (q, J=12.6, 12.0 Hz, 2H). UPLC/MS (method A): Rt 2.45 min. MS (ES) C₂₁H₃₂N₂O₃ requires 360, found 361 [M+H]⁺.

### Example 151: N-[(4-Fluorophenyl)methyl]-5-(4-methoxyphenyl)-3,3-dimethylmorpholine-4-carboxamide

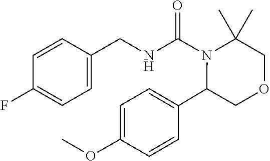 [SMILES: COc1ccc(C2COCC(C)(C)N2C(=O)NCc2ccc(F)cc2)cc1]

Following general procedure D (method B), XXVe (0.069 g, 0.310 mmol) and 4-fluorobenzylamine (0.118 g, 0.939 mmol) afforded the title compound as a white solid (0.046 g, 40%). ¹H NMR (400 MHz, CDCl₃) δ 7.28 (m, 2H), 6.94-6.78 (m, 6H), 5.04 (t, J=4.9 Hz, 1H), 4.54-4.42 (m, 1H), 4.24 (dd, J=14.8, 5.9 Hz, 1H), 4.13 (dd, J=14.7, 5.2 Hz, 1H), 3.96 (dd, J=11.6, 4.4 Hz, 1H), 3.82 (s, 3H), 3.60-3.48 (m, 3H), 1.49 (s, 3H), 1.42 (s, 3H). UPLC/MS (method A): Rt 2.18 min. MS (ES), C₂₁H₂₅FN₂O₃ requires 372, found 373 [M+H]⁺, 371 [M−H]⁻.

### Example 152: 5-(4-Methoxyphenyl)-3,3-dimethyl-N-(2,2,2-trifluoroethyl)morpholine-4-carboxamide

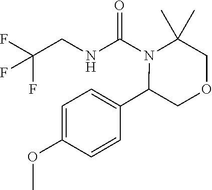 [SMILES: COc1ccc(C2COCC(C)(C)N2C(=O)NCC(F)(F)F)cc1]

Following general procedure D (method B), XXVe (0.05 g, 0.23 mmol) and 2,2,2-trifluoroethylamine hydrochloride (0.094 g, 0.7 mmol) afforded the title compound as a colorless oil (0.010 g, 13%). ¹H NMR (400 MHz, CDCl₃) δ 7.30-7.22 (m, 2H), 6.89 (d, J=8.6 Hz, 2H), 4.86 (t, J=5.2 Hz, 1H), 4.53 (dd, J=8.1, 4.6 Hz, 1H), 4.03 (dd, J=11.7, 4.5 Hz, 1H), 3.93-3.61 (m, 6H), 3.58-3.36 (m, 2H), 1.49 (s, 3H), 1.47 (s, 3H). UPLC/MS (method A): Rt 2.03 min. MS (ES) C₁₆H₂₁F₃N₂O₃ requires 346, found 347 [M+H]⁺.

### Example 153: 5-(4-Methoxyphenyl)-3,3-dimethyl-N-(3,3,3-trifluoropropyl)morpholine-4-carboxamide

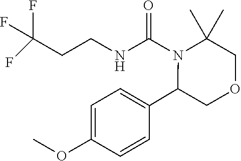 [SMILES: COc1ccc(C2COCC(C)(C)N2C(=O)NCCC(F)(F)F)cc1]

Following general procedure D (method B), XXVe (0.069 g, 0.310 mmol) and 4-fluorobenzylamine (0.118 g, 0.939 mmol) afforded the title compound as a transparent oil (0.022 g, 39%). ¹H NMR (400 MHz, CDCl₃) δ 7.32-7.24 (m, 2H), 6.89 (d, J=8.7 Hz, 2H), 4.90 (t, J=6.0 Hz, 1H), 4.49 (dd, J=8.5, 4.5 Hz, 1H), 4.01 (dd, J=11.6, 4.5 Hz, 1H), 3.81 (s, 3H), 3.63 (dd, J=11.5, 8.5 Hz, 1H), 3.57-3.41 (m, 2H), 3.39-3.13 (m, 2H), 2.18-1.95 (m, 2H), 1.50 (s, 3H), 1.43 (s, 3H). UPLC/MS (method A): Rt 2.09 min. MS (ES), C₂₁H₂₅FN₂O₃ requires 360, found 361 [M+H]⁺.

### Example 154: N-(2-Fluoroethyl)-5-(4-methoxyphenyl)-3,3-dimethylmorpholine-4-carboxamide

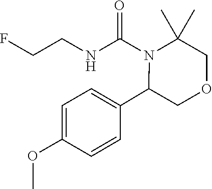 [SMILES: COc1ccc(C2COCC(C)(C)N2C(=O)NCCF)cc1]

Following general procedure D (method B), XXVe (0.05 g, 0.23 mmol) and 2-fluoroethylamine hydrochloride (0.07 g, 0.7 mmol) afforded the title compound as a colorless oil (0.030 g, 36%). ¹H NMR (400 MHz, CDCl₃) δ 7.38-7.10 (m, 2H), 6.87 (d, J=8.7 Hz, 2H), 5.04 (t, J=4.8 Hz, 1H), 4.51 (dd, J=8.6, 4.4 Hz, 1H), 4.38-4.12 (m, 2H), 3.98 (dd, J=11.4, 4.3 Hz, 1H), 3.79 (s, 3H), 3.61 (dd, J=11.5, 8.6 Hz, 1H), 3.52 (d, J=11.5 Hz, 1H), 3.45 (d, J=11.3 Hz, 1H), 3.40-3.33 (m, 1H), 3.34-3.25 (m, 1H), 1.47 (s, 3H), 1.42 (s, 3H). UPLC/MS (method A): Rt 1.75 min. MS (ES) C₁₆H₂₃FN₂O₃ requires 310, found 361 [M+H]⁺.

### Example 155: (5R) AND (5S)-5-(4-Methoxyphenyl)-3,3-dimethyl-N-[(1S)-1-methylpropyl]morpholine-4-carboxamide

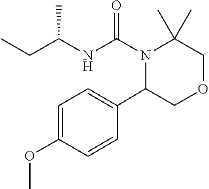 [SMILES: CC[C@H](C)NC(=O)N1C(c2ccc(OC)cc2)COCC1(C)C]

Following general procedure D (method B), XXVe (0.034 g, 0.155 mmol) and (S)-(+)-2-aminobutane (0.034 g, 0.470 mmol) afforded the title compound as a white solid (0.009 g, 18%). ¹H NMR (400 MHz, CDCl₃) δ 7.30 (d, J=8.9 Hz, 2H), 6.91-6.83 (m, 2H), 4.62 (d, J=8.3 Hz, 1H), 4.47-4.40 (m, 1H), 3.95-3.89 (m, 1H), 3.80 (s, 3H), 3.60-3.52 (m, 2H), 3.52-3.50 (m, 2H), 1.50-1.43 (m, 3H), 1.38 (s, 3H), 1.30-1.16 (m, 2H), 0.90-0.85 (m, 3H), 0.82-0.78 (m, 3H), 0.75-0.63 (m, 3H). UPLC/MS (method A): Rt 2.12 min. MS (ES), C₁₈H₂₈N₂O₃ requires 320, found 321 [M+H]⁺.

### Example 156: 5-(4-Methoxyphenyl)-3,3-dimethyl-N-pentylmorpholine-4-carboxamide tert-Butyl N-[2-[2-(4-methoxyphenyl)-2-oxoethoxy]-1,1-dimethylethyl]carbamate (XXIVe)

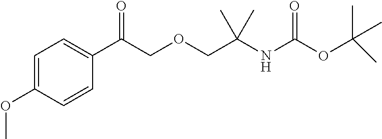 [SMILES: COc1ccc(C(=O)COCC(C)(C)NC(=O)OC(C)(C)C)cc1]

Following general procedure H (method A), XXIIIa (0.230 g, 1.0 mmol) and 4-methoxyphenylmagnesium bromide (0.230 g, 1.10 mmol, 0.5 M in THF) afforded XXIVe as a transparent oil (0.190 g, 56%). ¹H NMR (400 MHz, CDCl₃) δ 8.18-7.73 (m, 2H), 6.94 (d, J=8.9 Hz, 2H), 4.72 (s, 2H), 3.87 (s, 3H), 3.50 (s, 2H), 1.44 (s, 9H), 1.33 (s, 6H). UPLC/MS (method A): Rt 2.39 min. MS (ES) C₁₈H₂₇NO₅ requires 337, found 338 [M+H]⁺.

### 5-(4-Methoxyphenyl)-3,3-dimethylmorpholine (XXVe)

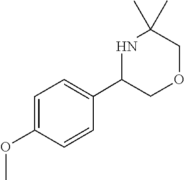 [SMILES: COc1ccc(C2COCC(C)(C)N2)cc1]

Following general procedure K, XXIVe (0.380 g, 1.13 mmol) afforded XXVe as a white solid (0.140 g, 56%). ¹H NMR (400 MHz, CDCl₃) δ 7.39-7.33 (m, 2H), 6.80 (d, J=8.8 Hz, 2H), 4.30 (s, 1H), 4.06 (t, J=12.0 Hz, 1H), 3.93 (dd, J=12.4, 3.9 Hz, 1H), 3.82-3.71 (m, 4H), 3.44 (d, J=12.3 Hz, 1H), 1.48 (s, 3H), 0.67 (s, 3H). UPLC/MS (method A): Rt 2.39 min. MS (ES) C₁₃H₁₉NO₂ requires 221, found 222 [M+H]⁺.

### 5-(4-Methoxyphenyl)-3,3-dimethyl-N-pentylmorpholine-4-carboxamide

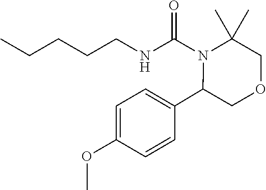 [SMILES: CCCCCNC(=O)N1C(c2ccc(OC)cc2)COCC1(C)C]

Following general procedure D (method A), XXVe (0.084 g, 0.25 mmol) and n-pentyl isocyanate (0.060 g, 0.6 mmol) afforded the title compound as a white solid (0.020 g, 20%). ¹H NMR (400 MHz, CDCl₃) δ 7.31-7.18 (m, 2H), 6.83 (d, J=8.7 Hz, 2H), 4.73 (bs, 1H), 4.42 (dd, J=9.3, 4.4 Hz, 1H), 3.91 (dd, J=11.5, 4.4 Hz, 1H), 3.76 (s, 3H), 3.60-3.31 (m, 3H), 2.96 (ddt, J=16.0, 13.2, 6.2 Hz, 2H), 1.43 (s, 3H), 1.37 (s, 3H), 1.19-1.11 (m, 4H), 1.05-0.91 (m, 2H), 0.79 (t, J=7.3 Hz, 3H). UPLC/MS (method A): Rt 2.27 min. MS (ES) C₁₉H₃₀N₂O₃ requires 334, found 335 [M+H]

### Example 157: N-[2-(Cyclopropylmethoxy)ethyl]-5-(4-methoxyphenyl)-3,3-dimethylmorpholine-4-carboxamide

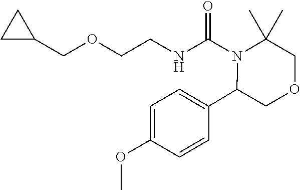 [SMILES: COc1ccc(C2COCC(C)(C)N2C(=O)NCCOCC2CC2)cc1]

Following general procedure D (method B), XXVe (0.050 g, 0.15 mmol) and 2-(cyclopropylmethoxy)-ethanamine (0.052 g, 0.45 mmol) afforded the title compound as a colorless oil (0.020 g, 28%). ¹H NMR (400 MHz, CDCl₃) δ 7.27 (d, J=8.5 Hz, 2H), 6.83 (d, J=8.7 Hz, 2H), 5.17 (bs, 1H), 4.51 (dd, J=8.7, 4.3 Hz, 1H), 3.95 (dd, J=11.5, 4.3 Hz, 1H), 3.77 (s, 3H), 3.63-3.55 (m, 1H), 3.54-3.40 (m, 2H), 3.32-3.17 (m, 4H), 3.13 (d, J=6.9 Hz, 2H), 1.44 (s, 3H), 1.40 (s, 3H), 0.94 (ddt, J=9.9, 7.0, 3.5 Hz, 1H), 0.58-0.37 (m, 2H), 0.14 (d, J=4.6 Hz, 2H). UPLC/MS (method A): Rt 2.05 min. MS (ES) C₂₀H₃₀N₂O₄ requires 362, found 363 [M+H]⁺.

### Example 158: 3,3-Dimethyl-5-(o-tolyl)-N-pentylmorpholine-4-carboxamide tert-Butyl N-[1,1-dimethyl-2-[2-(o-tolyl)-2-oxoethoxy]ethyl]carbamate (XXIVf)

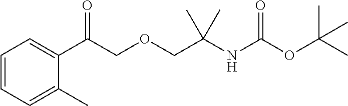 [SMILES: Cc1ccccc1C(=O)COCC(C)(C)NC(=O)OC(C)(C)C]

Following general procedure H (method A), XXIHa (0.230 g, 1.0 mmol) and o-tolylmagnesium bromide (0.230 g, 1.10 mmol, 0.5 M in THF) afforded XXIVf as a clear oil (0.120 g, 38%). ¹H NMR (400 MHz, CDCl₃) δ 7.58-7.53 (m, 1H), 7.43-7.36 (m, 1H), 7.29-7.22 (m, overlapped with solvent signal, 2H), 4.63 (s, 2H), 3.50 (s, 2H), 2.52 (s, 3H), 1.44 (s, 9H), 1.39-1.29 (m, 6H). UPLC/MS (method A): Rt 2.55 min. MS (ES) C₁₈H₂₇NO₄ requires 321, found 322 [M+H]⁺.

### 3,3-Dimethyl-5-(o-tolyl)morpholine (XXVf)

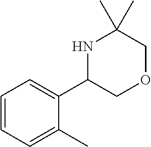

Following general procedure K, XXIVf (0.120 g, 0.37 mmol) afforded XXVf which was used in the next step without further purification. ¹H NMR (400 MHz, DMSO-d₆) δ 7.53 (d, J=6.7 Hz, 1H), 7.23-7.04 (m, 3H), 4.29 (dd, J=10.4, 3.2 Hz, 1H), 3.73 (dd, J=10.6, 3.2 Hz, 1H), 3.44 (d, J=10.1 Hz, 1H), 3.16 (d, J=10.6 Hz, 1H), 3.01 (t, J=10.6 Hz, 1H), 2.33 (s, 3H), 1.27 (s, 3H), 0.99 (s, 3H). UPLC/MS (method A): Rt 1.27 min. MS (ES) C₁₃H₁₉NO requires 205, found 206 [M+H]⁺.

### 3,3-Dimethyl-5-(o-tolyl)-N-pentylmorpholine-4-carboxamide

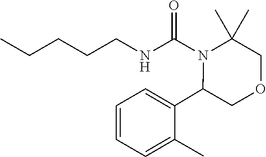 [SMILES: CCCCCNC(=O)N1C(c2ccccc2C)COCC1(C)C]

Following general procedure D (method A), XXVf (0.078 g, 0.38 mmol) and n-pentyl isocyanate (0.052 g, 0.46 mmol) afforded the title compound as a white solid (0.012 g, 10%). ¹H NMR (400 MHz, DMSO-d₆) δ7.47-7.42 (m, 2H), 7.11-7.05 (m, 3H), 7.01-6.91 (m, 1H), 4.60-4.53 (m, 1H), 3.76-3.62 (m, 1H), 3.51-3.45 (m, 1H), 3.39-3.34 (m, 1H), 3.18-3.07 (m, 1H), 2.87-2.69 (m, 2H), 2.36 (s, 3H), 1.26-1.20 (m, 5H), 1.19-1.06 (m, 4H), 1.05-0.96 (m, 2H), 0.84-0.72 (m, 3H). UPLC/MS (method A): Rt 2.51 min. MS (ES) C₁₉H₃₀N₂O₂ requires 318, found 319 [M+H]⁺.

### Example 159: 5-(6-Methoxy-3-pyridyl)-3,3-dimethyl-N-pentylmorpholine-4-carboxamide tert-Butyl N-[2-[2-(6-methoxy-3-pyridyl)-2-oxoethoxy]-1,1-dimethylethyl]carbamate (XXIVg)

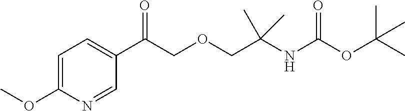 [SMILES: COc1ccc(C(=O)COCC(C)(C)NC(=O)OC(C)(C)C)cn1]

Following general procedure H (method B), XXIIIa (0.300 g, 1.31 mmol) and 5-bromo-2-methoxypyridine (0.226 g, 1.20 mmol) afforded XXIVg as a colorless oil (0.070 g, 19%). ¹H NMR (400 MHz, CDCl₃) δ 8.77 (d, J=2.4 Hz, 1H), 8.12 (dd, J=8.7, 2.4 Hz, 1H), 6.80 (d, J=8.7 Hz, 1H), 5.09 (s, 1H), 4.68 (s, 2H), 4.01 (s, 3H), 3.53 (s, 2H), 1.43 (s, 9H), 1.32 (s, 6H). UPLC/MS (method A): Rt 2.30 min. MS (ES) C₁₇H₂₆N₂O₅ requires 338, found 339 [M+H]⁺.

### 5-(6-Methoxy-3-pyridyl)-3,3-dimethylmorpholine (XXVg)

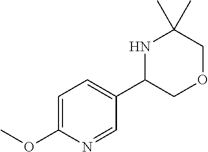 [SMILES: COc1ccc(C2COCC(C)(C)N2)cn1]

Following general procedure K, XXIVg (0.070 g, 0.21 mmol) afforded XXVg which was used in the next step without further purification. UPLC/MS (method A): Rt 1.01 min. MS (ES) C₁₂H₁₈N₂O₂ requires 222, found 223 [M+H]⁺.

### 5-(6-Methoxy-3-pyridyl)-3,3-dimethyl-N-pentylmorpholine-4-carboxamide

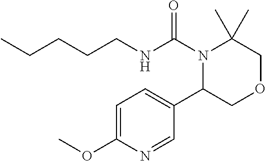 [SMILES: CCCCCNC(=O)N1C(c2ccc(OC)nc2)COCC1(C)C]

Following general procedure D (method A), XXVg (0.047 g, 0.21 mmol) and n-pentyl isocyanate (0.026 g, 0.23 mmol) afforded the title compound as a white powder (0.023 g, 33%). ¹H NMR (400 MHz, CDCl₃) δ 8.14 (d, J=2.5 Hz, 1H), 7.56 (dd, J=8.6, 2.5 Hz, 1H), 6.67 (d, J=8.5 Hz, 1H), 4.90 (t, J=5.5 Hz, 1H), 4.49 (dd, J=9.9, 4.1 Hz, 1H), 3.93-3.84 (m, 4H), 3.48 (s, 3H), 3.06-2.98 (m, 2H), 1.40 (s, 3H), 1.29 (s, 3H), 1.2-1.15 (m, 4H), 1.12-1.02 (m, 2H), 0.83 (t, J=7.3 Hz, 3H). UPLC/MS (method A): Rt 2.09 min MS (ES) C₁₈H₂₉N₃O₃ requires 335, found 336 [M+H]⁺.

### Example 160: 5-(2-Fluoro-4-methoxyphenyl)-3,3-dimethyl-N-pentylmorpholine-4-carboxamide tert-Butyl N-[2-[2-(2-fluoro-4-methoxyphenyl)-2-oxoethoxy]-1,1-dimethylethyl]carbamate (XXIVh)

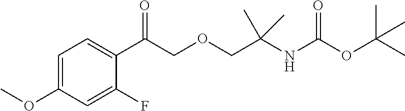 [SMILES: COc1ccc(C(=O)COCC(C)(C)NC(=O)OC(C)(C)C)c(F)c1]

Following general procedure H (method B), XXIIIa (0.250 g, 1.22 mmol) and 1-bromo-2-fluoro-4-methoxybenzene (0.280 g, 1.22 mmol) afforded compound XXIVh as white solid (0.210 g, 48%). ¹H NMR (400 MHz, CDCl₃) δ 7.96 (t, J=8.6 Hz, 1H), 6.79 (dd, J=8.9, 2.3 Hz, 1H), 6.61 (dd, J=13.0, 2.4 Hz, 1H), 4.66 (d, J=3.9 Hz, 2H), 3.86 (s, 3H), 3.50 (s, 2H), 1.44 (s, 9H), 1.34 (s, 6H). UPLC/MS (method A): Rt 2.55 min. MS (ES) C₁₈H₂₆FNO₅ requires 355, found 356 [M+H]⁺.

### 5-(2-Fluoro-4-methoxyphenyl)-3,3-dimethylmorpholine;2,2,2-trifluoroacetic acid (XXVh)

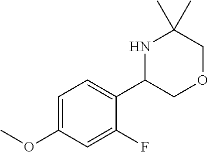 [SMILES: COc1ccc(C2COCC(C)(C)N2)c(F)c1]

Following general procedure K, XXIVh (0.210 g, 0.59 mmol) afforded XXVh which was used in the next step without further purification. ¹H NMR (400 MHz, CDCl₃) δ 7.44 (t, J=8.6 Hz, 1H), 6.70-6.55 (m, 2H), 4.67 (dd, J=11.1, 3.7 Hz, 1H), 4.13 (s, 2H), 3.78 (s, 3H), 3.53 (s, 2H), 1.28 (s, 6H). UPLC/MS (method A): Rt 1.31 min. MS (ES) C₁₃H₁₈FNO₂ requires 239, found 240 [M+H]⁺.

### 5-(2-Fluoro-4-methoxyphenyl)-3,3-dimethyl-N-pentylmorpholine-4-carboxamide

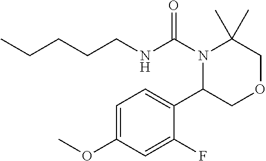 [SMILES: CCCCCNC(=O)N1C(c2ccc(OC)cc2F)COCC1(C)C]

Following general procedure D (method A), XXVh (0.105 g, 0.3 mmol) and n-pentyl isocyanate (0.041 g, 0.36 mmol) afforded the title compound as a colorless oil (0.050 g, 44%). ¹H NMR (400 MHz, CDCl₃) δ 7.30 (t, J=8.6 Hz, 1H), 6.64 (dd, J=8.5, 2.5 Hz, 1H), 6.58 (dd, J=12.2, 2.5 Hz, 1H), 4.82-4.72 (m, 2H), 3.95 (dd, J=11.4, 4.4 Hz, 1H), 3.77 (s, 3H), 3.56-3.41 (m, 3H), 3.00 (dh, J=13.4, 6.5 Hz, 2H), 1.44 (s, 3H), 1.39 (s, 3H), 1.30-1.14 (m, 4H), 1.11-0.98 (m, 2H), 0.82 (t, J=7.3 Hz, 3H). UPLC/MS (method A): Rt 2.39 min. MS (ES) C₁₉H₂₉FN₂O₃ requires 352, found 353 [M+H]⁺.

### Example 161: 5-(3-Fluoro-4-methoxyphenyl)-3,3-dimethyl-N-pentylmorpholine-4-carboxamide tert-Butyl N-[2-[2-(3-fluoro-4-methoxyphenyl)-2-oxoethoxy]-1,1-dimethyl-ethyl]carbamate (XXIVi)

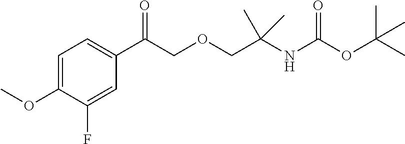 [SMILES: COc1ccc(C(=O)COCC(C)(C)NC(=O)OC(C)(C)C)cc1F]

Following general procedure H (method A), XXIIIa (0.300 g, 1.31 mmol) and 4-bromo-2-fluoroanisole (0.806 g, 3.93 mmol) afforded XXIVi as a colorless oil (0.192 g, 42%). ¹H NMR (400 MHz, CDCl₃) δ 7.76-7.66 (m, 2H), 7.00 (t, J=8.3 Hz, 1H), 5.14 (bs, 1H), 4.69 (s, 2H), 3.96 (s, 3H), 3.51 (s, 2H), 1.44 (s, 9H), 1.32 (s, 6H). UPLC/MS (method A): Rt 2.46 min. MS (ES) C₁₈H₂₆FNO₅ requires 355, found 356 [M+H]⁺.

### 5-(3-Fluoro-4-methoxyphenyl)-3,3-dimethylmorpholine (XXVi)

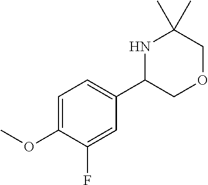 [SMILES: COc1ccc(C2COCC(C)(C)N2)cc1F]

Following general procedure K, XXIVi (0.192 g, 0.54 mmol) afforded XXVi as a white solid (0.118 g, 91%). ¹H NMR (400 MHz, CDCl₃) δ 7.24-7.16 (m, 1H), 7.14-7.06 (m, 1H), 6.90 (t, J=8.5 Hz, 1H), 4.19 (dd, J=10.7, 3.6 Hz, 1H), 3.92-3.79 (m, 4H), 3.51 (d, J=11.1 Hz, 1H), 3.44-3.27 (m, 2H), 2.11-2.01 (m, 1H), 1.38 (s, 3H), 1.03 (s, 3H). UPLC/MS (method A): Rt 1.25 min. MS (ES) C₁₃H₁₈FNO₂ requires 239, found 240 [M+H]⁺.

### 5-(3-Fluoro-4-methoxyphenyl)-3,3-dimethyl-N-pentylmorpholine-4-carboxamide

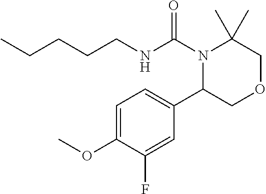 [SMILES: CCCCCNC(=O)N1C(c2ccc(OC)c(F)c2)COCC1(C)C]

Following general procedure D (method A), XXVi (0.050 g, 0.21 mmol) and n-pentyl isocyanate (0.026 g, 0.23 mmol) afforded the title compound as a white powder (0.017 g, 23%). ¹H NMR (400 MHz, CDCl₃) δ 7.15-7.01 (m, 2H), 6.89 (t, J=8.5 Hz, 1H), 4.82 (bs, 1H), 4.46 (dd, J=9.6, 4.2 Hz, 1H), 3.90 (dd, J=11.5, 4.3 Hz, 1H), 3.86 (s, 3H), 3.54-3.38 (m, 3H), 3.02 (qd, J=7.0, 3.4 Hz, 2H), 1.42 (s, 3H), 1.34 (s, 3H), 1.30-1.16 (m, 4H), 1.13-1.02 (m, 2H), 0.83 (t, J=7.3 Hz, 3H). UPLC/MS (method A): Rt 2.31 min MS (ES) C₁₉H₂₉FN₂O₃ requires 352, found 353 [M+H]⁺.

### Example 162: 5-(3,4-Difluorophenyl)-3,3-dimethyl-N-pentylmorpholine-4-carboxamide tert-Butyl N-[2-[2-(3,4-difluorophenyl)-2-oxoethoxy]-1,1-dimethyethyl]carbamate (XXIVj)

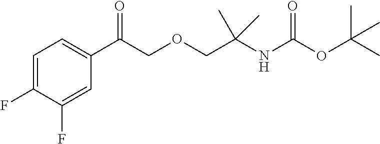 [SMILES: CC(C)(COCC(=O)c1ccc(F)c(F)c1)NC(=O)OC(C)(C)C]

Following general procedure G (Method A), XXIIIa (0.250 g, 1.09 mmol) and 4-bromo-1,2-difluoro-benzene (0.631 g, 3.27 mmol) afforded XXIVj as a colorless oil (0.146 g, 39%). ¹H NMR (400 MHz, CDCl₃) δ 7.80 (ddd, J=10.3, 7.7, 2.1 Hz, 1H), 7.72 (ddd, J=8.7, 4.2, 1.8 Hz, 1H), 7.30-7.22 (m, 1H), 5.10-4.91 (bs, 1H), 4.69 (s, 2H), 3.53 (s, 2H), 1.43 (s, 9H), 1.31 (s, 6H). UPLC/MS (method A): Rt 2.50 min. MS (ES) C₁₇H₂₃F₂NO₄ requires 343, found 344 [M+H]⁺.

### 5-(3,4-Difluorophenyl)-3,3-dimethylmorpholine (XXVj)

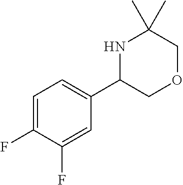 [SMILES: CC1(C)COCC(c2ccc(F)c(F)c2)N1]

Following general procedure K, XXIVj (0.146 g, 0.43 mmol) afforded XXVj as a white solid (0.065 g, 67%). ¹H NMR (400 MHz, CDCl₃) δ 7.34-7.27 (m, 1H), 7.16-7.05 (m, 2H), 4.22 (dd, J=10.7, 3.6 Hz, 1H), 3.85 (dd, J=11.2, 3.6 Hz, 1H), 3.53 (d, J=11.1 Hz, 1H), 3.36 (d, J=11.1 Hz, 1H), 3.28 (t, J=10.9 Hz, 1H), 2.12-2.05 (m, 1H), 1.38 (s, 3H), 1.06 (d, J=2.1 Hz, 3H). UPLC/MS (method A): Rt 1.34 min. MS (ES) C₁₂H₁₅F₂NO requires 227, found 228 [M+H]⁺.

### 5-(3,4-Difluorophenyl)-3,3-dimethyl-N-pentylmorpholine-4-carboxamide

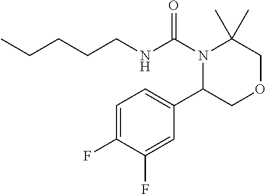 [SMILES: CCCCCNC(=O)N1C(c2ccc(F)c(F)c2)COCC1(C)C]

Following general procedure D (method A), XXVj (0.065 g, 0.29 mmol) and n-pentyl isocyanate (0.036 g, 0.32 mmol) afforded the title compound as a white powder (0.035 g, 35%). ¹H NMR (400 MHz, CDCl₃) δ 7.23-7.14 (m, 1H), 7.12-7.03 (m, 2H), 4.90 (bs, 1H), 4.51 (dd, J=10.0, 4.1 Hz, 1H), 3.88 (dd, J=11.5, 4.2 Hz, 1H), 3.54-3.35 (m, 3H), 3.04 (q, J=7.0 Hz, 2H), 1.39 (s, 3H), 1.32 (s, 3H), 1.30-1.17 (m, 4H), 1.15-1.05 (m, 2H), 0.83 (t, J=7.3 Hz, 3H). UPLC/MS (method A): Rt 2.41 min MS (ES) C₁₈H₂₆F₂N₂O₂ requires 340, found 341 [M+H]⁺.

### Example 163: 5-(4-Fluoro-3-methoxyphenyl)-3,3-dimethyl-N-pentylmorpholine-4-carboxamide tert-Butyl N-[2-[2-(4-fluoro-3-methoxyphenyl)-2-oxoethoxy]-1,1-dimethyl-ethyl]carbamate (XXIVk)

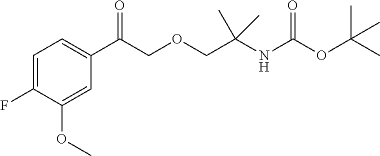 [SMILES: COc1cc(C(=O)COCC(C)(C)NC(=O)OC(C)(C)C)ccc1F]

Following general procedure H (method A), XXIIIa (0.250 g, 1.09 mmol) and 5-bromo-2-fluoroanisole (0.670 g, 3.27 mmol) afforded XXIVk as a colorless oil (0.110 g, 28%). ¹H NMR (400 MHz, CDCl₃) δ 7.61 (dd, J=8.3, 2.0 Hz, 1H), 7.48 (ddd, J=8.4, 4.3, 2.0 Hz, 1H), 7.13 (dd, J=10.6, 8.4 Hz, 1H), 5.13 (bs, 1H), 4.73 (s, 2H), 3.94 (s, 3H), 3.52 (s, 2H), 1.44 (s, 9H), 1.33 (s, 6H). UPLC/MS (method A): Rt 2.46 min. MS (ES) C₁₈H₂₆FNO₅ requires 355, found 356 [M+H]⁺.

### 5-(4-Fluoro-3-methoxyphenyl)-3,3-dimethylmorpholine (XXVk)

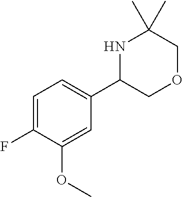 [SMILES: COc1cc(C2COCC(C)(C)N2)ccc1F]

Following general procedure K, XXIVk (0.110 g, 0.31 mmol) afforded XXVk as a white solid (0.074 g, quant.). UPLC/MS (method A): Rt 1.25 min. MS (ES) C₁₃H₁₈FNO₂ requires 239, found 240 [M+H]⁺.

### 5-(4-Fluoro-3-methoxyphenyl)-3,3-dimethyl-N-pentylmorpholine-4-carboxamide

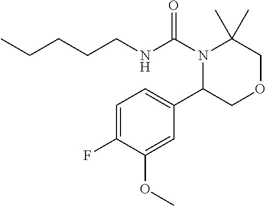 [SMILES: CCCCCNC(=O)N1C(c2ccc(F)c(OC)c2)COCC1(C)C]

Following general procedure D (method A), XXVk (0.080 g, 0.33 mmol) and n-pentyl isocyanate (0.042 g, 0.37 mmol) afforded the title compound as a white powder (0.032 g, 28%). ¹H NMR (400 MHz, CDCl₃) δ 7.04-6.93 (m, 2H), 6.89 (ddd, J=8.4, 4.3, 2.1 Hz, 1H), 4.79 (t, J=5.4 Hz, 1H), 4.47 (dd, J=9.5, 4.2 Hz, 1H), 3.92 (dd, J=11.5, 4.3 Hz, 1H), 3.88 (s, 3H), 3.52-3.45 (m, 3H), 3.02 (ddd, J=13.2, 7.1, 6.1 Hz, 2H), 1.43 (s, 3H), 1.37 (s, 3H), 1.31-1.15 (m, 4H), 1.10-1.01 (m, 2H), 0.82 (t, J=7.3 Hz, 3H). UPLC/MS (method A): Rt 2.29 min MS (ES) C₁₉H₂₉FN₂O₃ requires 352, found 353 [M+H]⁺.

### Example 164: Benzyl 3,3-dimethyl-5-phenyl-4-(4-phenylbutylcarbamoyl)piperazine-1-carboxylate Benzyl 3,3-dimethyl-5-oxopiperazine-1-carboxylate

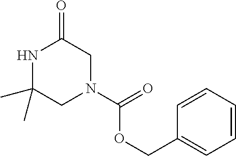 [SMILES: CC1(C)CN(C(=O)OCc2ccccc2)CC(=O)N1]

To a solution of 6,6-dimethylpiperazin-2-one (0.90 g, 7.02 mmol) and DIPEA (1.82, 14.04 mmol) in DCM (35 ml), CbzCl (2.39 g, 14.04 mmol) was added dropwise at 0° C. and the reaction mixture was stirred at RT for 3 h. The reaction mixture was diluted with DCM, washed with sat. aqueous NaHCO₃ solution and dried over Na₂SO₄. After concentration the residue was purified by column chromatography (SiO₂), eluting with DCM/MeOH (95:5) to afford the title compound as a white solid (1.55 g, 84%). ¹H NMR (400 MHz, DMSO-d₆) δ 8.09 (s, 1H), 7.66-7.10 (m, 5H), 5.12 (s, 2H), 3.89 (d, J=20.3 Hz, 2H), 3.40 (d, J=10.6 Hz, 2H), 1.13 (s, 6H). UPLC/MS (method A): Rt 1.63 min. MS (ES) C₁₄H₁₈N₂O₃ require 262, found 263 [M+H]⁺.

### O4-Benzyl O1-tert-butyl 2,2-dimethyl-6-oxopiperazine-1,4-dicarboxylate (XXIIId)

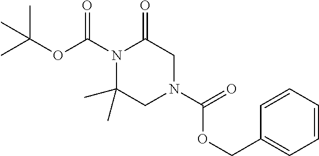 [SMILES: CC(C)(C)OC(=O)N1C(=O)CN(C(=O)OCc2ccccc2)CC1(C)C]

To a solution of benzyl 3,3-dimethyl-5-oxo-piperazine-1-carboxylate (1.2 g, 4.56 mmol) in anhydrous DMF (0.1 M, 30 mL) NaH 60% (0.22 g, 9.15 mmol) was added at 0° C. and the reaction mixture was stirred at RT for 30 min. A solution of Boc₂O (1.98 g, 9.15 mmol) in anhydrous DMF (15 mL) was added and the reaction mixture was stirred at RT for 72 h. The reaction mixture was quenched with brine, diluted and extracted with EA, washed with 5% aqueous solution of LiCl and dried over Na₂SO₄. After evaporation of the solvent, the residue was purified by column chromatography (SiO₂), eluting with Cy/EA (3:1) to afford XXIIId as a colorless oil (0.49 g, 29%). ¹H NMR (400 MHz, CDCl₃) δ 7.47-7.29 (m, 5H), 5.19 (s, 2H), 4.23 (s, 2H), 3.56 (s, 2H), 1.56 (s, 9H), 1.48-1.39 (m, 6H). UPLC/MS (method A): Rt 2.32 min. MS (ES) C₁₉H₂₆N₂O₅ requires 362, found 363 [M+H]⁺.

### Benzyl N-[2-(tert-butoxycarbonylamino)-2-methylpropyl]-N-phenacylcarbamate (XXIVI)

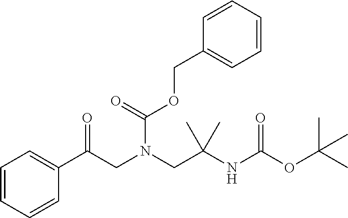 [SMILES: CC(C)(CN(CC(=O)c1ccccc1)C(=O)OCc1ccccc1)NC(=O)OC(C)(C)C]

Compound XXIVI was prepared according to general procedure H (method A), using XXIIId (0.365 g, 1.01 mmol) and PhMgBr (0.22 g, 1.23 mmol, 2.8 M in 2-MeTHF). The residue was purified by column chromatography (SiO₂), eluting with Cy/EA (75:25) to afford XXIVI as a colorless oil (0.27 g, 61%). ¹H NMR (400 MHz, DMSO-d₆) δ 8.04-7.85 (m, 2H), 7.73-7.63 (m, 1H), 7.60-7.45 (m, 2H), 7.42-7.29 (m, 2H), 7.29-7.14 (m, 3H), 6.46 (s, 0.6H), 6.41 (s, 0.4H), 5.11 (s, 0.8H), 5.03 (s, 1.2H), 4.85 (s, 1.2H), 4.78 (s, 0.8H), 3.55 (s, 0.8H), 3.51 (s, 1.2H), 1.25 (s, 5.4H), 1.21 (s, 3.6H), 1.20 (s, 3.6H), 1.15 (s, 2.4H). UPLC/MS (method A): Rt 2.66 min. MS (ES) C₂₅H₃₂N₂O₅ requires 440, found 441 [M+H]⁺.

### Benzyl 33-dimethyl-5-phenylpiperazine-1-carboxylate (XXVI)

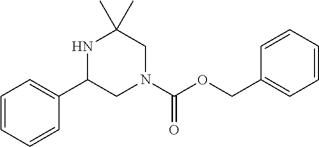 [SMILES: CC1(C)CN(C(=O)OCc2ccccc2)CC(c2ccccc2)N1]

Compound XXVI was prepared according to general procedure K using XXIVI (0.27 g, 0.61 mmol) and NaBH(OAc)₃ (0.39 g, 1.84 mmol). The residue was purified by column chromatography (SiO₂), eluting with Cy/EA (20: 80) to afford XXVI as a colorless oil (0.13 g, 66%). ¹H NMR (400 MHz, DMSO-d₆) δ 7.53-7.15 (m, 10H), 5.20-4.96 (m, 2H), 4.09-3.88 (m, 2H), 3.74 (d, J=12.6 Hz, 1H), 2.88-2.54 (m, 2H), 1.16-1.05 (m, 6H). UPLC/MS (method A): Rt 2.02 min. MS (ES) C₂₀H₂₄N₂O₂ requires 324, found 325 [M+H]⁺.

### Benzyl 3,3-dimethyl-5-phenyl-4-(4-phenylbutylcarbamoyl)piperazine-1-carboxylate

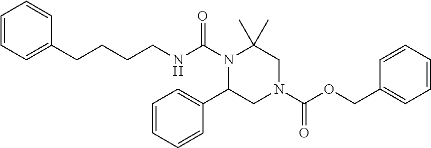 [SMILES: CC1(C)CN(C(=O)OCc2ccccc2)CC(c2ccccc2)N1C(=O)NCCCCc1ccccc1]

Following general procedure D (Method A), XXVI (0.065 g, 0.20 mmol) and 4-phenylbutyl isocyanate (0.039 g, 0.220 mmol) afforded the title compound as a colorless oil (0.089 g, 89%). ¹H NMR (400 MHz, DMSO-d₆) δ 7.52-7.21 (m, 12H), 7.16 (td, J=5.5, 2.9 Hz, 3H), 6.27 (d, J=18.9 Hz, 1H), 5.39-5.03 (m, 2H), 4.96 (t, J=3.9 Hz, 1H), 4.09 (dd, J=37.7, 13.1 Hz, 1H), 3.75 (dd, J=30.4, 12.7 Hz, 1H), 3.38 (d, J=13.3 Hz, 1H), 3.18-2.70 (m, 3H), 2.60-2.50 (m, 2H), 1.55-1.27 (m, 10H). UPLC/MS (method B): Rt 1.87 min. MS (ES) C₃₁H₃₇N₃O₃ requires 499, found 500 [M+H]⁺.

### Example 165: 2,2,4-Trimethyl-6-phenyl-N-(4-phenylbutyl)piperazine-1-carboxamide 2,2-Dimethyl-6-phenyl-N-(4-phenylbutyl)piperazine-1-carboxamide (XXVIa)

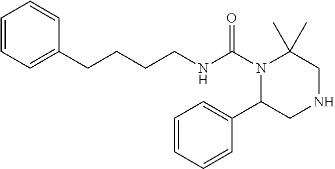 [SMILES: CC1(C)CNCC(c2ccccc2)N1C(=O)NCCCCc1ccccc1]

Following general procedure B (method E), benzyl 3,3-dimethyl-5-phenyl-4-(4-phenylbutylcarbamoyl)piperazine-1-carboxylate (0.080 g, 0.160 mmol) afforded XXVIa which was used in the next step without further purification. UPLC/MS (method A): Rt 1.90 min. MS (ES) C₂₃H₃₁N₃O require 365, found 366 [M+H]⁺.

### 2,2,4-Trimethyl-6-phenyl-N-(4-phenylbutyl)piperazine-1-carboxamide

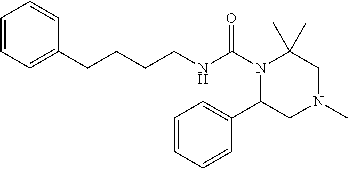 [SMILES: CN1CC(c2ccccc2)N(C(=O)NCCCCc2ccccc2)C(C)(C)C1]

To a solution of XXVIa (0.058 g, 0.16 mmol) in DCE (2 mL), 37% aq. formaldehyde solution (0.12 g, 4.0 mmol) and TFA (0,009 g, 0.08 mmol) were added followed by NaBH(OAc)₃ (0.10 g; 0.48 mmol) and the reaction was stirred at RT for 1 h. The mixture was quenched with the addition of MeOH, diluted with EA, washed with saturated aq. NaHCO₃ solution, brine and dried over Na₂SO₄. After concentration, the residue was purified by column chromatography (SiO₂), eluting with Cy/EA (60:40) to afford the title compound as a white solid (0.026 g, 42%). ¹H NMR (400 MHz, DMSO-d₆) δ 7.37-7.08 (m, 10H), 6.97 (t, J=5.4 Hz, 1H), 4.37 (dd, J=9.8, 3.5 Hz, 1H), 2.92-2.74 (m, 2H), 2.67-2.61 (m, 1H), 2.47-2.36 (m, 3H), 2.12 (s, 3H), 2.03-1.86 (m, 2H), 1.43-1.26 (m, 3H), 1.24 (s, 6H), 1.22-1.11 (m, 2H). UPLC/MS (method A): Rt 2.17 min. MS (ES) C₂₄H₃₃N₃O requires 379, found 380 [M+H]⁺.

### Example 166: 2,2,4-Trimethyl-5-phenyl-N-(4-phenylbutyl)piperazine-1-carboxamide Benzyl 2,2-dimethyl-5-oxopiperazine-1-carboxylate

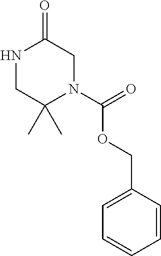 [SMILES: CC1(C)CNC(=O)CN1C(=O)OCc1ccccc1]

To a solution of 5,5-dimethylpiperazin-2-one (0.48 g, 3.75 mmol) and DIPEA (0.97 g, 7.49 mmol) in DCM (20 mL), CbzCl (1.28 g, 7.49 mmol) was added dropwise at 0° C. and the reaction mixture was stirred at RT for 3 h. The reaction mixture was diluted with DCM, washed with saturated aq. NaHCO₃ solution, dried over Na₂SO₄, concentrated and the residue purified by column chromatography (SiO₂), eluting with EA to afford the title compound as a white solid (0.65 g, 66%). ¹H NMR (400 MHz, DMSO-d₆) δ 8.19 (s, 1H), 7.54-7.20 (m, 5H), 5.08 (s, 2H), 3.91 (s, 2H), 3.13 (d, J=4.2 Hz, 2H), 1.36 (s, 6H). UPLC/MS (method A): Rt 1.65 min. MS (ES) C₁₄H₁₈N₂O₃ require 262, found 263 [M+H]⁺.

### O1-Benzyl O4-tert-butyl 2,2-dimethyl-5-oxopiperazine-1,4-dicarboxylate (XXIIIe)

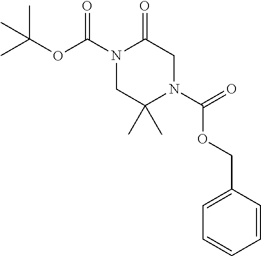 [SMILES: CC(C)(C)OC(=O)N1CC(C)(C)N(C(=O)OCc2ccccc2)CC1=O]

To a solution of benzyl 2,2-dimethyl-5-oxo-piperazine-1-carboxylate (0.250 g, 0.95 mmol) in anhydrous THF (6 mL) a solution of LiHMDS (0.63 mL, 0.63 mmol, 11.0 M in THF) was added dropwise at −78° C. and the reaction mixture was stirred for 30 min. A solution of Boc₂O (0.31 g, 1.43 mmol) in THF (4 mL) was added and the reaction mixture was stirred at RT for 1 h. The reaction mixture was quenched with the addition of saturated aq. NaHCO₃ solution, extracted with EA, washed with brine and dried over Na₂SO₄. After concentration, the residue was purified by column chromatography (SiO₂), eluting with Cy/EA (85:15) to afford XXIIIe as a colorless oil (0.27 g, 78%). ¹H NMR (600 MHz, DMSO-d₆) δ 7.47-7.27 (m, 5H), 5.08 (s, 2H), 4.16 (s, 2H), 3.77 (s, 2H), 1.46 (s, 9H), 1.37 (s, 3H), 1.36 (s, 3H). UPLC/MS (method A): Rt 2.36 min. MS (ES) C₁₉H₂₆N₂O₅ requires 362, found 363 [M+H]⁺.

### Benzyl N-[2-(tert-butoxycarbonylamino)-1,1-dimethylethyl]-N-phenacylcarbamate (XXIVn)

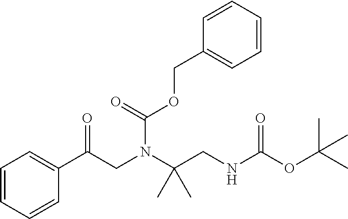 [SMILES: CC(C)(C)OC(=O)NCC(C)(C)N(CC(=O)c1ccccc1)C(=O)OCc1ccccc1]

To a solution of XXIIIe (0.215 g, 0.59 mmol) in anhydrous THF (6 mL) PhMgBr (0.140 g, 0.77 mmol, 2.9 M in 2-MeTHF) was added dropwise at −40° C. After 30 min the reaction mixture was quenched with the addition of H₂O and pH was adjusted to 10 by the use of a saturated aq. Na₂CO₃ solution (0.7 mL). The reaction mixture was stirred at RT for 2 h, extracted with EA, washed with brine and dried over Na₂SO₄. After concentration the residue was purified by column chromatography (SiO₂), eluting with Cy/EA (80:20) to afford XXIVn as a colorless oil (0.20 g, 77%). ¹H NMR (400 MHz, CDCl₃) δ 7.93 (d, J=7.6 Hz, 2H), 7.67-7.56 (m, 1H), 7.54-7.44 (m, 2H), 7.29-7.08 (m, 5H), 5.80 (bs, 1H), 5.11 (s, 2H), 4.81 (s, 2H), 3.65 (s, 2H), 1.46 (s, 9H), 1.40 (s, 6H). UPLC/MS (method B): Rt 1.61 min. MS (ES) C₂₅H₃₂N₂O₅ requires 440, found 441 [M+H]⁺.

### Benzyl 2,2-dimethyl-5-phenylpiperazine-1-carboxylate (XXVn)

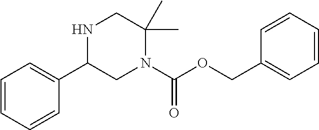 [SMILES: CC1(C)CNC(c2ccccc2)CN1C(=O)OCc1ccccc1]

Following general procedure K, XXIVn (0.20 g, 0.45 mmol) afforded XXVn which was used in the next step without further purification. UPLC/MS (method A): Rt 2.34 min. MS (ES) C₂₀H₂₄N₂O₂ requires 324, found 325 [M+H]⁺.

### Benzyl 2,2,4-trimethyl-5-phenylpiperazine-1-carboxylate (XXXIa)

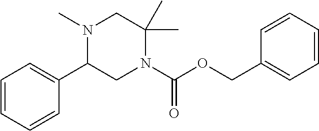 [SMILES: CN1CC(C)(C)N(C(=O)OCc2ccccc2)CC1c1ccccc1]

To a solution of XXVn (0.152 g, 0.45 mmol) in DCE (5 mL) formaldehyde 37% in H₂O (0.34 g; 1.09 mmol) and TFA (0,026 g; 0.23 mmol) were added followed by NaBH(OAc)₃ (0.29 g; 1.36 mmol) and the reaction was stirred at RT for 1 h. The mixture was quenched with MeOH, diluted with DCM, washed with saturated aq. NaHCO₃ solution, brine and dried over Na₂SO₄. After concentration the residue was purified by column chromatography (SiO₂), eluting with Cy/EA (85:15) to afford XXXIa as a colorless oil (0.123 g, 80%). ¹H NMR (400 MHz, DMSO-d₆) δ 7.67-7.08 (m, 10H), 5.11-4.92 (m, 2H), 3.71 (dd, J=12.2, 2.8 Hz, 1H), 3.12-2.93 (m, 2H), 2.64 (d, J=11.9 Hz, 1H), 2.19 (d, J=11.9 Hz, 1H), 1.93 (s, 3H), 1.48 (s, 3H), 1.41 (s, 3H). UPLC/MS (method B): Rt 2.00 min. MS (ES) C₂₁H₂₆N₂O₂ requires 338, found 339 [M+H]⁺.

### 1,5,5-trimethyl-2-phenylpiperazine (XXXIIa)

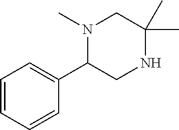

Following general procedure B (Method E), XXXIa (0.120 g, 0.355 mmol) afforded XXXIIa which was used in the next step without further purification. UPLC/MS (method A): Rt 1.32 min. MS (ES) C₁₃H₂₀N₂ require 204, found 205 [M+H]⁺.

### 2,2,4-Trimethyl-5-phenyl-N-(4-phenylbutyl)piperazine-1-carboxamide

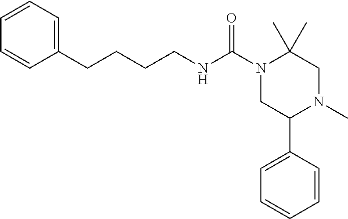 [SMILES: CN1CC(C)(C)N(C(=O)NCCCCc2ccccc2)CC1c1ccccc1]

Following general procedure D (method A), XXXIIa (0.072 g, 0.355 mmol) and 4-phenylbutyl isocyanate (0.068 g, 0.39 mmol) afforded the title compound as a white solid (0.080 g, 59%). ¹H NMR (400 MHz, DMSO-d₆) δ 7.58-7.20 (m, 7H), 7.19-7.06 (m, 3H), 6.47 (t, 1=5.5 Hz, 1H), 3.40 (dd, J=12.1, 3.1 Hz, 1H), 3.04-2.86 (m, 3H), 2.87-2.77 (m, 1H), 2.59-2.52 (m, 3H), 2.04 (d, J=11.5 Hz, 1H), 1.88 (s, 3H), 1.55-1.44 (m, 2H), 1.43 (s, 3H), 1.40-1.29 (m, 5H). UPLC/MS (method B): Rt 1.76 min. MS (ES) C₂₄H₃₃N₃O requires 379, found 380 [M+H]⁺.

### Example 167: 6-(4-Fluorophenyl)-2,2,4-trimethyl-N-pentylpiperazine-1-carboxamide hydrochloride Benzyl 5-(4-fluorophenyl)-3,3-dimethyl-4-(pentylcarbamoyl)piperazine-1-carboxylate

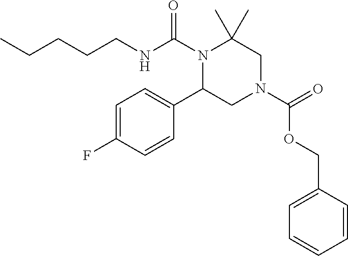 [SMILES: CCCCCNC(=O)N1C(c2ccc(F)cc2)CN(C(=O)OCc2ccccc2)CC1(C)C]

Following general procedure D (Method A), XXVm (0.080 g, 0.234 mmol) and pentylisocyanate (0.029 g, 0.257 mmol) afforded the title compound as a colorless oil (0.080 g, 75%). 1H NMR (400 MHz, DMSO-d₆) δ 7.52-7.23 (m, 7H), 7.22-7.08 (m, 2H), 6.33 (d, J=19.0 Hz, 1H), 5.10 (d, J=10.8 Hz, 2H), 4.87 (s, 1H), 4.10-3.63 (m, 2H), 3.43 (d, J=13.2 Hz, 1H), 3.05-2.71 (m, 3H), 1.50-1.00 (m, 12H), 0.82 (t, J=7.2 Hz, 3H). UPLC/MS (method B): Rt 1.74 min. MS (ES) C₂₆H₃₄FN₃O₃ requires 455, found 456 [M+H]⁺.

### 6-(4-Fluorophenyl)-2,2-dimethyl-N-pentylpiperazine-1-carboxamide (XXVIb)

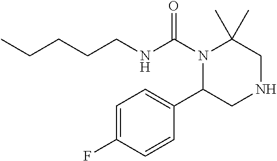 [SMILES: CCCCCNC(=O)N1C(c2ccc(F)cc2)CNCC1(C)C]

Following general procedure B (Method E), benzyl 5-(4-fluorophenyl)-3,3-dimethyl-4-(pentylcarbamoyl)piperazine-1-carboxylate (0.075 g, 0.165 mmol) afforded XXVIb which was used in the next step without further purification. UPLC/MS (method A): Rt 1.76 min. MS (ES) C₁₈H₂₈FN₃O requires 321, found 322 [M+H]⁺.

### 6-(4-Fluorophenyl)-2,2,4-trimethyl-N-pentylpiperazine-1-carboxamide hydrochloride

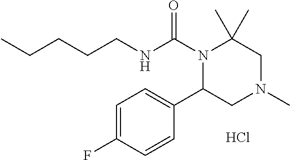 [SMILES: CCCCCNC(=O)N1C(c2ccc(F)cc2)CN(C)CC1(C)C ; Cl]

To a solution of XXVIb (0.055 g, 0.165 mmol) in DCE (2 ml) formaldehyde 37% in H₂O (0.124 g, 4.125 mmol) and TFA (0,009 g, 0.083 mmol) were added followed by NaBH(OAc)₃ (0.105 g, 0.495 mmol) and the reaction was stirred at RT for 1 h. The reaction mixture was neutralized by saturated aq. NaHCO₃ solution then extracted with DCM. The organic layers were dried over Na₂SO₄, concentrated and the residue was purified by column chromatography (SiO₂), eluting with DCM/EA (20:80) to afford the free base of the title compound as a white solid (0.026 g, 47%). To a solution of the free base of the title compound (0.017 g, 0.051 mmol) in Et₂O (1 mL) was added HCl (0.04 mL, 0.152 mmol, 4M in dioxane) and the reaction was stirred at RT for 1 h. The mixture was concentrated and the residue was triturated with Et₂O to afford the title compound as a white solid (0.016 g, 84%). ¹H NMR (400 MHz, DMSO-d₆) δ 10.30 (s, 1H), 7.44 (t, J=6.0 Hz, 1H), 7.41-7.35 (m, 2H), 7.23-7.09 (m, 2H), 4.46 (dd, J=11.8, 3.2 Hz, 1H), 3.37-3.23 (m, 2H), 3.06-2.87 (m, 2H), 2.86-2.65 (m, 5H), 1.38 (s, 3H), 1.30 (s, 3H), 1.20-0.99 (m, 4H), 1.01-0.84 (m, 2H), 0.77 (t, J=7.3 Hz, 3H). UPLC/MS (method A): Rt 1.68 min. MS (ES) C₂₀H₃₁FClN₃O₂ requires 363, found 364 [M+H]⁺.

### Example 168: N-[2-(Cyclopropylmethoxy)ethyl]-6-(4-fluorophenyl)-2,2,4-trimethylpiperazine-1-carboxamide hydrochloride Benzyl N-[2-(tert-butoxycarbonylamino)-2-methyl-propyl]-N-[2-(4-fluorophenyl)-2-oxoethyl]carbamate (XXIVm)

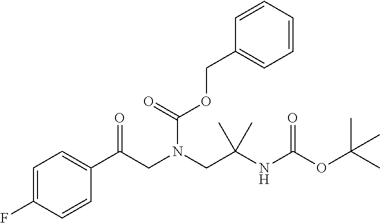 [SMILES: CC(C)(CN(CC(=O)c1ccc(F)cc1)C(=O)OCc1ccccc1)NC(=O)OC(C)(C)C]

Following general procedure H, XXIIId (0.380 g, 1.05 mmol) and 4-fluorophenylmagnesium bromide (0.5 M in THF) (0.25 g, 1.26 mmol) afforded XXIVm as a colorless oil (0.263 g, 55%). ¹H NMR (400 MHz, CDCl₃) δ 8.06-7.96 (m, 0.9H), 7.96-7.89 (m, 1.10H), 7.41 (s, 0.9H), 7.40 (s, 1.1H), 7.29-7.23 (m, 2H), 7.24-7.19 (m, 1H), 7.18-7.12 (m, 2H), 5.19 (s, 0.9H), 5.11 (s, 1.1H), 4.82 (bs, 0.55H), 4.75 (s, 0.9H), 4.71 (s, 1.1H), 4.56 (bs, 0.45H), 3.73-3.62 (m, 2H), 1.37 (s, 3.3H), 1.30 (s, 2.7H), 1.26 (s, 5H), 1.22 (s, 4H). UPLC/MS (method B): Rt 1.82 min. MS (ES) C₂₅H₃₁FN₂O₅ requires 458, found 459 [M+H]⁺.

### Benzyl 5-(4-fluorophenyl)-3,3-dimethylpiperazine-1-carboxylate (XXVm)

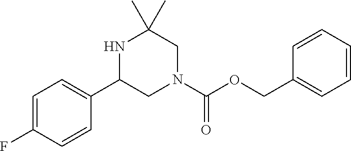 [SMILES: CC1(C)CN(C(=O)OCc2ccccc2)CC(c2ccc(F)cc2)N1]

Following general procedure K, XXIVm (0.26 g, 0.57 mmol) afforded XXVm as a colorless oil (0.165 g, 85%). ¹H NMR (400 MHz, CDCl₃) δ 7.39 (s, 7H), 7.07-6.96 (m, 2H), 5.22 (d, J=12.5 Hz, 1H), 5.16 (d, J=12.4 Hz, 1H), 4.32-3.77 (m, 3H), 2.88-2.52 (m, 2H), 1.25 (d, J=18.9 Hz, 3H), 1.19 (d, J=13.2 Hz, 3H). UPLC/MS (method A): Rt 1.03 min. MS (ES) C₂₀H₂₃FN₂O₂ requires 342, found 343 [M+H]⁺.

### Benzyl 4-[2-(cyclopropylmethoxy)ethylcarbamoyl]-5-(4-fluorophenyl)-3,3-dimethyl-piperazine-1-carboxylate

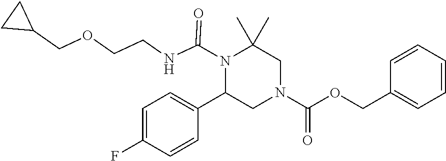 [SMILES: CC1(C)CN(C(=O)OCc2ccccc2)CC(c2ccc(F)cc2)N1C(=O)NCCOCC1CC1]

Following general procedure D (Method B), XXVm (0.078 g, 0.229 mmol) and 2-(cyclopropylmethoxy)ethan-1-amine (0.080 g, 0.695 mmol) afforded the title compound as a colorless oil (0.106 g, 95%). ¹H NMR (400 MHz, DMSO-d₆) δ 7.79-6.88 (m, 9H), 6.16 (d, J=17.4 Hz, 1H), 5.10 (d, J=15.0 Hz, 2H), 4.96 (s, 1H), 4.21-3.94 (m, 1H), 3.88-3.67 (m, 1H), 3.41 (d, J=13.3 Hz, 1H), 3.35-3.31 (m, 2H), 3.25-3.14 (m, 3H), 3.12-2.99 (m, 1H), 2.94-2.72 (m, 1H), 1.45 (s, 3H), 1.39 (s, 3H), 0.97-0.83 (m, 1H), 0.46-0.35 (m, 2H), 0.1-0.05 (m, 2H). UPLC/MS (method A): Rt 2.50 min. MS (ES) C₂₇H₃₄FN₃O₄ requires 483, found 484 [M+H]⁺.

### N-[2-(cyclopropylmethoxy)ethyl]-6-(4-fluorophenyl)-2,2-dimethyl-piperazine-1-carboxamide (XXVIc)

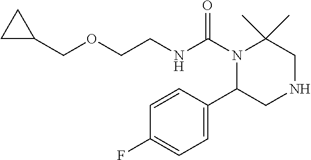 [SMILES: CC1(C)CNCC(c2ccc(F)cc2)N1C(=O)NCCOCC1CC1]

Following general procedure B (Method E), benzyl 4-[2-(cyclopropylmethoxy)ethylcarbamoyl]-5-(4-fluorophenyl)-3,3-dimethyl-piperazine-1-carboxylate (0.095 g, 0.196 mmol) afforded XXVIc which was used in the next step without further purification. UPLC/MS (method A): Rt 1.49 min. MS (ES) C₁₉H₂₈FN₃O₂ requires 349, found 350 [M+H]⁺.

### N-[2-(Cyclopropylmethoxy)ethyl]-6-(4-fluorophenyl)-2,2,4-trimethylpiperazine-1-carboxamide hydrochloride

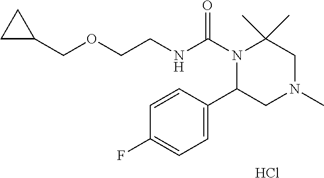 [SMILES: CN1CC(c2ccc(F)cc2)N(C(=O)NCCOCC2CC2)C(C)(C)C1 ; Cl]

To a solution of XXVIc (0.068 g, 0.196 mmol) in DCE (2 ml) formaldehyde 37% in H₂O (0.147 g; 4.90 mmol) and TFA (0,011 g; 0.098 mmol) were added followed by NaBH(OAc)₃ (0.125 g; 0.59 mmol) and the reaction was stirred at RT for 1 h. The reaction mixture was neutralized by saturated a. NaHCO₃ solution, diluted and extracted with DCM. The organic layers were dried over Na₂SO₄ and concentrated and purified by column chromatography (SiO₂), eluting with DCM/EtOAc (20: 80) to afford the free base of the title compound as a white solid (0.033 g, 46%). To a solution of the free base of the title compound (0.019 g, 0.052 mmol) in Et₂O (1 mL) was added HCl 4M in dioxane (0.04 ml, 0.157 mmol) and the reaction was stirred at RT for 1 h. The reaction was concentrated under reduced pressure and the residue was triturated with Et₂O to afford the title compound hydrochloride as a white solid (0.019 g, 90%). ¹H NMR (400 MHz, DMSO-d₆) δ 10.27 (s, 1H), 7.47 (t, J=5.8 Hz, 1H), 7.42-7.33 (m, 2H), 7.26-7.06 (m, 2H), 4.48 (dd, J=11.7, 3.2 Hz, 1H), 3.39 (s, 1H), 3.30 (s, 1H), 3.20-2.80 (m, 8H), 2.77 (d, J=3.9 Hz, 4H), 1.37 (s, 3H), 1.36 (s, 3H), 0.99-0.83 (m, 1H), 0.48-0.34 (m, 2H), 0.18-0.02 (m, 2H). UPLC/MS (method A): Rt 1.68 min. MS (ES) C₂₀H₃₁FClN₃O₂ requires 363, found 364 [M+H]⁺.

### Example 169: 4-Cyclopropyl-2,2-dimethyl-6-phenyl-N-(4-phenylbutyl)piperazine-1-carboxamide tert-Butyl 2,2-dimethyl-6-oxopiperazine-1-carboxylate (XXVIIa)

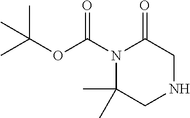 [SMILES: CC(C)(C)OC(=O)N1C(=O)CNCC1(C)C]

Following general procedure B (Method E), XXIIId (0.240 g, 0.662 mmol) afforded XXVIIa which was used in the next step without further purification. ¹H NMR (400 MHz, CDCl₃) δ 3.55 (s, 2H), 2.88 (s, 2H), 1.56 (s, 9H), 1.44 (s, 6H). UPLC/MS (method A): Rt 1.49 min. MS (ES) C₁₁H₂₀N₂O₃ requires 228, found 229 [M+H]⁺.

### tert-Butyl 4-cyclopropyl-2,2-dimethyl-6-oxopiperazine-1-carboxylate (XXVIIIa)

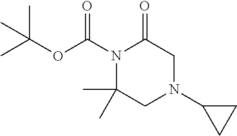 [SMILES: CC(C)(C)OC(=O)N1C(=O)CN(C2CC2)CC1(C)C]

Following general procedure I (Method C), XXVIIa (0.151 g, 0.662 mmol) and [(1-ethoxycyclopropyl)oxy]trimethylsilane (0.231 g; 1.32 mmol) afforded XXVIIIa as a colorless oil (0.120 g, 67%). ¹H NMR (400 MHz, CDCl₃) δ 3.33 (s, 2H), 2.66 (s, 2H), 1.74-1.63 (m, 1H), 1.40 (s, 6H), 0.55-0.46 (m, 2H), 0.47-0.37 (m, 2H). UPLC/MS (method A): Rt 2.39 min. MS (ES) C₁₄H₂₄N₂O₃ requires 268, found 269 [M+H]⁺.

### tert-Butyl N-[2-[cyclopropyl(phenacyl)amino]-1,1-dimethyl-ethyl]carbamate (XXIXa)

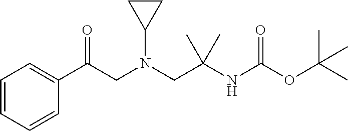 [SMILES: CC(C)(CN(CC(=O)c1ccccc1)C1CC1)NC(=O)OC(C)(C)C]

Following general procedure H (method A), XXVIIIa (0.120 g, 0.45 mmol) and PhMgBr (0.122 g, 0.671 mmol, 2.8 M in 2-MeTHF) afforded XXIXa as a yellow oil (0.128 g, 71%). ¹H NMR (400 MHz, CDCl₃) δ 7.99-7.90 (m, 2H), 7.62-7.52 (m, 1H), 7.52-7.42 (m, 2H), 4.80 (s, 1H), 4.25 (s, 2H), 3.13-3.01 (m, 2H), 2.69-2.51 (m, 1H), 1.34 (s, 9H), 1.28 (s, 6H), 0.53-0.41 (m, 4H). UPLC/MS (method B): Rt 1.90 min. MS (ES) C₂₀H₃₀N₂O₃ requires 346, found 347 [M+H]⁺.

### 1-Cyclopropyl-3,3-dimethyl-5-phenylpiperazine

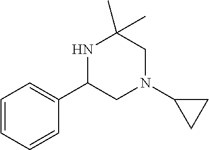 [SMILES: CC1(C)CN(C2CC2)CC(c2ccccc2)N1]

Following general procedure K, XXIXa (0.125 g, 0.361 mmol) afforded 1-cyclopropyl-3,3-dimethyl-5-phenyl-piperazine which was used in the next step without further purification. UPLC/MS (method A): Rt 1.63 min. MS (ES) C₁₈H₂₂N₂ requires 230, found 231 [M+H]⁺.

### 4-Cyclopropyl-2,2-dimethyl-6-phenyl-N-(4-phenylbutyl)piperazine-1-carboxamide

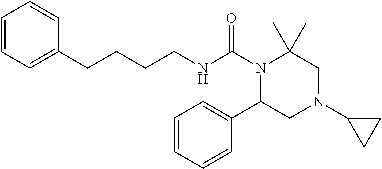 [SMILES: CC1(C)CN(C2CC2)CC(c2ccccc2)N1C(=O)NCCCCc1ccccc1]

Following general procedure D (method A), 1-cyclopropyl-3,3-dimethyl-5-phenyl-piperazine (0.083 g, 0.361 mmol) and 4-phenylbutyl isocyanate (0.070 g, 0.40 mmol) afforded the title compound as a white solid (0.053 g, 36%). ¹H NMR (400 MHz, DMSO-d₆) δ 7.39-7.30 (m, 2H), 7.28-7.06 (m, 8H), 6.94 (t, J=5.9 Hz, 1H), 4.30 (dd, J=9.6, 3.7 Hz, 1H), 2.93-2.72 (m, 3H), 2.58-2.53 (m, 1H), 2.42 (t, J=7.6 Hz, 2H), 2.32-2.21 (m, 2H), 1.60-1.48 (m, 1H), 1.32 (dq, J=8.9, 7.3, 6.8 Hz, 2H), 1.24 (s, 3H), 1.21-1.05 (m, 5H), 0.48-0.30 (m, 3H), 0.30-0.17 (m, 1H). UPLC/MS (method B): Rt 2.15 min. MS (ES) C₂₆H₃₅N₃O requires 405, found 406 [M+H]⁺.

### Example 170—Biological Activity Evaluation

The ability of exemplary compounds to inhibit acid ceramidase was measured. Experimental procedures and results are provided below.

### Part I: Assay Procedure

Cell lysates overexpressing acid ceramidase were used as the enzyme source for compound potency determination in a biochemical fluorescent assay. Briefly, compounds were preincubated with 10 pg protein of cell lysates in a dose-response manner for 1 hr at RT in the assay buffer containing 25 mM NaAC and 100 mM NaCl, pH 4.5. The reaction was initiated by the addition of substrate Rbm14-12 at a final concentration of 6.3 μM. The reaction was run at RT for 1 hr before it was stopped by the addition of the stopping buffer containing 20% methanol (v/v), 1 mg/ml NaIO₄, 0.1 M glycine, pH 10.6. The samples were incubated with the stopping buffer at RT for 1 hr to allow the fluorescent product to be formed. Finally the plate was read with SpectraMax i3 plate reader (Molecular Devices) at ex360 nm and em446 nm. Data were collected and used to determine the IC₅₀ values of compounds by curve fitting to the four-parameter inhibition equation.

### Part II: Results

Acid ceramidase inhibition values for tested compounds are provided in Table 1 below, along with c Log P and compound solubility in water. The symbol “A” indicates inhibition of less than 0.2 μM; the symbol “B” indicates inhibition in the range of 0.2 μM up to 1 μM; and the symbol “C” indicates inhibition of greater than 1 μM.

TABLE 1

| | | | Compound | |
|---|---|---|---|---|
| | | | Solubility | |
| | | | in Water | hACR |
| Example | Compound Structure | cLogP | (μM) | IC₅₀ |
| 1 | 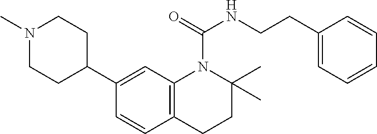 [SMILES: CN1CCC(c2ccc3c(c2)N(C(=O)NCCc2ccccc2)C(C)(C)CC3)CC1] | 5.4 | 33.2 | B |
| 2 | 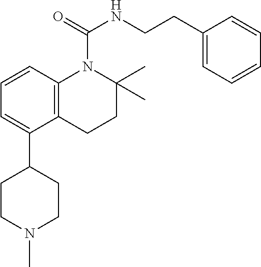 [SMILES: CN1CCC(c2cccc3c2CCC(C)(C)N3C(=O)NCCc2ccccc2)CC1] | 5.4 | 36.5 | A |
| 3 | 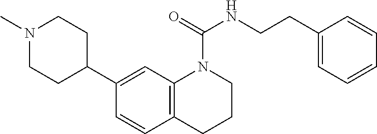 [SMILES: CN1CCC(c2ccc3c(c2)N(C(=O)NCCc2ccccc2)CCC3)CC1] | 4.4 | 38.7 | C |
| 4 | 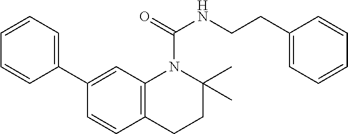 [SMILES: CC1(C)CCc2ccc(-c3ccccc3)cc2N1C(=O)NCCc1ccccc1] | 5.6 | 0.2 | B |
| 5 | 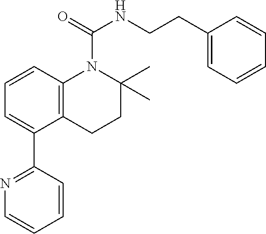 [SMILES: CC1(C)CCc2c(-c3ccccn3)cccc2N1C(=O)NCCc1ccccc1] | 5.6 | 2 3 | C |
| 6 | 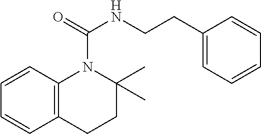 [SMILES: CC1(C)CCc2ccccc2N1C(=O)NCCc1ccccc1] | 4.8 | 3.2 | B |
| 7 | 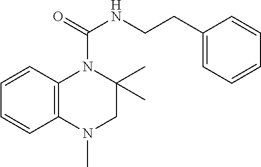 [SMILES: CN1CC(C)(C)N(C(=O)NCCc2ccccc2)c2ccccc21] | 4.6 | 6.7 | B |
| 8 | 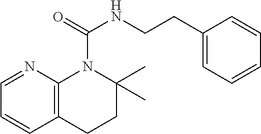 [SMILES: CC1(C)CCc2cccnc2N1C(=O)NCCc1ccccc1] | 4.3 | 27.4 | C |
| 9 | 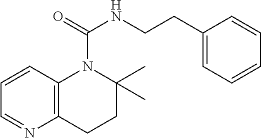 [SMILES: CC1(C)CCc2ncccc2N1C(=O)NCCc1ccccc1] | 4.3 | 25.2 | C |
| 10 | 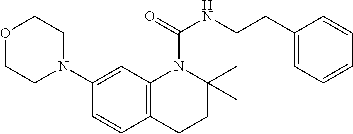 [SMILES: CC1(C)CCc2ccc(N3CCOCC3)cc2N1C(=O)NCCc1ccccc1] | 4.3 | 6.7 | C |
| 11 | 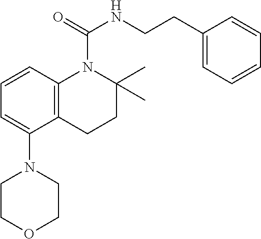 [SMILES: CC1(C)CCc2c(N3CCOCC3)cccc2N1C(=O)NCCc1ccccc1] | 4.3 | 8.1 | B |
| 12 | 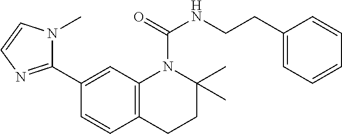 [SMILES: Cn1ccnc1-c1ccc2c(c1)N(C(=O)NCCc1ccccc1)C(C)(C)CC2] | 5.0 | 26.6 | C |
| 13 | 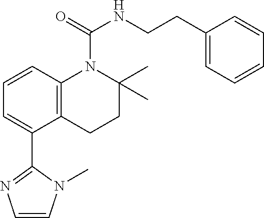 [SMILES: Cn1ccnc1-c1cccc2c1CCC(C)(C)N2C(=O)NCCc1ccccc1] | 5.0 | 33.6 | C |
| 14 | 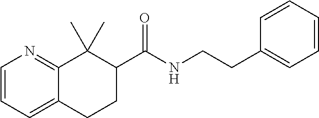 [SMILES: CC1(C)c2ncccc2CCC1C(=O)NCCc1ccccc1] | 3.2 | 24.0 | C |
| 15 | 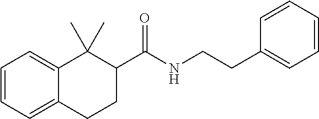 [SMILES: CC1(C)c2ccccc2CCC1C(=O)NCCc1ccccc1] | 4.6 | 0.9 | C |
| 16 | 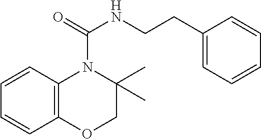 [SMILES: CC1(C)COc2ccccc2N1C(=O)NCCc1ccccc1] | 4.3 | 3.1 | C |
| 17 | 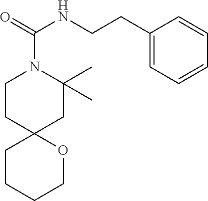 [SMILES: CC1(C)CC2(CCCCO2)CCN1C(=O)NCCc1ccccc1] | 3.3 | 28.5 | B |
| 18 | 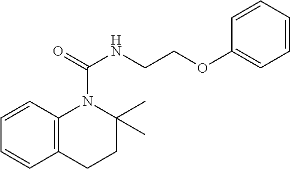 [SMILES: CC1(C)CCc2ccccc2N1C(=O)NCCOc1ccccc1] | 4.8 | 1.5 | B |
| 19 | 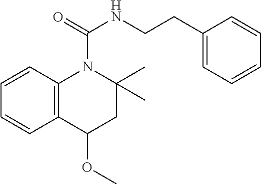 [SMILES: COC1CC(C)(C)N(C(=O)NCCc2ccccc2)c2ccccc21] | 4.0 | 6.5 | B |
| 20 | 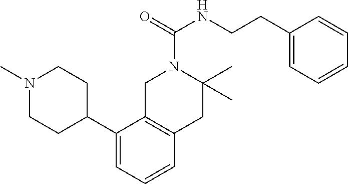 [SMILES: CN1CCC(c2cccc3c2CN(C(=O)NCCc2ccccc2)C(C)(C)C3)CC1] | 5.3 | 32.2 | B |
| 21 | 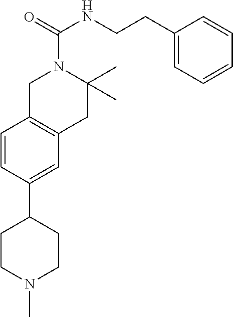 [SMILES: CN1CCC(c2ccc3c(c2)CC(C)(C)N(C(=O)NCCc2ccccc2)C3)CC1] | 5.3 | 34.2 | C |
| 22 | 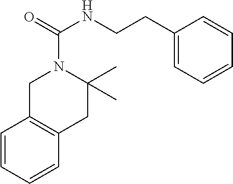 [SMILES: CC1(C)Cc2ccccc2CN1C(=O)NCCc1ccccc1] | 4.6 | 5.7 | B |
| 23 | 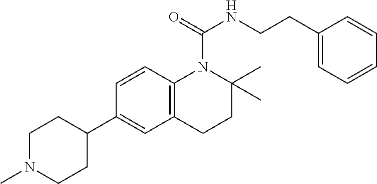 [SMILES: CN1CCC(c2ccc3c(c2)CCC(C)(C)N3C(=O)NCCc2ccccc2)CC1] | 5.4 | 33.6 | B |
| 24 | 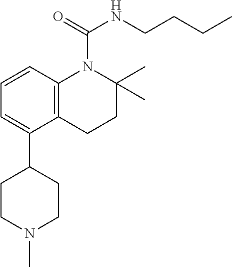 [SMILES: CCCCNC(=O)N1c2cccc(C3CCN(C)CC3)c2CCC1(C)C] | 4.7 | 40.6 | B |
| 25 | 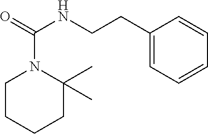 [SMILES: CC1(C)CCCCN1C(=O)NCCc1ccccc1] | 3.7 | 23.2 | B |
| 26 | 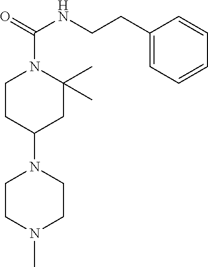 [SMILES: CN1CCN(C2CCN(C(=O)NCCc3ccccc3)C(C)(C)C2)CC1] | 1.6 | 43.7 | C |
| 27 | 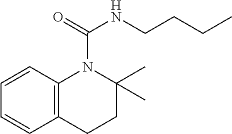 [SMILES: CCCCNC(=O)N1c2ccccc2CCC1(C)C] | 4.2 | 16.8 | B |
| 28 | 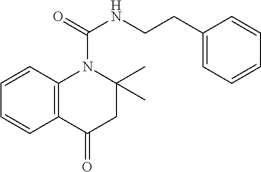 [SMILES: CC1(C)CC(=O)c2ccccc2N1C(=O)NCCc1ccccc1] | 4.3 | 3.1 | C |
| 29 | 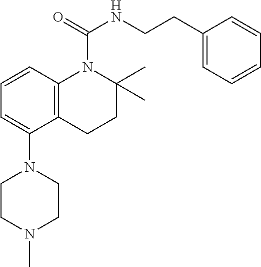 [SMILES: CN1CCN(c2cccc3c2CCC(C)(C)N3C(=O)NCCc2ccccc2)CC1] | 4.8 | 31.7 | C |
| 30 | 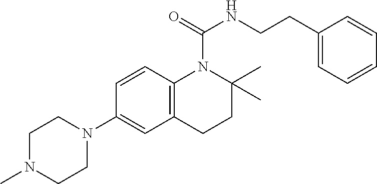 [SMILES: CN1CCN(c2ccc3c(c2)CCC(C)(C)N3C(=O)NCCc2ccccc2)CC1] | 4.8 | 36.4 | B |
| 31 | 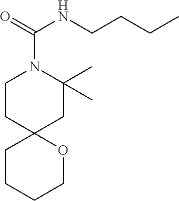 [SMILES: CCCCNC(=O)N1CCC2(CCCCO2)CC1(C)C] | 2.8 | 32.0 | C |
| 32 | 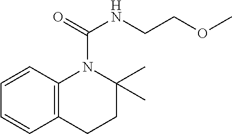 [SMILES: COCCNC(=O)N1c2ccccc2CCC1(C)C] | 3.0 | 28.7 | N/A |
| 33 | 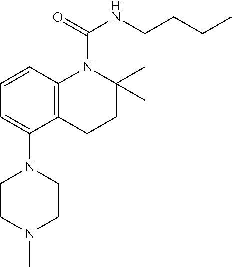 [SMILES: CCCCNC(=O)N1c2cccc(N3CCN(C)CC3)c2CCC1(C)C] | 4.1 | 22.7 | B |
| 34 | 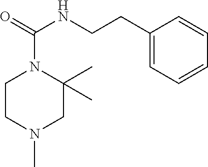 [SMILES: CN1CCN(C(=O)NCCc2ccccc2)C(C)(C)C1] | 3.3 | 23.6 | C |
| 35 | 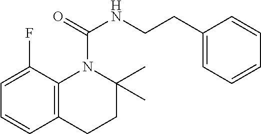 [SMILES: CC1(C)CCc2cccc(F)c2N1C(=O)NCCc1ccccc1] | 5.3 | 6.02 | B |
| 36 | 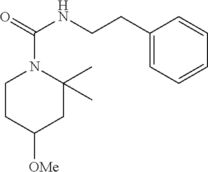 [SMILES: COC1CCN(C(=O)NCCc2ccccc2)C(C)(C)C1] | 2.4 | 33.4 | N/A |
| 37 | 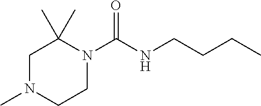 [SMILES: CCCCNC(=O)N1CCN(C)CC1(C)C] | 2.6 | 19.1 | C |
| 38 | 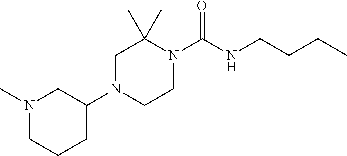 [SMILES: CCCCNC(=O)N1CCN(C2CCCN(C)C2)CC1(C)C] | 2.3 | 35.8 | C |
| 39 | 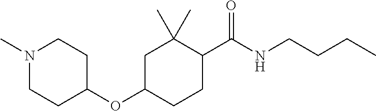 [SMILES: CCCCNC(=O)C1CCC(OC2CCN(C)CC2)CC1(C)C] | 1.5 | 34.8 | C |
| 40 | 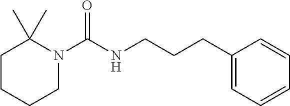 [SMILES: CC1(C)CCCCN1C(=O)NCCCc1ccccc1] | 4.1 | 23.4 | A |
| 41 | 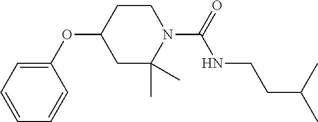 [SMILES: CC(C)CCNC(=O)N1CCC(Oc2ccccc2)CC1(C)C] | 4.2 | 3.7 | B |
| 42 | 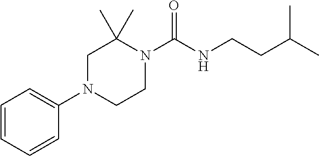 [SMILES: CC(C)CCNC(=O)N1CCN(c2ccccc2)CC1(C)C] | 4.4 | 7.0 | C |
| 43 | 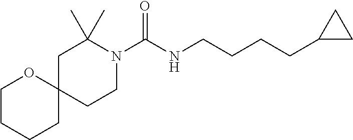 [SMILES: CC1(C)CC2(CCCCO2)CCN1C(=O)NCCCCC1CC1] | 3 7 | 16.5 | B |
| 44 | 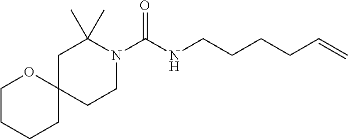 [SMILES: C=CCCCCNC(=O)N1CCC2(CCCCO2)CC1(C)C] | 3.2 | 20.8 | B |
| 45 | 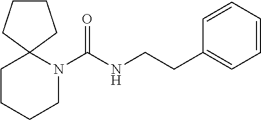 [SMILES: O=C(NCCc1ccccc1)N1CCCCC12CCCC2] | 4.2 | 6.0 | B |
| 46 | 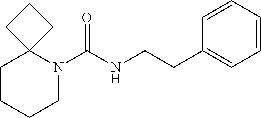 [SMILES: O=C(NCCc1ccccc1)N1CCCCC12CCC2] | 3.8 | 17.5 | C |
| 47 | 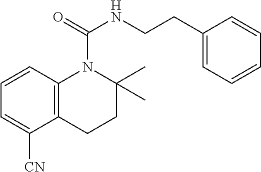 [SMILES: CC1(C)CCc2c(C#N)cccc2N1C(=O)NCCc1ccccc1] | 4.9 | 1.7 | B |
| 48 | 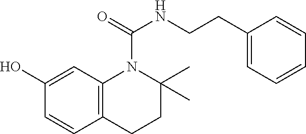 [SMILES: CC1(C)CCc2ccc(O)cc2N1C(=O)NCCc1ccccc1] | 4.9 | 0.6 | B |
| 49 | 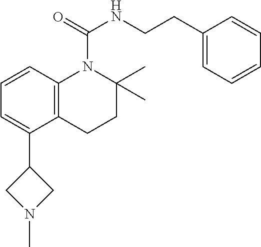 [SMILES: CN1CC(c2cccc3c2CCC(C)(C)N3C(=O)NCCc2ccccc2)C1] | 4.5 | 35.3 | B |
| 50 | 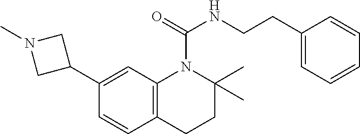 [SMILES: CN1CC(c2ccc3c(c2)N(C(=O)NCCc2ccccc2)C(C)(C)CC3)C1] | 4.5 | 32.2 | A |
| 51 | 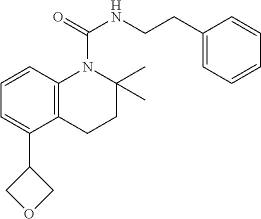 [SMILES: CC1(C)CCc2c(C3COC3)cccc2N1C(=O)NCCc1ccccc1] | 4.1 | 0.9 | B |
| 52 | 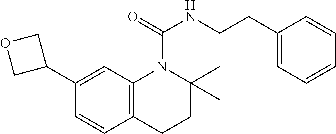 [SMILES: CC1(C)CCc2ccc(C3COC3)cc2N1C(=O)NCCc1ccccc1] | 4.1 | 5.0 | B |
| 53 | 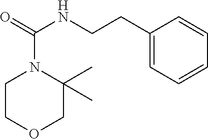 [SMILES: CC1(C)COCCN1C(=O)NCCc1ccccc1] | 2.7 | 25.6 | C |
| 54 | 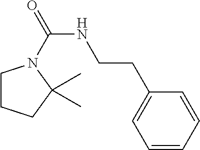 [SMILES: CC1(C)CCCN1C(=O)NCCc1ccccc1] | 3.2 | 25.6 | C |
| 55 | 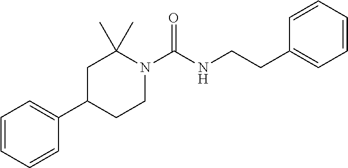 [SMILES: CC1(C)CC(c2ccccc2)CCN1C(=O)NCCc1ccccc1] | 5 2 | 0.4 | A |
| 56 | 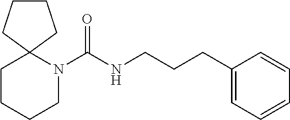 [SMILES: O=C(NCCCc1ccccc1)N1CCCCC12CCCC2] | 4.5 | 4.9 | A |
| 57 | 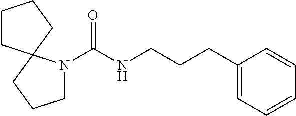 [SMILES: O=C(NCCCc1ccccc1)N1CCCC12CCCC2] | 3.9 | 19.4 | C |
| 58 | 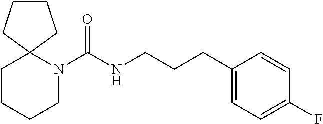 [SMILES: O=C(NCCCc1ccc(F)cc1)N1CCCCC12CCCC2] | 4.6 | 3.0 | A |
| 59 | 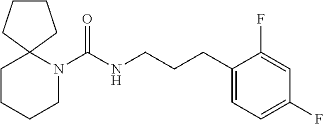 [SMILES: O=C(NCCCc1ccc(F)cc1F)N1CCCCC12CCCC2] | 4.7 | 1.1 | A |
| 60 | 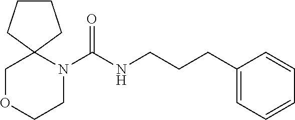 [SMILES: O=C(NCCCc1ccccc1)N1CCOCC12CCCC2] | 3.4 | 25.2 | C |
| 61 | 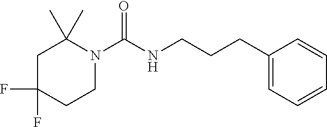 [SMILES: CC1(C)CC(F)(F)CCN1C(=O)NCCCc1ccccc1] | 3.4 | 25.7 | A |
| 62 | 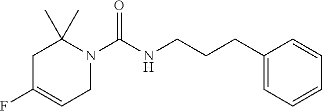 [SMILES: CC1(C)CC(F)=CCN1C(=O)NCCCc1ccccc1] | 3.8 | 15.0 | C |
| 63 | 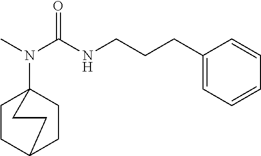 [SMILES: CN(C(=O)NCCCc1ccccc1)C12CCC(CC1)CC2] | 4.0 | 3.1 | A |
| 64 | 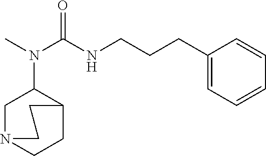 [SMILES: CN(C(=O)NCCCc1ccccc1)C1CN2CCC1CC2] | 2.5 | 35.4 | C |
| 65 | 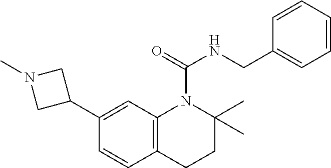 [SMILES: CN1CC(c2ccc3c(c2)N(C(=O)NCc2ccccc2)C(C)(C)CC3)C1] | 4.2 | 40.2 | B |
| 66 | 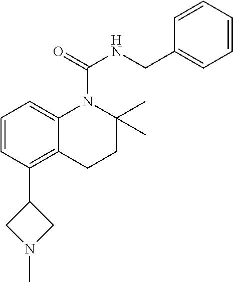 [SMILES: CN1CC(c2cccc3c2CCC(C)(C)N3C(=O)NCc2ccccc2)C1] | 4.2 | 38.5 | B |
| 67 | 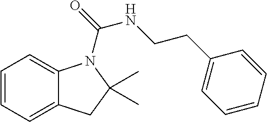 [SMILES: CC1(C)Cc2ccccc2N1C(=O)NCCc1ccccc1] | 4.4 | 8.3 | C |
| 68 | 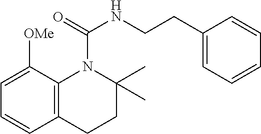 [SMILES: COc1cccc2c1N(C(=O)NCCc1ccccc1)C(C)(C)CC2] | 4.9 | 8.2 | A |
| 69 | 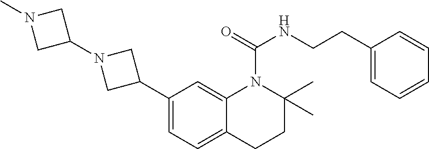 [SMILES: CN1CC(N2CC(c3ccc4c(c3)N(C(=O)NCCc3ccccc3)C(C)(C)CC4)C2)C1] | 3.3 | 35.5 | C |
| 70 | 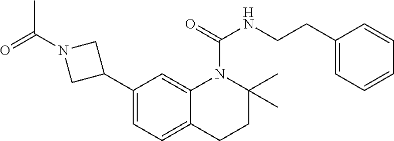 [SMILES: CC(=O)N1CC(c2ccc3c(c2)N(C(=O)NCCc2ccccc2)C(C)(C)CC3)C1] | 3.1 | 15.6 | B |
| 71 | 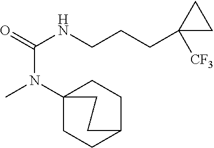 [SMILES: CN(C(=O)NCCCC1(C(F)(F)F)CC1)C12CCC(CC1)CC2] | 3.4 | 5.9 | B |
| 72 | 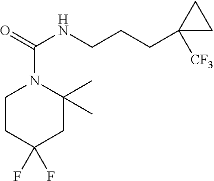 [SMILES: CC1(C)CC(F)(F)CCN1C(=O)NCCCC1(C(F)(F)F)CC1] | 2.8 | 21.4 | B |
| 73 | 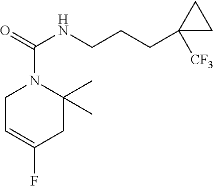 [SMILES: CC1(C)CC(F)=CCN1C(=O)NCCCC1(C(F)(F)F)CC1] | 3.2 | 4.3 | C |
| 74 | 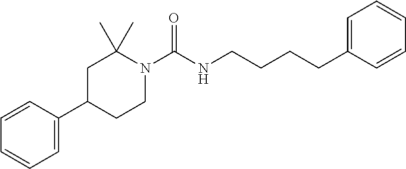 [SMILES: CC1(C)CC(c2ccccc2)CCN1C(=O)NCCCCc1ccccc1] | 5.2 | 0.2 | A |
| 75 | 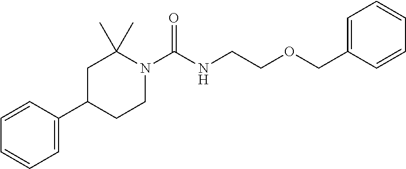 [SMILES: CC1(C)CC(c2ccccc2)CCN1C(=O)NCCOCc1ccccc1] | 3.8 | 7.1 | C |
| 76 | 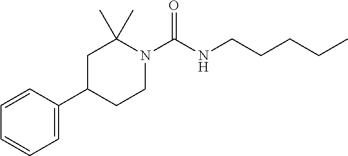 [SMILES: CCCCCNC(=O)N1CCC(c2ccccc2)CC1(C)C] | 4.1 | 2 7 | C |
| 77 | 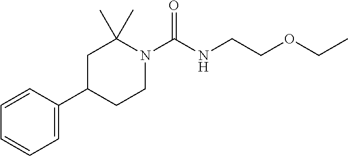 [SMILES: CCOCCNC(=O)N1CCC(c2ccccc2)CC1(C)C] | 2.6 | 64.3 | C |
| 78 | 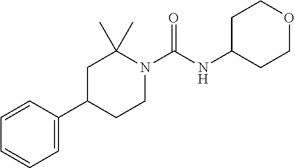 [SMILES: CC1(C)CC(c2ccccc2)CCN1C(=O)NC1CCOCC1] | 2.2 | ND | C |
| 79 | 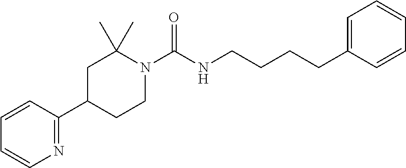 [SMILES: CC1(C)CC(c2ccccn2)CCN1C(=O)NCCCCc1ccccc1] | 4.2 | 6.4 | C |
| 80 | 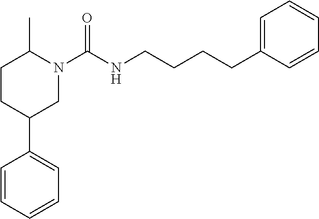 [SMILES: CC1CCC(c2ccccc2)CN1C(=O)NCCCCc1ccccc1] | 5.0 | ND | A |
| 81 | 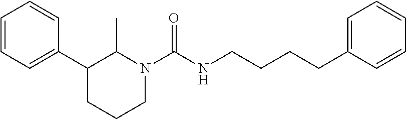 [SMILES: CC1C(c2ccccc2)CCCN1C(=O)NCCCCc1ccccc1] | 5.0 | ND | B |
| 82 | 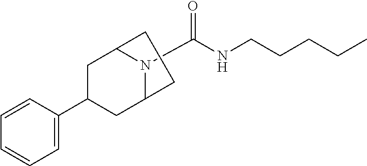 [SMILES: CCCCCNC(=O)N1C2CCC1CC(c1ccccc1)C2] | 4.1 | ND | C |
| 83 | 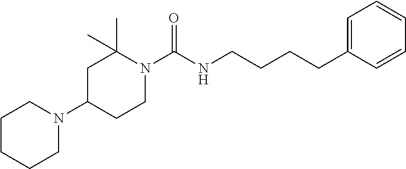 [SMILES: CC1(C)CC(N2CCCCC2)CCN1C(=O)NCCCCc1ccccc1] | 4.0 | >100 | A |
| 84 | 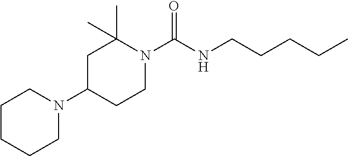 [SMILES: CCCCCNC(=O)N1CCC(N2CCCCC2)CC1(C)C] | 3.0 | ND | A |
| 85 | 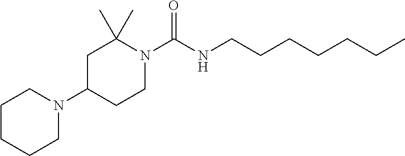 [SMILES: CCCCCCCNC(=O)N1CCC(N2CCCCC2)CC1(C)C] | 3.9 | ND | C |
| 86 | 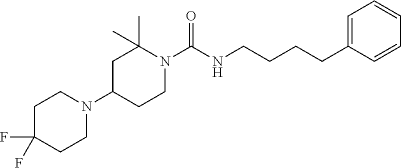 [SMILES: CC1(C)CC(N2CCC(F)(F)CC2)CCN1C(=O)NCCCCc1ccccc1] | 3.9 | 31.0 | A |
| 87 | 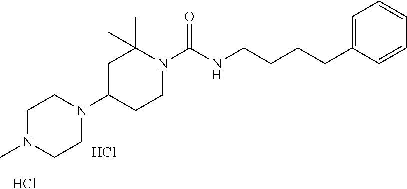 [SMILES: CN1CCN(C2CCN(C(=O)NCCCCc3ccccc3)C(C)(C)C2)CC1 ; Cl ; Cl] | 3.0 | ND | A |
| 88 | 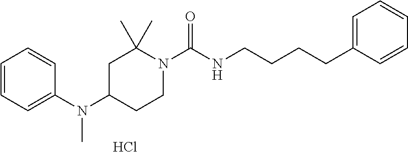 [SMILES: CN(c1ccccc1)C1CCN(C(=O)NCCCCc2ccccc2)C(C)(C)C1 ; Cl] | 4.7 | 0.01 | C |
| 89 | 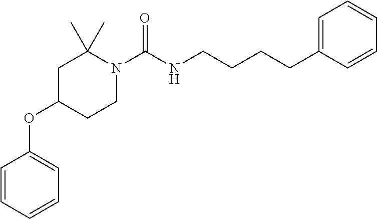 [SMILES: CC1(C)CC(Oc2ccccc2)CCN1C(=O)NCCCCc1ccccc1] | 4.6 | 0.01 | A |
| 90 | 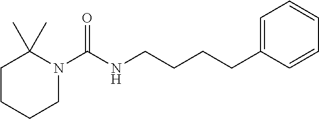 [SMILES: CC1(C)CCCCN1C(=O)NCCCCc1ccccc1] | 3.9 | ND | C |
| 91 | 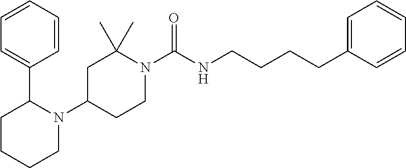 [SMILES: CC1(C)CC(N2CCCCC2c2ccccc2)CCN1C(=O)NCCCCc1ccccc1] | 5.6 | ND | C |
| 92 | 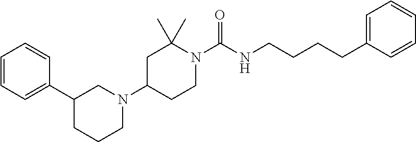 [SMILES: CC1(C)CC(N2CCCC(c3ccccc3)C2)CCN1C(=O)NCCCCc1ccccc1] | 5.3 | ND | C |
| 93 | 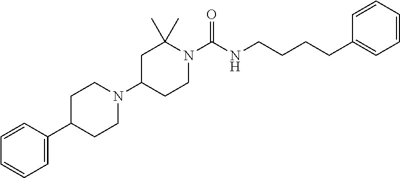 [SMILES: CC1(C)CC(N2CCC(c3ccccc3)CC2)CCN1C(=O)NCCCCc1ccccc1] | 5.3 | ND | A |
| 94 | 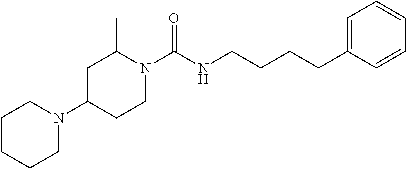 [SMILES: CC1CC(N2CCCCC2)CCN1C(=O)NCCCCc1ccccc1] | 3.8 | ND | A |
| 95 | 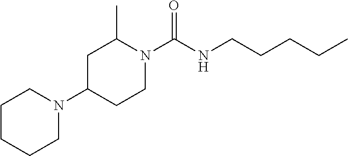 [SMILES: CCCCCNC(=O)N1CCC(N2CCCCC2)CC1C] | 2.8 | ND | B |
| 96 | 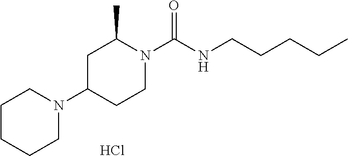 [SMILES: CCCCCNC(=O)N1CCC(N2CCCCC2)C[C@H]1C ; Cl] | 2.8 | ND | A |
| 97 | 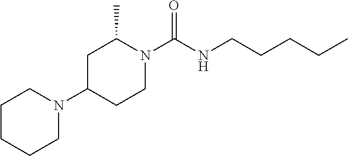 [SMILES: CCCCCNC(=O)N1CCC(N2CCCCC2)C[C@@H]1C] | 2.8 | ND | B |
| 98 | 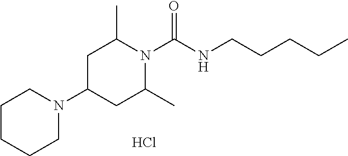 [SMILES: CCCCCNC(=O)N1C(C)CC(N2CCCCC2)CC1C ; Cl] | 3.1 | ND | A |
| 99 | 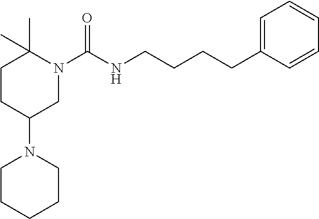 [SMILES: CC1(C)CCC(N2CCCCC2)CN1C(=O)NCCCCc1ccccc1] | 4.5 | ND | A |
| 100 | 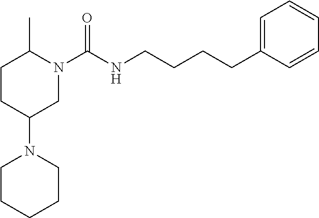 [SMILES: CC1CCC(N2CCCCC2)CN1C(=O)NCCCCc1ccccc1] | 4.3 | ND | B |
| 101 | 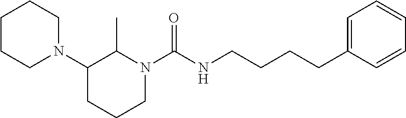 [SMILES: CC1C(N2CCCCC2)CCCN1C(=O)NCCCCc1ccccc1] | 4.3 | ND | B |
| 102 | 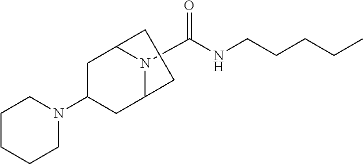 [SMILES: CCCCCNC(=O)N1C2CCC1CC(N1CCCCC1)C2] | 2.9 | ND | C |
| 103 | 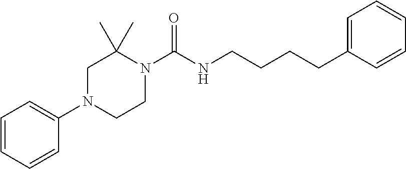 [SMILES: CC1(C)CN(c2ccccc2)CCN1C(=O)NCCCCc1ccccc1] | 4.6 | ND | A |
| 104 | 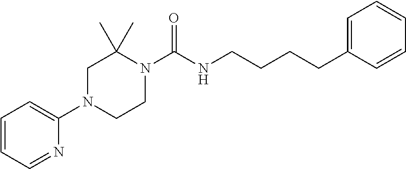 [SMILES: CC1(C)CN(c2ccccn2)CCN1C(=O)NCCCCc1ccccc1] | 4.0 | ND | C |
| 105 | 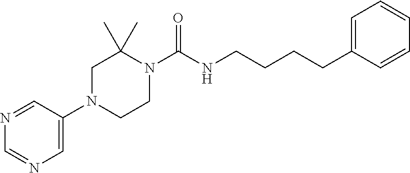 [SMILES: CC1(C)CN(c2cncnc2)CCN1C(=O)NCCCCc1ccccc1] | 2.8 | ND | C |
| 106 | 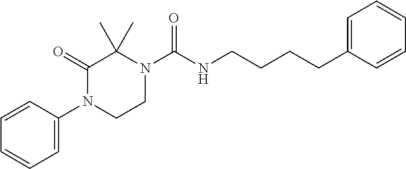 [SMILES: CC1(C)C(=O)N(c2ccccc2)CCN1C(=O)NCCCCc1ccccc1] | 3.8 | ND | A |
| 107 | 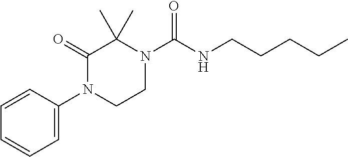 [SMILES: CCCCCNC(=O)N1CCN(c2ccccc2)C(=O)C1(C)C] | 2.8 | ND | B |
| 108 | 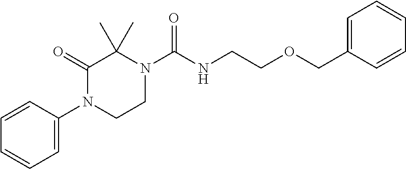 [SMILES: CC1(C)C(=O)N(c2ccccc2)CCN1C(=O)NCCOCc1ccccc1] | 2.5 | ND | C |
| 109 | 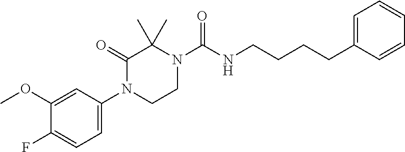 [SMILES: COc1cc(N2CCN(C(=O)NCCCCc3ccccc3)C(C)(C)C2=O)ccc1F] | 4.0 | ND | A |
| 110 | 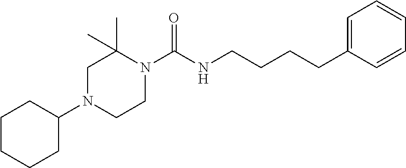 [SMILES: CC1(C)CN(C2CCCCC2)CCN1C(=O)NCCCCc1ccccc1] | 4.8 | ND | A |
| 111 | 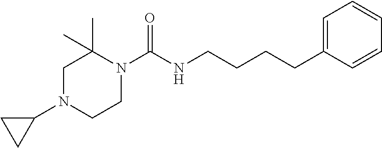 [SMILES: CC1(C)CN(C2CC2)CCN1C(=O)NCCCCc1ccccc1] | 3.4 | ND | A |
| 112 | 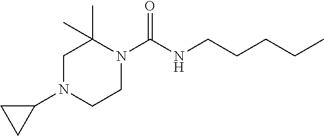 [SMILES: CCCCCNC(=O)N1CCN(C2CC2)CC1(C)C] | 2.4 | ND | A |
| 113 | 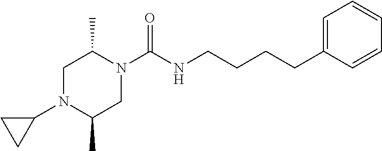 [SMILES: C[C@@H]1CN(C(=O)NCCCCc2ccccc2)[C@@H](C)CN1C1CC1] | 3.6 | ND | B |
| 114 | 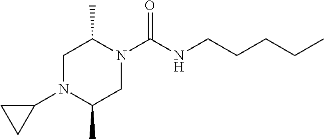 [SMILES: CCCCCNC(=O)N1C[C@@H](C)N(C2CC2)C[C@@H]1C] | 2.6 | ND | C |
| 115 | 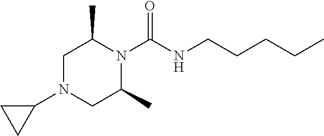 [SMILES: CCCCCNC(=O)N1[C@H](C)CN(C2CC2)C[C@@H]1C] | 2.6 | 69.1 | A |
| 116 | 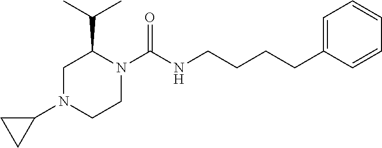 [SMILES: CC(C)[C@@H]1CN(C2CC2)CCN1C(=O)NCCCCc1ccccc1] | 4.1 | ND | B |
| 117 | 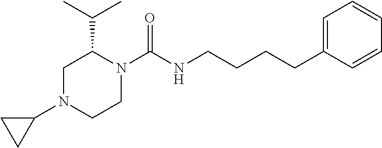 [SMILES: CC(C)[C@H]1CN(C2CC2)CCN1C(=O)NCCCCc1ccccc1] | 4.1 | ND | C |
| 118 | 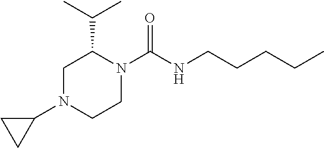 [SMILES: CCCCCNC(=O)N1CCN(C2CC2)C[C@@H]1C(C)C] | 3.0 | ND | C |
| 119 | 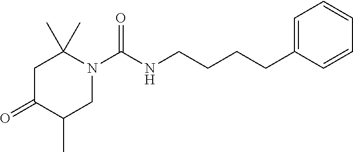 [SMILES: CC1CN(C(=O)NCCCCc2ccccc2)C(C)(C)CC1=O] | 3.2 | ND | C |
| 120 | 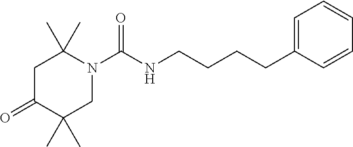 [SMILES: CC1(C)CN(C(=O)NCCCCc2ccccc2)C(C)(C)CC1=O] | 3.6 | ND | C |
| 121 | 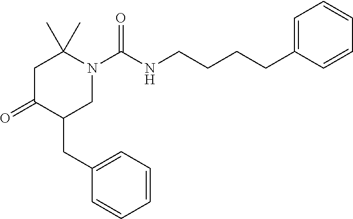 [SMILES: CC1(C)CC(=O)C(Cc2ccccc2)CN1C(=O)NCCCCc1ccccc1] | 4.7 | ND | A |
| 122 | 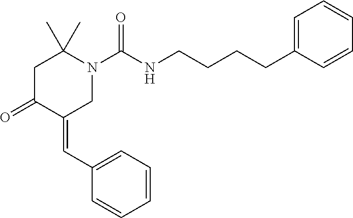 [SMILES: CC1(C)CC(=O)/C(=C/c2ccccc2)CN1C(=O)NCCCCc1ccccc1] | 4.7 | ND | A |
| 123 | 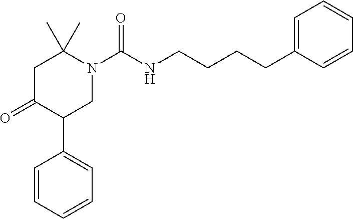 [SMILES: CC1(C)CC(=O)C(c2ccccc2)CN1C(=O)NCCCCc1ccccc1] | 4.2 | ND | C |
| 124 | 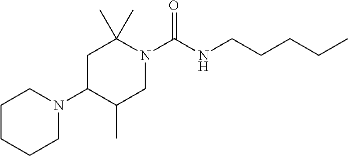 [SMILES: CCCCCNC(=O)N1CC(C)C(N2CCCCC2)CC1(C)C] | 3.4 | ND | A |
| 125 | 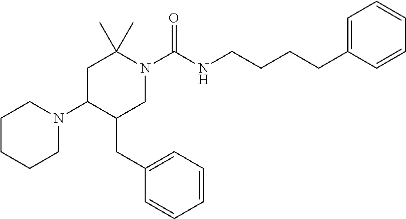 [SMILES: CC1(C)CC(N2CCCCC2)C(Cc2ccccc2)CN1C(=O)NCCCCc1ccccc1] | 5.9 | ND | A |
| 126 | 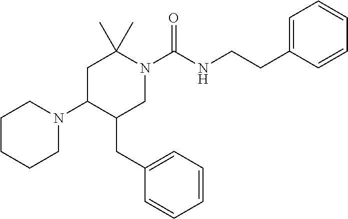 [SMILES: CC1(C)CC(N2CCCCC2)C(Cc2ccccc2)CN1C(=O)NCCc1ccccc1] | 5.0 | ND | A |
| 127 | 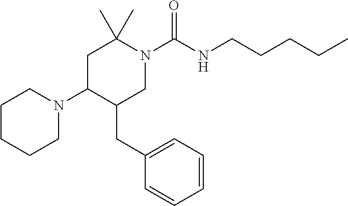 [SMILES: CCCCCNC(=O)N1CC(Cc2ccccc2)C(N2CCCCC2)CC1(C)C] | 4.9 | ND | A |
| 128 | 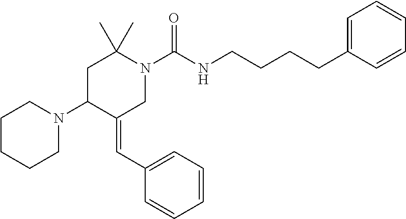 [SMILES: CC1(C)CC(N2CCCCC2)/C(=C/c2ccccc2)CN1C(=O)NCCCCc1ccccc1] | 5.9 | ND | A |
| 129 | 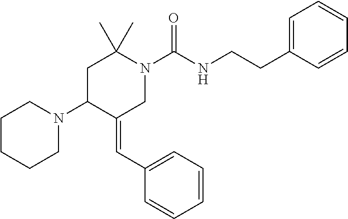 [SMILES: CC1(C)CC(N2CCCCC2)/C(=C/c2ccccc2)CN1C(=O)NCCc1ccccc1] | 5.0 | ND | B |
| 130 | 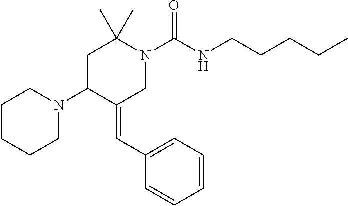 [SMILES: CCCCCNC(=O)N1C/C(=C\c2ccccc2)C(N2CCCCC2)CC1(C)C] | 4.8 | ND | A |
| 131 | 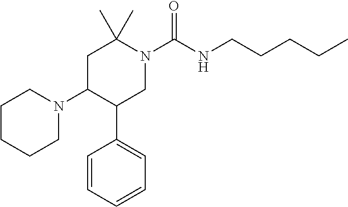 [SMILES: CCCCCNC(=O)N1CC(c2ccccc2)C(N2CCCCC2)CC1(C)C] | 4.4 | ND | A |
| 132 | 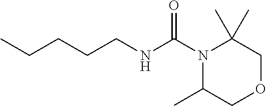 [SMILES: CCCCCNC(=O)N1C(C)COCC1(C)C] | 2.0 | 88.3 | A |
| 133 | 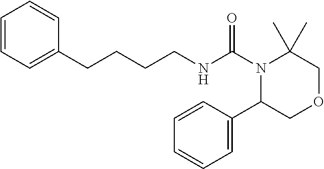 [SMILES: CC1(C)COCC(c2ccccc2)N1C(=O)NCCCCc1ccccc1] | 4.3 | ND | A |
| 134 | 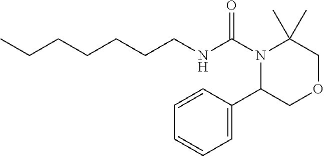 [SMILES: CCCCCCCNC(=O)N1C(c2ccccc2)COCC1(C)C] | 4.1 | ND | A |
| 135 | 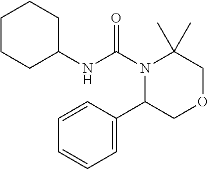 [SMILES: CC1(C)COCC(c2ccccc2)N1C(=O)NC1CCCCC1] | 3.3 | ND | B |
| 136 | 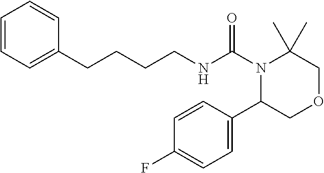 [SMILES: CC1(C)COCC(c2ccc(F)cc2)N1C(=O)NCCCCc1ccccc1] | 4.5 | ND | A |
| 137 | 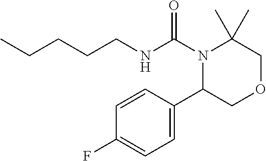 [SMILES: CCCCCNC(=O)N1C(c2ccc(F)cc2)COCC1(C)C] | 3.4 | 93 | A |
| 138 | 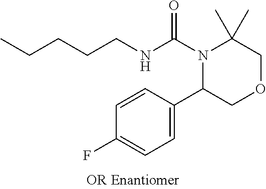 [SMILES: CCCCCNC(=O)N1C(c2ccc(F)cc2)COCC1(C)C] | 3.4 | ND | A |
| 139 | 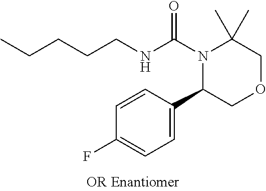 [SMILES: CCCCCNC(=O)N1[C@H](c2ccc(F)cc2)COCC1(C)C] | 3.4 | ND | A |
| 140 | 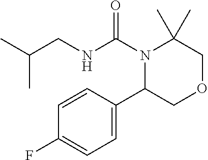 [SMILES: CC(C)CNC(=O)N1C(c2ccc(F)cc2)COCC1(C)C] | 2.8 | ND | A |
| 141 | 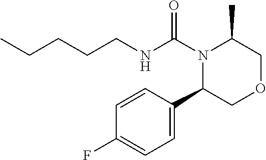 [SMILES: CCCCCNC(=O)N1[C@@H](C)COC[C@H]1c1ccc(F)cc1] | 3.2 | ND | B |
| 142 | 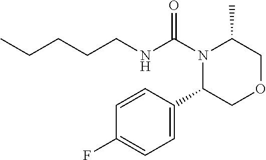 [SMILES: CCCCCNC(=O)N1[C@H](C)COC[C@@H]1c1ccc(F)cc1] | 3.2 | ND | B |
| 143 | 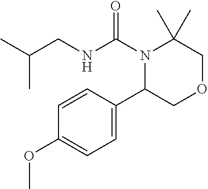 [SMILES: COc1ccc(C2COCC(C)(C)N2C(=O)NCC(C)C)cc1] | 2.6 | ND | A |
| 144 | 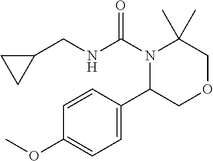 [SMILES: COc1ccc(C2COCC(C)(C)N2C(=O)NCC2CC2)cc1] | 2.3 | 97.8 | A |
| 145 | 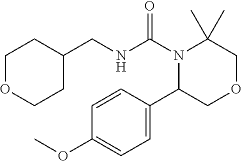 [SMILES: COc1ccc(C2COCC(C)(C)N2C(=O)NCC2CCOCC2)cc1] | 1.9 | 91.3 | B |
| 146 | 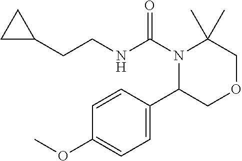 [SMILES: COc1ccc(C2COCC(C)(C)N2C(=O)NCCC2CC2)cc1] | 2.6 | ND | A |
| 147 | 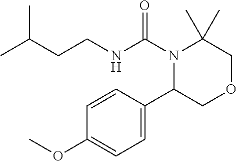 [SMILES: COc1ccc(C2COCC(C)(C)N2C(=O)NCCC(C)C)cc1] | 3.0 | ND | A |
| 148 | 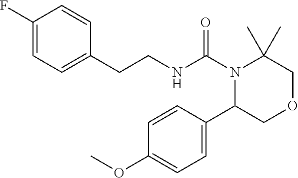 [SMILES: COc1ccc(C2COCC(C)(C)N2C(=O)NCCc2ccc(F)cc2)cc1] | 3.5 | ND | A |
| 149 | 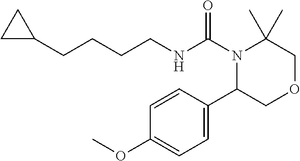 [SMILES: COc1ccc(C2COCC(C)(C)N2C(=O)NCCCCC2CC2)cc1] | 3.6 | ND | A |
| 150 | 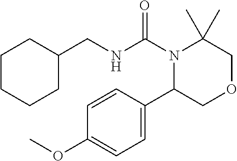 [SMILES: COc1ccc(C2COCC(C)(C)N2C(=O)NCC2CCCCC2)cc1] | 3.6 | ND | A |
| 151 | 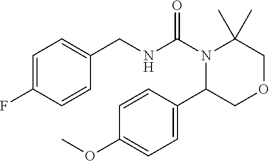 [SMILES: COc1ccc(C2COCC(C)(C)N2C(=O)NCc2ccc(F)cc2)cc1] | 3.2 | ND | A |
| 152 | 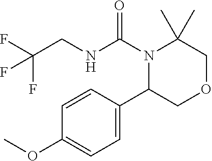 [SMILES: COc1ccc(C2COCC(C)(C)N2C(=O)NCC(F)(F)F)cc1] | 2.4 | ND | C |
| 153 | 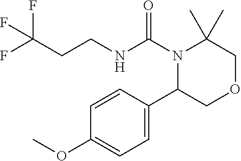 [SMILES: COc1ccc(C2COCC(C)(C)N2C(=O)NCCC(F)(F)F)cc1] | 2.2 | 79.2 | B |
| 154 | 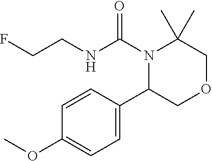 [SMILES: COc1ccc(C2COCC(C)(C)N2C(=O)NCCF)cc1] | 1.8 | 92.9 | B |
| 155 | 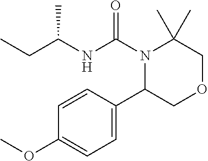 [SMILES: CC[C@H](C)NC(=O)N1C(c2ccc(OC)cc2)COCC1(C)C] | 2.7 | ND | A |
| 156 | 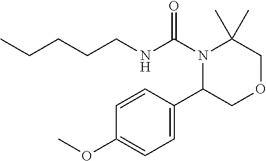 [SMILES: CCCCCNC(=O)N1C(c2ccc(OC)cc2)COCC1(C)C] | 3.2 | 91 | A |
| 157 | 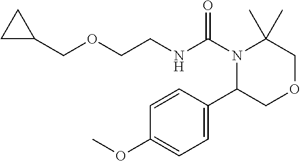 [SMILES: COc1ccc(C2COCC(C)(C)N2C(=O)NCCOCC2CC2)cc1] | 2.1 | >100 | A |
| 158 | 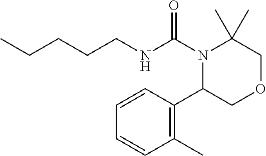 [SMILES: CCCCCNC(=O)N1C(c2ccccc2C)COCC1(C)C] | 3.7 | ND | A |
| 159 | 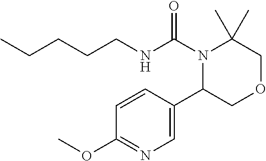 [SMILES: CCCCCNC(=O)N1C(c2ccc(OC)nc2)COCC1(C)C] | 2.6 | ND | A |
| 160 | 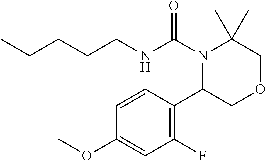 [SMILES: CCCCCNC(=O)N1C(c2ccc(OC)cc2F)COCC1(C)C] | 3.4 | ND | A |
| 161 | 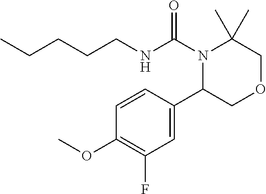 [SMILES: CCCCCNC(=O)N1C(c2ccc(OC)c(F)c2)COCC1(C)C] | 3.4 | ND | A |
| 162 | 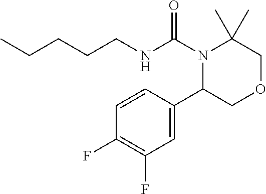 [SMILES: CCCCCNC(=O)N1C(c2ccc(F)c(F)c2)COCC1(C)C] | 3.6 | ND | A |
| 163 | 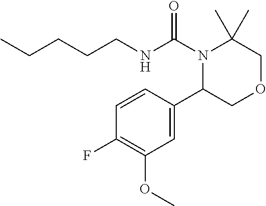 [SMILES: CCCCCNC(=O)N1C(c2ccc(F)c(OC)c2)COCC1(C)C] | 3.4 | ND | A |
| 164 | 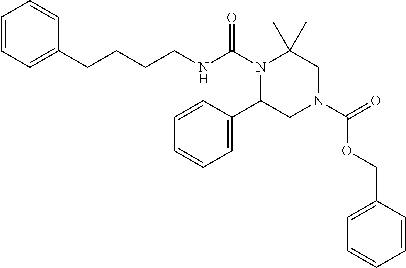 [SMILES: CC1(C)CN(C(=O)OCc2ccccc2)CC(c2ccccc2)N1C(=O)NCCCCc1ccccc1] | 6.0 | ND | C |
| 165 | 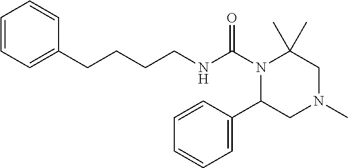 [SMILES: CN1CC(c2ccccc2)N(C(=O)NCCCCc2ccccc2)C(C)(C)C1] | 4.5 | 71.6 | A |
| 166 | 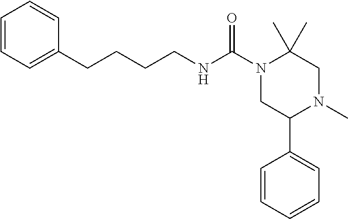 [SMILES: CN1CC(C)(C)N(C(=O)NCCCCc2ccccc2)CC1c1ccccc1] | 4.5 | ND | A |
| 167 | 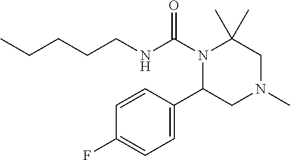 [SMILES: CCCCCNC(=O)N1C(c2ccc(F)cc2)CN(C)CC1(C)C] | 3.7 | ND | A |
| 168 | 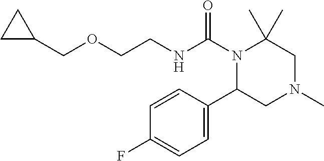 [SMILES: CN1CC(c2ccc(F)cc2)N(C(=O)NCCOCC2CC2)C(C)(C)C1] | 2.6 | ND | A |
| 169 | 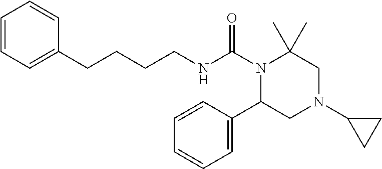 [SMILES: CC1(C)CN(C2CC2)CC(c2ccccc2)N1C(=O)NCCCCc1ccccc1] | 5.0 | ND | A |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound selected from the group consisting of a compound of formula (I-C), formula (I-D), formula (I-E), formula (I-F), formula (I-G), formula (I-H), and formula (I-B):
wherein the compound of formula (I-C) is: 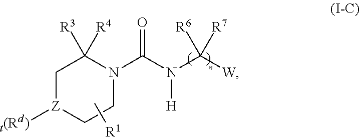
or a pharmaceutically acceptable salt thereof, wherein:
Z is selected from the group consisting of C, CH, N, and O; wherein
(i) when Z is C, t=1 (if R^{d} is oxo) or 2, when Z is CH, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered heterocyclyl), or (3-7 membered monocyclic heterocyclylene)-(3-7 membered monocyclic heterocyclyl) is optionally substituted; and
at least one of R¹ and R^{d} is cyclyl or substituted cyclyl;
(ii) when Z is O, t is 0;
R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
(iii) when Z is N, t is 1;
R¹ is selected from the group consisting of hydrogen, C₁₋₆alkyl, phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted;
R^{d} is selected from the group consisting of hydrogen, C₁₋₃alkyl, C₁₋₆alkyl, phenyl, C₃₋₇cycloalkyl, 5-6 membered heteroaryl, 3-7 membered monocyclic saturated heterocyclyl, —O—C₁₋₆alkyl, —O-phenyl, —O-(3-7 membered monocyclic heterocyclyl), —C(O)OR^{f}, —N(R^{f})₂, and (3-7 membered monocyclic heterocyclylene)-(3-7 membered monocyclic heterocyclyl), wherein the C₁₋₆alkyl, phenyl, C₃₋₇cycloalkyl, 3-7 membered monocyclic saturated heterocyclyl, —O—C₁₋₆alkyl, —O-phenyl, or (3-7 membered monocyclic heterocyclylene)-(3-7 membered monocyclic heterocyclyl) is optionally substituted;
wherein at least one of R¹ and R^{d} is cyclyl or substituted cyclyl, wherein R¹ is selected from the group consisting of phenyl, 3-7 membered monocyclic heterocyclyl, C₃₋₇cycloalkyl, and 5-6 membered heteroaryl, wherein the phenyl, C₃₋₇cycloalkyl, 5-6 membered heteroaryl, or 3-7 membered monocyclic heterocyclyl is optionally substituted, or R^{d} is selected from the group consisting of phenyl, C₃₋₇cycloalkyl, 5-6 membered heteroaryl, 3-7 membered monocyclic heterocyclyl, and (3-7 membered monocyclic heterocyclylene)-(3-7 membered monocyclic heterocyclyl), wherein the phenyl, C₃₋₇cycloalkyl, 5-6 membered heteroaryl, 3-7 membered monocyclic heterocyclyl, or (3-7 membered monocyclic heterocyclylene)-(3-7 membered monocyclic heterocyclyl) is optionally substituted;
R³ is optionally substituted C₁₋₆alkyl;
R⁴ is hydrogen or C₁₋₃alkyl; or
R³ and R⁴ can be taken together to form C₃₋₆cycloalkyl;
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
n is an integer selected from 3 to 5; and
W is an optionally substituted phenyl;
wherein the compound of formula (I-D) is: 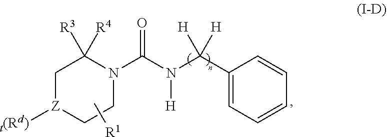
or a pharmaceutically acceptable salt thereof, wherein:
Z is selected from the group consisting of C, CH, N, and O; wherein
when Z is C, t=1 (if R^{d} is oxo), or 2, when Z is CH, t=1, and when Z is O, t=0;
when Z is C, CH, or N, R¹ is hydrogen or optionally substituted phenyl;
when Z is O, R¹ is optionally substituted phenyl;
R³ is C₁₋₃alkyl;
R⁴ is hydrogen or C₁₋₃alkyl;
R^{d} is independently, for each occurrence, selected from the group consisting of hydrogen, C₁₋₆alkyl, phenyl, 3-7 membered monocyclic heterocyclyl, wherein the C₁₋₆alkyl, phenyl, or 3-7 membered monocyclic heterocyclyl is optionally substituted;
n is 4;
wherein at least one of R¹ and R^{d} is cyclyl or substituted cyclyl, wherein R¹ is optionally substituted phenyl, or R^{d} is optionally substituted phenyl or optionally substituted 3-7 membered monocyclic heterocyclyl;
wherein the compound of formula (I-E) is: 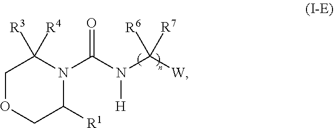
or a pharmaceutically acceptable salt thereof, wherein:
R¹ is C₁₋₆alkyl or optionally substituted phenyl;
R³ and R⁴ are independently C₁₋₆alkyl;
R⁶ and R⁷ are independently, for each occurrence, selected from the group consisting of hydrogen, C₁₋₆alkyl, C₁₋₆haloalkyl, and halogen;
n is an integer selected from 1 to 5; and
W is selected from the group consisting of methyl, an optionally substituted phenyl, and an optionally substituted C₃₋₇cycloalkyl;
wherein the compound of formula (I-F) is: 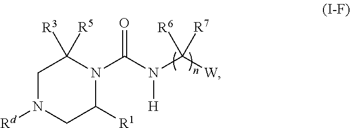
or a pharmaceutically acceptable salt thereof, wherein:
R¹ is C₁₋₆alkyl;
R³ is C₁₋₆alkyl;
R⁴ is C₁₋₆alkyl or hydrogen;
R⁶ and R⁷ are independently, for each occurrence, selected from the group consisting of hydrogen, C₁₋₆alkyl, C₁₋₆haloalkyl, and halogen;
n is an integer selected from 1 to 6;
W is selected from the group consisting of methyl, an optionally substituted phenyl, and an optionally substituted C₃₋₇cycloalkyl; and
R^{d} is C₃₋₇cycloalkyl optionally substituted with C₁₋₆alkyl or halogen;
wherein the compound of formula (I-G) is: 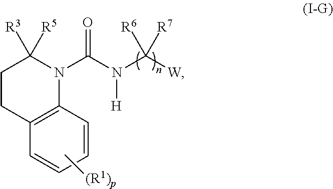
or a pharmaceutically acceptable salt thereof, wherein:
R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl; wherein at least one of R¹ is selected from the group consisting of phenyl, 3-7 membered monocyclic heterocyclyl, and 5-6 membered heteroaryl;
p is an integer selected from 1 to 2; wherein,
R³ is C₁₋₂alkyl;
R⁴ is hydrogen or C₁₋₂alkyl;
wherein R³ and R⁴ can be taken together to form C₃₋₅cycloalkyl;
R^{a} is independently, for each occurrence, selected from the group consisting of hydrogen, C₁₋₆alkyl, phenyl, and 3-7 membered monocyclic heterocyclyl;
R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
n is an integer selected from 0 to 6; and
when n is an integer selected from 1 to 6, W is selected from the group consisting of methyl, methylene (i.e., 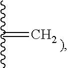
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 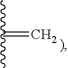
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered monocyclic heterocyclyl and 5-6 membered heteroaryl are optionally substituted;
wherein the compound of formula (I-H) is: 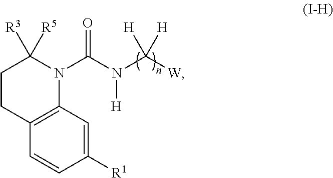
or a pharmaceutically acceptable salt thereof, wherein:
R¹ is an optionally substituted 3-7 membered monocyclic heterocyclyl (e.g., 3-7 membered monocyclic heterocyclyl optionally substituted with C₁₋₆alkyl);
R³ and R⁴ are independently C₁₋₂alkyl; wherein R³ and R⁴ can be taken together to form C₃₋₅cycloalkyl;
n is 1 to 6; and
W is an optionally substituted phenyl;
wherein the compound of formula (I-B) is: 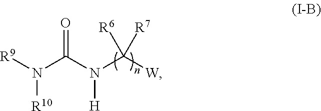
or a pharmaceutically acceptable salt thereof, wherein:
R⁹ and R¹⁰ are independently selected from the group consisting of hydrogen, C₁₋₆alkyl, C₁₋₆alkylene-phenyl, 7-8 membered bridged bicyclic cycloalkyl, 7-8 membered bridged bicyclic heterocyclyl, and 3-7 membered monocyclic heterocyclyl; and
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
n is an integer selected from 0 to 6; and
when n is an integer selected from 1 to 6, W is selected from the group consisting of methyl, methylene (i.e., 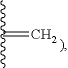
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 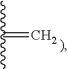
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered monocyclic heterocyclyl and 5-6 membered heteroaryl are optionally substituted.

2. The compound of claim 1, wherein the compound is a compound of formula (I-C) or formula (I-D), wherein R⁴ is hydrogen or methyl.

3. The compound of claim 1 or 2, wherein the compound is a compound of formula (I-C) or formula (I-D), wherein R^{d} is selected from the group consisting of hydrogen, methyl, phenyl, and

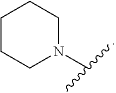 [SMILES: *N1CCCCC1]

4. The compound of any one of claims 1-3, wherein the compound is a compound of formula (I-C) or formula (I-D), wherein Z is CH and R^{d} is selected from the group consisting of hydrogen, phenyl, and

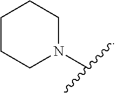 [SMILES: *N1CCCCC1]

5. The compound of any one of claims 1-3, wherein the compound is a compound of formula (I-C) or formula (I-D), wherein Z is N and R^{d} is methyl or phenyl.

6. The compound of claim 1, wherein the compound is a compound of formula (I-G) or formula (I-H), wherein R¹ is

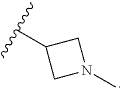 [SMILES: *C1CN(C)C1]

7. The compound of claim 1, wherein the compound is a compound of formula (I-B), wherein R⁹ is C₁₋₆alkyl.

8. The compound of claim 1 or 7, wherein the compound is a compound of formula (I-B), wherein R⁹ is methyl.

9. The compound of any one of claims 1, 7, and 8, wherein the compound is a compound of formula (I-B), wherein R¹⁰ is selected from the group consisting of C₁₋₆alkylene-phenyl, 3-7 membered monocyclic heterocyclyl, 7-8 membered bridged bicyclic cycloalkyl, and 7-8 membered bridged bicyclic heterocyclyl, wherein the 3-7 membered monocyclic heterocyclyl is optionally substituted with methyl.

10. The compound of claim 9, wherein R¹⁰ is selected from the group consisting of

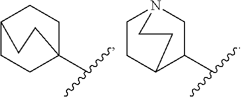

11. The compound of any one of claims 1-10, wherein R³ and R⁴ are methyl.

12. A compound of Formula (II): 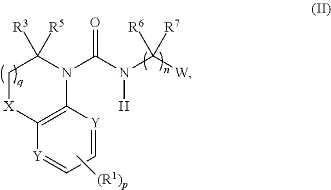
or a pharmaceutically acceptable salt thereof, wherein:
R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl;
p is an integer selected from 0 to 2;
R³ and R⁴ are independently selected from hydrogen or C₁₋₂alkyl, or R³ and R⁴ can be taken together to form C₃₋₄cycloalkyl;
X is selected from the group consisting of CR^{b}₂, NR^{a}, and O;
each Y is independently selected from C(R²)₂ or N;
R² is selected from the group consisting of hydrogen, C₁₋₆alkyl, halogen, cyano, —O—R^{c}, phenyl, 3-7 membered monocyclic heterocyclyl, C₃₋₇cycloalkyl, and 5-6 membered heteroaryl;
R^{a} is independently, for each occurrence, selected from the group consisting of hydrogen, C₁₋₆alkyl, phenyl, and 3-7 membered monocyclic heterocyclyl;
R^{c} is independently, for each occurrence, selected from the group consisting of C₁₋₆alkyl, C₁₋₆haloalkyl, phenyl, C₃₋₇cycloalkyl, 3-7 membered monocyclic heterocyclyl optionally substituted with C₁₋₆alkyl, 5-6 membered heteroaryl, phenyl, and C₁₋₆alkylene-N(R^{a})₂;
R^{b} is independently, for each occurrence, selected from the group consisting of hydrogen, C₁₋₆alkyl, phenyl, —O—C₁₋₆alkyl, —O-phenyl, —O-(3-7 membered monocyclic heterocyclyl), 3-7 membered monocyclic heterocyclyl, (3-7 membered monocyclic heterocyclylene)-(3-7 membered monocyclic heterocyclyl) or two R^{b} can be taken together to form oxo;
q is an integer selected from 0 or 1;
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
n is an integer selected from 0 to 6; and
when n is an integer selected from 1 to 6, W is selected from the group consisting of methyl, methylene (i.e., 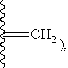
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 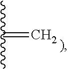
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered monocyclic heterocyclyl and 5-6 membered heteroaryl are optionally substituted.

13. The compound of claim 12, wherein R³ and R⁴ are hydrogen or methyl, or R³ and R⁴ are taken together to form cyclopropylene.

14. The compound of claim 12 or 13, wherein R³ and R⁴ are methyl.

15. The compound of claim 12 or 13, wherein R³ and R⁴ are hydrogen.

16. A compound of formula (III): 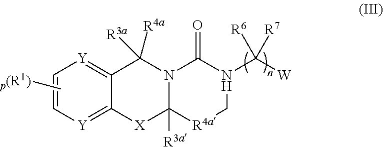
or a pharmaceutically acceptable salt thereof, wherein:
R¹ is selected from the group consisting of C₁₋₆alkyl, halogen, cyano, —O—R^{c}, phenyl, 3-7 membered monocyclic heterocyclyl, C₃₋₇cycloalkyl, and 5-6 membered heteroaryl;
p is an integer selected from 0 to 2;
R^{3a} and R^{4a} are independently selected from C₁₋₂alkyl, or R^{3a} and R^{4a} can be taken together to form C₃₋₄cycloalkyl; and R^{3a′} and R^{4a′} are independently selected from hydrogen and C₁₋₂alkyl or R^{3a′} and R^{4a′} can be taken together to form C₃₋₄cycloalkyl; or
R^{3a′} and R^{4a′} are independently selected from C₁₋₂alkyl, or R^{3a′} and R^{4a′} can be taken together to form C₃₋₄cycloalkyl; and R^{3a} and R^{4a} are independently selected from hydrogen and C₁₋₂alkyl or R^{3a} and R^{4a} can be taken together to form C₃₋₄cycloalkyl;
X is selected from the group consisting of CR^{b}₂, NR^{a}, and O;
each Y is independently selected from C(R²)₂ and N;
R² is selected from the group consisting of hydrogen, C₁₋₆alkyl, halogen, cyano, —O—R^{c}, phenyl, 3-7 membered monocyclic heterocyclyl, C₃₋₇cycloalkyl, and 5-6 membered heteroaryl;
R^{b} is independently, for each occurrence, selected from the group consisting of hydrogen, C₁₋₆alkyl, phenyl, —O—C₁₋₆alkyl, —O-phenyl, —O-(3-7 membered monocyclic heterocyclyl), 3-7 membered monocyclic heterocyclyl, (3-7 membered monocyclic heterocyclylene)-(3-7 membered monocyclic heterocyclyl) or two R^{b} can be taken together to form oxo; and
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
R^{a} is independently, for each occurrence, selected from the group consisting of hydrogen, C₁₋₆alkyl, phenyl, and 3-7 membered monocyclic heterocyclyl;
R^{c} is independently, for each occurrence, selected from the group consisting of C₁₋₆alkyl, C₁₋₆haloalkyl, phenyl, C₃₋₇cycloalkyl, 3-7 membered monocyclic heterocyclyl optionally substituted with C₁₋₆alkyl, 5-6 membered heteroaryl, phenyl, and C₁₋₆alkylene-N(R^{a})₂;
n is an integer selected from 0 to 6; and
when n is an integer selected from 1 to 6, W is selected from the group consisting of methyl, methylene (i.e., 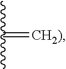
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 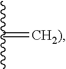
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and −O—(C₁₋₆alkylene)-phenyl are optionally substituted (e.g., with one or more halogens or CF₃);
wherein any aforementioned 3-7 membered heterocyclyl and 5-6 membered heteroaryl are optionally substituted.

17. The compound of claim 16, wherein R^{3a} and R^{4a} are methyl, and R^{3a′} R^{4a′} are hydrogen.

18. The compound of claim 16, wherein R^{3a′} and R^{4a′} are methyl, and R^{3a} and R^{4a} are hydrogen.

19. The compound of any one of claims 12-18, wherein p is 1.

20. The compound of any one of claims 12-19, wherein R¹ is selected from the group consisting of cyano, halogen, 3-7 membered monocyclic heterocyclyl, and 5-6 membered heteroaryl, wherein the 3-7 membered monocyclic heterocyclyl and 5-6 membered heteroaryl are optionally substituted with one or more substituents selected from the group consisting of methyl, —C(O)CH₃, or 3-7 membered monocyclic heterocyclyl.

21. The compound of any one of claims 12-20, wherein R¹ is selected from the group consisting of cyano, halogen,

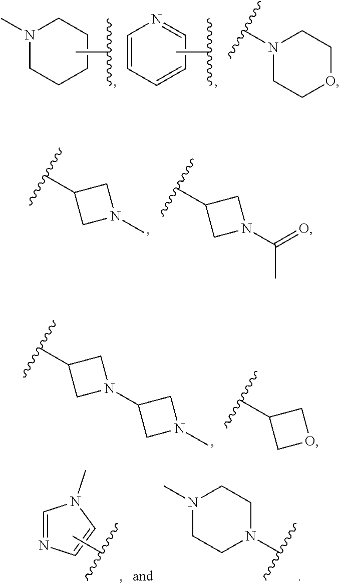

22. The compound of any one of claims 12-21, wherein R¹ is selected from the group consisting of cyano, halogen,

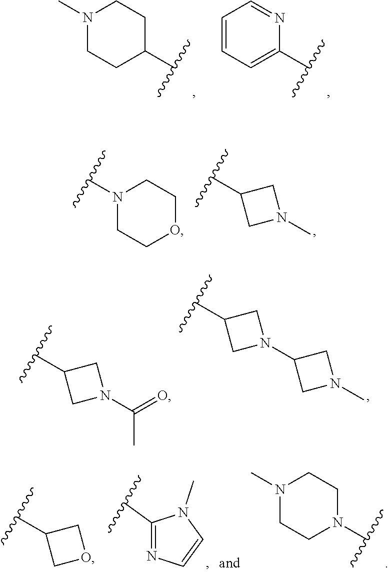

23. The compound of any one of claims 12-20, wherein R¹ is 3-7 membered monocyclic heterocyclyl, wherein the 3-7 membered monocyclic heterocyclyl is optionally substituted with methyl.

24. The compound of any one of claims 12-23, wherein R¹ is

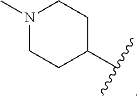 [SMILES: *C1CCN(C)CC1]

25. The compound of any one of claims 12-18, wherein p is 0.

26. The compound of any one of claims 12-25, wherein X is selected from the group consisting of CH₂, NCH₃, O, CH—O—C₁₋₆alkyl, C═O, and N-(3-7 membered monocyclic heterocyclyl), CH-(3-7 membered monocyclic heterocyclyl), wherein the 3-7 membered monocyclic heterocyclyl is optionally substituted with methyl.

27. The compound of any one of claims 12-25, wherein X is CR^{b}₂.

28. The compound of any one of claims 12-27, wherein X is CH₂.

29. The compound of any one of claims 12-28, wherein each Y is C(R²)₂.

30. The compound of any one of claims 12-28, wherein one Y is C(R²)₂ and the other Y is N.

31. The compound of any one of claims 12-30, wherein each R² is independently selected from the group consisting of hydrogen, cyano, fluorine, —OCH₃,

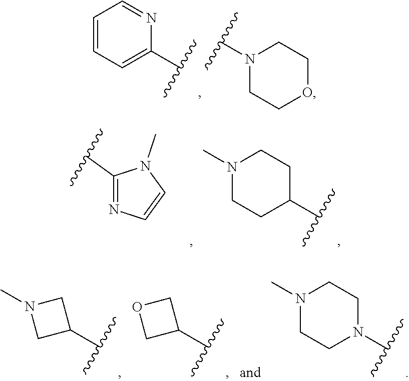

32. The compound of any one of claims 12-28, wherein each Y is N.

33. A compound of formula (IV): 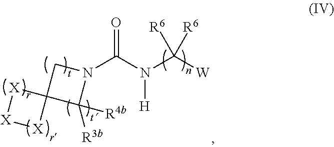
or a pharmaceutically acceptable salt thereof, wherein
R^{3b} and R^{4b} are independently, for each occurrence, selected from hydrogen and C₁₋₂alkyl; wherein at least one of R^{3b} and R^{4b} on the carbon adjacent to the nitrogen is selected from C₁₋₂alkyl;
X is independently, for each occurrence, selected from the group consisting of CR^{b}₂, NR^{a}, and O;
R^{b} is independently, for each occurrence, selected from the group consisting of hydrogen, C₁₋₆alkyl, phenyl, —O—C₁₋₆alkyl, —O-phenyl, —O-(3-7 membered monocyclic heterocyclyl), 3-7 membered monocyclic heterocyclyl, (3-7 membered monocyclic heterocyclylene)-(3-7 membered monocyclic heterocyclyl) or two R^{b} can be taken together to form oxo;
r, r′, t, and t′ are independently, for each occurrence, 1 or 2;
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
R^{a} is independently, for each occurrence, selected from the group consisting of hydrogen, C₁₋₆alkyl, phenyl, and 3-7 membered monocyclic heterocyclyl;
n is an integer selected from 0 to 6; and
when n is an integer selected from 1 to 6, W is selected from the group consisting of methyl, methylene (i.e., 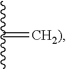
halogen, phenyl, C₃₋₇cycloalkyl, 3-7 membered monocyclic heterocyclyl, 5-6 member heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 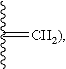
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned 3-7 membered heterocyclyl and 5-6 membered heteroaryl are optionally substituted.

34. The compound of claim 33, wherein 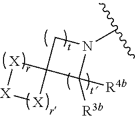
is selected from the group consisting of: 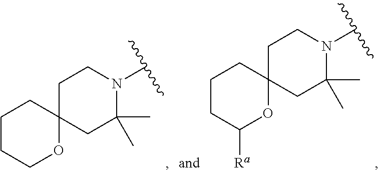
wherein R^{a} is as defined in claim 33.

35. The compound of claim 33 or 34, wherein 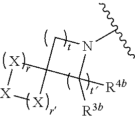
is

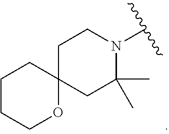 [SMILES: *N1CCC2(CCCCO2)CC1(C)C]

36. The compound of claim 33 or 34, wherein R^{a} is selected from methyl and

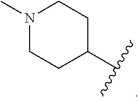 [SMILES: *C1CCN(C)CC1]

37. The compound of any one of claims 33-36, wherein each of R^{3b} and R^{4b} on the carbon adjacent to the nitrogen is methyl.

38. The compound of any one of claims 33-37, wherein X is independently for each occurrence selected from the group consisting of CH₂, O, and NR^{a}.

39. A compound of formula (V): 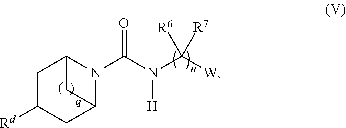
or a pharmaceutically acceptable salt thereof, wherein
q is an integer selected from 1 and 2;
R^{d} is independently, for each occurrence, selected from the group consisting of hydrogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, or (3-7 membered monocyclic heterocyclylene)-(3-7 membered monocyclic heterocyclyl); and
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
R^{f} is independently, for each occurrence, selected from the group consisting of hydrogen, C₁₋₆alkyl, —(C₁₋₆alkylene)-phenyl, and phenyl;
n is an integer selected from 0 to 6; and
when n is an integer selected from 1 to 6, W is selected from the group consisting of methyl, methylene (i.e., 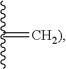
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃),
when n is 0, W is selected from the group consisting of methyl, methylene (i.e., 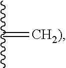
halogen, C₃₋₇cycloalkyl, 3-7 membered saturated monocyclic heterocycly, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl, wherein the aforementioned methyl, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl are optionally substituted (e.g., with one or more halogens or CF₃);
wherein any aforementioned C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), and (3-7 membered monocyclic heterocyclylene)-(3-7 membered monocyclic heterocyclyl) are optionally substituted.

40. The compound of claim 39, wherein q is 2.

41. The compound of claim 39 or 40, wherein R^{d} is selected from phenyl and

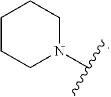 [SMILES: *N1CCCCC1]

42. A compound of formula (VI): 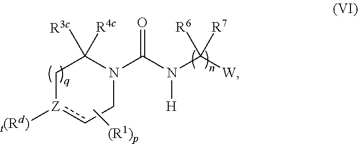
or a pharmaceutically acceptable salt thereof, wherein [symbol]
denotes a single bond or a double bond;
R¹ is selected from the group consisting of C₁₋₆alkyl, halogen, cyano, oxo, —O—R^{c}, phenyl, —(C₁₋₆alkylene)-phenyl, —(C₁₋₆alkenyl)-phenyl, 3-7 membered monocyclic heterocyclyl, C₃₋₇cycloalkyl, and 5-6 membered heteroaryl;
R^{3c} and R^{4c} are independently selected from hydrogen or C₁₋₃alkyl, wherein at least one of R^{3c} or R^{4c} is C₁₋₃alkyl, or R^{3c} and R^{4c} can be taken together to form C₃₋₆cycloalkyl;
Z is selected from the group consisting of CH, N, and O, wherein when Z is C, t=1 or 2, when Z is CH, t=1, when Z is N, t=1, and when Z is O, t=0;
R^{c} is selected from the group consisting of C₁₋₆alkyl, C₁₋₆haloalkyl, C₃₋₇cycloalkyl, 3-7 membered monocyclic heterocyclyl, 5-6 membered heteroaryl, phenyl, and C₁₋₆alkylene-N(R^{a})₂;
R^{d} is independently, for each occurrence, selected from the group consisting of hydrogen, halogen, oxo, C₁₋₆alkyl, phenyl, C₃₋₇cycloalkyl, 5-6 membered heteroaryl, 3-7 membered monocyclic heterocyclyl, —O—C₁₋₆alkyl, —O-phenyl, —O-(3-7 membered monocyclic heterocyclyl), —C(O)OR^{f}, —N(R^{f})₂, or (3-7 membered monocyclic heterocyclylene)-(3-7 membered monocyclic heterocyclyl);
p is an integer selected from 0 to 3;
q is an integer selected from 0 or 1;
R⁶ and R⁷ are independently, for each occurrence, selected from the group consisting of hydrogen, C₁₋₆alkyl, C₁₋₆haloalkyl, and halogen; or R⁶ and R⁷ can be taken together to form C₃₋₇cycloalkylene;
R^{f} is independently, for each occurrence, selected from the group consisting of hydrogen, C₁₋₆alkyl, —(C₁₋₆alkylene)-phenyl, and phenyl;
n is an integer selected from 0 to 6; and
when n is an integer selected from 1 to 6, W is selected from the group consisting of methyl, methylene (i.e., 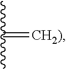
halogen, phenyl, C₃₋₇cycloalkyl, 3-7 membered monocyclic heterocyclyl, 5-6 membered heteroaryl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl,
when n is 0, W is selected from the group consisting of methyl, methylene (i.e., 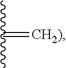
halogen, C₃₋₇cycloalkyl, 3-7 membered saturated monocyclic heterocyclyl, —O—C₁₋₆alkyl, —O—C₁₋₆haloalkyl, —O-phenyl, —O—(C₁₋₆alkylene)-C₃₋₇cycloalkyl, and —O—(C₁₋₆alkylene)-phenyl.

43. The compound of claim 42, wherein R¹ is selected from the group consisting of cyano, halogen, methyl, oxo, phenyl, 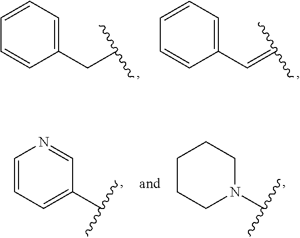
wherein the aforementioned phenyl, 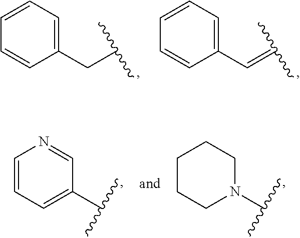
are optionally substituted with 1-2 substituents independently, for each occurrence, selected from the group consisting of halogen, C₁-C₆alkyl, and —O—C₁-C₆alkyl.

44. The compound of claim 42 or 43, wherein R¹ is selected from the group consisting of cyano, fluorine, methyl, oxo, phenyl,

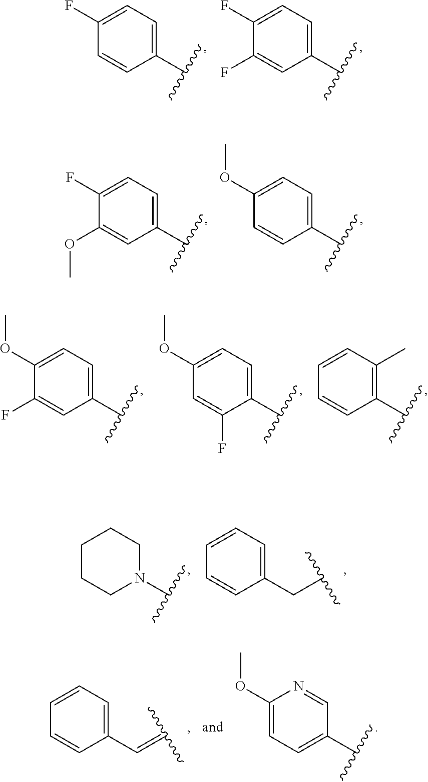

45. The compound of any one of claims 42-44, wherein R^{3c} and R^{4c} are independently selected from hydrogen or C₁₋₃alkyl, or R^{3c} and R^{4c} can be taken together to form C₃₋₅cycloalkyl.

46. The compound of any one of claims 42-45, wherein R^{3c} and R^{4c} are independently selected from hydrogen, methyl and isopropyl.

47. The compound of any one of claims 42-46, wherein each of R^{3c} and R^{4c} is methyl.

48. The compound of any one of claims 42-45, wherein R^{3c} and R^{4c} are taken together to form cyclobutyl or cyclopentyl.

49. The compound of any one of claims 42-48, wherein Z is CH.

50. The compound of any one of claims 42-48, wherein Z is N.

51. The compound of any one of claims 42-50, wherein R^{d} is selected from the group consisting of hydrogen, methyl, —O—(C₁₋₆alkyl), 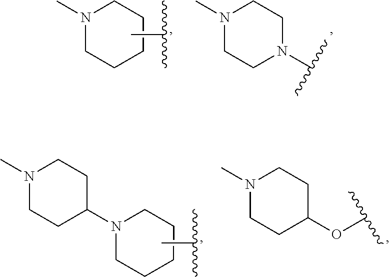
O-phenyl, phenyl, 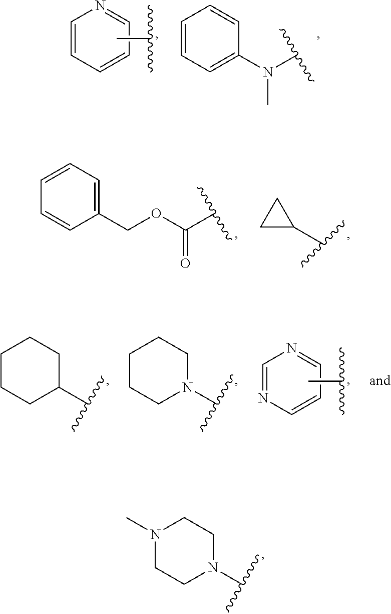
wherein the aforementioned —O—(C₁₋₆alkyl), 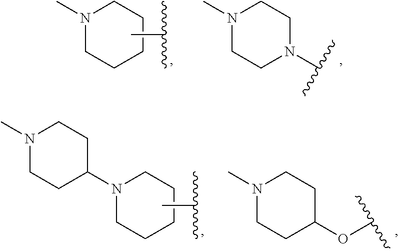
phenyl, O-phenyl, 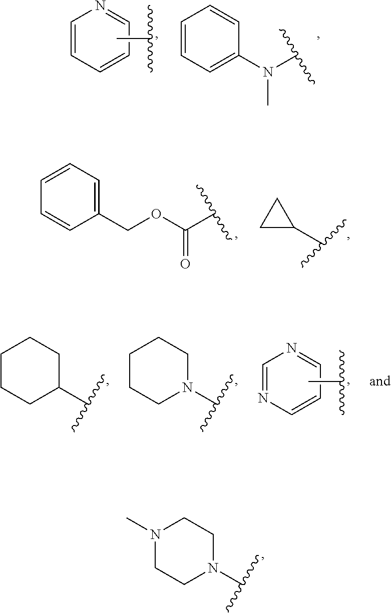
are optionally substituted with 1-2 substituents independently, for each occurrence, selected from the group consisting of halogen, C₁₋₆alkyl, —O—C₁₋₆alkyl, and phenyl.

52. The compound of any one of claims 42-51, wherein R^{d} is selected from the group consisting of hydrogen, methyl, —O—CH₃, phenyl, O-phenyl,

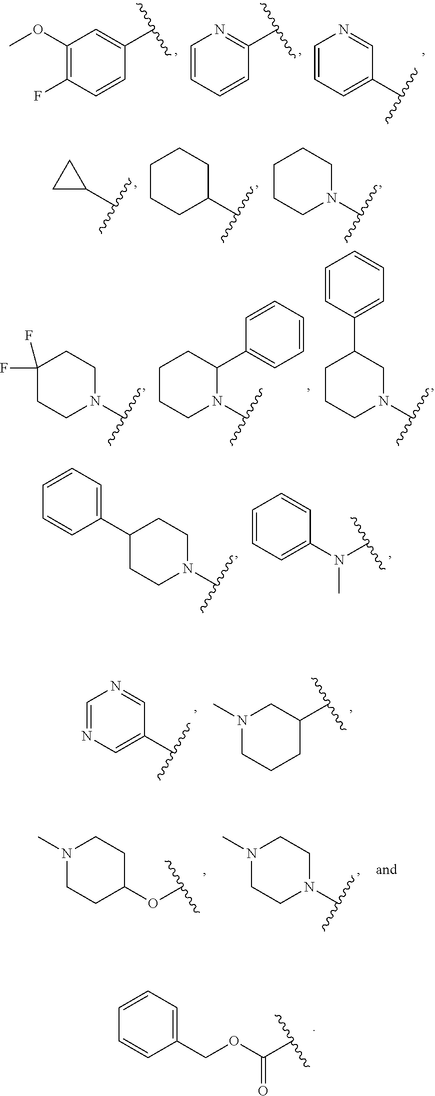

53. The compound of any one of claims 42-48, wherein Z is C.

54. The compound of any one of claims 42-48 and 53, wherein R^{d} is fluorine.

55. The compound of any one of claims 42-48, wherein Z is O.

56. The compound of any one of claims 1-55, wherein R⁶ and R⁷ are selected from the group consisting of hydrogen, methyl, and halogen.

57. The compound of any one of claims 1-56, wherein R⁶ and R⁷ are hydrogen.

58. The compound of any one of claims 12-57, wherein n is 1.

59. The compound of any one of claims 12-57, wherein n is 2.

60. The compound of any one of claims 12-57, wherein n is 3.

61. The compound of any one of claims 12-57, wherein n is 4.

62. The compound of any one of claims 12-57, wherein n is 6.

63. The compound of any one of claims 12-57, wherein n is 0.

64. The compound of any one of claims 12-63, wherein W is selected from the group consisting of methyl, ethenyl, halogen, phenyl, C₃₋₇cycloalkyl, 3-7 membered heterocyclyl, 5-6 membered heteroaryl, —O—C₁₋₆alkyl, —O—(C₁₋₆alkylene)-C₃₋₇cycloalkyl, —O-phenyl, and —O—(C₁₋₆alkylene)-phenyl, wherein methyl, ethenyl, phenyl, C₃₋₇cycloalkyl, 3-7 membered heterocyclyl, 5-6 membered heteroaryl, —O—C₁₋₆alkyl, —O—(C₁₋₆alkylene)-C₃₋₇cycloalkyl, —O-phenyl, and —O—(C₁₋₆alkylene)-phenyl are optionally substituted with halogen or —CF₃.

65. The compound of any one of claims 12-64, wherein W is selected from the group consisting of methyl, ethenyl, fluorine, —CF₃, cyclopropyl, cyclohexyl, phenyl, —O-phenyl, 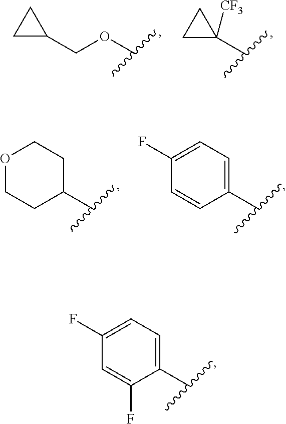 [SMILES: *OCC1CC1 ; *C1(C(F)(F)F)CC1 ; *C1CCOCC1 ; *c1ccc(F)cc1 ; *c1ccc(F)cc1F]
and —OCH₃.

66. The compound of any one of claims 12-65, wherein W is phenyl.

67. The compound of any one of claims 1-66, wherein any aforementioned 3-7 membered monocyclic heterocyclyl, and 5-6 membered heteroaryl are optionally substituted with 1-4 substituents independently, for each occurrence, selected from the group consisting of —CH₂N(R^{a})₂, cyano, C₁₋₆alkyl, halogen, and —O—C₁₋₆alkyl, wherein R^{a} is selected from the group consisting of hydrogen, C₁₋₆alkyl, phenyl, and 3-7 membered heterocyclyl.

68. The compound of any one of claims 1-66, wherein any aforementioned 3-7 membered monocyclic heterocyclyl, and 5-6 membered heteroaryl at R^{a}, R^{b}, R^{d}, R¹, R², R₉, or R¹⁰ are optionally substituted with 1-3 substituents independently, for each occurrence, selected from the group consisting of —CH₂N(R^{a})₂, cyano, C₁₋₆alkyl, halogen, and —O—C₁₋₆alkyl, wherein R^{a} is independently, for each occurrence, selected from the group consisting of hydrogen, C₁₋₆alkyl, phenyl, and 3-7 membered monocyclic heterocyclyl.

69. The compound of any one of claims 1-68, wherein any aforementioned 3-7 membered monocyclic heterocyclyl and 5-6 membered heteroaryl at R^{a}, R^{b}, R^{d}, R¹, R², R⁹, or R¹⁰ are optionally substituted with methyl.

70. A compound of formula (I-Aa) 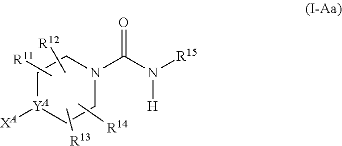
or a pharmaceutically acceptable salt thereof, wherein
Y^{A} is independently selected from CH and N;
X^{A} is independently selected from hydrogen, C₁-C₆alkyl, optionally substituted C₃-C₇cycloalkyl, optionally substituted 3-7 membered monocyclic heterocyclyl, phenyl, C₁-C₆alkyl-(5-6 membered aryl), 5-6 membered heteroaryl, optionally substituted C₁-C₆alkyl-(5-6 membered heteroaryl), and (3-7 membered monocyclic heterocyclylene)-(3-7 membered monocyclic heterocyclyl);
R¹¹, R¹², R¹³ and R¹⁴ are independently selected from hydrogen, cyano, C₁-C₆alkyl, optionally substituted C₃-C₇cycloalkyl, optionally substituted 3-7 membered monocyclic heterocyclyl, optionally substituted 5-6 membered aryl, C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring, wherein R¹¹, R¹², R¹³ and R¹⁴ are not all hydrogen when R¹¹, R¹², R¹³ and R¹⁴ are attached to the carbon atoms linked to the nitrogen of the urea;
or any of R¹¹, R¹², R¹³ and/or R¹⁴ can be taken together with the carbon atoms to which they are attached to form an optionally substituted 3-6 membered spiro carbocyclic or spiro heterocyclic ring,
or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,
or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and
R¹⁵ is independently selected from C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

71. A compound of formula (I-Ab) 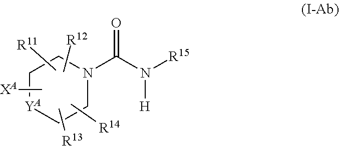
or a pharmaceutically acceptable salt thereof, wherein
Y^{A} is independently selected from CH₂, —C═O, O, and NR^{Ac}, wherein R^{Ac} is independently selected from H, optionally substituted C₁-C₆alkyl, optionally substituted C₃-C₆cycloalkyls, optionally substituted C₃-C₆heterocycloalkyls;
X^{A} is independently selected from hydrogen, C₁-C₆alkyl, optionally substituted C₃-C₇cycloalkyl, optionally substituted 3-7 membered monocyclic heterocyclyl, phenyl, C₁-C₆alkyl-(5-6 membered aryl), optionally substituted 5-6 membered heteroaryl, optionally substituted C₁-C₆alkyl-(5-6 membered heteroaryl), and (3-7 membered monocyclic heterocyclylene)-(3-7 membered monocyclic heterocyclyl);
R¹¹, R¹², R¹³ and R¹⁴ are independently selected from hydrogen, cyano, C₁-C₆alkyl, optionally substituted C₃-C₇cycloalkyl, optionally substituted 3-7 membered monocyclic heterocyclyl, optionally substituted 5-6 membered aryl, C₁-C₆alkyl-(5-6 membered aryl), optionally substituted 5-6 membered heteroaryl, optionally substituted C₁-C₆alkyl-(5-6 membered heteroaryl), halogen, ═CR^{Aa}R^{Ab}, —OR^{Aa}, —NR^{Aa}R^{Ab}, —C(═O)R^{Aa}, —C(═O)—OR^{Aa}, —C(═O)—NR^{Aa}R^{Ab}, —OC(═O)R^{Aa}, —OC(═O)NR^{Aa}R^{Ab}, wherein each of R^{Aa} and R^{Ab} is independently selected from hydrogen, optionally substituted C₁-C₆alkyl, optionally substituted C₃-C₇cycloalkyl, optionally substituted 3-7 membered monocyclic heterocyclyl, optionally substituted 5-6 membered aryl, optionally substituted C₁-C₆alkyl-(5-6 membered aryl), optionally substituted 5-6 membered heteroaryl, optionally substituted C₁-C₆alkyl-(5-6 membered heteroaryl), or R^{Aa} and R^{Ab} can be taken together with the nitrogen atom to which they are bound to form a heterocycloalkyl, wherein R¹¹, R¹², R¹³ and R¹⁴ can be attached to any carbon atom of the ring to which they are connected and may be connected to the same carbon atom or to different carbon atoms of the ring, wherein R¹¹, R¹², R¹³ and R¹⁴ are not all hydrogen when R¹¹, R¹², R¹³ and R¹⁴ are attached to the carbon atoms linked to the nitrogen of the urea;
or any of R¹¹, R¹², R¹³ and/or R⁴ can be taken together with the carbon atoms to which they are attached to form an optionally substituted 3-6 membered spiro carbocyclic or spiro heterocyclic ring,
or any two of R¹¹, R¹², R¹³ and/or R¹⁴ can be taken together with the carbon atoms to which they are attached to form an optionally substituted 5-6 membered cycloalkyl, an optionally substituted 5-6 membered heterocyclyl, an optionally substituted 5-6 membered aryl, or an optionally substituted 5-6 membered heteroaryl,
or any of R¹¹, R¹², R¹³ and/or R¹⁴ can be taken together with the carbon atoms to which they are attached to form an optionally substituted 5- to 7-membered bridged carbo-cyclic or bridged hetero-cyclic ring; and
R¹⁵ is independently selected from C₁-C₆alkyl, optionally substituted C₁-C₆heteroalkyl, optionally substituted C₃-C₇cycloalkyl, optionally substituted 3-7 membered monocyclic heterocyclyl, optionally substituted C₁-C₆alkyl-(5-6 membered aryl), C₁-C₆alkyl-(5-6 membered heteroaryl), optionally substituted C₁-C₆heteroalkyl-(5-6 membered aryl), and optionally substituted C₁-C₆heteroalkyl-(5-6 membered heteroaryl).

72. The compound of any one of claims 1-5, wherein the compound is a compound of formula (I-C) or formula (I-D), wherein R³ is methyl.

73. The compound of any one of claims 1-5 and 72, wherein the compound is a compound of formula (I-C) or formula (I-D), wherein Rt is hydrogen or phenyl.

74. The compound of any one of claims 1-5 and 72-73, wherein the compound is a compound of formula (I-C), wherein n is 4.

75. The compound of any one of claims 1-5 and 72-74, wherein the compound is a compound of formula (I-C), wherein W is phenyl.

76. The compound of claim 1, wherein the compound is a compound of formula (I-E), wherein R¹ is methyl or phenyl optionally substituted with halogen or —OCH₃.

77. The compound of claim 1 or 76, wherein the compound is a compound of formula (I-E), wherein W is methyl or cyclopropyl.

78. The compound of any one of claims 1, 76, and 77, wherein the compound is a compound of formula (I-E), wherein n is 4.

79. The compound of any one of claims 1, 76, and 77, wherein the compound is a compound of formula (I-E), wherein n is 1.

80. The compound of claim 1, wherein the compound is a compound of formula (I-F), wherein R¹ is methyl.

81. The compound of claim 1 or 80, wherein the compound is a compound of formula (I-F), wherein R^{d} is cyclopropyl.

82. The compound of any one of claims 1, 80, and 81, wherein the compound is a compound of formula (I-F), wherein n is 4.

83. The compound of any one of claims 1 and 80-82, wherein the compound is a compound of formula (I-F), wherein W is methyl.

84. The compound of claim 1, wherein the compound is a compound of formula (I-G) or formula (I-H), wherein n is 2.

85. The compound of claim 1 or 84, wherein the compound is a compound of formula (I-G) or formula (I-H), wherein W is phenyl.

86. The compound of claim 70 or 71, wherein R¹⁵ is C₁-C₆alkyl or C₁-C₆alkyl-(5-6 membered aryl).

87. The compound of any one of claims 70, 71, and 86, wherein R¹¹, R¹², R¹³, and R¹⁴ are independently selected from hydrogen, C₁-C₆alkyl, and optionally substituted phenyl.

88. The compound of any one of claims 70, 71, 86, and 87, wherein X^{A} is selected from the group consisting of: C₁-C₆alkyl, C₃-C₇cycloalkyl, and phenyl.

89. A pharmaceutical composition comprising the compound of any one of claims 1-88 and a pharmaceutically acceptable carrier.

90. A method of treating a subject with cancer and in need thereof, the method comprising administering to the subject a therapeutically effective amount of the compound of any one of claims 1-88 or a pharmaceutical composition of claim 89.

91. The method of claim 90, wherein the cancer is glioblastoma.

92. A method of treating a subject with a lysosomal storage disorder and in need thereof, the method comprising administering to the subject a therapeutically effective amount of the compound of any one of claims 1-88 or a pharmaceutical composition of claim 89.

93. The method of claim 92, wherein the lysosomal storage disorder is selected from the group consisting of: Krabbe disease, Fabry disease, Tay-Sachs disease, Pompe disease, Hunter's syndrome, Niemann Pick disease Types A and B, and Gaucher disease.

94. The method of claim 92, wherein the lysosomal storage disorder is Fabry disease.

95. A method of treating a subject with a neurodegenerative disorder and in need thereof, the method comprising administering to the subject a therapeutically effective amount of the compound of any one of claims 1-88 or a pharmaceutical composition of claim 89.

96. The method of claim 95, wherein the neurodegenerative disorder is selected from the group consisting of: Alzheimer's disease, Parkinson's disease, Huntington's disease, amyotrophic lateral sclerosis, Lewy body disease, dementia, and multiple system atrophy.

97. The method of claim 95, wherein the neurodegenerative disorder is Parkinson's disease.

98. The method of claim 95, wherein the neurodegenerative disorder is Lewy body disease.

99. The method of claim 95, wherein the neurodegenerative disorder is dementia.

100. The method of claim 95, wherein the neurodegenerative disorder is multiple system atrophy.

101. A method of treating a subject with an inflammatory disorder and in need thereof, the method comprising administering to the subject a therapeutically effective amount of the compound of any one of claims 1-88 or a pharmaceutical composition of claim 89.

102. The method of any one of claims 90-101, wherein the subject is human.

103. A compound of any one of claims 1-88 or a pharmaceutical composition of claim 89 for use in a method of treating a subject with cancer and in need thereof, the method comprising administering to the subject a therapeutically effective amount of the compound or the pharmaceutical composition.

104. A compound of any one of claims 1-88 or a pharmaceutical composition of claim 89 for use in a method of treating a subject with a lysosomal storage disorder and in need thereof, the method comprising administering to the subject a therapeutically effective amount of the compound or the pharmaceutical composition.

105. A compound of any one of claims 1-88 or a pharmaceutical composition of claim 89 for use in a method of treating a subject with a neurodegenerative disorder and in need thereof, the method comprising administering to the subject a therapeutically effective amount of the compound or the pharmaceutical composition.

106. A compound of any one of claims 1-88 or a pharmaceutical composition of claim 89 for use in a method of treating a subject with an inflammatory disorder and in need thereof, the method comprising administering to the subject a therapeutically effective amount of the compound or the pharmaceutical composition.

107. A compound of any one of claims 1-88 or a pharmaceutical composition of claim 89 for the manufacture of a medicament for treating a subject with cancer and in need thereof, the method comprising administering to the subject a therapeutically effective amount of the compound or the pharmaceutical composition.

108. A compound of any one of claims 1-88 or a pharmaceutical composition of claim 89 for the manufacture of a medicament for treating a subject with a lysosomal storage disorder and in need thereof, the method comprising administering to the subject a therapeutically effective amount of the compound or the pharmaceutical composition.

109. A compound of any one of claims 1-88 or a pharmaceutical composition of claim 89 for the manufacture of a medicament for treating a subject with a neurodegenerative disorder and in need thereof, the method comprising administering to the subject a therapeutically effective amount of the compound or the pharmaceutical composition.

110. A compound of any one of claims 1-88 or a pharmaceutical composition of claim 89 for the manufacture of a medicament for treating a subject with an inflammatory disorder and in need thereof, the method comprising administering to the subject a therapeutically effective amount of the compound or the pharmaceutical composition.
